(19) United States
(12) Patent Application Publication
(10) Pub. No.: US 2016/0045575 A1
(43) Pub. Date: Feb. 18, 2016

(54) FACTOR VIII MUTATION REPAIR AND TOLERANCE INDUCTION AND RELATED cDNAs, COMPOSITIONS, METHODS AND SYSTEMS

(71) Applicant: Tom E. HOWARD, REDONDO BEACH, CA (US)

(72) Inventor: Tom E. HOWARD, REDONDO BEACH, CA (US)

(21) Appl. No.: 14/737,333

(22) Filed: Jun. 11, 2015

Related U.S. Application Data
(63) Continuation-in-part of application No. 14/649,910, filed on Jun. 4, 2015.
(60) Provisional application No. 62/011,019, filed on Jun. 11, 2014.
(60) Provisional application No. 61/734,678, filed on Dec. 7, 2012.
(60) Provisional application No. 61/888,424, filed on Oct. 8, 2013.

(51) Int. Cl. A61K 38/46

(52) U.S. Cl. CPC A61K 38/465; A61K 48/00

(57) ABSTRACT

The present disclosure relates to methods, systems, and compositions to repair one or more mutations in a Factor VIII gene sequence of a subject by introducing into a cell of the subject one or more polynucleotides encoding a DNA scission enzyme (DNA-SE) and one or more repair vehicles (RVs) containing at least a cDNA-repair sequence (RS) such that insertion of the cDNA-RS through homologous recombination with the F8 gene of the subject (sF8) provides a repaired F8 gene (rF8), the repaired F8 gene (rF8) upon expression forming a functional FVIII conferring improved coagulation functionality to the FVIII protein encoded by the sF8. The present disclosure also relates to cells derived using the methods, systems and compositions described.

### CROSS REFERENCE TO RELATED APPLICATIONS

This application claims priority to U.S. Provisional Application 62/011,019, entitled “Factor VIII mutation repair and tolerance induction” and filed on Jun. 11, 2014, and is also a continuation-in-part application of U.S. Non-Provisional application Ser. No. 14/649,910, filed on Jun. 4, 2015, which, in turn, is a U.S. national stage entry of International Patent Application No. PCT/US2013/073751, filed on Dec. 6, 2013, which, in turn, claims priority from U.S. Provisional Application No. 61/734,678, filed on Dec. 7, 2012, and U.S. Provisional Application No. 61/888,424, filed on Oct. 8, 2013. All such applications are incorporated herein by reference in their entirety.

### STATEMENT OF GOVERNMENT GRANT

The U.S. government has certain rights in the inventions pursuant to Grant Nos grant #1R41MD008156-01A1 and 1R41MD008808-01 awarded by the National Institutes of Health (NIH).

### FIELD

The present disclosure relates to gene mutation repairs and related materials, methods and systems, and in particular relates to Factor VIII mutation repair and tolerance induction and related cDNAs compositions, methods and systems.

### BACKGROUND

Factor VIII (FVIII) is a blood-clotting protein, also known as anti-hemophilic factor (AHF), encoded by a Factor VIII gene (F8 gene or F8).

Certain mutations in the F8 gene (F8) result in production of a dysfunctional version of the Factor VIII protein (qualitative deficiency), and/or in production of Factor VIII in insufficient amounts (quantitative deficiency) which cause hemophilia in subjects having the mutations.

Despite developments of various options to manage hemophilia, prophylaxis and treatment of hemophilia in subjects remains challenging.

### SUMMARY

Provided herein are methods and systems and related cDNA, polynucleotides, vehicles and compositions which allow in several embodiments to selectively target and repair one or more mutations in the sequence of Factor VIII gene of a subject, and in particular the one or more mutations of the Factor VIII gene resulting in hemophilia.

According to a first aspect, a method for repairing one or more mutations in a Factor VIII gene (F8 gene) sequence of a subject is described. The method comprises introducing into a cell of the subject one or more polynucleotides encoding a DNA scission enzyme (DNA-SE) such as a nuclease or nickase and one or more repair vehicles (RVs) containing at least a cDNA-repair sequence (RS) comprising a repaired version of the F8 gene sequence of the subject comprising the one or more mutations within a cDNA sequence encoding for a truncated Factor VIII.

The DNA-SE is selected to be capable of targeting a portion of the F8 gene of the subject and to create a first break in one strand of the F8 gene and a second break in the other strand of the F8 gene for subsequent repair by the cDNA-RS. The cDNA-RS is comprised in each of the one or more repair vehicles (RVs) flanked by an upstream flanking sequence (uFS) and a downstream flanking sequence (dFS) to form a DNA donor within the RVs. The upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene.

In the method, introducing into a cell of the subject one or more polynucleotides encoding a DNA scission enzyme (DNA-SE) and one or more repair vehicles (cDNA-RS) is performed to allow insertion of the cDNA-RS through homologous recombination of the upstream flanking sequence (uFS) and the downstream flanking sequence (dFS) with the subject's F8 gene (sF8) to provide a repaired F8 gene (rF8). In the method, the repaired F8 gene (rF8) upon expression forms functional FVIII that confers improved coagulation functionality to the FVIII protein encoded by the sF8 without the repair.

According to a second aspect, a system for repairing one or more mutations in a Factor VIII gene (F8 gene) sequence of a subject is described. The system comprises one or more polynucleotides encoding a DNA scission enzyme (DNA-SE) herein described and one or more repair vehicles (RVs) herein described.

In the system, the DNA scission enzyme (DNA-SE), and the and one or more repair vehicles (RVs) are selected and configured so that upon insertion of the cDNA-RS through homologous recombination of the upstream flanking sequence (uFS) and the downstream flanking sequence (dFS) of the DNA donor sequence with the subject's F8 gene (sF8) a repaired F8 gene (rF8) is provided. In the system, the repaired F8 gene (rF8) upon expression forms functional FVIII that confers improved coagulation functionality to the FVIII protein encoded by the sF8 without the repair.

According to a third aspect, a cDNA is described configured to be used as a cDNA-RS in methods and systems of the disclosure for repairing one or more mutations in a Factor VIII gene (F8 gene) sequence of a subject. The cDNA encodes a truncated Factor VIII polypeptide consisting essentially of the amino acid sequence encoded by each of exons 1, 2, 3, 4, 5, 6, 7, 8, 9, 10, 11, 12, 13, 14, 15, 16, 17, 18, 19, 20, 21, 22, 23, 24, 25, 26 of a F8 gene or an in frame combination thereof. In some embodiments, the each of the exons has a sequence of a corresponding exon in the F8 gene of the subject.

According to a fourth aspect a repair vehicle (RV) is described configured to be used in methods and systems of the disclosure for repairing one or more mutations in a Factor VIII gene (F8 gene) sequence of a subject. The repair vehicle is a polynucleotide configured for use in combination with a DNA scission enzyme (DNA-SE) selected to target a portion of the F8 gene of the subject and to create a first break in one strand of the F8 gene and a second break in the other strand of the F8 gene. The repair vehicle comprises a cDNA-repair sequence (RS) comprising a repaired version of the F8 gene sequence of the subject comprising the one or more mutations within a cDNA sequence encoding for a truncated Factor VIII. In the repair vehicle (RV), the cDNA-RS is flanked by an upstream flanking sequence (uFS) and a downstream flanking sequence (dFS) to form a DNA donor within the RV. The upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene.

According to a fifth aspect a polynucleotide encoding a DNA scission enzyme (DNA-SE) is described configured for use in methods and systems of the disclosure for repairing one or more mutations in a Factor VIII gene (F8 gene) sequence of a subject. The DNA scission enzyme is selected to be capable of targeting a portion of the F8 gene of the subject and to create a first break in one strand of the F8 gene and a second break in the other strand of the F8 gene for subsequent repair by the cDNA-RS.

According to a sixth aspect, a cell is described comprising one or more repair vehicles (RVs) herein described and one or more polynucleotide encoding a DNA scission enzyme (DNA-SE) herein described.

According to a seventh aspect, a composition for repairing one or more mutations in a Factor VIII gene (F8 gene) sequence of a subject is described. The composition comprises one or more polynucleotides encoding a DNA scission enzyme (DNA-SE) herein described and one or more repair vehicles (RVs) herein described together with a suitable excipient. In some embodiments, the composition is a pharmaceutical composition for treatment of hemophilia and/or promotion of immune tolerance to a Factor VIII replacement protein in a subject and the suitable excipient is a pharmaceutically acceptable excipient.

Methods and systems and related cDNA, polynucleotides, vehicles and compositions are expected in several embodiments to provide a repaired F8 gene and corresponding functional Factor VIII in a subject with hemophilia in a form and amount remedying the qualitative and/or quantitative deficiencies of the Factor VIII of the subject, thus allowing treatment of the hemophilia in the subject.

Methods and systems and related cDNA, polynucleotides, vehicles and compositions are expected in several embodiments to provide a repaired F8 and corresponding functional Factor VIII formed by sequences of the subject thus minimizing production of Factor VIII inhibitor in the subject.

Methods and systems and related cDNA, polynucleotides, vehicles and compositions are expected in several embodiments to provide a repaired F8 gene expressing a functional FVIII which allows inducing immune tolerance to a FVIII replacement product ((r)FVIII) in a subject having a FVIII deficiency and who will be administered, is being administered, or has been administered a (r)FVIII product.

The methods and systems and related cDNA, polynucleotides, vehicles and compositions herein described, can be used in connection with applications wherein repair of mutations in Factor VIII gene of a subject is desired, in particular in connection with treatment and/or prophylaxis of various forms of hemophilia and in particular hemophilia A, in subjects. Exemplary applications comprise medical applications, biological analysis, research and diagnostics including but not limited to clinical, therapeutic and pharmaceutical applications, and additional applications identifiable by a skilled person.

The details of one or more embodiments of the disclosure are set forth in the accompanying drawings and the description below. Other features and objects will be apparent from the description and drawings, and from the appended claims.

### BRIEF DESCRIPTION OF THE DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 is a schematic illustration of the wild-type and intron-22-inverted FVIII loci (F8 & F8_{I22I}) and their expressed protein products (FVIII_{FL} & FVIII_{B} for F8 and FVIII_{I22I} & FVIII_{B} for F8_{I22I}).

FIG. 2 is a schematic illustration of a TALEN-mediated genomic editing that can be used to repair the human intron-22 (I22)-inverted F8 locus, F8_{I22I}.

FIG. 3 shows a functional heterodimeric TALEN, comprised of its left and right monomer subunits (TALEN-L and TALEN-R), targeting the human F8 gene.

FIG. 4 shows a functional heterodimeric TALEN, comprised of its left and right monomer subunits (TALEN-L and TALEN-R) targeting the canine F8 gene

FIG. 5 illustrates the TALEN approach linking Exon 22 of the F8 gene to a nucleic acid encoding a truncated FVIII polypeptide encoding exons 23-26.

FIG. 6 illustrates the TALEN approach linking Intron 22 to a F8 3′ splice acceptor site operably linked to a nucleic acid encoding a truncated FVIII polypeptide.

FIG. 7 shows a comparison of expected genomic DNA, spliced RNA and proteins pre and post repair.

FIG. 8 shows PCR primer design to confirm correct integration of exons 23-26 to repair the human intron-22 (I22)-inverted F8 locus, F8_{I22I}.

FIG. 9 illustrates the donor plasmid targeting the F8 Exon22/Intron22 junction using a TALEN, ZFN, CRISPR/Cas, CRISPR-PN, and CRISPR-RFN approach.

FIG. 10 illustrates the donor plasmid targeting the F8 Exon1/Intron1 junction using a TALEN, ZFN, CRISPR/Cas, CRISPR-PN, and CRISPR-RFN approach.

FIG. 11 illustrates the donor plasmid targeting the F8 Intron 22 region using a TALEN, ZFN, CRISPR/Cas, CRISPR-PN, and CRISPR-RFN approach.

FIG. 12 illustrates the donor plasmid targeting the F8 Intron 1 region using a TALEN, ZFN, CRISPR/Cas, CRISPR-PN, and CRISPR-RFN approach.

FIG. 13 illustrates the CRISPR/Cas9-mediated F8 repair strategy targeting intron 1.

FIG. 14 illustrates examples of severe HA-causing F8 mutations that can be cured with the exon-21 targeted CasPN therapeutics of our personalized 3′ gene repair system.

FIG. 15 is a schematic diagram of exon-21 targeted, CasPN mediated personalized repair of the intron-22 inversion mutation (F8I22I).

FIG. 16 is a schematic diagram of the repair vehicle, donor sequence used in the repair of FIG. 15.

FIGS. 17A-B show[[s]] a series of graphs displaying results obtained from flow cytometry using CRISPR/Cas9 plasmids pH0007, pH0009 as well as a repair plasmid (labeled as “Donor”).

FIG. 18 is an image of an agarose gel electrophoresis assay displaying results from a T7E1 assay done on cells transfected with CRISPR/Cas9 plasmids pH0007, pH0009, pH0011 and pH0013.

FIG. 19 is a bar graph showing estimated NHEJ rates for CRISPR constructs pH0007, pH0009, pH0011 and pH0013.

FIG. 20 is an image of an agarose gel electrophoresis assay displaying results from a RFLP assay done on cells transfected with CRISPR/Cas9 plasmids pH0007, pH0009 as well as a repair plasmid (labeled as “Donor”).

FIG. 21 is a bar graph showing the percentage of homologous recombination in cells following Intron 22-targeted CRISPR treatment.

### DETAILED DESCRIPTION

Provided herein are methods and systems and related cDNA, polynucleotides, vehicles and compositions which allow in several embodiments to selectively target and repair one or more mutations in the sequence of Factor VIII gene of a subject.

The term “Factor VIII” or “FVIII” as used herein indicates an essential cofactor in the blood coagulation pathway provided by a large plasma glycoprotein that functions in the blood coagulation cascade as a cofactor for the factor IXa-dependent activation of factor X. Factor VIII is tightly associated in the blood with von Willebrand factor (VWF), which serves as a protective carrier protein for factor VIII. In particular Factor VIII circulates in the bloodstream in an inactive form, bound to von Willebrand factor (VWF). Upon injury, FVIII is activated. The activated protein (FVIIIa) interacts with coagulation factor IX, leading to clotting as will be understood by a skilled person.

FVIII is encoded in a subject by a F8 gene containing 26 exons and spanning 186 kb (Gitschier, et al. Nature 314: 738-740, 1985). In human the F8 gene is located in the X chromosome. In some subjects (e.g. humans, monkeys, rats) the sequences F8 gene also contains an F8A gene and an F8B gene within intron 22. The F8A gene is intron-less, is contained entirely in intron 22 of the F8 gene in reverse orientation to the F8 gene, and is therefore transcribed in the opposite direction to F8. The F8B gene is also located in intron 22 and is transcribed in opposite direction from F8A gene; its first exon lies within intron 22 and is spliced to exons 23-26.

The term “orientation” with reference to a gene indicates the direction of the 5′ →3′ DNA strand which provides the sense strand in the double stranded polynucleotide comprising the gene. Accordingly, 5′->3′ DNA strand is designated, for a given gene, as ‘sense’, ‘plus’ or ‘coding’ strand when its sequence is identical to the sequence of the premessenger (premRNA), except for uracil (U) in RNA, instead of thymine (T) in DNA. An antisense strand is instead the 3′->5′ strand complementary to the sense strand in a double stranded polynucleotide coding for the gene. The antisense transcribed by the RNA polymerase and is also designated as “template” DNA. Accordingly two genes or sequences thereof within the F8 genomic locus encoded by a same polynucleotide are in a same orientation when their respective sense strands are located on a same strand of the polynucleotide and are in in reverse or opposite orientation when respective sense strands are located on different strand of the polynucleotide. Accordingly two genes or coding sequences within the F8 genomic locus encoded by a same polynucleotide are in a same orientation when their respective sense strands are located on a same strand of the polynucleotide. Two genes or coding sequences within the F8 genomic locus are in reverse or opposite orientation when their respective sense strands are located on the opposing strand of the polynucleotide.

FVIII is synthesized primarily in the liver of s subject and the primary translation product of 2332 amino acids undergoes extensive post-translational modification, including N- and 0-linked glycosylation, sulfation, and proteolytic cleavage. The latter event divides the initial multi-domain protein (A1-A2-B-A3-C1-C2) into a heavy chain (A1-A2-B) and a light chain (A3-C1-C2) and the protein is secreted as a two-chain molecule associated through a metal ion bridge (Lenting et al., The life cycle of coagulation FVIII in view of its structure and function. Blood 1998; 92: 3983-96).

Mutations in the F8 gene can result in production of a dysfunctional version of the Factor VIII protein (qualitative deficiency), and/or in production of Factor VIII in insufficient amounts (quantitative deficiency) causing hemophilia in subjects having the mutations.

Accordingly, a Factor VIII is indicated as functional when it is produced in a form and an amount allowing a coagulation functionality comparable with the coagulation functionality of the wild type FVIII protein in a healthy subject. FVIII function is evaluated by routine clinical laboratory methods that are well established in the art and apparent to one of ordinary skill in the art (Barrowcliffe T W, Raut S, Sands D, Hubbard A R: Coagulation and chromogenic assays of factor VIII activity: general aspects, standardization, and recommendations. Semin Thromb Hemost 2002 June; 28(3):247-256).

A non-functional Factor VIII instead indicates an FVIII protein functioning aberrantly or FVIII proteins present in circulating blood in a reduced or absent amount, leading to the reduction of or absence of the ability to clot in response to injury by the subject. FVIII function is evaluated by routine clinical laboratory methods that are well established in the art and apparent to one of ordinary skill in the art (Barrowcliffe T W, Raut S, Sands D, Hubbard A R: Coagulation and chromogenic assays of factor VIII activity: general aspects, standardization, and recommendations. Semin Thromb Hemost 2002 June; 28(3):247-256).

Over 2100 different hemophilia A (HA)-causing mutations have thus far been identified in the F8 loci of unrelated patients which result in the expression of a non-functional and/or deficient FVIII protein. In particular, defects within the F8 affect about one in 5000 newborn males (Jones et al., Identification and removal of promiscuous CD4+ T cell epitope from the C1 domain of factor VIII. J. Throm. Haemost. 2005; 3: 991-1000).

Mutations of the F8 gene resulting in a non-functional Factor VIII include point mutations, deletions, insertion and inversion as will be understood by a skilled person. For example, of the 2100 unique mutations identified in human F8 gene, over 980 of them being missense mutations, i.e., a point mutation wherein a single nucleotide is changed, resulting in a codon that codes for a different amino acid than its wild-type counterpart (see HAMSTeRS Database: at the http:// web page: hadb.org.uk/WebPages/PublicFiles/Mutation Summary.htm). One of the most common mutations resulting in a non-functional and/or deficient FVIII protein includes inversion of intron 22, which leads to a severe type of HA.

Accordingly, a mutation in an F8 gene of a subject resulting in a non-functional Factor VIII results in an F8 gene comprising at least one Factor VIII functional coding sequence and at least one Factor VIII non-functional coding sequence.

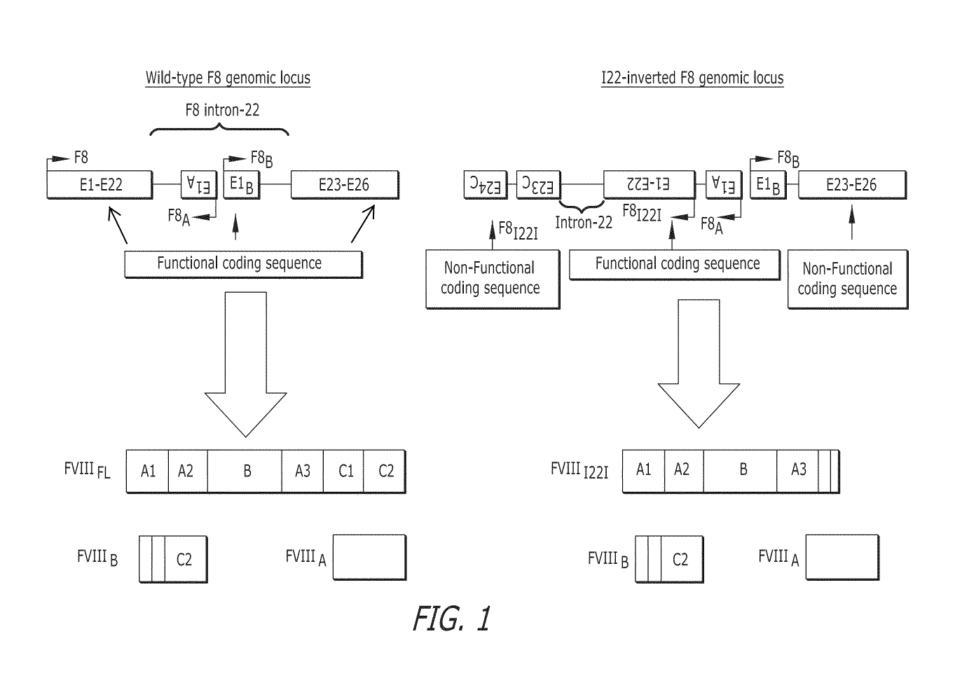

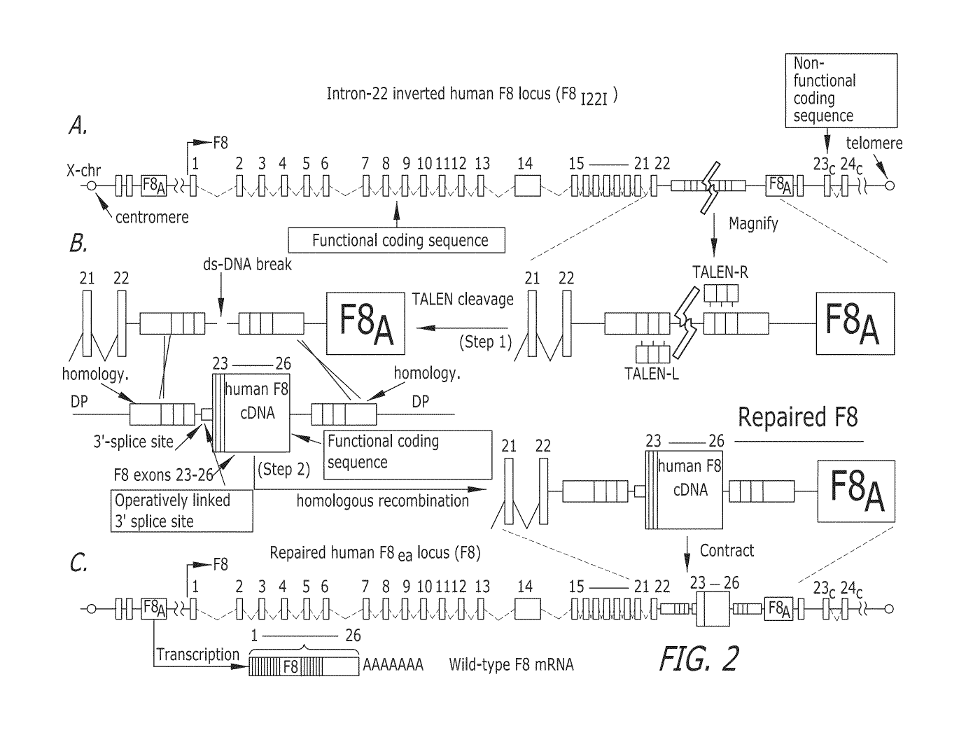

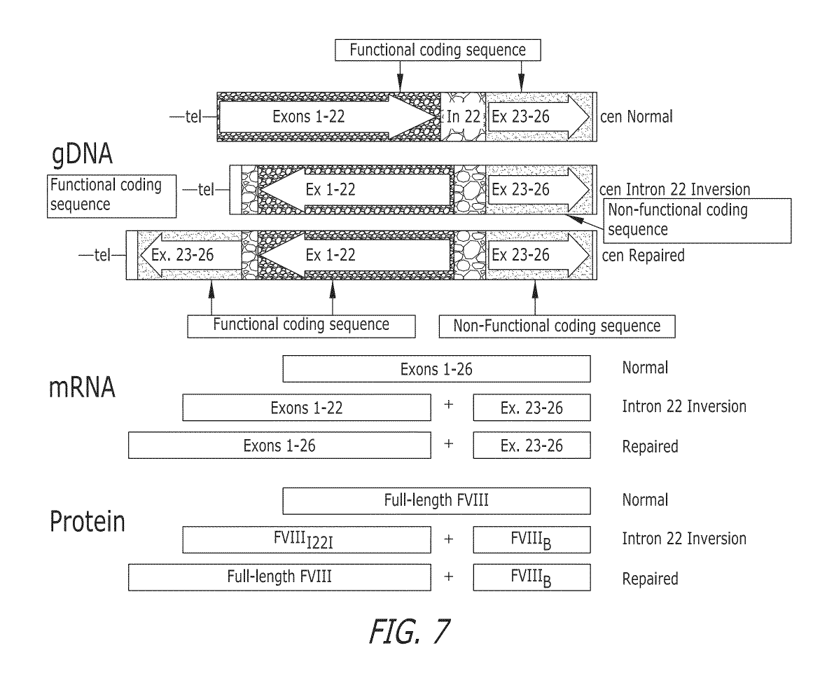

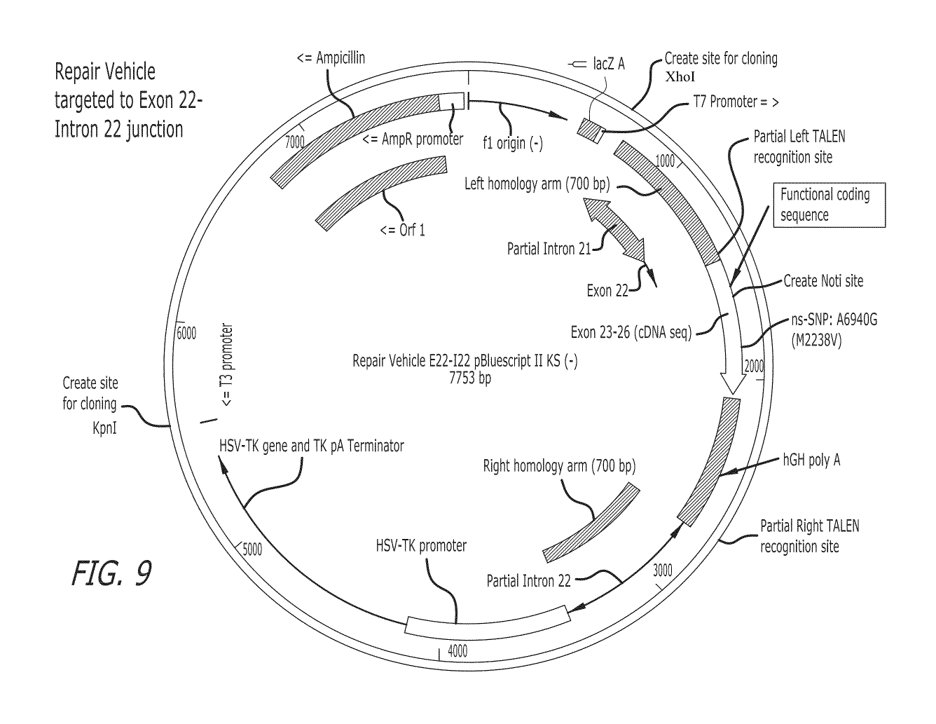

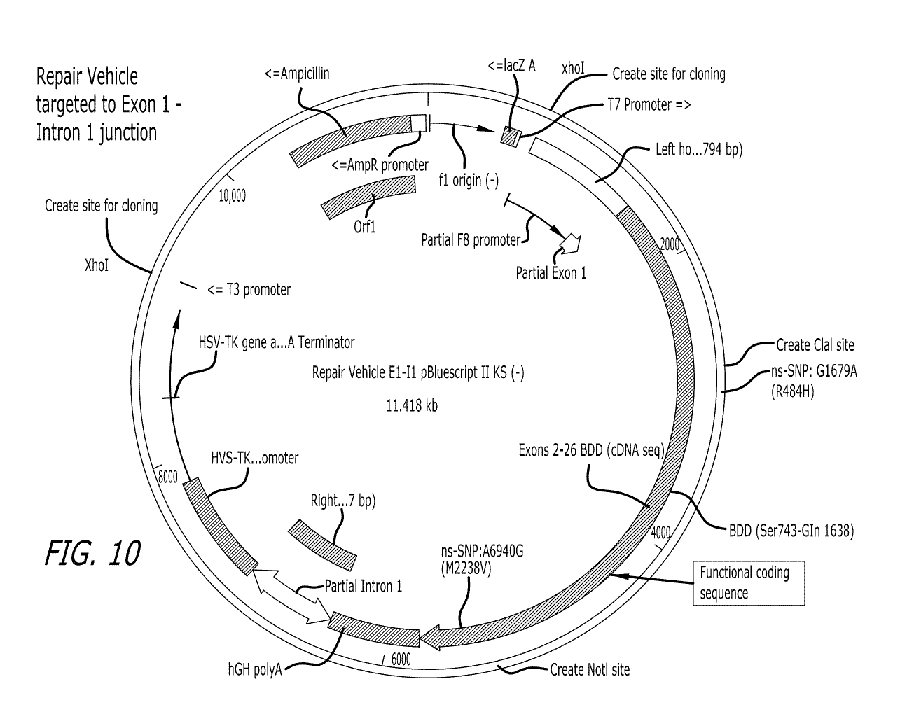

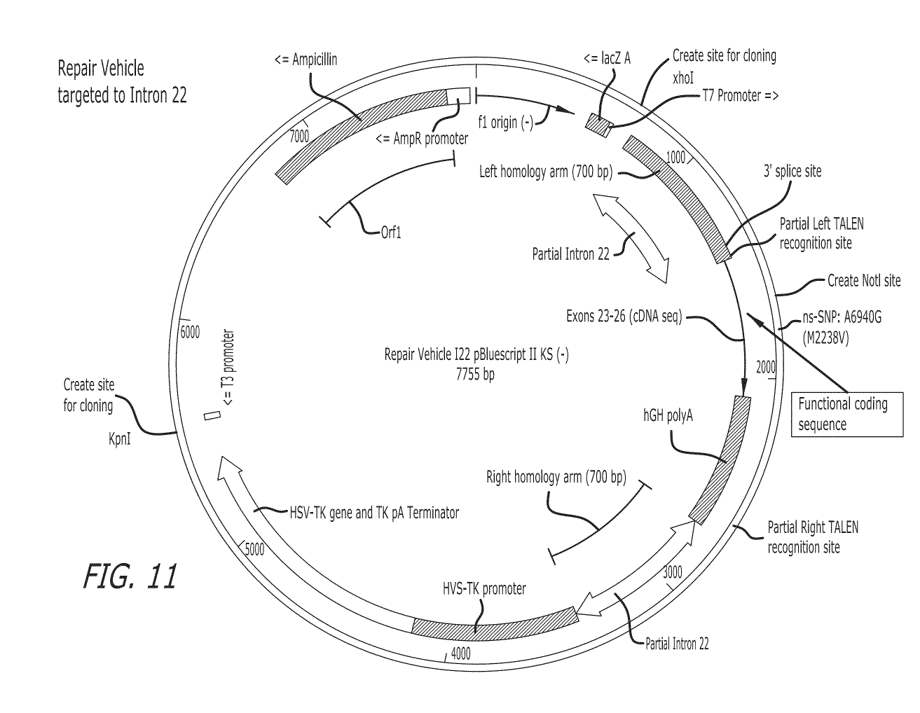

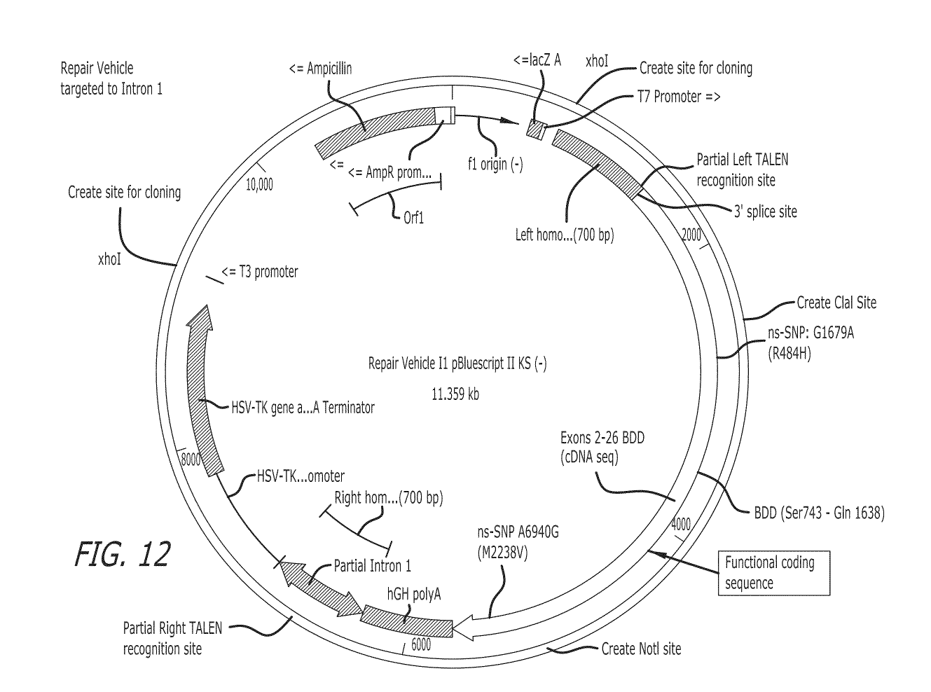

The wording “functional coding sequence” of Factor VIII refers to an F8 gene sequence that is configured to be transcribed and contains one or more exons of the F8 gene with an open reading frame resulting in a functional Factor VIII or in a portion thereof. Exemplary functional coding sequences comprise the sequence of E1-E22 and E23-E26 of the wild type F8 genomic locus in FIG. 1, the sequence of E1-E22 of the Intron-22 inverted F8 locus of FIG. 1, the sequence of human F8 cDNA of FIG. 2, the sequence of Exons 1-22 and Ex 23-26 of the normal F8 gene in FIG. 7, the sequence of Ex 1-22 of the Intron 22 inversion of the F8 gene in FIG. 7, the sequence of Ex 1-22 and Ex 23-26 of the repaired F8 gene of FIG. 7, the cDNA sequence of Exons 23-26 of the repair vehicle of FIG. 9, the cDNA sequence of Exons 2-26 of the repair vehicle of FIG. 10, the cDNA sequence of Exons 23-26 of the repair vehicle of FIG. 11, the cDNA sequence of Exons 2-26 of the repair vehicle of FIG. 12, the cDNA of exons 23-26 of the repair vehicle of Table 51, the cDNA sequence of exons 23-26 of the repair vehicle of Table 52, the cDNA sequence of exons 2-26 or 2-13 of the repair vehicle of Tables 53 and 54, respectively.

Functional coding sequences can include introns or be formed by exons only or a portion thereof. Exemplary functional coding sequences comprise the sequence of E1-E22 and E23-E26 of the wild type F8 genomic locus in FIG. 1, the sequence of E1-E22 of the Intron-22 inverted F8 locus of FIG. 1, Exons 1-22 and respective intervening introns of the Intron-22 inversion human F8 locus of FIG. 2, the sequence of Exons 1-22 and Exons 23-26 of the normal F8 gene in FIG. 7, the sequence of Exons 1-22 of the Intron 22 inversion of the F8 gene in FIG. 7, the sequence of Exons 1-22 and Exons 23-26 of the repaired F8 gene of FIG. 7.

Functional coding sequences can be included in the same orientation as the wild type F8 gene or in an opposite orientation as the wild type F8 gene. Exemplary functional coding sequences in a same orientation as the wild type F8 gene comprise the sequence of E1-E22 and E23-E26 of the wild type F8 genomic locus in FIG. 1, the sequence of Exons 1-22 and Exons 23-26 of the normal F8 gene in FIG. 7, the cDNA sequence of Exons 2-26 of the repair vehicle of FIG. 10, the cDNA sequence of Exons 2-26 of the repair vehicle of FIG. 12, the cDNA of exons 23-26 of the repair vehicle of Table 51, the cDNA sequence of exons 23-26 of the repair vehicle of Table 52, the cDNA sequence of exons 2-26 or 2-13 of the repair vehicle of Tables 53 and 54, respectively. Exemplary functional coding sequences in an opposite orientation as compared to wild type F8 gene comprise the sequence of E1-E22 of the Intron-22 inverted F8 locus of FIG. 1, the sequence of human F8 cDNA of FIG. 2, the sequence of Ex 1-22 of the Intron 22 inversion of the F8 gene in FIG. 7, the sequence of Ex 1-22 and Ex 23-26 of the repaired F8 gene of FIG. 7, the cDNA sequence of Exons 23-26 of the repair vehicle of FIG. 9, the cDNA sequence of Exons 2-26 of the repair vehicle of FIG. 10, the cDNA sequence of Exons 23-26 of the repair vehicle of FIG. 11, the cDNA sequence of Exons 2-26 of the repair vehicle of FIG. 12.

The wording “non-functional coding sequence” of the F8 gene refers to an F8 gene sequence that is not configured to be transcribed and/or contains one or more exons of the F8 gene with an open reading frame resulting in a non-functional Factor VIII or in a portion thereof. In particular, coding sequences can be non-functional, and therefore result in a non-functional Factor VIII, due to point mutations resulting in a sequence coding for an amino acid, in an insertion or deletion of coding sequences resulting in frame shift or a different open reading frame, with respect to an open reading frame (such as the open reading frame of the wild type F8 gene), which results in a functional Factor VIII.

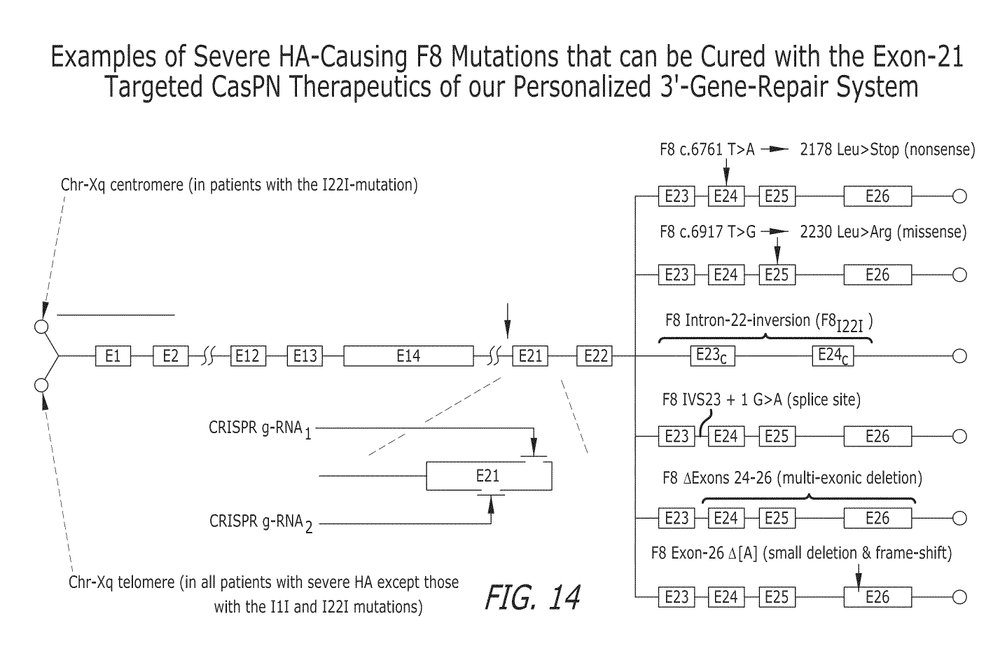

Exemplary non-functional coding sequences resulting from F8 gene mutations comprise the sequence of E24 in the case of a F8 c.6761 T>A nonsense mutation that results in a stop codon at codon 2178 in place of the leucine (Leu)-encoding codon that is present at codon 2178 in the non-mutated form of the F8 gene as seen in FIG. 14, the sequence of E25 in the case of a F8 c.6917 T>G missense mutation that results in a codon encoding arginine (Arg) at codon 2230 in place of the leucine (Leu)-encoding codon that is present at that codon 2230 in the non-mutated form of the F8 gene as seen in FIG. 14, the sequence of sequence of E24, E25 and E26 in the case of a F8 IVS-23+1 G>A splice site mutation that results in a non-functional pre-mRNA splice site immediately downstream of exon 23 of the F8 gene as seen in FIG. 14, sequence of E26 in the case of a F8 Exon 26 del. [A] small deletion and frameshift mutation that results in a frameshift of the gene-encoding sequence which changes the downstream sequence by a single base-pair deletion frameshift and introduction of a novel terminating stop codon in the gene-encoding sequence as seen in FIG. 14.

Non-functional coding sequences can be included in the same orientation as the wild type F8 gene or in an opposite orientation of the wild type F8 gene. Exemplary non-functional coding sequences in a same orientation of the wild type F8 gene comprise the sequence of E1B and the sequence of E23-E26 of the Intron-22 inverted F8 genomic locus of FIG. 1, the sequence of exons 23c and 24c of the Intron-22 inverted human locus of FIG. 2A, the sequence of Exons 23-26 of the Intron 22 Inversion of the F8 gene in FIG. 7, the sequence of E24 in the case of a F8 c.6761 T>A nonsense mutation that results in a stop codon at codon 2178 in place of the leucine (Leu)-encoding codon that is present at codon 2178 in the non-mutated form of the F8 gene as seen in FIG. 14, the sequence of E25 in the case of a F8 c.6917 T>G missense mutation that results in a codon encoding arginine (Arg) at codon 2230 in place of the leucine (Leu)-encoding codon that is present at that codon 2230 in the non-mutated form of the F8 gene as seen in FIG. 14, the sequence of sequence of E24, E25 and E26 in the case of a F8 IVS-23+1 G>A splice site mutation that results in a non-functional pre-mRNA splice site immediately downstream of exon 23 of the F8 gene as seen in FIG. 14, sequence of E26 in the case of a F8 Exon 26 del.[A] small deletion and frameshift mutation that results in a frameshift of the gene-encoding sequence which changes the downstream sequence by a single base-pair deletion frameshift and introduction of a novel terminating stop codon in the gene-encoding sequence as seen in FIG. 14. Exemplary non-functional coding sequences comprise in opposite orientation of the wild type F8 gene comprise the sequence of exons E23C and E24C of the Intron-22 inverted F8 genomic locus of FIG. 1.

In embodiments, herein described non-functional coding sequences are replaced by a cDNA-repair sequence (RS).

The term cDNA or complementary DNA indicates double-stranded DNA that can be synthesized from a messenger RNA (mRNA) template in a reaction catalysed by the enzyme reverse transcriptase. Accordingly cDNA can be synthesized from mature (fully spliced) mRNA using the enzyme reverse transcriptase or be synthesized synthetically based on the mRNA sequence as will be understood by a skilled person.

The terms “polynucleotide”, “oligonucleotide” and “nucleic acid,” are used interchangeably and refer to an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Exemplary functional groups that can be comprised in an analog include methyl groups and hydroxyl groups and additional groups identifiable by a skilled person. In general, an analogue of a particular nucleotide has the same base-pairing specificity; i.e., an analogue of A will base-pair with T.

Exemplary monomers of a polynucleotide comprise deoxyribonucleotide, and ribonucleotides. The term “deoxyribonucleotide” refers to the monomer, or single unit, of DNA, or deoxyribonucleic acid. Each deoxyribonucleotide comprises three parts: a nitrogenous base, a deoxyribose sugar, and one or more phosphate groups. The nitrogenous base is typically bonded to the 1′ carbon of the deoxyribose, which is distinguished from ribose by the presence of a proton on the 2′ carbon rather than an —OH group. The phosphate group is typically bound to the 5′ carbon of the sugar. The term “ribonucleotide” refers to the monomer, or single unit, of RNA, or ribonucleic acid. Ribonucleotides have one, two, or three phosphate groups attached to the ribose sugar.

Accordingly, the term “polynucleotide”, “oligonucleotide includes nucleic acids of any length, and in particular DNA, RNA, analogs thereof, and fragments thereof. Polynucleotides can typically be provided in single-stranded form or double-stranded form (herein also duplex form, or duplex).

A “single-stranded polynucleotide” refers to an individual string of monomers linked together through an alternating sugar phosphate backbone. In particular, the sugar of one nucleotide is bond to the phosphate of the next adjacent nucleotide by a phosphodiester bond. Depending on the sequence of the nucleotides, a single-stranded polynucleotide can have various secondary structures, such as the stem-loop or hairpin structure, through intramolecular self-base-paring. A hairpin loop or stem loop structure occurs when two regions of the same strand, usually complementary in nucleotide sequence when read in opposite directions, base-pairs to form a double helix that ends in an unpaired loop. The resulting lollipop-shaped structure is a key building block of many RNA secondary structures. The term “small hairpin RNA” or “short hairpin RNA” or “shRNA” as used herein indicate a sequence of RNA that makes a tight hairpin turn and can be used to silence gene expression via RNAi.

A “double-stranded polynucleotide”, “duplex polynucleotide” refers to two single-stranded polynucleotides bound to each other through complementarily binding. The duplex typically has a helical structure, such as double-stranded DNA (dsDNA) molecule or double stranded RNA, is maintained largely by non-covalent bonding of base pairs between the strands, and by base stacking interactions.

In embodiments, herein described a cDNA-repair sequence (RS) is a double stranded polynucleotide comprising a repaired version of the entire F8 gene non-functional coding sequence of the subject or of a portion thereof. In particular in methods and compositions herein described the cDNA-RS comprise at least a repaired version the portion of the non-functional sequence of the Factor VIII of the subject comprising the one or more mutations in the Factor VII of the subject. In some embodiments, cDNA-RS described herein further comprises introns and/or exons located upstream and/or downstream to the non-functional coding sequence. In embodiments described herein, the cDNA-RS is designed so that once recombined into the desired region in the F8 genomic locus it remains in-frame with functional coding upstream and downstream functional coding sequences.

Accordingly in methods systems and related cDNA vehicles and compositions herein described a cDNA-RS are designed based on the one or more mutations within the subject's F8 gene targeted for replacement and repair. For example, when repairing a point mutation, the cDNA-RS includes only a small number of replacement nucleotide sequences compared with, for example, a cDNA-RS derived for repairing an inversion such as an intron 22 inversion. Therefore, a cDNA-RS can be of any length, for example between 2 and 10,000 nucleotides in length (or any integer value there between or there above), e.g. between about 100 and 1,000 nucleotides in length (or any integer there between), between about 200 and 500 nucleotides in length (or any integer there between). Exemplary cDNA-RS herein described comprise the sequence of human F8 cDNA of FIG. 2, the cDNA sequence of Exons 23-26 of the repair vehicle of FIG. 9, the cDNA sequence of Exons 2-26 of the repair vehicle of FIG. 10, the cDNA sequence of Exons 23-26 of the repair vehicle of FIG. 11, the cDNA sequence of Exons 2-26 of the repair vehicle of FIG. 12, the cDNA sequence of exons 23-26 of the repair vehicle of Table 51, the cDNA sequence of exons 23-26 of the repair vehicle of Table 52, the cDNA sequence of exons 2-26 or 2-13 of the repair vehicle of Tables 53 and 54, respectively.

In an embodiment, the gene mutation targeted for repair is a point mutation, and the cDNA-RS includes a nucleic acid sequence that replaces the point mutation with a functional sequence for Factor VIII that does not include the point mutation, for example, the wild-type F8 sequence. In one embodiment, the gene mutation targeted for repair is a deletion and the cDNA-RS includes a nucleic acid sequence that replaces the deletion with a functional Factor VIII sequence that does not include the deletion, for example, a corresponding F8 sequence of the wild-type F8 sequence.

In one embodiment, the gene mutation targeted for repair is an inversion, and the cDNA-RS includes a nucleic acid sequence that encodes a truncated FVIII polypeptide that, upon insertion into the F8 genome, repairs the inversion and provides for the production of a functional FVIII protein. In one embodiment, the gene mutation targeted for repair is an inversion of intron 1. In one embodiment, the gene mutation targeted for repair is an inversion of intron 22, and the donor sequence includes a nucleic acid that encodes all of exons 23-25 and the coding sequence of exon-26 to be inserted in frame with the inverted exons 1-22 in opposite orientation with the F8 gene.

In the methods and compositions described herein, the cDNA-RS can contain sequences that are homologous, but not identical (for example, contain nucleic acid sequence encoding wild-type amino acids or differing ns-SNP amino acids), to subject's genomic sequences in the region of interest, thereby stimulating homologous recombination to insert a non-identical sequence in the region of interest.

The term “homologous” and “homology” when referred to protein or polynucleotide sequences is defined in terms of sequence similarities and percent identity between sequences. Accordingly homologous sequences indicate sequences having a percent identify of at least 80% versus sequences with a percentage identify lower than 80%, which are instead indicated as non-homologous. The terms “percent homology” and “sequence similarity” are often used interchangeably. Sequence regions that are homologous are also called conserved.

Thus, in certain embodiments, portions of the cDNA-RS that are homologous to sequences in the region of interest exhibit between about 80 to about 99% sequence identity to the subject's genomic sequence that is replaced. In other embodiments, the homology between the cDNA-RS and the subject's genomic sequence is higher than 99%, for example if only 1 nucleotide differs as between the cDNA-RS and the subject's genomic sequences of over 100 contiguous base pairs. In certain cases, a non-homologous portion of the cDNA-RS contains sequences not present in the region of interest, such that new sequences are introduced into the region of interest. In these instances, the non-homologous sequence is generally flanked by sequences of 50-1,000 base pairs, or any number of base pairs greater than 1,000, that are homologous or identical to the subject's sequences in the region of interest. In other embodiments, the cDNA-RS containing non-homologous sequence is inserted into the subject's genome by homologous recombination mechanisms.

Accordingly, cDNA-RS herein described can be comprised within a cDNA sequence encoding for a truncated Factor VIII. The term “truncated FVIII polypeptide” refers to a polypeptide that contains less than the full length of FVIII protein. The truncated FVIII polypeptide is encoded in a portion of the full length F8 gene such as a partial F8 cDNA replacement sequence (cDNA-RS). For example, for FVIII polypeptide that is truncated from the corresponding 5′ end of the oligonucleotide sequence, a variable amount of the oligonucleotide sequence can be missing from the 5′ end of the gene. In one embodiment, the truncated FVIII polypeptide is encoded by exons 23-26. In one embodiment, the truncated FVIII polypeptide is encoded by exons 2-26. In one embodiment, the truncated FVIII polypeptide is encoded by exons 15-26.

In embodiments herein described the cDNA-RS are designed in combination with the selection of DNA scission Enzyme (DNA-SE) and the related target site.

A DNA scission enzyme indicates an enzyme that catalyzes the hydrolytic cleavage of phosphodiester linkages in the DNA backbone in a specific target site. DNA scission refers to the breaking of the chemical bonds between adjacent nucleotides on a nucleotide strand or sequence. DNA scission enzymes comprise nucleases and nickases. “Nucleases” or “Deoxyribonucleases” are enzymes capable of hydrolyzing phosphodiester bonds that link nucleotides. A wide variety of deoxyribonucleases are known, which differ in their substrate specificities, chemical mechanisms, and biological functions. DNA-SEs described herein break the genomic DNA at a target site on the F8 gene upstream from a region to be replaced by a repair vehicle comprising a cDNA-RS. The target site is preferentially located about 50-100 base pairs upstream of the desired region to be replaced on the F8 genomic locus so as to optimize recombination by the repair vehicle, donor plasmid, or editing cassette comprising the cDNA-RS. In studies, it was seen that when a target site is located about 50-100 base pairs upstream of the desired region to be replaced on the F8 genomic locus, optimal recombination was observed by the repair vehicle, donor plasmid, or editing cassette comprising the cDNA-RS. Following recombination of the repair vehicle, donor plasmid, or editing cassette into the target site, expression of the repaired F8 gene segment results in expression of a repaired and functional FVIII protein. DNA-SEs described herein comprise nucleases or nickases coupled to nucleotide sequences that specifically guide the nuclease or nickase to the target site. DNA-SEs described herein include heterodimeric nucleases that bind to specific regions of the F8 gene, nucleases or nickases guided to specific sites of the F8 gene by short RNA sequences or combinations thereof. Exemplary nucleases include transcription activator-like effector nuclease (TALEN), a zinc finger nuclease (ZFN), a CRISPR (Clustered Regularly Interspaced Short Palindromic Repeats)-associated (Cas) nuclease, Paired CRISPR, or CRISPR with ZFN. “Nickases” are enzyme that causes nicks (breaks in one strand) of double stranded nucleic acid, allowing it to unwind. An exemplary nickase is Cas9n (the D10A mutant nickase version of Cas9).

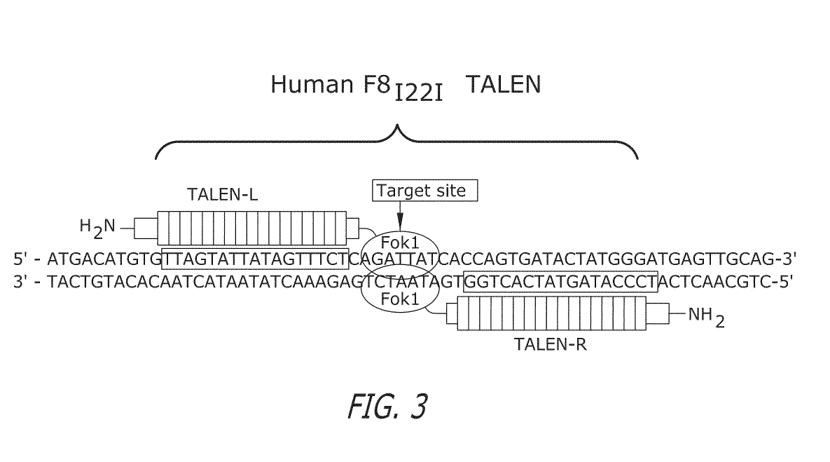

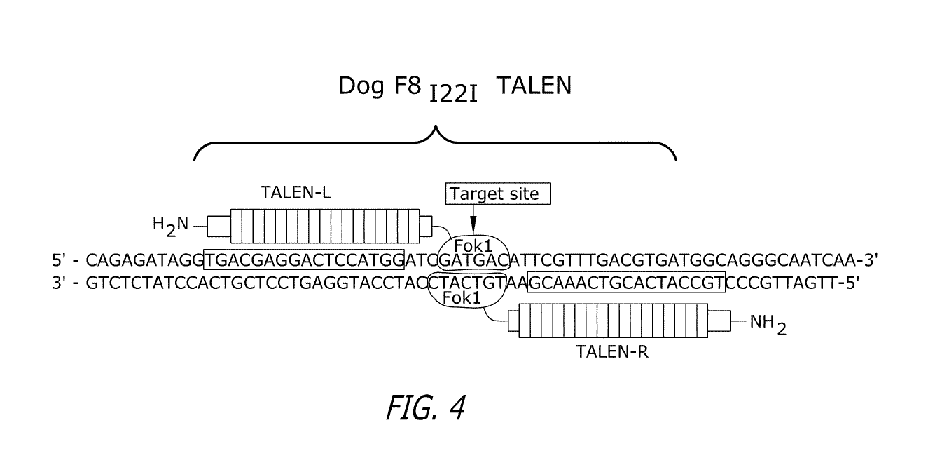

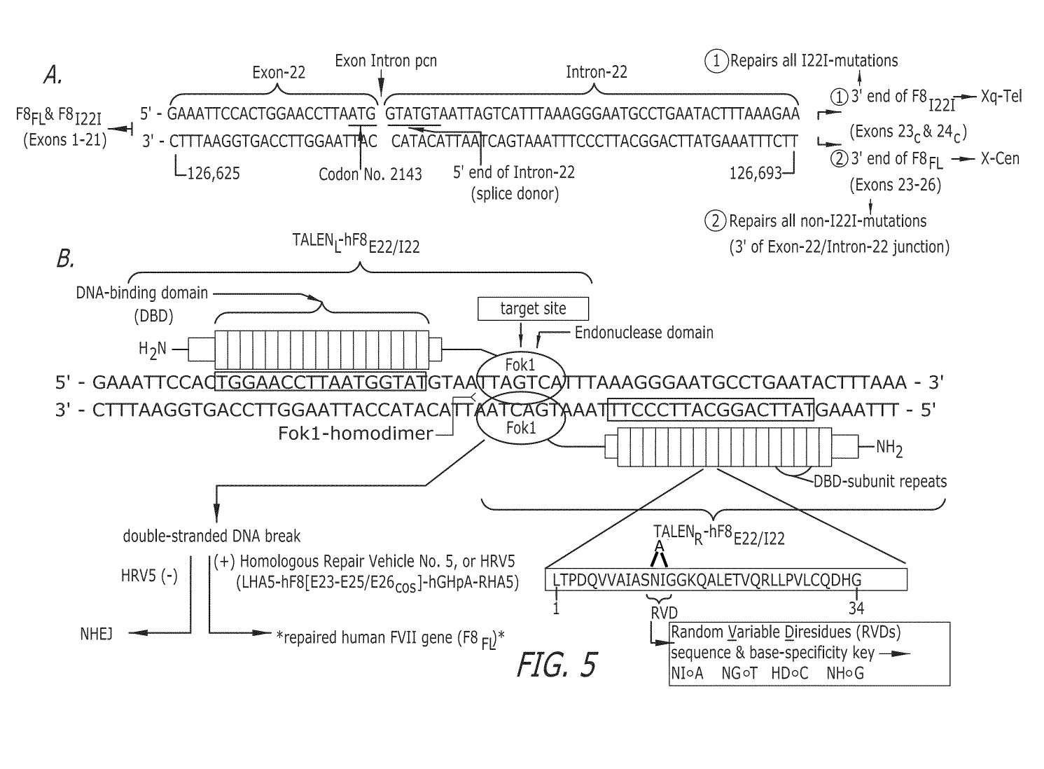

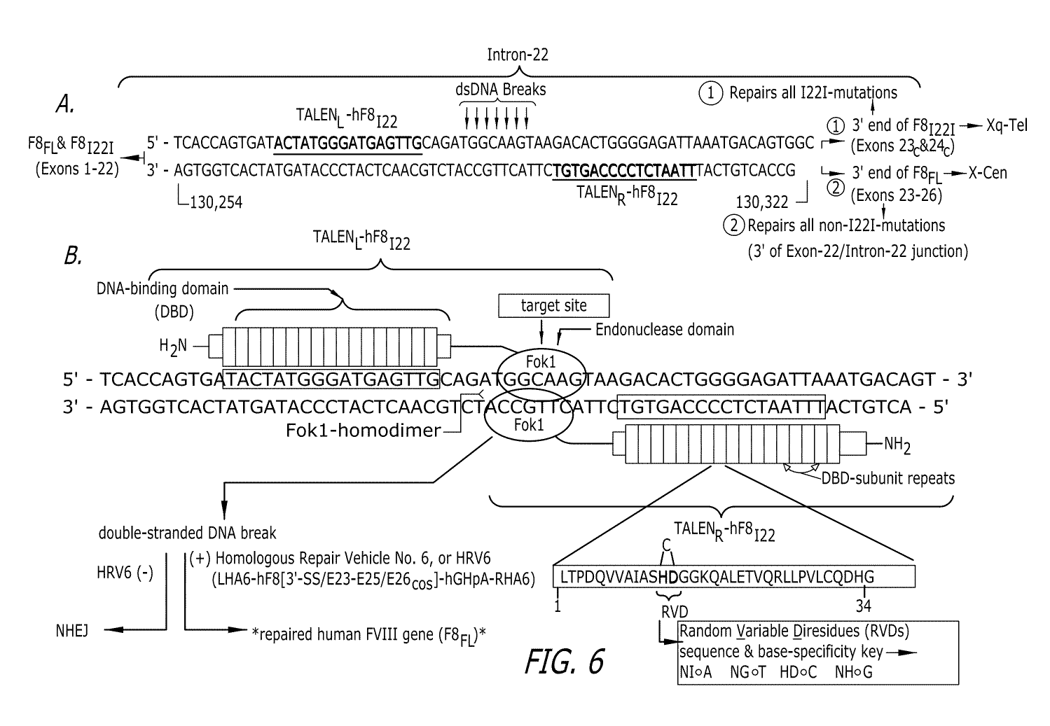

In embodiments described herein, DNA-SEs are designed to comprise multiple elements to efficiently target a specific target site within the F8 gene and function as heterodimers or heterodimeric nucleases; Such DNA-SEs are referenced in FIG. 2, FIG. 3, FIG. 4, FIG. 5 and FIG. 6 as TALEN_{L} and TALEN_{R}. Such heterodimeric nucleases comprise two monomers (a left monomer and a right monomer) that each comprise a nuclear localization signal, a monomer subunit for binding to a specific region of the F8 gene and a Fok1 nuclease domain. Further, the monomer subunit for binding of the left monomer binds upstream (5′) of the target site, while the monomer subunit of the right monomer binds to a region downstream (3′) of the target site, as depicted in FIG. 3 by TALEN_{L} and TALEN_{R}. In such embodiments, a double-stranded break in the DNA of the target region is mediated by dimerization of the Fok-1 nucleases. The monomer binding subunits are designed such that off-target binding non-specific DNA breaks are minimized and such that the location of the target site is optimally placed upstream from a region to be replaced by a repair vehicle comprising a cDNA-RS.

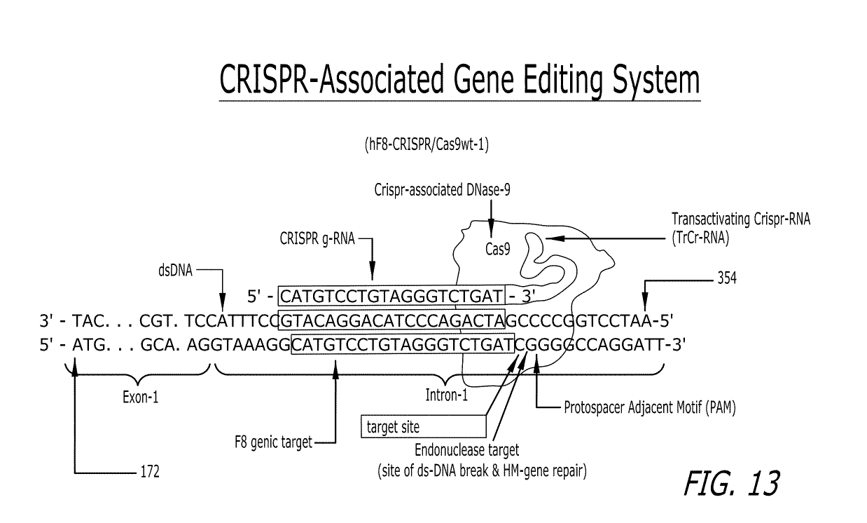

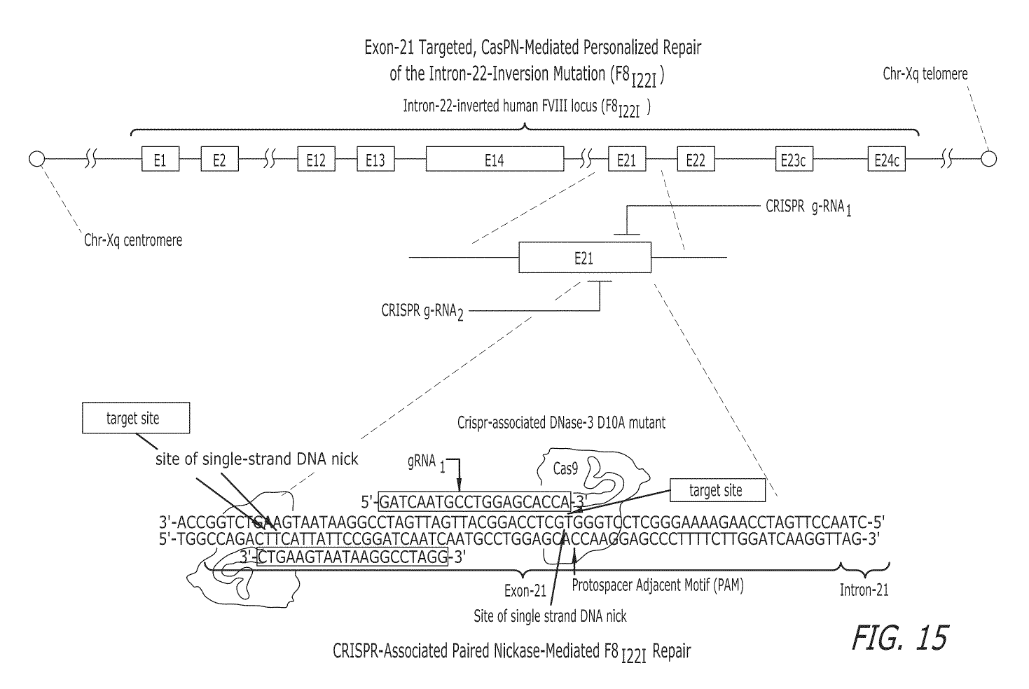

In embodiments described herein, DNA-SEs are designed to efficiently target a specific target site within the F8 gene by using a short RNA to guide a nuclease to the desired target site; such a DNA-SE is referenced in FIG. 13 as the CRISPR-Associated Gene Editing system. Such DNA-SEs comprise at least a complementary single strand RNA (CRISPR RNA, labeled as CRISPR g-RNA in FIG. 13, for example) that localizes a Cas9 nuclease to a target site on F8 gene. The CRISPR RNA binds to a region upstream of a desired target site, allowing the Cas9 nuclease to cause a double-strand break. The CRISPR RNA is designed such that off-target binding non-specific DNA breaks are minimized and such that the location of the target site is optimally placed upstream from a region to be replaced by a repair vehicle comprising a cDNA-RS. In embodiments described herein, such a DNA-SE is modified to further minimize off-target DNA scission events by modifying the CRISPR-Associated Gene editing system DNA-SE described above to carry a mutated Cas9 that functions as a nickase (Cas9-nickase); such a DNA-SE is referenced in FIG. 14 and in FIG. 15. In such embodiments, CRISPR RNA (labeled as CRISPR gRNA₁ in FIG. 15) that is longer in length than the CRISPR RNA of the DNA-SE referenced in FIG. 13 is used to guide a first Cas9-nickase to a target site. The Cas9-nickase then makes a single strand break in the DNA at the target site. A second Cas9-nickase is guided to a second target on the complementary DNA strand site by a second CRISPR RNA (labeled as CRISPR g-RNA₂ in FIG. 15) and the second Cas9-nickase makes a single strand break in the complementary DNA strand. The two nicking target sites can be separated by 0-30 nucleotides.

In the methods and compositions set forth herein, the DNA-SEs that targets a mutation in F8 for repair are, for example, a transcription activator-like effector nuclease (TALEN), a zinc finger nuclease (ZFN), a CRISPR (Clustered Regularly Interspaced Short Palindromic Repeats)-associated (Cas) nuclease, Paired CRISPR, or CRISPR with ZFN, as described in detail below

In the methods and systems and related compositions set forth herein, the DNA-SEs is selected for the DNA-SE ability to target a mutation in the F8 gene for repair cleaving the F8 gene sequence for subsequent repair by the cDNA-RS. In particular in methods and systems and related compositions herein described a DNA-SE is for the capability of creating a first break in one strand of the F8 gene and a second break in the other strand of the F8 gene defining a target site located in a position of the F8 gene configured to allow replacement of the F8 gene non-functional coding sequence by a cDNA-RS.

In methods and systems herein described, the DNA-SE has a target site upstream of the F8 gene nonfunctional coding sequence.

The wording “upstream” as used herein refers to a position in a polynucleotide relative to a 5′ end of the reference point in the polynucleotide. Therefore a sequence or series of nucleotide residues that is “upstream” relative to a site, region or sequence indicates a sequence or series of nucleotides before the 5′ end site, region or sequence of the polynucleotide in a 5′ to 3′ direction. Accordingly, making reference to the exemplary illustration of FIG. 7, Exons 1-22 are located upstream of Exons 23-26 at the normal genomic DNA (gDNA). Additionally, making reference to FIG. 3, TALEN-L binds to a nucleotide sequence upstream of the target site.

The wording “downstream” as used herein refers to a position in a polynucleotide relative to a 3′ end of the reference point in the polynucleotide. Therefore a sequence or series of nucleotide residues that is “downstream” relative to a site, region or sequence indicates a sequence or series of nucleotides after the 3′ end site, region or sequence of the polynucleotide in a 5′ to 3′ direction. Accordingly, making reference to the exemplary illustration of FIG. 7, Exons 23-26 are located downstream of Exons 1-22 at the genomic DNA (gDNA). Additionally, making reference to FIG. 13, the Protospacer Adjacent Motif (PAM) is downstream of the target site.

In methods and systems herein described, the cDNA-RS is designed to provide a repaired version of the F8 gene nonfunctional coding sequence or a portion thereof encompassing the one or more mutations to be repaired in frame with the F8 gene functional coding sequence upstream of the DNA-SE target site.

A sequence or series of nucleotide residues that is “in-frame” or “in frame” with a F8 gene functional sequence refers to a sequence or series of nucleotide residues that does not cause a shift in the open reading frame of the F8 functional sequence. An open reading frame (ORF) is the part of a reading frame of a coding sequence that encodes for a protein or peptide according to the standard genetic code, in this case a functional Factor VIII. An ORF is a continuous stretch of DNA beginning with a start codon, usually methionine (ATG), and ending with a stop codon (TAA, TAG or TGA in most genomes) as will be understood by a skilled person. Accordingly, sequence or series of nucleotide residues is “out of frame” or “out-of-frame” with an F8 functional sequence when to the sequence or series of nucleotide residues causes a shift in the open reading frame of the F8 functional sequence thus resulting in a sequence coding for a non-functional Factor VIII.

For example in some embodiments, the cDNA-RS provides a repaired version of the F8 nonfunctional sequence in a same orientation with the wild type F8 gene. In some embodiments, the cDNA-RS provides a repaired version of the F8 nonfunctional sequence in opposite orientation with the wild type F8 gene in frame with the functional sequence of the F8 gene following the inversion. In particular in some embodiments the cDNA-RS for the inversion of intron 22 provides repaired version of the F8 non-functional sequence downstream the inverted exons 1-22 encompassing sequences for exons 23-26 in opposite orientation to the F8 gene.

In embodiments, herein described selection of a suitable DNA-SE is performed by selecting a target site among candidate target sites on the F8 gene based on the one or more mutations of the F8 gene to be repaired and based on the features of the cDNA-RS to be used on the repair and/or the related donor sequence comprising the cDNA-RS flanked by flanking sequence is homologous to nucleic acid sequences of the F8 gene.

The wording “flanked” as used herein refers to a position relative to ends of a reference item. More specifically, in referring to a polynucleotide sequences, “flanked” refers to having a sequences upstream and downstream the end of the polynucleotide sequences. In particular, a flanked referenced polynucleotide has a first sequence or series of nucleotide residues positioned adjacent to the 5′ end of the referenced polynucleotide and a second sequence or series of nucleotide residues positioned adjacent to the 3′ end of the referenced polynucleotide. For example, in FIG. 2B, the human F8 cDNA is flanked by a left homology arm (homology′) and a right homology arm (homology_{L}).

In some embodiments, selection based on the one or more mutations of the F8 gene to be repaired can be performed with algorithms or other means directed to minimize off-target effects associated with the DNA-SEs. For example, in some embodiments a program such as PROGNOS can be used to identify the target site. The PROGNOS algorithm locates for example potential TALEN off-target sites by searching through the genome for sequences similar to the intended TALEN design. It ranks these similar sequences according to various features of TALEN-DNA interactions, including RVD base preferences, polarity of TALEN specificity (5′ end is more specific), context dependent compensation of strong RVDs (such as NN and HD), and a model of dimeric TALEN interactions. The PROGNOS model has been shown to accurately predict the majority of all known TALEN off-target sites as discussed in Fine et al. Nucleic Acids Research 2013, incorporated herein by reference. As another example, an algorithm employed for ranking potential CRISPR off-target sites disclosed in Hsu et al. Nature Biotech 2013, incorporate herein by reference, uses a position-weight-matrix (PWM) to determine the importance of different types of mismatches at each position in the target sequence (both the DNA bases targeted by the guide strand as well as the protospacer adjacent motif sequence). This PWM was derived by experimentally observing the drop in nuclease activity at a target site of artificial guide strands (relative to a perfectly matched guide strand) containing different types of mismatches. This PWM is then used to screen potential sites in the genome with homology to the intended target and assign them a score indicating their likelihood of off-target activity.

In embodiments herein described a target site is selected based on the features of a cDNA-RS used for repair. Factors influencing the location of the target site include the desired length and sequence of cDNA-RS, proximity of the target site to upstream and downstream functional coding sequences, proximity of the target site to upstream and downstream non-functional coding sequences, likelihood of off-target or non-specific DNA scission, likelihood of off-target or non-specific homologous recombination of the cDNA-RS, homology to off-target genomic sites and nature of the DNA scission enzyme used.

In particular in some embodiments the target site is selected to have a location relative to the desired region of replacement on the F8 genomic locus that optimizes the recombination rate of the cDNA-RS. For instance, in some embodiments, the target site is selected to be from 50-100 nucleotides upstream of the desired region of replacement on the F8 genomic locus so as to optimize the recombination of the cDNA-RS following scission of the genomic DNA. Location of the target site within about 50-100 base pairs upstream of the desired region to be replaced on the F8 genomic locus results in optimal recombination by the repair vehicle, donor plasmid, or editing cassette comprising the cDNA-RS. Optimal recombination is an important aspect as it results in an increase in the likelihood that the cDNA-RS will be incorporated at the targeted site within an individual cell and/or population of cells following exposure to the cDNA-RS. Also, following recombination of the repair vehicle, donor plasmid, or editing cassette into the target site, expression of the repaired F8 gene segment results in expression of a repaired and functional FVIII protein. Thus, conditions promoting optimal recombination greatly contribute towards achieving optimal expression of a repaired and functional protein for treatment and/or induction of immune tolerance.

In embodiments herein described a target site is also be selected based on the features of the donor DNA comprising the cDNA-RS flanked by an upstream flanking sequence (uFS) and a downstream flanking sequence (dFS).

In particular, in embodiments herein described in a donor sequence, the cDNA-RS is flanked on each side by regions of nucleic acids which are homologous to the subject's F8 gene that are called flanking sequences. Each of the flanking sequence can include about 20, 50, 75, 100, 200, 300, 400, 500, 600, 700, 800, 900, 1000 or more nucleotides homologous to regions within the subject's F8 gene. In particular, the upstream flanking sequence (uFS) is homologous to a nucleic acid sequence upstream of the first break in the one strand of the F8 gene by a selected DNA-SE and the downstream flanking sequence (dFS) homologous to a nucleic acid sequences downstream of the second break in the other strand of the F8 gene by the selected DNA-SE.

In some embodiments, each of the homologous regions flanking the donor sequence is between about 200 to about 1,200 nucleotides, e.g. between 400 and about 1000, between about 600 and about 900, or between about 800 and about 900 nucleotides. Thus, each donor sequence includes a cDNA-RS replacing an endogenous mutation in the subject's F8 gene, and 5′ and 3′ flanking sequences which are homologous to the F8 gene. In preferred embodiments the length of the homologous regions flanking the donor sequence are between 700-800 nucleotides in length. Exemplary homologous regions or arms are the left and right homology arms shown in FIG. 9, FIG. 10, FIG. 11 and FIG. 12.

In some embodiments, the cDNA-RS is comprised within an editing cassette together with one or more transcriptional elements and the upstream flanking sequence (uFS) and downstream flanking sequence (dFS) are located adjacent at the 5′ end and at 3′ end of the editing cassette, respectively.

The wording “adjacent” as used herein refers to a location and/or position nearest in space or position; immediately adjoining without intervening space. More specifically, when referring to a sequence or series of nucleotide residues that is “adjacent” to a site or sequence, “adjacent” refers to a location and/or position next to or proximate to the reference site or position without intervening nucleotide residues. An example is seen in FIG. 9 where the left homology arm (700 bp) is located adjacent to Exons 23-26 (cDNA sequence).

In some embodiments, where the cDNA-RS codes for the 3′ terminal sequence of the F8 gene the cDNA-RS is within an editing cassette also comprising a sequence for a polyA site at the 3′ end of the cDNA-RS sequence. In some embodiments where the target site is on a portion of the F8 gene having downstream intron sequences, the 3′ terminal sequence of the F8 gene the cDNA-RS is within an editing cassette also comprising a splice acceptor at the 5′ end of the cDNA-RS sequence. In particular in some embodiment the editing cassette comprise (i) a nucleic acid encoding a truncated FVIII polypeptide or (ii) a native F8 3′ splice acceptor site operably linked to a nucleic acid encoding a truncated FVIII polypeptide that contains a non-mutated portion of the FVIII protein.

As used throughout, “operably linked” is defined as a functional linkage between two or more elements. In particular, the term “operably linked” or “operably connected” indicates an operating interconnection between two elements finalized to the expression and translation of a sequence. Functional linkages between elements in the sense of the present disclosure are identifiable by a skilled person. For example, an operable linkage between a polynucleotide of interest and a regulatory sequence (i.e., a promoter) comprise a functional link that allows for expression of the polynucleotide of interest. Another example of operable linkage is provided by a control sequence ligated to a coding sequence in such a way that expression of the coding sequence is achieved under conditions compatible with the control sequences. Operably linked elements are contiguous or non-contiguous and comprise polynucleotides in a same or different reading frame. In an embodiment, each of the operably linked polynucleotide is comprised within the editing cassette. The cassette additionally contains at least one additional gene to be co-transformed into the organism (e.g. a selectable marker gene). One or more additional genes can also be provided on multiple expression cassettes that can further comprise a plurality of restriction sites and/or recombination sites for insertion of other polynucleotides.

In embodiments herein described, editing cassettes refers to a mobile genetic element that contains a gene and a sequence used to repair an F8 non-functional coding sequence. Editing cassettes carry at least a cDNA-repair sequence (RS) flanked by an upstream flanking sequence (uFS) and a downstream flanking sequence (dFS) to form a DNA donor. The cDNA-RS is a repaired version of the F8 non-functional F8 gene sequence. The upstream flanking sequence (uFS) is homologous to a nucleic acid sequence upstream of a target site on the F8 gene and the downstream flanking sequence (dFS) is homologous to a nucleic acid sequences downstream of a target site on the F8 gene. In embodiments described herein, the cDNA-RS of the editing cassette is designed and oriented such that when recombined into the desired region on the F8 gene, it is in-frame with upstream and downstream functional coding sequences. Exemplary editing cassettes include the sequence comprising the left homology arm, cDNA of Exons 23-26, the human growth hormone polyadenylation signal sequence and the right homology arm of the plasmid in FIG. 9, the sequence comprising the left homology arm, cDNA of Exons 2-26, the human growth hormone polyadenylation signal sequence and the right homology arm of the plasmid in FIG. 10, the sequence comprising the left homology arm, cDNA of Exons 23-26, the human growth hormone polyadenylation signal sequence and the right homology arm of the plasmid in FIG. 11, the sequence comprising the left homology arm, cDNA of Exons 2-26, the human growth hormone polyadenylation signal sequence and the right homology arm of the plasmid in FIG. 12.

In embodiments herein described, following identification of a target site a DNA-SE is configured for binding to the F8 gene at the selected target site. The DNA-SE is modified to target a target site that is preferentially located about 50-100 base pairs upstream of the desired region to be replaced on the F8 genomic locus so as to optimize recombination by the repair vehicle, donor plasmid, editing cassette comprising the cDNA-RS. Location of the target site within about 50-100 base pairs upstream of the desired region to be replaced on the F8 genomic locus results in optimal recombination by the repair vehicle, donor plasmid, or editing cassette comprising the cDNA-RS. Optimal recombination is an important aspect as it results in an increase in the likelihood that the cDNA-RS will be incorporated at the targeted site within an individual cell and/or population of cells following exposure to the cDNA-RS. Also, following recombination of the repair vehicle, donor plasmid, or editing cassette into the target site, expression of the repaired F8 gene segment results in expression of a repaired and functional FVIII protein. Thus, conditions promoting optimal recombination greatly contribute towards achieving optimal expression of a repaired and functional protein for treatment and/or induction of immune tolerance. DNA-SEs described herein are modified to comprise nucleases or nickases coupled to nucleotide sequences that specifically guide the nuclease or nickase to the target site. DNA-SEs described herein include heterodimeric nucleases that bind to specific regions of the F8 gene, nucleases or nickases guided to specific sites of the F8 gene by short RNA sequences or combinations thereof. A DNA-SE can be designed and assembled using molecular techniques commonly known and available to one of ordinary skill in the art and as described in Ran, F. A. et al. Genome engineering using the CRISPR-Cas9 system. Nat Protoc 8, 2281-2308 (2013).

In embodiments described herein, polynucleotides and vectors comprising the DNA-SE and the DNA donor are provided for introduction into a cell of a subject having a mutated F8 gene. In particular the DNA-SE comprises nucleases or nickases coupled to nucleotide sequences that specifically guide the nuclease or nickase to the target site. DNA-SEs described herein include heterodimeric nucleases that bind to specific regions of the F8 gene, nucleases or nickases guided to specific sites of the F8 gene by short RNA sequences or combinations thereof. The polynucleotides and vectors comprising the DNA-SE and DNA donor vary in design and function as a function of the type of gene editing system that is utilized. For instance, different polynucleotides and vectors are used for TALENs, CRISPR/Cas9 nuclease, CRISPR/Cas9n nickase, and CRISPR/Cas9 RFN.

In embodiments herein described, a “donor plasmid” refers to a mobile genetic element in the form of a plasmid, vector, sequence or strand that is be used as a means to deliver or donate a polynucleotide sequence to a specific genomic site. The donor plasmid contains DNA and/or cDNA. Embodiments of donor plasmids described herein consist of at least the following elements: a cDNA-RS for repair of a non-functional F8 coding sequence flanked by an upstream flanking sequence (uFS) and a downstream flanking sequence (dFS). The upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene. Donor plasmids are designed and configured to optimally integrate by homologous recombination at a target site following DNA scission by a DNA-SE. The cDNA-RS of donor plasmid designed and oriented such that when recombined into the desired region on the F8 gene, it is in-frame with upstream and downstream functional coding sequences. Exemplary donor plasmids include the plasmids referenced in FIG. 9, FIG. 10, FIG. 11 and FIG. 12.

In embodiments herein described the DNA donor is comprised within a repair vehicle (RV). The RV can be a sequence of DNA in the form of a circular plasmid. The RV can be a linear sequence of DNA. The RV provides the template, through which by homologous recombination, a targeted DNA sequence can be introduced into the genomic DNA of the subject at the site of a targeted double strand break. In addition to a cDNA-RS, optionally an editing cassette and flanking sequences of the DNA donor, a RV can also contain sequences important for the preparation of the DNA sequence in bacteria, such as an antibiotic resistance gene for ampicillin, an antibiotic resistance gene for kanamycin, and/or other antibiotic resistance genes. The RV can also contain intervening DNA sequences important for the integrity of the plasmid or linear sequence of DNA, such as sequences that are located between antibiotic-resistance gene-encoding sequences and cDNA-RS, and which intervening DNA sequences can contain gene-encoding sequences or alternatively can contain sequences that do not encode for a gene.

In methods and systems herein described polynucleotides coding for a DNA-SE and one or more repair vehicles are introduced into a cell of a subject having a mutated F8 for a time and under condition allowing homologous recombination of the upstream flanking sequence (uFS) and the downstream flanking sequence (dFS) of the donor DNA to corresponding sequences of the F8 gene.

In particular, in some embodiments herein described, the targeting and repair of a mutated F8 gene in a subject, by introducing into a subject's cell one or more plasmids encoding a DNA-SE that specifically targets the F8 mutation of the subject. Each subject's mutation for targeting and repair can be determined using techniques known in the art. The identified mutation in the subject is then directly targeted by DNA-SE for correction according e.g. by selecting a DNA-SE target site at the 5′ of the mutated non-functional F8 gene sequence. Alternatively, the subject's F8 gene mutations can be corrected by targeting a region of the F8 gene upstream (or 5′) from the non-functional coding sequence (e.g. where the mutation occurred), and adding back the corresponding downstream coding regions of the F8 gene. For example, intron 14 could be targeted by the DNA-SE. This allows for gene repair of downstream mutations (i.e. missense mutations in exon 15 to exon 26) and inversions (such as the intron 22 inversion), due to the replacement of exons 15 to 26 with the cDNA-RS discussed above. In other embodiments, the F8 gene can be targeted at additional regions upstream, in order to capture an increasing proportion of F8 gene mutations. Thus, the DNA-SE can be engineered to specifically target a subject's F8 mutation, or alternatively, can target regions upstream of a subject's F8 mutation, in order to correct the mutation in combination with a donor sequence which provides cDNA-RS, which is a partial F8 gene during homologous recombination that replaces, and thus repairs, the mutated portion of the subject's F8 gene and possibly includes functional coding sequences upstream of the non-functional coding sequence of the mutated F8 gene.

In particular in some embodiments of methods and systems herein described the repairing is performed introducing into a cell of the subject one or more nucleic acids encoding a DNA scission enzyme (DNA-SE) having a DNA-SE target site located upstream from a 5′ end of at least one Factor VIII non-functional coding sequence to be repaired, the DNA-SE target site located about 50 bp to about 100 bp upstream from a 5′ end of the Factor VIII non-functional coding sequence to be repaired; and introducing into the cell of the subject a cDNA repair editing cassette comprising a cDNA repair sequence (cDNA-RS) coding for a repaired version of the Factor VIII non-functional coding sequence, the cDNA repair sequence in frame with the Factor VIII functional coding sequence. In those embodiments, location of the target site within about 50-100 base pairs upstream of the desired region to be replaced on the F8 genomic locus results in optimal recombination by the repair vehicle, donor plasmid, or editing cassette comprising the cDNA-RS. Optimal recombination is an important aspect as it results in an increase in the likelihood that the cDNA-RS will be incorporated at the targeted site within an individual cell and/or population of cells following exposure to the cDNA-RS. Also, following recombination of the repair vehicle, donor plasmid, or editing cassette into the target site, expression of the repaired F8 gene segment results in expression of a repaired and functional FVIII protein. Thus, conditions promoting optimal recombination greatly contribute towards achieving optimal expression of a repaired and functional protein for treatment and/or induction of immune tolerance.

Also in those embodiments the cDNA repair editing cassette within a DNA donor where the cDNA repair editing cassette is flanked by an upstream flanking sequence (uFS) homologous to a genomic nucleic acid sequence of at least 200 bp from the DNA-SE target site and a downstream flanking sequence (dFS) homologous to a genomic nucleic acid sequences of at least 200 bp downstream of the DNA-SE target site. In those embodiments introducing one more nucleic acids encoding a DNA scission enzyme (DNA-SE) and introducing a cDNA repair editing cassette is performed to allow homologous recombination of the upstream flanking sequence (uFS) and the downstream flanking sequence (dFS) with corresponding genomic sequences of the Factor VIII gene of the subject.

In some embodiments, the DNA-SE target site is adjacent to a 3′ end of the Factor VIII functional coding sequence, and in particular the 3′ end of the functional coding sequence can be a 3′ end of a Factor VIII exon.

In some embodiments, the upstream flanking sequence (uFS) is homologous to a genomic nucleic acid sequence of at least about 400 bp from the DNA-SE target site and the downstream flanking sequence (dFS) is homologous to a genomic nucleic acid sequences of at least about 400 bp downstream of the DNA-SE target site.

In some embodiments, the upstream flanking sequence (uFS) is homologous to a genomic nucleic acid sequence of at least about 400-800 bp from the DNA-SE target site and the downstream flanking sequence (dFS) is homologous to a genomic nucleic acid sequences of at least about 400-800 bp downstream of the DNA-SE target site.

In some embodiments, the uFS is homologous to a genomic nucleic acid sequence of at least about 800-3000 bp from the DNA-SE target site and the dFS is homologous to a genomic nucleic acid sequences of at least about 800-3000 bp downstream of the DNA-SE target site.

In some embodiments, the cDNA repair sequence (cDNA-RS) encodes for one or more repaired Factor VIII non-functional sequence consisting essentially of the amino acid sequence encoded by exons 1, 2, 3, 4, 5, 6, 7, 8, 9, 10, 11, 12, 13, 14, 15, 26, 17, 18, 19, 20, 21, 22, 23, 24, 25, 26, or an in frame portion or combination thereof.

In some embodiments, the methods and compositions set forth herein, the DNA-SEs that targets a mutation in F8 for repair are, for example, a transcription activator-like effector nuclease (TALEN), a zinc finger nuclease (ZFN), a CRISPR (Clustered Regularly Interspaced Short Palindromic Repeats)-associated (Cas) nuclease (CasN), a pair of wild-type CasN each containing its own CRISPR-single-guide-RNA (CRISPR-sgRNA) targeting a deep intronic sequence of a F8 intron flanking the two sides of a large F8 exonic duplication (to repair a HA-causing F8 mutation comprised of a large duplication of one or more F8 exons by introducing a double-stranded DNA (dsDNA) break on each side of large exonic duplication such that intervening genomic DNA sequence comprising the duplication can be deleted, thereby restoring the transcriptional and post-transcriptional functionality to the repair F8 sequence), a pair of missense mutant Cas nickases—each capable of introducing only a single-stranded DNA (ssDNA) break—using paired CRISPR guide RNAs, or CRISPR with RFN, as described in detail below.

To minimize off-target effects associated with the DNA-SEs, a program such as PROGNOS is used. The PROGNOS algorithm locates for example potential TALEN off-target sites by searching through the genome for sequences similar to the intended TALEN design. It ranks these similar sequences according to various features of TALEN-DNA interactions, including RVD base preferences, polarity of TALEN specificity (5′ end is more specific), context dependent compensation of strong RVDs (such as NN and HD), and a model of dimeric TALEN interactions. The PROGNOS model has been shown to accurately predict the majority of all known TALEN off-target sites as discussed in Fine et al. Nucleic Acids Research 2013, incorporated herein by reference in their entirety.

The algorithm employed for ranking potential CRISPR off-target sites described in Hsu et al. Nature Biotech 2013, incorporate herein by reference, uses a position-weight-matrix (PWM) to determine the importance of different types of mismatches at each position in the target sequence (both the DNA bases targeted by the guide strand as well as the protospacer adjacent motif sequence). This PWM was derived by experimentally observing the drop in nuclease activity at a target site of artificial guide strands (relative to a perfectly matched guide strand) containing different types of mismatches. This PWM is then used to screen potential sites in the genome with homology to the intended target and assign them a score indicating their likelihood of off-target activity.

In some embodiments the DNA-SE is Transcription Activator-Like Effector Nucleases (TALENs) which provides an alternative to zinc finger nucleases (ZFNs) for certain types of genome editing. The C-terminus of the TALEN component carries nuclear localization signals (NLSs), allowing import of the protein to the nucleus. Downstream of the NLSs, an acidic activation domain (AD) is also present, which is probably involved in the recruitment of the host transcriptional machinery. The central region harbors a series of nearly identical 34/35 amino acids modules repeated in tandem. Residues in positions 12 and 13 are highly variable and are referred to as repeat-variable di-residues (RVDs). Studies of TALENs such as AvrBs3 from X. axonopodis pv. vesicatoria and the genomic regions (e.g., promoters) they bind, led two teams to “crack the TALE code” by recognizing that each RVD in a repeat of a particular TALE determines the interaction with a single nucleotide. Most of the variation between TALEs relies on the number (ranging from 5.5 to 33.5) and/or the order of the quasi-identical repeats. Estimates using design criteria derived from the features of naturally occurring TALEs suggest that, on average, a suitable TALEN target site can be found every 35 base pairs in genomic DNA. Compared with ZFNs, the cloning process of TALENs is easier, the specificity of recognized target sequences is higher, and off-target effects are lower. In one study, TALENs designed to target chemokine receptor 5 (CCR5) were shown to have very little activity at the highly homologous chemokine receptor 2 (CCR2) locus, as compared with CCR5-specific ZFNs that had similar activity at the two sites.

FIG. 2 and FIG. 3 provide exemplary illustrations outlining the use of a repair vehicle encoding a TALEN nuclease that is used to repair the F8 gene in, for example, a human with an intron-22 (I22)-inverted F8 locus, F8I22I. As illustrated in FIG. 2(A), the major transcription unit of the F8I22I locus consists of 24 exons, which are designated exons 1-22 (a functional coding sequence) and exons 23C & 24C (a non-functional coding sequence). The first 22 are the same as exons 1-22 of the wild-type FVIII structural locus (F8) but the last two (exon-23C & exon-24C) are cryptic and non-functional in non-hemophilic individuals as well as in patients whose HA is caused by F8 gene abnormalities other than the I22I-mutation. As illustrated in FIG. 2(B) the strategy to repair the I22I-mutation consists of introducing in the cell of the subject a repair vehicle encoding a functional TALEN—which is a heterodimeric nuclease comprised of a monomer subunit that binds 5′ of the desired genome editing site (TALEN-L) and one that binds 3′ of it (TALEN-R)—that is specific for a DNA sequence that is present in only a single copy per haploid human genome, which is approximately 1 kb downstream of the 3′-end of exon-22. Upon expression, once both monomers are bound to this specific sequence, their individual Fok1 nuclease domains dimerize to form the active enzyme that catalyzes a double-stranded (ds) break in the DNA between their binding sites. If a ds-DNA break occurs in the presence of a second nucleic acid, for example a cDNA-RS (a functional coding sequence) comprising a native FVIII 3′ splice acceptor site operably linked to a nucleic acid encoding a truncated FVIII polypeptide encoding exons 23-26 (i.e., a “donor plasmid (DP)” or donor sequence), which is flanked by a stretch of DNA with a left homology (HL) arm and right homology (HL) arm that have identical DNA sequences to that in the native chromosomal DNA 5′ and 3′ of the region flanking the break-point, homologous recombination (HR) occurs very efficiently. Following HR, the cDNA-RS segment between the left and right homology arms (which as shown in FIG. 2 contains a partial human F8 cDNA that contains, in-frame, all of exons 23-25 and the coding sequence of exon-26, with a functional 3′-splice site at its 5′-end) becomes permanently ligated/inserted into the chromosome. Since the cDNA-RS fused at its 5′-end to a functional 3′-splice site, this TALEN catalyzes repair and converts F8I22I into wild-type F8-like locus and restore its ability to drive synthesis of a full-length fully functional wild-type FVIII protein. FIG. 3 shows the details of a functional heterodimeric TALEN, comprised of left and right monomer subunits (TALEN-L and TALEN-R), bound to its target “editing” sequence in intron-22 (I22) of the human FVIII structural locus (F8), ˜1 kb downstream of the 3′-end of exon-22 (FIG. 3).

Likewise, FIG. 4 shows a functional heterodimeric TALEN targeting a F8 mutation in canine, comprised of its left and right monomer subunits (TALEN-L and TALEN-R), bound to its target “editing” sequence in the I22 of the canine F8 structural locus (cF8), ˜0.25 kb downstream of the 3′-end of exon-22. Because the target binding sequence of each monomer is the same in both a wild-type canine F8 (cF8) and an I22-inverted F8 gene (cF8-I22I), this TALEN edits each locus equally well. Following binding of this TALEN's monomeric subunits to their target I22-sequences in the cF8-I22I locus of a dog with severe HA caused by the I22I-mutation, their individual Fok1 nuclease domains are able to form a homo-dimer, i.e. the active form of the enzyme, which catalyzes a double-stranded (ds) break in the DNA between the monomer binding sites; this site is labeled as the target site. If a ds-DNA break occurs in the presence of a donor sequence or plasmid, which contains a stretch of DNA with left and right arms that have identical DNA sequences to that in the native chromosomal DNA, in the region flanking the break-point (see FIG. 3 for the human F8 locus), homHR occurs very efficiently. Following HR, the DNA segment between the left and right homology arms (which contains a partial cF8 cDNA that contains, in-frame, all of exons 23-25 and the coding sequence of exon-26, with a functional 3′-splice site at its 5′-end) becomes permanently ligated/inserted into the canine X-chromosome. Because the DNA segment between the left and right homology arms comprises a partial cF8 cDNA (which, as shown in FIG. 2 for the human F8-I22I, contains, in-frame, all of canine exons 23-25 and the coding sequence of canine exon-26) fused at its 5′-end to a functional 3′-splice site, this TALEN catalyzes repair and converts cF8-I22I into a wild-type cF8-like locus that restores its ability to drive synthesis of a full-length fully functional wild-type canine FVIII.

FIG. 5 illustrates a TALEN-mediated strategies to repair the human Factor VIII (FVIII) gene (F8) mutations in >50% of all patients with severe hemophilia-A (HA), including the highly recurrent intron-22 (I22)-inversion (I22I)-mutation. FIG. 5 highlights the TALEN approach linking Exon 22 of the F8 gene to a nucleic acid including exons 23-26 encoding a truncated FVIII polypeptide. Panel A of FIG. 5 shows the specific F8 genomic DNA sequence (spanning positions 126,625-126,693) within which a double-stranded DNA break (DSDBs) is introduced (designated “Endonuclease domain” and “target site” in Panel B) by this strategy's functional TALEN dimer. The left and right TALEN protein sequences for the variable DNA-binding domain are listed as Seq. ID. No. 4 and Seq. ID. No. 6, respectively. An example of DNA sequences encoding the left and right TALEN DNA-binding domains are listed as Seq. ID. No 5 and Seq. ID. No. 7, respectively. Because of the degeneracy of the genetic code, there are many possible constructs that can be used to encode TALEN DNA-binding domains. In some embodiments, the codons are optimized for expression of the DNA constructs. Panel A in FIG. 5 also shows the F8 genomic DNA sequence containing (i) the recognition sites for the left (TALEN_{L}-hF8_{E22/I22}) and right (TALEN_{R}-hF8_{E22/I22}) TALEN monomers comprising F8-TALEN-5 and (ii) the intervening spacer region within which the F8-TALEN-5's endonuclease activity creates the double-stranded DNA breaks (DSDBs) required for inducing the physiologic cellular machinery that mediates the homology-dependent DNA repair pathway. Panel A in FIG. 5 also shows important orienting landmarks, including the following: (i) Nucleotide coordinates of this region (based on the February, 2009, human genome assembly [UCSC Genome Browser: http://genome.ucsc.edu/]) are numbered with respect to the wild-type F8 transcription unit, where the initial (5′-most) base of the F8 pre-mRNA (5′-base of exon-1 [E1]) is designated +1 or 1 (note that this base corresponds to X-chromosome position 154,250,998) and includes the appropriate intronic sequence bases in calculating the genomic base positioning; (ii) Relative location of the X-chromosome's centromere (X-Cen) and its long-arm telomere (Xq-Tel), as transcription of the wild-type F8 locus and all of its mutant alleles causing HA with the exception of its two recurrent intronic inversions, the intron-1 (I1)-inversion (I1I)- and the I22I-mutations is oriented towards X-Cen. Transcription of the I1- and I22-inverted F8 loci, in contrast, are oriented towards Xq-Tel. This strategy repairs (i) the highly recurrent I22I-mutation—also designated F8_{I22I}—which causes ˜45% of all unrelated patients with severe hemophilia-A (HA) and (ii) mutant F8 loci in ˜20% of all other patients with severe HA, who are either known or found to have any one of the >200 distinct mutations that have been found (according to the HAMSTeRS database of HA-causing F8 mutations) thus far to reside down-stream (i.e., 3′) of exon-22 (E22). The last codon of exon 22 encodes methionine (Met [M]) as translated residue 2,143 (2,124 in the mature FVIII protein secreted into plasma). Most mutations repaired are “previously known” (literature and/or HAMSTeRS or other databases), some have never been identified previously; the F8 abnormalities in this latter category are “private” (found only in this particular) to the patient/family.

Panel B in FIG. 5 shows the functional aspects of the TALENs including the overall DNA-binding domain (DBD) and the DBD-subunit repeats of the left and right monomers (TALEN_{L}-hF8_{E22/I22} and TALEN_{R}-hF8_{E22/I22}). Also shown are the (i) specific DNA sequences recognized by each TALEN monomer (shown in bold font immediately below each DBD-subunit); (ii) the spacer region between the DNA recognition sequences of the TALEN monomers contains the sequence within which the dimerized Fok1 catalytic domains, which form a functional endonuclease, introduce a double-stranded DNA break (DSDB); this site is indicated as the target site. As shown in the lower left portion of FIG. 5, the introduction of a DSDB in the presence of homologous repair vehicle no. 5 (HRV5), the nucleotide sequence of which is provided below as Seq. ID. No. 12, results in the in-frame integration, immediately 3′ to exon 22, of the partial human F8 cDNA comprising exons 23, 24 and 25 and the protein coding sequence, or CDS, of exon 26 (designated hF8[E23-E25/E26_{CDS}]). In one embodiment, the TALEN constructs depicted in FIG. 5 can be used to repair all I22I inversion mutations (See #1 pathway). In another embodiment, the same constructs can be used to repair non-I22I F8 mutations that occur 3′ (i.e. downstream) of the exon-22/intron-22 junction (See #2 pathway).

FIG. 6 illustrates a TALEN-mediated strategy to repair the human F8 mutations in >50% of all patients with severe HA, including the highly recurrent I22I-mutation. FIG. 6 highlights the TALEN approach linking intron-22 of the F8 to a nucleic acid encoding a truncated FVIII polypeptide encoding exons 23-26. Panel A shows the specific F8 genomic DNA sequence within which a DSDB is introduced (designated “Endonuclease domain” in Panel B and “target site”) by this strategy's functional TALEN dimer. The left and right TALEN protein sequences for the variable DNA-binding domain are listed as Seq. ID. No. 8 and Seq. ID. No. 10, respectively. Examples of DNA sequences encoding the left and right TALEN DNA-binding domains are listed as Seq. ID. No. 9 and Seq. ID. No. 11, respectively. Because of the degeneracy of the genetic code, there are many possible constructs that can be used to encode TALEN DNA-binding domains. In some embodiments, the codons are optimized for expression of the DNA constructs. Panel A in FIG. 6 also shows important orienting landmarks, including the: (i) nucleotide coordinates of this region (based on the February, 2009, human genome assembly available at the UCSC Genome Browser: http://genome.ucsc.edu/) are numbered with respect to the wild-type F8 transcription unit, where the initial (5′-most) base of the F8 pre-mRNA (5′ most base of exon-1 [E1]) is designated +1 or 1 (note that this base corresponds to X-chromosome position 154,250,998) and includes the appropriate intronic sequence bases in calculating the genomic base positioning; (ii) relative location of the X-chromosome's centromere (X-Cen) and its long-arm telomere (Xq-Tel), as transcription of the wild-type F8 locus and all of its mutant alleles causing HA with the exception of its two recurrent intronic inversions, I1I- and the I22I-mutations—is oriented towards X-Cen; Transcription of the I1- and I22-inverted F8 loci, in contrast, is oriented towards Xq-Tel. This strategy repairs (i) the highly recurrent I22I-mutation—also designated F8_{I22I}—which causes ˜45% of all unrelated patients with severe HA and (ii) mutant F8 loci in ˜20% of all other patients with severe HA, who are either known or found to have any one of the >200 distinct mutations that have been found (according to the HAMSTeRS database of HA-causing F8 mutations) thus far to reside down-stream (i.e., 3′) of exon-22 (E22). The last codon of E22 entirely encodes methionine (Met [M]) as translated residue 2,143 (2,124 in the mature FVIII secreted into plasma). Most mutations repaired are “previously known” (literature and/or HAMSTeRS or other databases), but some have never been identified previously. The F8 abnormalities in this latter category are “private” (found only in this particular) to the patient/family.

Panel B in FIG. 6 shows the functional aspects of the TALENs including the overall DBD and the DBD-subunit repeats of the left and right monomers (TALEN_{L}-hF8_{I22} and TALEN_{R}-hF8_{I22}). Also shown are the (i) specific DNA sequences recognized by each TALEN monomer (shown in bold font immediately below each DBD-subunit); (ii) the spacer region between the DNA recognition sequences of the TALEN monomers contains the sequence within which the dimerized Fok1 catalytic domains, which form a functional endonuclease, introduce a DSDB; this site is indicated as the target site. As shown in the lower left portion of FIG. 6, the introduction of a DSDB in the presence of a homologous repair vehicle, the nucleotide sequence of which is listed as Seq. ID. No. 13, results in the integration into intron-22 of a native F8 3′ splice acceptor site operably linked to a nucleic acid encoding F8 exons-23, 24 and 25 and the protein coding sequence, or CDS, of exon-26 (designated hF8[E23-E25/E26_{CDS}]). In one embodiment, the TALEN constructs depicted in FIG. 6 can be used to repair all I22I inversion mutations (See #1 pathway). In another embodiment, the same constructs are used to repair non-I22I F8 mutations that occur 3′ (i.e. downstream) of the exon-22/intron-22 junction (See #2 pathway).

FIG. 7 shows a comparison of expected genomic DNA, spliced RNA and proteins pre and post repair. Several examples of functional and non-functional coding sequences are depicted in the gDNA panel of FIG. 7. Example functional coding sequences include exons 1-22 and exons 22-23 of the wild-type F8 genomic DNA (Normal), exons 1-22 of the I22I mutant F8 genomic DNA (I22I), and exons 1-22 of the I22I mutant F8 genomic DNA and exons 23-26 of the wild-type F8 cDNA (Repaired). Example non-functional coding sequences include exons 23-26 of the I22I mutant F8 genomic DNA (I22I) and exons 23-26 of the I22I mutant F8 genomic DNA (right, Repaired).

In some embodiments, nucleic acids encoding nucleases specifically target intron-1, intron-14, or intron-22. In some embodiments, nucleic acids encoding nucleases specifically target the exon-1/intron-1 junction; exon-14/intron-14 junction; or the exon-22/intron-22 junction.

FIG. 9 illustrates an example of a donor plasmid that can be used to repair the F8 at the exon-22/intron-22 junction using a TALEN, ZFN, CRISPR/Cas, CRISPR-PN, and CRISPR-RFN approach. The donor plasmid contains the cDNA sequence for exons 23-26 of the F8 (labeled as functional coding sequence) and a polyadenylation signal sequence flanked by two regions of homology to the F8. The left homology region contains a DNA sequence (approximately 700 base pairs) that is homologous to part of intron-21 and exon-22 of the F8. The right homology region contains a DNA sequence (approximately 700 base pairs) that is homologous to part of intron-22 of the F8. Upon successful homologous recombination into the F8 locus, the integrated construct expresses the resulting mRNA encoding the wild-type (corrected) version of the FVIII. The sequence of the plasmid depicted in FIG. 9 is listed as Seq. ID. No. 12. The annotation of Seq. ID. No. 12 is provided in Table 1 below.

TABLE 1
Repair vehicle targeted to the Exon 22 - Intron 22 junction of F8
LOCUS RepairVehicle 7753 bp DNA linear
FEATURES Location/Qualifiers

| | |
|---|---|
| misc_feature | 21 . . . 327 |
| | /note=“f1 origin (−)” |
| misc_feature | 6765 . . . 7625 |
| | /note=“<= Ampicillin” |
| misc_feature | 471 . . . 614 |
| | /label=<= lacZ A |
| misc_feature | 626 . . . 644 |
| | /note=“T7 promoter =>” |
| misc_feature | 5564 . . . 5583 |
| | /note=“T3 promoter =>” |
| misc_feature | 6765 . . . 7625 |
| | /note=“<= Orf1” |
| misc_feature | 7667 . . . 7695 |
| | /note=“<= AmpR promoter” |
| misc_feature | 658 . . . 740 |
| | /note=“MCS” |
| misc_feature | 1446 . . . 2072 |
| | /note=“Exons 23-26 (cDNA seq)” |
| misc_feature | 1730 . . . 1737 |
| | /note=“Create NotI site” |
| misc_feature | 2082 . . . 2707 |
| | /note=“hGH polyA” |
| misc_feature | 1785 . . . 1787 |
| | /note=“ns-SNP: A6940G (M2238V)” |
| misc_feature | 3408 . . . 4160 |
| | /note=“HSV-TK promoter ” |
| misc_feature | 4161 . . . 5546 |
| | /note=“HSV-TK gene and TK pA Terminator ” |
| misc_feature | 741 . . . 745 |
| | /note=“Create site for cloning” |
| misc_feature | 5547 . . . 5551 |
| | /note=“Create site for cloning” |
| misc_feature | 746 . . . 1445 |
| | /note=“Left homolgy arm (700 bp)” |
| misc_feature | 1290 . . . 1445 |
| | /note=“Exon 22” |
| misc_feature | 1433 . . . 1445 |
| | /note=“Partial Left TALEN recognition site” |
| misc_feature | 2708 . . . 3407 |
| | /note=“Right homology arm (700 bp)” |
| misc_feature | 2708 . . . 2716 |
| | /note=“Partial Right TALEN recognition site” |
| misc_feature | 2708 . . . 3407 |
| | /note=“Partial Intron 22” |
| misc_feature | 746 . . . 1289 |
| | /note=“Partial Intron 21” |
| source | 1 . . . 7753 |
| | /dnas_title=“RepairVehicle E22-I22 pBluescript” |

FIG. 10 illustrates an example of a donor plasmid that can be used to repair the F8 using a TALEN, ZFN, CRISPR/Cas, CRISPR-PN, and CRISPR-RFN approach. The donor plasmid contains the cDNA sequence for exons2-26 of the F8 (labeled as functional coding sequence) flanked by two regions of homology to the F8. The left homology region contains a DNA sequence that is homologous to part of the F8 promoter and part of exon-1. The right homology region contains a DNA sequence that is homologous to part of intron-1. Upon successful homologous recombination into the F8, the integrated construct expresses the resulting mRNA encoding the wild-type (corrected) version of the FVIII. The donor sequence is cloned into plasmid (p)BlueScript-II KS-minus (pBS-II-KS[−]). The donor plasmid is used with a TALEN, ZFN, CRISPR/Cas, CRISPR-PN, and CRISPR-RFN genomic editing strategy. The sequence of the plasmid depicted in FIG. 10 is listed as Seq. ID. No. 13. The annotation of Seq. ID. No. 13 is provided in Table 2 below.

TABLE 2
Repair vehicle targeted to the Exon 1 - Intron 1 junction of F8
LOCUS RepairVehicle 11418 bp DNA linear
FEATURES Location/Qualifiers

| | |
|---|---|
| misc_feature | 21 . . . 327 |
| | /note=“f1 origin (−)” |
| misc_feature | 10430 . . . 11290 |
| | /note=“<= Ampicillin” |
| misc_feature | 471 . . . 614 |
| | /label=<= lacZ A |
| misc_feature | 626 . . . 644 |
| | /note=“T7 promoter =>” |
| misc_feature | 9229 . . . 9248 |
| | /note=“<= T3 promoter” |
| misc_feature | 10430 . . . 11290 |
| | /note=“<= Orf1” |
| misc_feature | 11332 . . . 11360 |
| | /note=“<= AmpR promoter” |
| misc_feature | 658 . . . 740 |
| | /note=“MCS” |
| misc_feature | 5780 . . . 6405 |
| | /note=“hGH polyA” |
| misc_feature | 7073 . . . 7825 |
| | /note=“HSV-TK promoter ” |
| misc_feature | 7826 . . . 9211 |
| | /note=“HSV-TK gene and TK pA Terminator ” |
| misc_feature | 740 . . . 745 |
| | /note=“Create site for cloning” |
| misc_feature | 1540 . . . 5770 |
| | /note=“Exons 2-26 BDD (cDNA seq)” |
| misc_feature | 2664 . . . 2669 |
| | /note=“Create ClaI site” |
| misc_feature | 2903 . . . 2905 |
| | /note=“ns-SNP: G1679A (R484H)” |
| misc_feature | 3680 . . . 3685 |
| | /note=“BDD (Ser743 - Gln1638)” |
| misc_feature | 5428 . . . 5435 |
| | /note=“Create NotI site” |
| misc_feature | 5768 . . . 5768 |
| | /dnas_title=“Stop” |
| | /vntifkey=“21” |
| | /label=Stop |
| misc_feature | 5483 . . . 5485 |
| | /note=“ns-SNP: A6940G (M2238V)” |
| insertion_seq | 3934 . . . 5770 |
| | /dnas_title=“Tg” |
| | /vntifkey=“14” |
| | /label=Tg |
| misc_feature | 9212 . . . 9217 |
| | /note=“Create site for cloning” |
| misc_feature | 9212 . . . 9212 |
| | /note=“MCS” |
| misc_feature | 746 . . . 1539 |
| | /note=“Left homolgy arm (794bp)” |
| misc_feature | 746 . . . 1237 |
| | /note=“Partial F8 promoter” |
| misc_feature | 1238 . . . 1539 |
| | /note=“Partial Exon 1” |
| misc_feature | 6406 . . . 7072 |
| | /note=“Right homology arm (667 bp)” |
| misc_feature | 6406 . . . 7072 |
| | /note=“Partial intron 1” |
| source | 1 . . . 11418 |
| | /dnas_title=“RepairVehicle E1-I1 pBluescript” |

FIG. 11 illustrates an example of a donor plasmid that is used to repair the F8 in intron-22 using a TALEN, ZFN, CRISPR/Cas, CRISPR-PN, and CRISPR-RFN approach. The donor plasmid contains a 3′ splice site, the cDNA sequence for exons 23-26 of the F8 (labeled as functional coding sequence), and a polyadenylation signal sequence flanked by two regions of homology to the F8. The left homology region contains a DNA sequence (approximately 700 base pairs) that is homologous to part of intron-22 of the F8. The right homology region contains a DNA sequence (approximately 700 base pairs) that is homologous to part of intron-22 of the F8. Upon successful homologous recombination into the F8 locus, the integrated construct expresses the resulting mRNA encoding the wild-type (corrected) version of the FVIII. The sequence of the plasmid depicted in FIG. 11 is listed as Seq. ID. No. 14. The annotation of Seq. ID. No. 14 is provided in Table 3 below.

TABLE 3
Repair vehicle targeted to Intron 22 of F8
LOCUS RepairVehicle 7755 bp DNA linear
FEATURES Location/Qualifiers

| | |
|---|---|
| misc_feature | 21 . . . 327 |
| | /note=“f1 origin (−)” |
| misc_feature | 6767 . . . 7627 |
| | /note=“<= Ampicillin” |
| misc_feature | 471 . . . 614 |
| | /label=<= lacZ A |
| misc_feature | 626 . . . 644 |
| | /note=“T7 promoter =>” |
| misc_feature | 5566 . . . 5585 |
| | /note=“T3 promoter =>” |
| misc_feature | 6767 . . . 7627 |
| | /note=“<= Orf1” |
| misc_feature | 7669 . . . 7697 |
| | /note=“<= AmpR promoter” |
| misc_feature | 658 . . . 740 |
| | /note=“MCS” |
| misc_feature | 1448 . . . 2074 |
| | /note=“Exons 23-26 (cDNA seq)” |
| misc_feature | 1732 . . . 1739 |
| | /note=“Create NotI site” |
| misc_feature | 2084 . . . 2709 |
| | /note=“hGH polyA” |
| misc_feature | 1787 . . . 1789 |
| | /note=“ns-SNP: A6940G (M2238V)” |
| misc_feature | 3410 . . . 4162 |
| | /note=“HSV-TK promoter ” |
| misc_feature | 4163 . . . 5548 |
| | /note=“HSV-TK gene and TK pA Terminator ” |
| misc_feature | 741 . . . 745 |
| | /note=“Create site for cloning” |
| misc_feature | 5549 . . . 5553 |
| | /note=“Create site for cloning” |
| misc_feature | 746 . . . 1445 |
| | /note=“Left homology arm (700 bp)” |
| misc_feature | 1437 . . . 1445 |
| | /note=“Partial Left TALEN recognition site” |
| misc_feature | 2710 . . . 3409 |
| | /note=“Right homolgy arm (700 bp)” |
| misc_feature | 2710 . . . 2719 |
| | /note=“Partial Right TALEN recognition site” |
| misc_feature | 746 . . . 1445 |
| | /note=“Partial Intron 22” |
| misc_feature | 2710 . . . 3409 |
| | /note=“Partial Intron 22” |
| misc_feature | 1446 . . . 1447 |
| | /note=“3′ spice site” |
| source | 1 . . . 7755 |
| | /dnas_title=“RepairVehicle I22 pBluescript” |

FIG. 12 illustrates an example of a donor plasmid that is used to repair the F8 in intron-1 using a TALEN, ZFN, CRISPR/Cas, CRISPR-PN, and CRISPR-RFN approach. The donor plasmid contains a 3′ splice site, the cDNA sequence of the F8 for exons 2-26 lacking the B-domain (B-domain deleted (BDD) version of the F8) (labeled as functional coding sequence), and a polyadenylation signal sequence flanked by two regions of homology to the F8. The left homology region contains a DNA sequence (approximately 700 base pairs) that is homologous to part of exon-1 and intron-1 of the F8 gene. The right homology region contains a DNA sequence (approximately 700 base pairs) that is homologous to part of intron-1 of the F8. Upon successful homologous recombination into the F8 locus, the integrated construct expresses the resulting mRNA encoding the wild-type (corrected) version of the FVIII. The sequence of the plasmid depicted in FIG. 12 is listed as Seq. ID. No. 15. The annotation of Seq. ID. No. 15 is provided in Table 4 below.

TABLE 4
Repair vehicle targeted to Intron 1 of F8
LOCUS RepairVehicle 11359 bp DNA linear
FEATURES Location/Qualifiers

| | |
|---|---|
| misc_feature | 21 . . . 327 |
| | /note=“f1 origin (−)” |
| misc_feature | 10371 . . . 11231 |
| | /note=“<= Ampicillin” |
| misc_feature | 471 . . . 614 |
| | /label=<= lacZ A |
| misc_feature | 626 . . . 644 |
| | /note=“T7 promoter =>” |
| misc_feature | 9170 . . . 9189 |
| | /note=“<= T3 promoter” |
| misc_feature | 10371 . . . 11231 |
| | /note=“<= Orf1” |
| misc_feature | 11273 . . . 11301 |
| | /note=“<= AmpR promoter” |
| misc_feature | 658 . . . 740 |
| | /note=“MCS” |
| misc_feature | 874 . . . 1187 |
| | /note=“Exon 1” |
| misc_feature | 1436 . . . 1445 |
| | /note=“Partial Left TALEN recognition site” |
| misc_feature | 5688 . . . 6313 |
| | /note=“hGH polyA” |
| misc_feature | 6314 . . . 7013 |
| | /note=“Right homology arm (700 bp)” |
| misc_feature | 6314 . . . 6322 |
| | /note=“Partial Right TALEN recognition site” |
| misc_feature | 7014 . . . 7766 |
| | /note=“HSV-TK promoter ” |
| misc_feature | 7767 . . . 9152 |
| | /note=“HSV-TK gene and TK pA Terminator ” |
| misc_feature | 746 . . . 1445 |
| | /note=“Left homolgy arm (700 bp)” |
| misc_feature | 746 . . . 873 |
| | /note=“Partial F8 promoter” |
| misc_feature | 740 . . . 745 |
| | /note=“Create site for cloning” |
| misc_feature | 6314 . . . 7013 |
| | /note=“Partial Intron 1” |
| misc_feature | 1448 . . . 5678 |
| | /note=“Exons 2-26 BDD (cDNA seq)” |
| misc_feature | 1446 . . . 1447 |
| | /note=“3′ spice site” |
| misc_feature | 2572 . . . 2577 |
| | /note=“Create ClaI site” |
| misc_feature | 2811 . . . 2813 |
| | /note=“ns-SNP: G1679A (R484H)” |
| misc_feature | 3588 . . . 3593 |
| | /note=“BDD (Ser743 - Gln1638)” |
| misc_feature | 5336 . . . 5343 |
| | /note=“Create NotI site” |
| misc_feature | 5676 . . . 5676 |
| | /dnas_title=“Stop” |
| | /vntifkey=“21” |
| | /label=Stop |
| misc_feature | 5391 . . . 5393 |
| | /note=“ns-SNP: A6940G (M2238V)” |
| insertion_seq | 3842 . . . 5678 |
| | /dnas_title=“Tg” |
| | /vntifkey=“14” |
| | /label=Tg |
| misc_feature | 9153 . . . 9158 |
| | /note=“Create site for cloning” |
| misc_feature | 9153 . . . 9153 |
| | /note=“MCS” |
| source | 1 . . . 11359 |
| | /dnas_title=“RepairVehicle I1 pBluescript” |

In one embodiment, the integration matrix component for each of the distinct homologous donor plasmid is either a cDNA that is linked to the immediately upstream exon or a cDNA that has a functional 3′-intron-splice-junction so that the cDNA sequence is linked through the RNA intermediate following removal of the intron. In one embodiment, the donor plasmid is personalized, on an individual basis, so that each patient's gene that is repaired expresses the form of the FVIII that they are maximally tolerant of.

In some embodiments the DNA-SE used for F8 targeting is a ZFN. ZFNs are hybrid proteins containing the zinc-finger DNA-binding domain present in transcription factors and the non-specific cleavage domain of the endonuclease Fok1. (Li et al., In vivo genome editing restores hemostasis in a mouse model of hemophilia, Nature 2011 Jun. 26; 475(7355):217-21).

The same sequences targeted by the TALEN approach, discussed above, can also be targeted by the ZFN approach for genome editing. ZFNs are a class of engineered DNA-binding proteins that facilitate targeted editing of the genome by creating DSDB at user-specified locations. Each ZFN consists of two functional domains: 1) a DBD comprised of a chain of two-finger modules, each recognizing a unique hexamer (6 bp) sequence of DNA, wherein two-finger modules are stitched together to form a ZFN, each with specificity of ≧24 bp, and 2) a DNA-cleaving domain comprised of the nuclease domain of Fok 1. The DNA-binding and DNA-cleaving domains are fused together and recognize the targeted genomic sequences, allowing the Fok1 domains to form a heterodimeric enzyme that cleaves the DNA by creating double stranded breaks.

ZFNs can be readily made by using techniques known in the art (Wright D A, et al. Standardized reagents and protocols for engineering zinc finger nucleases by modular assembly. Nat Protoc. 2006; 1(3):1637-52). Engineered ZFNs can stimulate gene targeting at specific genomic loci in animal and human cells. The construction of artificial zinc finger arrays using modular assembly has been described. The archive of plasmids encoding more than 140 well-characterized zinc finger modules together with complementary web-based software for identifying potential zinc finger target sites in a gene of interest has also been described. These reagents enable easy mixing-and-matching of modules and transfer of assembled arrays to expression vectors without the need for specialized knowledge of zinc finger sequences or complicated oligonucleotide design (Wright D A, et al. Standardized reagents and protocols for engineering zinc finger nucleases by modular assembly. Nat Protoc. 2006; 1(3):1637-52). Any gene in any organism can be targeted with a properly designed pair of ZFNs. Zinc-finger recognition depends only on a match to the target DNA sequence (Carroll, D. Genome engineering with zinc-finger nucleases. Genetics Society of America, 2011, 188(4), pp 773-782).

In some embodiments the DNA-SE used for F8 gene targeting comprises Clustered Regularly Interspaced Short Palindromic Repeats (CRISPR) and CRISPR Associated (Cas) Nucleases based on CRISPR technology. (Mali P, Yang L, Esvelt K M, Aach J, Guell M, DiCarlo J E, Norville J E, Church G M. RNA-guided human genome engineering via Cas9. Science. 2013 Feb. 15; 339(6121):823-6; Gasiunas G, Barrangou R, Horvath P, Siksnys V. Cas9-crRNA ribonucleoprotein complex mediates specific DNA cleavage for adaptive immunity in bacteria. Proc Natl Acad Sci USA. 2012 Sep. 25; 109(39):E2579-86. Epub 2012 Sep. 4).

The Clustered Regularly Interspaced Short Palindromic Repeats (CRISPR) and CRISPR Associated (Cas) system was discovered in bacteria and functions as a defense against foreign DNA, either viral or plasmid. In bacteria, the endogenous CRISPR/Cas system targets foreign DNA with a short, complementary single-stranded RNA (CRISPR RNA or crRNA) that localizes the Cas9 nuclease to the target DNA sequence. The DNA target sequence can be on a plasmid or integrated into the bacterial genome. The crRNA can bind on either strand of DNA and the Cas9 cleaves both strands (double strand break, DSB). A recent in vitro reconstitution of the Streptococcus pyogenes type II CRISPR system demonstrated that crRNA fused to a normally trans-encoded tracrRNA is sufficient to direct Cas9 protein to sequence-specifically cleave target DNA sequences matching the crRNA. The fully defined nature of this two-component system allows it to function in the cells of eukaryotic organisms such as yeast, plants, and even mammals. By cleaving genomic sequences targeted by RNA sequences, such a system greatly enhances the ease of genome engineering.

The crRNA targeting sequences are transcribed from DNA sequences known as protospacers. Protospacers are clustered in the bacterial genome in a group called a CRISPR array. The protospacers are short sequences (˜20 bp) of known foreign DNA separated by a short palindromic repeat and kept like a record against future encounters. To create the CRISPR targeting RNA (crRNA), the array is transcribed and the RNA is processed to separate the individual recognition sequences between the repeats. In the Type II system, the processing of the CRISPR array transcript (pre-crRNA) into individual crRNAs is dependent on the presence of a trans-activating crRNA (tracrRNA) that has sequence complementary to the palindromic repeat. When the tracrRNA hybridizes to the short palindromic repeat, it triggers processing by the bacterial double-stranded RNA-specific ribonuclease, RNase III. Any crRNA and the tracrRNA can then both bind to the Cas9 nuclease, which then becomes activated and specific to the DNA sequence complimentary to the crRNA. (Mali P, Yang L, Esvelt K M, Aach J, Guell M, DiCarlo J E, Norville J E, Church G M. RNA-guided human genome engineering via Cas9. Science. 2013 Feb. 15; 339(6121):823-6; Gasiunas G, Barrangou R, Horvath P, Siksnys V. Cas9-crRNA ribonucleoprotein complex mediates specific DNA cleavage for adaptive immunity in bacteria. Proc Natl Acad Sci USA. 2012 Sep. 25; 109(39):E2579-86. Epub 2012 Sep. 4).

The DSDB induced by the TALEN approach overlaps with the 6 distinct sites of DSDB induced by Cas9, via targeting by 6 distinct CRISPR-guide RNAs [F8-CRISPR/Cas9-1 (F8-Ex1/Int1), F8-CRISPR/Cas9-2 (F8-Int1), F8-CRISPR/Cas9-3 (F8-Ex14/Int1 4), F8-CRISPR/Cas9-4 (F8-Int14), F8-CRISPR/Cas9-5 (F8-Ex22/Int22), F8-CRISPR/Cas9-6 (F8-Int22)]. This allows use of the same 6 distinct homologous donor sequences with all three genome editing approaches, including the TALEN nuclease, ZFN, and the Cas nuclease.

FIG. 13 illustrates a CRISPR/Cas9-mediated strategy to repair the human Factor VIII (FVIII) gene (F8) mutations in ˜95% of all patients with severe hemophilia-A (HA), including the highly recurrent intron-1 (I1)-inversion (I1I)-mutation as well as the intron-22 (I22)-inversion (I22I)-mutation. FIG. 13 shows the specific F8 genomic DNA sequence (spanning genic base positions 172-354 at intron 1) within which a double-stranded (ds)-DNA break is introduced (designated “Endonuclease target” or “target site” in this panel) by this strategy's wild-type (wt) CRISPR/Cas9 ds-DNase in which both of its endonuclease domains are catalytically functional (“hF8-CRISPR/Cas9 wt-1”). This panel also shows important orienting landmarks, including the following: (i) Nucleotide coordinates of this region (based on the February, 2009, human genome assembly [UCSC Genome Browser: http://genome.ucsc.edu/]) are numbered with respect to the wild-type F8 transcription unit, where the initial (5′-most) base of the F8 pre-mRNA (5′-base of exon-1 [E1]) is designated +1 or 1 (note that this base corresponds to X-chromosome position 154,250,998) and include the appropriate intronic sequence bases in calculating the genomic base positioning; (ii) Relative location of the X-chromosome's centromere (X-Cen) and its long-arm telomere (Xq-Tel), as transcription of the wild-type F8 locus and all of its mutant alleles causing HA with the exception of its two recurrent intronic inversions, the I1I- and the I22I-mutations—is oriented towards X-Cen. Transcription of the I1- and I22-inverted F8 loci, in contrast, are oriented towards Xq-Tel. This strategy repairs (i) the highly recurrent I22I-mutation—also designated F8_{I22I}—which causes ˜45% of all unrelated patients with severe hemophilia-A (HA) and (ii) mutant F8 loci in ˜90-95% of all other patients with severe HA, who are either known or found to have any one of the >1,500 distinct mutations that have been found (according to the HAMSTeRS database of HA-causing F8 mutations) thus far to reside down-stream (i.e., 3′) of exon-1 (E1). The last codon of E1 partially encodes the translated residue 48 (29 in the mature FVIII protein secreted into plasma). Most mutations repaired are “previously known” (literature and/or HAMSTeRS or other databases). Some have never been identified previously. These F8 abnormalities in this latter category are “private” (found only in this particular) to the patient/family. Finally, FIG. 13 shows the functional aspects of hF8-CRISPR/Cas9 wt-1 including the overall DNA-binding domain of the CRISPR-associated guide (g)RNA as well as the (i) Protospacer adjacent motif (PAM), which is the site at which the DNase function of Cas9 introduces the ds-DNA break (DSDB); and (ii) The Transactivating Crispr-RNA (TrCr-RNA), which is covalently attached the gRNA as is what brings the Cas9 endonuclease to the genomic DNA target for digestion. The introduction of a DSDB in the presence of a homologous repair vehicle, results in the in-frame integration, immediately 3′ to E1, of one of either two partial human F8 cDNAs comprising either (i) exons 2-25 and the protein coding sequence, or CDS, of exon 26 (designated hF8[E2-E25/E26_{CDS}]), which effects repair of the F8 gene such that it now encodes a full-length wild-type FVIII protein; or (ii) Exons 2-13 entirely linked next to the very 5′-most end of exon-14 (E14), which in turn is linked covalently to the very 3′-most end of E14 (i.e., a B-domain-deleted [BDD]-F8 cDNA), which is then covalently linked to Exons 15-25 entirely, and then the protein coding sequence, or CDS, of exon 26 (designated hF8[E2-E13/E14-BDD/E15-E25/E26_{CDS}]), which effects repair of the F8 gene such that it now encodes a BDD-engineered FVIII protein, which is fully functional in FVIII:C activity. The homologous repair vehicle is selected to have a F8 cDNA with the appropriate alleles at all ns-SNP sites so that the patient can receive a “matched” gene repair or at least a least mismatched repair.

The left homology arm of the homologous repair vehicle for Homologous Repair Vehicle No. 1 (HRV1) for hF8-CRISP/Cas9 wt-1 is listed as Seq. ID. No. 17 and comprises the first 1114 bases of the human F8 genomic DNA (which is shown here as single-stranded and representing the sense strand) and contains 800 bp of the immediately 5′-promoter region of the human F8 gene and all 314 bp of the F8 exon-1 (E1), including its 171 bp 5′-UTR and its 143 bp of protein (en)coding sequence (CDS). The actual left homologous arm (LHA) of the homologous repair vehicle (HRV1), which is used for this CRISPR/Cas9-mediated F8 gene repair (that occurs at the E1/intron-1 [I1] junction of a given patient's endogenous mutant F8), contains at least 500 bp of this genomic DNA sequence (i.e., from it's very 3′-end, which corresponds to the second base of the codon for translated residue 48 of the wild-type FVIII protein and residue 29 of the mature FVIII protein found in the circulation) and could include it all, if, for example, we find that full-length F8 gene repair can be effected efficiently in the future. In this instance, the integration matrix would then follow the LHA of this HRV1, and be covalently attached to it, and this integration matrix contains (in-frame with each other and with the 3′-end of the patient's native exon-1, which is utilized in situ, along with his native F8 promoter, to regulate expression of the repaired F8 gene), all of F8 exons 2-25, and the protein CDS of exon-26, followed by the functional mRNA 3′-end forming signals of the human growth hormone gene (hGH-pA). The F8 cDNA from exons 2-25 and the CDS of exon-26 to be used in the homologous repair vehicle is listed as Seq. ID. No. 18 and follows the left homology arm, and in this example represents the haplotype (H)3 encoding wild-type variant of F8, which can be used to cure, for example, patients with the I1I-mutation and the I22I-mutation, that arose on an H3-background haplotype. This following protein encoding cDNA sequence contains 6,909 bp of the entire 7,053 bp of F8 protein encoding sequence (i.e., the first 144 bp of protein CDS from FVIII, from its initiator methionine, is not shown, as this is contained in exon-1, which is provided by the patient's own endogenous exon-1, providing it is not mutant and thus precluding the repair event). The right homology arm of the homologous repair vehicle for the cas nuclease approach is listed as Seq. ID. No. 19 and includes 1109 bases of human F8 genomic DNA (which is shown here as single-stranded and representing the sense strand) from the F8 gene intron 1.

In some embodiments, the DNA-SE is a CRISPR Paired Nickase. A single CRISPR nuclease targets a total of 22 bp of DNA sequence, which is much less than what is targeted by dimeric TALENs (30-40 bp) or ZFNs (30-36 bp); as a result, some CRISPR nucleases can have substantial off-target activity throughout the rest of the genome. The Cas9 protein has two nuclease domains (an HNH domain and a RuvC domain) which each cleave one of the strands of the DNA helix in order to cause a double-strand break. By inactivating one of the nuclease domains in Cas9 (through the amino acid mutation D10A or H840A), the Cas9 molecule becomes a ‘nickase’ which can only cause a break in one strand of DNA thereby creating a nick rather than a double-strand break. However, by targeting to Cas9-nickase molecules to nearby regions of DNA, offset nicks can in effect cause a double-strand break with DNA overhangs similar to how the two FokI dimers in ZFNs and TALENs come together to create a double-strand DNA break with overhanging bases. Guidelines for how to orient the paired target sites for Cas9-nickases were developed by Ran F A, Hsu P D et al. Cell 2013, incorporated herein by reference, and it was shown that similar on-target activity was able to be achieved by correctly oriented paired Cas9-nickases as by a single Cas9-nuclease. Importantly, it was also shown that at sites previously identified as having off-target activity when using a certain guide strand with the Cas9 nuclease that when using the Cas9-nickase the off-target activity was reduced 1400 fold. The hypothesis for the reduction in off-target activity is that although at the previously identified off-target site there was homology to one of the guide strands (which allowed off-target activity using the Cas9-nuclease), in that region of the genome there was not also homology to the other guide strand in the pair; binding of a single Cas9-nickase does not induce DNA mutations, it is only when both guide strands bind in proper orientation that nicks are made in both DNA strands to create a double strand break which can lead to mutations through the NHEJ pathway. By creating the requirement that both guide strands bring the two nickases to the same region of the genome, the effective targeting length of the paired Cas9-nickase system is 44 bp, compared to 22 bp of the Cas9-nuclease system, greatly enhancing specificity in large genomes such as the human genome.

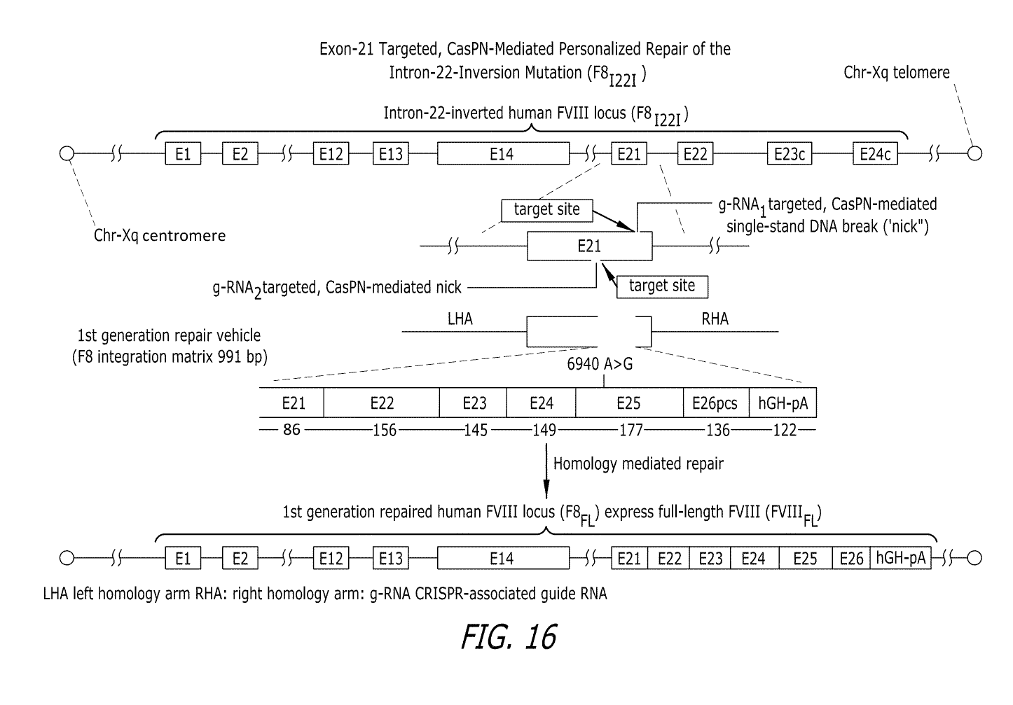

Example of repair at the exon21/intron-21 junction (the 3′-end of exon-21), using paired nickase are described below. Repair of the F8 at exon-21/intron-21 junction, i.e. the 3′-end of exon-21 would correct HA in patients with mutations in exons 22, 23, 24, 25, or 26, as well as the common I22I mutation. Examples of known patient mutations in exons 22-26 are detailed in FIG. 14, including, but not limited to (i) the F8 c.6761 T>A nonsense mutation that results in a stop codon at codon 2178 in place of the leucine (Leu)-encoding codon that is present at codon 2178 in the non-mutated form of the F8; (ii) the F8 c.6917 T>G missense mutation that results in a codon encoding arginine (Arg) at codon 2230 in place of the leucine (Leu)-encoding codon that is present at that codon 2230 in the non-mutated form of the F8; (iii) the F8-I22I mutation that is detailed above; (iv) the F8 IVS-23+1 G>A splice site mutation that results in a non-functional pre-mRNA splice site immediately downstream of exon-23 of the F8; (v) the F8 del exons 24-26 multi-exonic deletion mutation that results in deletion of exons 24-26 of the F8; and (vi) the F8 exon-26 del.[A] small deletion and frameshift mutation that results in a frameshift of the gene-encoding sequence which changes the downstream sequence by a single base-pair deletion frameshift and introduction of a novel terminating stop codon in the gene-encoding sequence. Creating the double-strand break at exon-21/intron-21 junction can be accomplished by using DNA-SE including such as TALENs, Cas9-nuclease, paired Cas9-nickases, or RNA-guided FokI Nucleases disclosed herein. An example of how to create such a break in F8 with paired Cas9-nickases is illustrated in FIG. 15. Specifically, Cas9-nickases are shown binding near the exon-21/intron-21 junction of F8. The Cas9-nickases create nicks on both strands of F8 DNA, thereby generating a double-strand break that will trigger homology directed repair; the site of the break is indicated as the “target site.” An engineered homologous repair vehicle (HRV) disclosed herein is then introduced to the cells along with the DNA-SE in order to be used as a template in the homology directed repair pathway. An example of a RV to be used at the exon-21/intron-21 junction is shown here FIG. 16. Regardless of the mechanism used to create the DNA-break at the exon-21/intron-21 junction the same RV can be used to alter the gene sequence. This RV has a LHA corresponding to the sequence 5′ of the DNA break labeled as “target break” (exon-21 and a portion of intron-20), the cDNA sequence encoding the downstream exons of the F8 (exons 22-26), a polyadenylation signal (such as the signal from the hGH gene labeled as “target break,” hGH-pA), and aRHA corresponding to the sequence 3′ of the DNA break (intron-21). After homology directed repair takes place, the gDNA sequence now contains a healthy copy of exons 22-26 fused to exon-21, allowing expression of the full-length F8. The RV can also contain SNPs in order to haplotypically match a certain patient; an example SNP (6940 A>G) is shown here.

In some embodiments the DNA-SE comprises CRISPR-RNA-guided Fok1 nucleases (CRISPR-RFN). Although the paired Cas9-nickases dramatically increased the specificity of CRISPR systems, low levels of off-target activity were still observed at some sites (Ran F A and Hsu P D et al. Cell 2013), presumably due to the occasional repair of DNA nicks through the error-prone NHEJ pathway rather than the error free base-excision-repair pathway. In contrast to a Cas9-nickase, which will cut one strand of DNA even in the absence of its corresponding pair, the FokI nuclease requires dimerization in order to cleave DNA; the presence of a single FokI monomer will not make any modification to the DNA. The Cas9 molecule can have all of its DNA cleavage activity removed by mutating both DNA cleavage domains (using the amino acid substitutions D10A and H840A) which is known as “dead” Cas9 or dCas9. When the FokI domain is fused to dCas9, two properly oriented guide strands can bring the two FokI domains in close proximity where they can dimerize and create a double-strand break, in a similar manner to ZFNs and TALENs. Tsai S Q et al (Nature Biotech 2014), incorporated herein by reference, determined that with correct orientation of guide strands and fusing FokI to the N-terminus of dCas9, double-strand breaks can be made efficiently by these RNA-guided FokI Nucleases, termed “RFNs”. Tsai et al further characterized the off-target activity of these RFNs and found that they had even lower levels of off-target activity than the paired Cas9-nickases targeted to the same locations; in almost all cases the off-target activity of the RFNs was below the detection limit of the deep-sequencing-based assay employed. A further method in which RFNs reduce off-target activity is that they are more limited in what orientations they can efficiently cleave DNA compared to paired Cas9-nickases. This reduces the possibility for off-target sites, but also limits the types of sequences which can be targeted by RFNs; several 3′ ends of the exons in the F8 gene did not contain the required sequence motifs to be able to be effectively targeted by RFNs. Overall, RFNs have benefits and drawbacks compared to the paired Cas9-nickases, but nonetheless represent another addition to the toolkit of nucleases available to create double-strand breaks in order to trigger homology-directed repair.

In methods and systems and related cDNA, vehicles and composition herein descried the gene targeting and repair approaches using the different nucleases of the disclosure can be carried out using many different target cells. For example, the transduced cells can include endothelial cells, hepatocytes, or stem cells. In one embodiment, the cells can be targeted in vivo. In one embodiment, the cells can be targeted using ex vivo approaches and reintroduced into the subject.

In one embodiment, the target cells from the subject are endothelial cells. In one embodiment, the endothelial cells are blood outgrowth endothelial cells (BOECs). Characteristics that render BOECs attractive for gene repair and delivery include the: (i) ability to be expanded from progenitor cells isolated from blood, (ii) mature endothelial cell, stable, phenotype and normal senescence (˜65 divisions), (iii) prolific expansion from a single blood sample to 1019 BOECs, (iv) resilience, which unlike other endothelial cells, permits cryopreservation and hence multiple doses for a single patient prepared from a single isolation. Methods of isolation of BOECs are known, where the culture of peripheral blood provides a rich supply of autologous, highly proliferative endothelial cells, also referred to as blood outgrowth endothelial cells (BOECs). Bodempudi V, et al., Blood outgrowth endothelial cell-based systemic delivery of antiangiogenic gene therapy for solid tumors. Cancer Gene Ther. 2010 December; 17(12):855-63.

Studies in animal models have revealed properties of blood outgrowth endothelial cells that indicate that they are suitable for use in ex vivo gene repair strategies. For example, a key finding concerning the behavior of canine blood outgrowth endothelial cells (cBOECs) is that cBOECs persist and expand within the canine liver after infusion. Milbauer L C, et al. Blood outgrowth endothelial cell migration and trapping in vivo: a window into gene therapy. 2009 April; 153(4):179-89. Whole blood clotting time (WBCT) in the HA model was also improved after administration of engineered cBOECs. WBCT dropped from a pretreatment value of under 60 min to below 40 min and sometimes below 30 min. Milbauer L C, et al., Blood outgrowth endothelial cell migration and trapping in vivo: a window into gene therapy. 2009 April; 153(4):179-89.

In one embodiment, the target cells from the subject are hepatocytes. In one embodiment, the cell is a liver sinusoidal endothelial cell (LSECs). Liver sinusoidal endothelial cells (LSEC) are specialized endothelial cells that play important roles in liver physiology and disease. Hepatocytes and liver sinusoidal endothelial cells (LSECs) are thought to contribute a substantial component of FVIII in circulation, with a variety of extra-hepatic endothelial cells supplementing the supply of FVIII.

In one embodiment, the present disclosure targets LSEC cells, as LSEC cells likely represent the main cell source of FVIII. Shahani, T, et al., Activation of human endothelial cells from specific vascular beds induces the release of a FVIII storage pool. Blood 2010; 115(23):4902-4909. In addition, LSECs are believed to play a role in induction of immune tolerance. Onoe, T, et al., Liver sinusoidal endothelial cells tolerize T cells across MHC barriers in mice. J Immunol 2005; 175(1):139-146. Methods of isolation of LSECs are known in the art. Karrar, A, et al., Human liver sinusoidal endothelial cells induce apoptosis in activated T cells: a role in tolerance induction. Gut. 2007 February; 56(2): 243-252.

In one embodiment, the transduced cells from the subject are stem cells. In one embodiment, the stem cells are induced pluripotent stem cells (iPSCs). Induced pluripotent stem cells (iPSCs) are a type of pluripotent stem cell artificially derived from a non-pluripotent cell, typically an adult somatic cell, by inducing expression of specific genes and factors important for maintaining the defining properties of embryonic stem cells. Induced pluripotent stem cells (iPSCs) have been shown in several examples to be capable of site specific gene targeting by nucleases. Ru, R. et al. Targeted genome engineering in human induced pluripotent stem cells by penetrating TALENs. Cell Regeneration. 2013, 2:5; Sun N, Zhao H. Seamless correction of the sickle cell disease mutation of the HBB gene in human induced pluripotent stem cells using TALENs. Biotechnol Bioeng. 2013 Aug. 8. Induced pluripotent stem cells (iPSCs) can be isolated using methods known in the art. Lorenzo, IM. Generation of Mouse and Human Induced Pluripotent Stem Cells (iPSC) from Primary Somatic Cells. Stem Cell Rev. 2013 August; 9(4):435-50.

As discussed above, a number of different cells types can be targeted for repair. However, in some cases, pure populations of some cell types may not promote sufficient homing and implantation upon reintroduction to provide extended and sufficient expression of the corrected F8 gene. Therefore, some cell types may be co-cultured with different cell types to help promote cell properties (i.e. ability of cells to engraft in the liver).

In one embodiment, the transduced cells are from blood outgrowth endothelial cells (BOECs) that have been co-cultured with additional cell types. In one embodiment, the transduced cells are from blood outgrowth endothelial cells (BOECs) that have been co-cultured with hepatocytes or liver sinusoidal endothelial cell (LESCs) or both. In one embodiment, the transduced cells are from blood outgrowth endothelial cells (BOECs) that have been co-cultured with induced pluripotent stem cells (iPSCs).

In embodiments of methods and systems herein described and related vehicles composition methods and systems, the polynucleotide encoding for the DNA-SE and repair vehicles RVs comprising the DNA donor can be delivered to the cells with methods of nucleic acid delivery well known in the art. (See, e.g., WO 2012051343). In the methods provided herein, the described nuclease encoding nucleic acids can be introduced into the cell as DNA or RNA, single-stranded or double-stranded and can be introduced into a cell in linear or circular form. In one embodiment, the nucleic acids encoding the nuclease are introduced into the cell as mRNA. The donor sequence can introduced into the cell as DNA single-stranded or double-stranded and can be introduced into a cell in linear or circular form. If introduced in linear form, the ends of the nucleic acids can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

The nucleic acids can be introduced into a cell as part of a vector molecule having additional sequences such as, for example, replication origins, promoters and genes encoding antibiotic resistance. Moreover, the nucleic acids can be introduced as naked nucleic acid, as nucleic acid complexed with an agent such as a liposome or poloxamer, or can be delivered by viruses (e.g., adenovirus, AAV, herpesvirus, retrovirus, lentivirus).

The nucleic acids can be delivered in vivo or ex vivo by any suitable means. Methods of delivering nucleic acids are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824.

Any vector systems can be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824. Furthermore, any of these vectors can comprise one or more of the sequences needed for treatment. Thus, when one or more nucleic acids are introduced into the cell, the nucleases and/or donor sequence nucleic acids can be carried on the same vector or on different vectors. When multiple vectors are used, each vector can comprise a sequence encoding a nuclease, a nickase, or a donor sequence nucleic acid. Alternatively, two or more of the nucleic acids can be contained on a single vector.

Conventional viral and non-viral based gene transfer methods can be used to introduce nucleic acids encoding the nucleic acids in cells (e.g., mammalian cells) and target tissues. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially {e.g., Transfectam™ and Lipofectin™). Cationic and neutral lipids that are suitable for efficient receptor-recognition lipofection of polynucleotides include those of Feigner, WO 91/17424, WO 91/16024.

The preparation of lipid:nucleic acid complexes, including targeted liposomes such as immunolipid complexes, is well known to one of skill in the art (see, e.g., Crystal, Science 270:404-410 (1995); Blaese et al, Cancer Gene Ther. 2:291-297 (1995); Behr et al, Bioconjugate Chem. 5:382-389 (1994); Remy et al, Bioconjugate Chem. 5:647-654 (1994); Gao et al, Gene Therapy 2:710-722 (1995); Ahmad et al, Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology 27(7):643).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cz's-acting long terminal repeats with packaging capacity for up to 6-10 kb of foreign sequence. The minimum cz's-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al, J. Virol. 66:2731-2739 (1992); Johann et al, J. Virol. 66:1635-1640 (1992); Sommerfelt et al., Virol. 176:58-59 (1990); Wilson et al, J. Virol. 63:2374-2378 (1989); Miller et al, J. Virol. 65:2220-2224 (1991); PCT US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al, Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al, Mol Cell. Biol. 5:3251-3260 (1985); Tratschin, et al, Mol. Cell. Biol. 4:2072-2081 (1984); Hermonat & Muzyczka, PNAS 81:6466-6470 (1984); and Samulski et al, J. Virol. 63:03822-3828 (1989).

At least six viral vector approaches are currently available for gene transfer in clinical trials, which utilize approaches that involve complementation of defective vectors by genes inserted into helper cell lines to generate the transducing agent. pLASN and MFG-S are examples of retroviral vectors that have been used in clinical trials (Dunbar et al, Blood 85:3048-305 (1995); Kohn et al, Nat. Med. 1:1017-102 (1995); Malech et al, PNAS 94:22 12133-12138 (1997)). PA317/pLASN was the first therapeutic vector used in a gene therapy trial. (Blaese et al, Science 270:475-480 (1995)). Transduction efficiencies of 50% or greater have been observed for MFG-S packaged vectors. (Ellem et al, Immunol Immunother. 44(1):10-20 (1997); Dranoff et al, Hum. Gene Ther. 1:111-2 (1997). Recombinant adeno-associated virus vectors (rAAV) are an alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al, Lancet 351:9117 1702-3 (1998), Kearns et al, Gene Ther. 9:748-55 (1996)). Other AAV serotypes, including AAV1, AAV2, AAV3, AAV4, AAV5, AAV6, AAV7, AAV8, AAV9 and AAVrh.lO and any novel AAV serotype can also be used in accordance with the present disclosure. In a particular embodiment, the vector is based on a hepatotropic adeno-associated virus vector, serotype 8 (see, e.g., Nathwani et al., Adeno-associated viral vector mediated gene transfer for hemophilia B, Blood 118(21):4-5, 2011).

Replication-deficient recombinant adenoviral vectors (Ad) can be produced at high titer and readily infect a number of different cell types. Most adenovirus vectors are engineered such that a transgene replaces the Ad E1 a, E1 b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al, Hum. Gene Ther. 7:1083-9 (1998)). Additional examples of the use of adenovirus vectors for gene transfer in clinical trials include Rosenecker et ah, Infection 24:1 5-10 (1996); Sterman et ah, Hum. Gene Ther. 9:7 1083-1089 (1998); Welsh et ah, Hum. Gene Ther. 2:205-18 (1995); Alvarez et al, Hum. Gene Ther. 5:597-613 (1997); Topf et al, Gene Ther. 5:507-513 (1998); Sterman et al, Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many applications, it is desirable that the g vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et ah, Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This can be used with other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Vectors (e.g., retroviruses, adenoviruses, liposomes, etc.) containing the nucleic acids described herein can also be administered directly to an organism for transduction of cells in vivo. Alternatively, naked DNA can be administered.

Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of the nucleic acids described herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985.

The nucleic acids encoding the monomers of the DNA scission enzymes can be expressed either on separate expression constructs or vectors, or can be linked in one open reading frame. Expression of the nuclease can be under the control of a constitutive promoter or an inducible promoter.

Administration can be by any means in which the polynucleotides are delivered to the desired target cells. For example, both in vivo and ex vivo methods are contemplated. In one embodiment, the nucleic acids are introduced into a subject's cells that have been explanted from the subject, and reintroduced following F8 gene repair.

For in vivo administration, for example, intravenous injection of the nucleic acids to the portal vein is a method of administration. Other in vivo administration modes include, for example, direct injection into the lobes of the liver or the biliary duct and intravenous injection distal to the liver, including through the hepatic artery, direct injection into the liver parenchyma, injection via the hepatic artery, and/or retrograde injection through the biliary tree. Ex vivo modes of administration include transduction in vitro of resected hepatocytes or other cells of the liver, followed by infusion of the transduced, resected hepatocytes back into the portal vasculature, liver parenchyma or biliary tree of the human patient, see e.g., Grossman et ah, (1994) Nature Genetics, 6:335-341.

If ex vivo methods are employed, cells or tissues can be removed and maintained outside the body according to standard protocols well known in the art. The compositions can be introduced into the cells via any gene transfer mechanism as described above, such as, for example, calcium phosphate mediated gene delivery, electroporation, microinjection, proteoliposomes, or viral vector delivery. The transduced cells can then be infused (e.g., in a pharmaceutically acceptable carrier) or homotopically transplanted back into the subject per standard methods for the cell or tissue type. Standard methods are known for transplantation or infusion of various cells into a subject.

In some embodiments, the one or more mutations cause hemophilia in the subject and the repair results in treatment of the hemophilia in the subject. The term “treatment” as used herein indicates any activity that is part of a medical care for, or deals with, a condition, medically or surgically.

The term “subject” as used herein is meant an individual and refers to a single biological organism such animals and in particular higher animals and in particular vertebrates such as mammals and in particular human beings. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Thus, veterinary uses and medical formulations are contemplated herein. In some embodiments, the subject is a mammal such as a primate, for example, a human.

The term “haemophilia” indicates a group of hereditary genetic disorders that impair the body's ability to control blood clotting, which is used to stop bleeding when a blood vessel is broken.

Haemophilia A (HA) (clotting factor VIII deficiency) is the most common form of the disorder, present in about 1 in 5,000-10,000 male births and is caused by loss-of-function mutations in the X-linked Factor (F) VIII gene. Haemophilia B (HB) (factor IX deficiency) occurs in around 1 in about 20,000-34,000 newborn male births.

The levels of functional FVIII in circulation determine the severity of the disease, with plasma levels 5-25% of normal being mild, 1-5% being moderate, and <1% being severe (Brettler et al., Clinical aspects of and therapy for hemophilia A. Churchill Livingstone, New York, N.Y. 1995; pp. 1648-63). As such, only a small amount of circulating protein is necessary to provide protection from spontaneous bleeding episodes.

The I22I-mutation of the F8 accounts for ˜45% of severe HA and is caused by an intra-chromosomal recombination within the gene. FIG. 1 shows a schematic illustration of the wild-type and I22I F8 loci (F8 & F8I22I). Indicated in FIG. 1 are the exon-1B (E1B) and exon-1 to exon-22 (E1-E22) functional coding sequences as well as the exons-23C (E23C), -24C (E24C), and exon-23 (E23C), exon-24C (E24C) and exon-23 (E23) to exon-26 (E26) non-functional coding sequences. Transcription from the F8 promoter of both the F8 (wild-type) & F8I22I loci, which is normally functioning in both forms, yields polyadenylated mRNAs. The F8 (wild-type) mRNA has 26 exons, exon-1 (E1) to exon-22 (E22) and exon-23 (E23) to exon-26 (E26), all of which encode the amino acids found in the FVIII. Conversely, the F8I22I mRNA has at least 24 exons, E1-E22 (they are the same in F8 and thus encode FVIII amino acid sequence), and E23C & E24C (they are cryptic and encode no FVIII amino acid sequence). The sequence of intron-22, in both F8 & F8I22I, contains a bi-directional promoter that transcribes two additional mRNAs from the two genes: F8A, which is oriented oppositely to that of F8 & F8I22I and contains a single exon (box designated E1A), and F8B, which contains five exons that are oriented similarly transcriptionally to that of F8 & F8I22I and contains a single non-F8 first exon within I22 (box designated E1B) followed by four additional exons, which are identical to E23-E26 of F8. The F8A mRNA encodes the FVIIIA protein, which is now known as HAP40 (a cytoskeleton-interacting protein involved in endocytosis and thus functionally unrelated to the coagulation system) and has no FVIII amino acid sequence. The F8B mRNA encodes FVIII B, a protein with unknown function that has 8 non-FVIII amino acid residues at its N-terminus followed by 208 residues that represent FVIII residues 2125-2332.

Infusion of replacement plasma-derived (pd) or recombinant (r) FVIII is the standard of care to manage this chronic disease. Currently available rFVIII replacement products include the commercially available Kogenate® (Bayer) and Helixate® (ZLB Behring), Recombinate® (Baxter) and Advate® (Baxter), and the B-domain deleted Refacto® (Pfizer) and Xyntha® (Pfizer). Patients unable to be treated with FVIII experience more painful, joint bleeding and over time, a greater loss of mobility than patients whose HA is able to be managed with FVIII. Infusion of replacement FVIII, however, is not a cure for HA. Spontaneous bleeding remains a serious problem especially for those with severe HA, defined as circulating levels of FVIII coagulant activity (FVIII: C) below 1% of normal. Furthermore, the formation of anti-FVIII antibodies occurs in about 20% of all patients and more often in certain subpopulations of HA patients, such as African Americans (Viel K R, Ameri A, Abshire T C, et al. Inhibitors of factor VIII in black patients with hemophilia. N Engl J Med. 360: 1618-27, 2009). There is therefore also a critical need to identify ways to avoid FVIII inhibitor development and to abate a FVIII inhibitor response.

In some embodiments herein described, the methods and compositions described herein are directed to treating a subject with hemophilia and in particular hemophilia A comprising selectively targeting and replacing a portion of the subject's genomic F8 gene sequence containing a mutation in the gene with a partial F8 cDNA replacement sequence (cDNA-RS). In one embodiment, the resultant repaired F8 gene containing the cDNA-RS, upon expression, produces functional FVIII that confers improved coagulation functionality to the encoded FVIII protein of the subject. The levels of functional FVIII in circulation are believed to obviate or reduce the need for infusions of replacement FVIII in the subject. In one embodiment, expression of functional FVIII reduces whole blood clotting time (WBCT). In one embodiment, the repaired F8 gene, upon expression, provides for the immune tolerance induction (ITI) to an administered replacement FVIII protein product. In one embodiment, the subject is a human.

In one aspect, a method of treating hemophilia A in a subject is provided comprising introducing into a cell of the subject one or more repair vehicles (RV) containing at least a cDNA-RS and one or more plasmids encoding a DNA scission enzyme (DNA-SE) such as a nuclease or nickase. The DNA-SE targets a portion of the F8 gene containing a mutation that causes hemophilia A and creates a first break in one strand of the F8 gene and a second break in the other strand of the F8 gene for subsequent repair by the cDNA-RS. In some embodiments, the first break and the second break are a double-stranded DNA break. In other embodiments, the first break and the second break are off-set paired and complementary single-stranded DNA nicks. The cDNA-RS comprises (i) a nucleic acid encoding a truncated FVIII polypeptide or (ii) a native F8 3′ splice acceptor site operably linked to a nucleic acid encoding a truncated FVIII polypeptide. The RV further comprises flanking sequences comprising an upstream flanking sequence (uFS) that is homologous to the nucleic acid sequences upstream of the first break in the DNA of the subject's F8 gene and a downstream flanking sequence (dFS) that is homologous to the nucleic acid sequences downstream of the second break in the DNA of the subject's F8 gene. The 5′ end of the cDNA-RS is flanked by the uFS and the 3′ end of the cDNA-RS is flanked by dFS to form a donor sequence that is a portion of the RV. After insertion of the cDNA-RS through homologous recombination into the subject's F8 gene (sF8), a repaired F8 gene (rF8) is formed, which upon expression forms functional FVIII that confers improved coagulation functionality to the FVIII protein encoded by the sF8 without the repair.

In one aspect, methods and systems for repairing F8 gene can be used to induce immune tolerance to a FVIII replacement product (FVIIIrp) such as a recombinant FVIII (rFVIII) or a plasma derived FVIII (pdFVIII) in a subject having a FVIII deficiency and who will be administered, is being administered, or has been administered a replacement FVIII product is disclosed. The method comprises introducing into cells of the subject one or more RVs encoding a cDNA-RS and one or more plasmids encoding a DNA-SE. The DNA-SE targets a portion of the F8 gene containing a mutation that causes hemophilia A and creates a first break in one strand of the F8 gene and a second break in the other strand of the F8 gene for subsequent repair by the cDNA-RS. In some embodiments, the first break and the second break are a double-stranded DNA break. In other embodiments, the first break and the second break are off-set paired and complementary single-stranded DNA nicks. The cDNA-RS comprises (i) a nucleic acid encoding a truncated FVIII polypeptide or (ii) a native F8 3′ splice acceptor site operably linked to a nucleic acid encoding a truncated FVIII polypeptide. The RV further comprises flanking sequences comprising an upstream flanking sequence (uFS) that is homologous to the nucleic acid sequences upstream of the first break in the DNA of the subject's F8 gene and a downstream flanking sequence (dFS) that is homologous to the nucleic acid sequences downstream of the second break in the DNA of the subject's F8 gene. The 5′ end of the cDNA-RS is flanked by the uFS and the 3′ end of the cNDA-RS is flanked by dFS to form a donor sequence that is a portion of the RV. After insertion of the cDNA-RS through homologous recombination into the subject's F8 gene (sF8), a repaired F8 gene (rF8) is formed, which upon expression forms functional FVIII that provides immune tolerance induction (ITI) to an administered replacement FVIII protein product. In some cases, the person administered the cells may have no anti-FVIII antibodies or have anti-FVIII antibodies as detected by ELISA or Bethesda assays. In one embodiment, the truncated FVIII polypeptide amino acid sequence shares homology with a portion of the FVIIIrp's amino acid sequence. In one embodiment, the truncated FVIII polypeptide amino acid sequence shares homology with a similar portion of the FVIIIrp's amino acid sequence. In one embodiment, the truncated FVIII polypeptide amino acid sequence shares complete homology with a similar portion of the FVIIIrp's amino acid sequence.

In some embodiments, the repaired version of the Factor VIII non-functional coding sequence comprises Factor VIII exons of a replacement FVIII protein product and the repair results in inducing immune tolerance to the FVIII replacement product.

In some embodiments disclosed herein, the cDNA, polynucleotides repair vehicles plasmids and vehicles herein described are provided as a part of systems to repair F8 gene in a subject. The systems can be provided in the form of a kits of part. In a kit of parts, the cDNA, polynucleotides repair vehicles plasmids and vehicles herein described and other reagents to repair one or more mutations of the F8 gene can be comprised in the kit independently. The cDNA, polynucleotides repair vehicles plasmids and vehicles herein described can be included in one or more compositions, and each capture agent can be in a composition together with a suitable excipient.

In some embodiments, additional components of the system include reagents, antibodies and enzymes that can be used to verify proper integration and expression of the cDNA-RS. Proper integration can be assessed through a variety of means that would be apparent to one of ordinary skill in the art, including DNA sequencing by Sanger technique or by next-generation sequencing techniques of the desired genomic DNA site of cDNA-RS integration to ensure proper integration of the donor sequence. Expression of a repaired FVIII can be assessed through a variety of means that would be apparent to one of ordinary skill in the art including using ELISA assays to measure repaired FVIII expression both intracellularly expressed and secreted into the medium and commercially-available coagulation and FVIII assays for measuring coagulation activity.

In particular, in some embodiments components of the kit are provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (e.g. Chromogenix Coamatic Factor VIII kit, available from Diapharma (http://www.diapharrna.com/asp/productdetails.asp?ID100080) can be used for measuring FVIII activity).

In some embodiments, the cDNA, polynucleotides repair vehicles plasmids and vehicles herein described herein described can be included in pharmaceutical compositions together with an excipient or diluent. In particular, in some embodiments, disclosed are pharmaceutical compositions which contain at least one cDNA, polynucleotides repair vehicles plasmids and vehicles herein described in combination with one or more compatible and pharmaceutically acceptable excipients, and in particular with pharmaceutically acceptable diluents or excipients. In those pharmaceutical compositions the multi-ligand capture agent can be administered as an active ingredient for treatment or prevention of a condition in an individual.

The term “excipient” as used herein indicates an inactive substance used as a carrier for the active ingredients of a medication. Suitable excipients for the pharmaceutical compositions herein described include any substance that enhances the ability of the body of an individual to absorb the multi-ligand capture agents or combinations thereof. Suitable excipients also include any substance that can be used to bulk up formulations with the peptides or combinations thereof, to allow for convenient and accurate dosage. In addition to their use in the single-dosage quantity, excipients can be used in the manufacturing process to aid in the handling of the peptides or combinations thereof concerned. Depending on the route of administration, and form of medication, different excipients can be used. Exemplary excipients include, but are not limited to, antiadherents, binders, coatings, disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation.

Further details concerning the identification of the suitable carrier agent or auxiliary agent of the compositions, and generally manufacturing and packaging of the kit, can be identified by the person skilled in the art upon reading of the present disclosure.

### EXAMPLES

The methods and system herein disclosed are further illustrated in the following examples, which are provided by way of illustration and are not intended to be limiting.

In particular, the following examples illustrate exemplary embodiments in accordance with exemplary procedures in accordance to the present disclosure. A person skilled in the art will appreciate the applicability of the features described in detail for the exemplified embodiments to different methods, different applications and different reaction conditions and reagents in accordance with the present disclosure.

### Example 1

### Ex Vivo Gene Repair

Examples are provided of an ex vivo gene repair strategies that can be performed without the use of viral vectors. Genetic materials are delivered to restore secretion of a wild-type full-length FVIII to lymphoblastoid cells derived from a human HA patient with the F8_{I22I}, using electroporation and TALENs. A similar strategy can be used as an example to repair the naturally-occurring I22I-mutation in cells from an animal model of HA (dogs of the HA canine colony located at the University of North Carolina in Chapel Hill). Canine (adipose) tissue, which can be induced to acquire many properties of hepatocytes, can be used.

Use of autologous cells is an attractive therapy for several reasons as levels of blood clotting proteins needed to maintain hemostasis may be more readily produced by expansion of large populations of cells ex vivo and reintroduction into the patient. Repair of the F8I22I gene residing in a B-lymphoblastoid cell-line derived from a patient with severe HA caused by the I22I-mutation is effected by using electroporation to deliver (i) two distinct mRNAs encoding a highly specific heterodimeric TALEN that targets a single human genome site located in F8 near the 5′-end of I22 and (ii) the corresponding donor plasmid that carries the “editing cassette”, which is comprised of a functional 3′-intron splice site ligated immediately 5′ of a partial F8 cDNA matched in sequence with the wild-type sequence of exons 23-26 in the patient's own F8I22I locus, flanked by “left” and “right” homology arms.

The use of viral-free methods to derive autologous cells of various phenotypes and to stably introduce genetic information into the genome is attractive. These methods can be effectively used to successfully “repair” the F8I22I, which arises through a highly-recurrent mutational event essentially restricted to the male germ-line. This same F8 abnormality, which is widely known as the I22I-mutation, occurs naturally in dogs, and results in spontaneous bleeding. Two large colonies of HA dogs have been established, one at the University of North Carolina in Chapel Hill. Investigation of F8I22I at the molecular genetic, biochemical, and cellular levels to characterize its expression products have been studied in order to determine the immune response to replacement FVIII. Extensive sequencing efforts and analyses of the F8I22I and its mRNA transcripts allow for an innovative gene repair strategy that exploits nuclease technology, for example, transcription activator-like effector TALEN technology to repair the I22I-mutation.

Lymphoblastoid cells derived from HA patient with the I22I-mutation is obtained. The left (TALEN-L) and right (TALEN-R) monomers comprising the heterodimeric TALEN is shown in FIG. 3, which was specifically designed to cleave within the human F8 I22-sequence, ˜1 kb downstream of the 3′-end of exon-22. In alternative embodiments, the TALENs target sequences throughout the FVIII gene, with replacement of the corresponding FV8 gene sequence on the donor sequence.

An example of a sequence that can be targeted includes a sequence within intron 22

(SEQ. ID No. 1)

(tactatgggatgagttgcagatggcaagtaagacactggggagatta
aat),

where the underlined regions of sequence are recognized by the left TAL Effector DNA-binding domain and the right TAL Effector DNA-binding domain). Another example of a sequence that can be targeted includes a sequence at the junction of exon 22 with intron 22

(SEQ. ID No. 2)

(tggaaccttaatggtatgtaattagtcatttaaagggaatgcctga
ata),

where the underlined regions of sequence are recognized by the left TAL Effector DNA-binding domain and the right TAL Effector DNA-binding domain). Another example of a sequence that can be targeted within intron 22 is depicted in FIG. 3

(SEQ. ID No. 3)

(ttagtattatagtttctcagattatcaccagtgatactatggga),

where the underlined regions of sequence are recognized by the left TAL Effector DNA-binding domain and the right TAL Effector DNA-binding domain). The two TALEN expression plasmids that target these sequences (or the mRNA) are co-transfected with the donor plasmid. The donor plasmid contains flanking homology regions to the intron 22 locus, which allows for recombination of the donor plasmid into the chromosome. The cDNA of exons 23 to 26 of the F8 gene is contained between the flanking homology regions of the donor plasmid. The donor plasmid can also contain a suicide gene (such as the thymidine kinase gene from the herpes simplex virus), which allows counter-selection to avoid random and multi-copy integration into the genome.

Electroporation (AMAXA Nucleofection system) and chemical transfection (with a commercial reagent optimized to this cell type) can be used as transfection methods for the lymphoblastoid cells. A plasmid containing the green fluorescent protein (GFP) gene is introduced into the cells using both methods. The cells are analyzed by fluorescent microscopy to obtain an estimate of transfection efficiency, and the cells are observed by ordinary light microscopy to determine the health of the transfected cells. Any transfection method that gives a desirable balance of high transfection efficiency and preservation of cell health in the lymphoblastoid cells can be used. The TALEN mRNAs and the gene repair donor plasmid is then introduced into the lymphoblastoid cells using a transfection method. The TALENs for the human lymphoblastoid cells and their target site are shown in FIG. 3.

Repair of the F8I22I in the adipose tissue-derived hepatocyte-like cells from the I22I HA canine animal model is effected using electroporation to deliver mRNAs encoding an analogous TALEN that targets the 5′-end of I22 in canine F8 and an analogous donor plasmid carrying a “splice-able” cDNA spanning canine F8 exons 23-26.

Adipose tissue is collected from these FVIII deficient dogs by standard liposuction. Stromal cells from the adipose tissue are reprogrammed into induced pluripotent stem cells (iPSC), as described by Sun et al. (“Feeder-free derivation of induced pluripotent stem cells from adult human adipose stem cells” Proc Natl Acad Sci USA. 106: 720-5, 2009) with two modifications: (i) mRNA of the reprogramming factors are used in place of lentiviral vectors and (ii) the reprogramming is performed under conditions of hypoxia, 5% 02, and in the presence of small molecules that have been found to increase the reprogramming efficiency. Once produced and characterized, pluripotent canine cells are obtained.

The defective FVIII sequence in iPSC is replaced by the correct sequence using site-specific TALE nucleases (see FIG. 4). The iPSC with repaired Factor VIII are differentiated into hepatocytes using well established protocols (see, for example, Hay et al. “Direct differentiation of human embryonic stem cells to hepatocyte-like cells exhibiting functional activities” Cloning Stem Cells. 9: 51-62, 2007; Si-Tayeb et al. “Highly efficient generation of human hepatocyte-like cells from induced pluripotent stem cells” Hepatology. 51: 297-305, 2010; and Cayo et al. “JD induced pluripotent stem cell-derived hepatocytes faithfully recapitulate the pathophysiology of familial hypercholesterolemia” Hepatology. May 31, 2012). In short, small colonies of iPSC are induced to differentiate for the first 3 days into definitive endoderm by treatment with 50 ng/mL Wnt3a and 100 ng/mL Activin A, and then into the hepatocyte lineage by 20 ng/mL BMP4. Two expression plasmids necessary to produce mRNAs encoding a functional TALEN are obtained. These are designed to cleave and yield a double-stranded DNA break at only a single site within the canine genome, located within canine F8 I22, ˜0.3 kb downstream of the 3′-end of exon-22. The left (TALEN-L) and right (TALEN-R) monomers comprising this heterodimeric TALEN is shown above in FIG. 4.

A donor plasmid containing the sequence of the 3′-end of canine F8 intron-22 and all of canine F8 exon-22 as the left homologous sequence and the 5′-end of canine F8 intron-23 as the right homologous sequence to provide an adequate length of genomic DNA for efficient homologous recombination at the target site (i.e., the TALEN cut site) is created. The TALEN mRNAs and the gene repair donor plasmid are introduced into the pluripotent canine cells using a transfection method described herein.

Likewise, in humans, human iPSCs are electroporated with the human F8 TALENs & donor plasmid described above, to assess candidate genome-editing tools (which were designed to be equally capable of “editing” the I22-sequence in the wild-type and I22-inverted F8 loci, F8 and F8I22I, respectively) for their efficiency of site-specific gene repair. The genomic DNA at the repaired F8 loci, as well as the mRNAs and expression products synthesized by, the cells described above are assessed before and after electroporation.

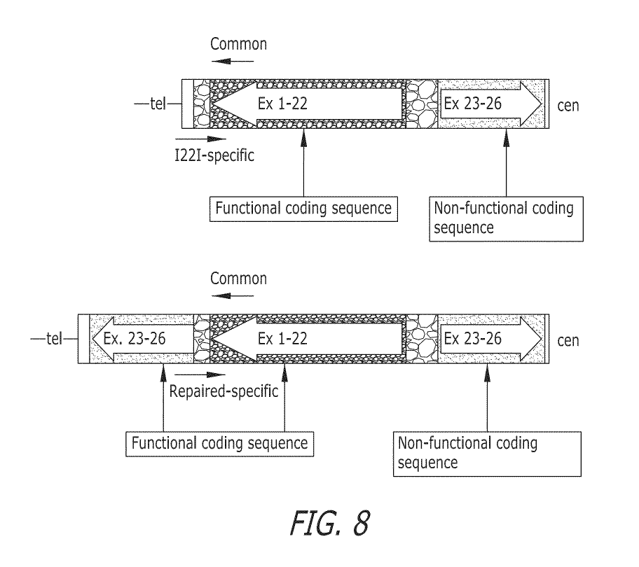

The TALEN gene repair method described above inserts F8 exons 23-26 immediately downstream (telomeric) to F8 exons 1-22 to encode a FVIII protein. Genomic DNA, spliced mRNA, and protein sequences differ among normal, repaired, and unrepaired cells (see FIG. 5). Gene repair is verified in genomic DNA through the use of PCR. Specific PCR primers are designed to amplify across the homologous recombination target sequence in unrepaired and repaired cells. A common primer is placed toward the end of exon-22. An I22I-specific primer is placed in the sequence telomeric to exon-22 in the I22I-inverted cells. A Repaired-specific primer is placed in the inserted exon 23-26 sequence. Primer design is shown in FIG. 8. In FIG. 8, Exons 1-22 (top schematic) and Exons 1-22 and 23-26 (left, bottom schematic) represent functional coding sequences, while Exons 23-26 (top schematic) and Exons 23-26 (right, bottom schematic) represent non-functional coding sequences. Separate sets of primers are designed for human and canine sequences.

Characterization of the genomic DNA at the repaired F8 loci, as well as the mRNAs and expression products synthesized by, the cells described above, before and after electroporation are performed.

A quantitative RT-PCR test that specifically detects and quantifies the mRNA transcripts from normal and I22I cells is used. The quantitative RT-PCR test uses three separate primer sets: one set to detect exons 1-22, one set to detect exons 23-26, and one set that spans the exon-22/exon-23 junction. mRNA is purified from cells before and after transfection. The existing primer design to probe mRNA from the human cells is used. Primers against canine sequences are designed using the same strategy and then the mRNA from the canine cells is probed using these new primers. An increased signal from the exon-22/exon-23 junction reaction in repaired cells, relative to unrepaired cells should be observed.

Monoclonal antibody ESH8, which is specific for the C2-domain of the FVIII protein, is be used. NIH3T3 cells were transfected with expression constructs encoding full-length and I22I F8 genes and then assayed by flow cytometry. Signal from the ESH8 antibody was high in cells transfected with the full-length construct but virtually absent in cells transfected with the I22I construct. The ESH8 antibody is used to test transfected cells. There should be an increased signal in repaired cells relative to unrepaired cells. Secreted FVIII levels, as measured by ELISA, are dramatically lower in I22I cells relative to normal cells. Whole-cell lysates and supernates from transfected cells are obtained and tested for FVIII concentration by ELISA. There should be an increase in FVIII concentration in the supernates from repaired cells relative to unrepaired cells.

In another example, canine blood outgrowth endothelial cells (cBOECs) and canine iPSCs derived from canine adipose tissue can be transfected with TALENs that target the F8I22I canine gene and a plasmid repair vehicle that carries exons 23-26 of cF8. TALENs are expected to make DSBs in the F8I22I DNA at the target site to allow “homologous recombination and repair” of the canine F8 I22I gene by insertion of exons 23-26 of the canine F8. The TALENS are designed to cleave and yield a DSB at only a single site within the canine genome, located within canine F8 I22, (˜0.3 kb) downstream of the 3′-end of exon-22. The donor plasmid contains the sequence of canine F8 exons 23-26 flanked by the 3′-end of canine F8 intron-22 and all of canine F8 exon-22 as the left homologous sequence and the 5′-end of canine F8 intron-23 as the right homologous sequence to provide an adequate length of genomic DNA for efficient homologous recombination at the target site.

Feasibility of deriving canine iPSCs is well established. An mRNA transcript that enables expression of the so called “Yamanaka” genes coding for transcription factors OCT4, SOX2, KLF4 and C-MYC to induce iPSCs from canine adipose derived stem cells (hADSCs). iPSCs have been transfected using Nucleofector. For transfection, Qiagen's Polyfect transfection reagents can be used with TALENs for many cell types, including BOECs. Transfection methods can be assessed using commercial reagents and transfected cells can be analyzed by fluorescent microscopy to obtain an estimate of transfection efficiency, while viability can be determined by Trypan Blue dye exclusion. The transfection method that gives the best balance of high transfection efficiency and preservation of cell health can be used.

Prior to commencing transfection with the TALENS and repair plasmid, the cleavage activity of the TALENs against the target site can be analyzed. This can be done by monitoring TALEN induced mutagenesis (Non-Homologous End Joining Repair) via a T7 Endonuclease assay. To assess potential risk of unintended genomic modification induced by the selected repair method, off-site activity is analyzed following transfection. In silico identification based on homologous regions within the genome can be used to identify the top 20 alternative target sites containing up to two mismatches per target half-site. PCR primers can be synthesized for the top 20 alternative sites and Surveyor Nuclease (Cel-I) assays (Transgenomics, Inc.) can be performed for each potential off-target site.

Transfection for expression and secretion of FVIII can be assessed in the various cell types before and after transfection. Genomic DNA is isolated from cells before and after transfection. Purified genomic DNA is used as template for PCR. Primers are designed for amplification from a FVIII I22I-specific primer only in unrepaired cells, and amplification from the repaired-specific primer only in repaired cells. RT-PCR can specifically detect and quantify the mRNA hF8 transcripts from normal and I22I cells. The quantitative RT-PCR test uses three separate primer sets: one set to detect exons 1-22, one set to detect exons 23-26, and one set that spans the exon-22/exon-23 junction. mRNA is purified from cells before and after transfection, with an increased signal from the exon-22/exon-23 junction reaction in repaired cells, relative to unrepaired cells. Flow-cytometry based assays may also be used for FVIII protein in peripheral blood mononuclear cells (PBMCs).

iPSCs derived from canine adipose tissue engineered can be conditioned to secrete FVIII to hepatocyte-like tissue. Canine iPSCs are conditioned toward hepatocyte like cells using a three step protocol as described by Chen et al. that incorporates hepatocyte growth factor (HGF) in the endodermal induction step (Chen Y F, Tseng C Y, Wang H W, Kuo H C, Yang V W, Lee O K. Rapid generation of mature hepatocyte-like cells from human induced pluripotent stem cells by an efficient three-step protocol. Hepatology. 2012 April; 55(4):1193-203).

Subpopulations of cBOECs are segregated and expanded and then characterized for the expression of endothelial markers, such as Matrix Metalloproteinases (MMPs), and cell-adhesion molecules (JAM-B, JAM-C, Claudin 3, and Claudin 5) using RT-PCR. Detailed RT-PCR methods, including primers for detecting expression of mRNA transcripts of the cell-adhesion molecules of interest and detailed immunohistochemistry methods to detect the proteins of interest, including a list of high affinity antibodies have been published by Geraud et al. (Geraud C, et al. Unique cell type-specific junctional complexes in vascular endothelium of human and rat liver sinusoids. PLoS One. 2012; 7(4):e34206). Antibodies that detect JAM-B, JAM-C, Claudin 3, and Claudin 5 may be purchased from LifeSpan Biosciences (www.lsbio.com).

One subpopulation of co-cultured cBOECs can be prepared and segregated early (before ˜4 passages of outgrowth). Later segregation of the subpopulation can occur after ˜10 passages. After 1 week of co-culture, two cBOECs subpopulations can be compared for expression and secretion of FVIII, and suitability for engraftment in the canine liver. Co-culturing of hepatocytes can be done with several cell types including human umbilical vein endothelial cells (HUVECs). cBOECs can be used as surrogates for HUVECS in this system. Once the repaired cBOECs (with the repaired FVIII gene) are obtained, the cells can be used to induce immune tolerance in canines with high titer-antibodies to FVIII.

### Example 2

### Protocol for Factor VIII Gene Repair in Humans

### Obtaining a Blood Sample

A protocol for gene repair of the F8 gene in blood outgrowth endothelial cells (BOECs) is described in the following example. First, a blood sample is obtained, with 50-100 mL of patient blood samples obtained by venipuncture and collection into commercially-available, medical-grade collecting devices that contain anticoagulants reagents, following standard medical guidelines for phlebotomy. Anticoagulant reagents that are used include heparin, sodium citrate, and/or ethylenediaminetetraacetic acid (EDTA). Following blood collection, all steps proceed with standard clinical practices for aseptic technique.

Isolating Appropriate Cell Populations from Blood Sample

Procedures for isolating and growing blood outgrowth endothelial cells (BOECs) have been described in detail by Hebbel and colleagues (Lin, Y., Weisdorf, D. J., Solovey, A. & Hebbel, R. P. Origins of circulating endothelial cells and endothelial outgrowth from blood. J Clin Invest 105, 71-77 (2000)). Peripheral blood mononuclear cells (PBMCs) are purified from whole blood samples by differential centrifugation using density media-based separation reagents. Examples of such separation reagents include Histopaque-1077, Ficoll-Paque, Ficoll-Hypaque, and Percoll. From these PBMCs multiple cell populations can be isolated, including BOECs. PBMCs are resuspended in EGM-2 medium without further cell subpopulation enrichment procedures and placed into 1 well of a 6-well plate coated with type I collagen. This mixture is incubated at 37° C. in a humidified environment with 5% CO2. Culture medium is changed daily. After 24 hours, unattached cells and debris are removed by washing with medium. This procedure leaves about 20 attached endothelial cells plus 100-200 other mononuclear cells. These non-endothelial mononuclear cells die within the first 2-3 weeks of culture.

### Cell Culture for Growing Target Cell Population

BOECs cells are established in culture for 4 weeks with daily medium changes but with no passaging. The first passaging occurs at 4 weeks, after approximately a 100-fold expansion. In the next step, 0.025% trypsin is used for passaging cells and tissue culture plates coated with collagen-I as substrate. Following this initial 4-week establishment of the cells in culture, the BOECs are passaged again 4 days later (day 32) and 4 days after that (day 36), after which time the cells should number 1 million cells or more.

### In Vitro Gene Repair

In order to affect gene repair in BOECs, cells are transfected with 0.1-10 micrograms per million cells of each plasmid encoding left and right TALENs and 0.1-10 micrograms per million cells of the repair vehicle plasmid. Transfection is done by electroporation, liposome-mediated transfection, polycation-mediated transfection, commercially available proprietary reagents for transfection, or other transfection methods using standard protocols. Following transfection, BOECs are cultured as described above for three days.

### Selection of Gene-Repaired Clones

Using the method of limiting serial dilution, the BOECs are dispensed into clonal subcultures, and grown as described above. Cells are examined daily to determine which subcultures contain single clones. Upon growth of the subcultures to a density of >100 cells per subculture, the cells are trypsinized, re-suspended in medium, and a 1/10 volume of the cells is used for colony PCR. The remaining 9/10 of the cells are returned to culture. Using primers that detect productively repaired F8 genes, each 1/10 volume of colonies are screened by PCR for productive gene repair. Colonies that exhibit productive gene repair are further cultured to increase cell numbers. Using the top 20 predicted potential off-site targets of the TALENs, each of the colonies selected for further culturing is screened for possible deleterious off-site mutations. The colonies exhibiting the least number of off-site mutations are chosen for further culturing.

Preparation of Cells for Re-Introduction into Patients by Conditioning and/or Outgrowth

Prior to re-introducing the cells into patients, the BOECs are grown in culture to increase the cell numbers. In addition to continuing cell culture in the manner described above, other methods can be used to condition the cells to increase the likelihood of successful engraftment of the BOECs in the liver sinusoidal bed of the recipient patient. These other methods include: 1) co-culturing the BOECs in direct contact with hepatocytes, wherein the hepatocytes are either autologous patient-derived cells, or cells from another donor; 2) co-culturing the BOECs in conditioned medium taken from separate cultures of hepatocytes, wherein the hepatocytes that yield this conditioned medium are either autologous patient-derived cells, or cells from another donor; or 3) culturing the BOECs as spheroids in the absence of other cell types.

Co-culturing endothelial cells with hepatocytes is described further in the primary scientific literature (e.g. Kim, Y. & Rajagopalan, P. 3D hepatic cultures simultaneously maintain primary hepatocyte and liver sinusoidal endothelial cell phenotypes. PLoS ONE 5, e15456 (2010)). Culturing endothelial cells as spheroids is also described in the scientific literature (e.g. Korff, T. & Augustin, H. G. Tensional forces in fibrillar extracellular matrices control directional capillary sprouting. J Cell Sci 112 (Pt 19), 3249-3258 (1999)). Upon growing the colonies of cells to a total cell number of at least 1 billion cells, the number of cells needed for injection (>50 million cells) into the patient are separated from the remainder of the cells and used in the following step for injection into patients. The remainder of the cells are aliqouted and banked using standard cell banking procedures.

Injection of Gene-Repaired BOECs into Patients

BOECs that have been chosen for injection into patients are resuspended in sterile saline at a dose and concentration that is appropriate for the weight and age of the patient. Injection of the cell sample is performed in either the portal vein or other intravenous route of the patient, using standard clinical practices for intravenous injection.

### Example 3

### Nuclease Sites for Repair at Different Exon-Intron Junctions

Because mutations causing Hemophilia A occur throughout the FVIII gene, different repair strategies may be employed at different exon-intron junctions in order to allow the use of repair vehicles which correct a wider range of patient mutations. All gene repairs employ the methodology described herein of using a DNS scission enzyme (DNA-SE) such as a zinc finger nuclease, a TALEN, or a CRISPR to induce a double-strand break near the 3′ end of an exon, thereby allowing homologous recombination to incorporate a therapeutic repair vehicle encoding the cDNA for the downstream exons of the gene into the genome in order to be operably linked to the 3′ end of that exon.

In order to choose CRISPR target sites in exons 1-22, several considerations were taken into account. The ˜100 bp of the 3′ end of each exon (hg19 human genome build) were searched for CRISPR/Cas9 binding sites using an online algorithm described by Hsu et al. in Nature Biotechnology 2013, incorporated herein by reference. Single guide RNAs (sgRNAs) were chosen based on low potential for off-target activity, the proximity of the cleavage site to the 3′ end of the exon, and guidelines for increasing the likelihood of high on-target activity (Wang T et al., Science 2014). Paired nickases were chosen by adding the additional consideration that they be orientated to create 5′ overhangs and be spaced apart within the recommended range for optimal activity (Shen B, et al., Nature Methods 2014).

In order to choose TALEN binding sites in exons 1-22, several considerations were taken into account. The ˜100 bp of the 3′ end of each exon (hg19 human genome build) were searched for TALEN binding sites using the SAPTA algorithm as described by Lin Y, Fine E J, et al. in Nucleic Acids 2014, incorporated herein by reference. Potential binding sites were then screened using the TALEN v2.0 algorithm of the PROGNOS tool as described by Fine E J et al. in Nucleic Acids Research 2013, incorporated herein by reference to ensure that no highly scored potential off-target sites existed in the human genome.

Sequences listed in Table 5 below contain identified binding sites for CRISPRs within exons 1-22 respectively. If a homologous sequence in the canine genome (canFam3 build) exists that permits the possibility of CRISPR/Cas9 cleavage using the same guide strand as used for the human exon, it is listed with any mismatches in lowercase bold; if no reasonable homology exists, it is listed as “N/A”.

TABLE 5

| FVIII Gene | Genome Editing | Genomic Target of SG/PG RNAs | Target of SG/PG RNAs in Dogs |
|---|---|---|---|
| (Region) | (Desired Activity) | (DNA Sequence) | (DNA Sequence) |
| Exon 1 | single nuclease | 5′-AAGATACTACCTGGGTGCAGtGG | 5′-AAaATACTACCTcGGTGCAGtGG |
| | | (SEQ. ID. NO.: 20) | (SEQ. ID. NO.: 1659) |
| | paired nickase (5′) | 5′-CACTAAAGCAGAATCGCAAAaGG | N/A |
| | | (SEQ. ID. NO.: 21) | |
| | paired nickase (3′) | 5′-AAGATACTACCTGGGTGCAGtGG | N/A |
| | | (SEQ. ID. NO.: 22) | |
| Exon 2 | single nuclease | 5′-TTTTCAACATCGCTAAGCCAaGG | N/A |
| | | (SEQ. ID. NO.: 23) | |
| | paired nickase (5′) | 5′-AGTCTTTTTGTACACGACTGaGG | N/A |
| | | (SEQ. ID. NO.: 24) | |
| | paired nickase (3′) | 5′-TTTTCAACATCGCTAAGCCAaGG | N/A |
| | | (SEQ. ID. NO.: 25) | |
| Exon 3 | single nuclease | 5′-ATGCTGTTGGTGTATCCTACtGG | 5′-AcGCTGTTGGTGTATCCTAttGG |
| | | (SEQ. ID. NO.: 26) | (SEQ. ID. NO.: 567) |
| | paired nickase (5′) | 5′-CAGCATGAAGACTGACAGGAtGG | N/A |
| | | (SEQ. ID. NO.: 27) | |
| | paired nickase (3′) | 5′-ATGCTGTTGGTGTATCCTACtGG | N/A |
| | | (SEQ. ID. NO.: 28) | |
| Exon 4 | single nuclease | 5′-GACTTGAATTCAGGCCTCATtGG | 5′-GACcTGAATTCAGGCCTCATtGG |
| | | (SEQ. ID. NO.: 29) | (SEQ. ID. NO.: 568) |
| | paired nickase (5′) | 5′-TATGAGTAGGTAAGGCACAGtGG | N/A |
| | | (SEQ. ID. NO.: 30) | |
| | paired nickase (3′) | 5′-GACTTGAATTCAGGCCTCATtGG | N/A |
| | | (SEQ. ID. NO.: 31) | |
| Exon 5 | single nuclease | 5′-AAGTAGTATAAATTTGTGCAaGG | N/A |
| | | (SEQ. ID. NO.: 32) | |
| | paired nickase (5′) | 5′-AAGTAGTATAAATTTGTGCAaGG | N/A |
| | | (SEQ. ID. NO.: 33) | |
| | paired nickase (3′) | 5′-CTTTTTGCTGTATTTGATGAaGG | N/A |
| | | (SEQ. ID. NO.: 34) | |
| Exon 6 | single nuclease | 5′-CAGTCAATGGTTATGTAAACaGG | 5′-CcaTCAATGGcTATGTAAACaGG |
| | | (SEQ. ID. NO.: 36) | (SEQ. ID. NO.: 87) |
| | paired nickase (5′) | 5′-GACTGTGTGCATTTTAGGCCaGG | N/A |
| | | (SEQ. ID. NO.: 37) | |
| | paired nickase (3′) | 5′-CAGTCAATGGTTATGTAAACaGG | N/A |
| | | (SEQ. ID. NO.: 38) | |
| Exon 7 | single nuclease | 5′-CAAACACTCTTGATGGACCTtGG | N/A |
| | | (SEQ. ID. NO.: 39) | |
| | paired nickase (5′) | 5′-GCGAGATTTCCAAGGACGCCtGG | N/A |
| | | (SEQ. ID. NO.: 40) | |
| | paired nickase (3′) | 5′-CAAACACTCTTGATGGACCTtGG | N/A |
| | | (SEQ. ID. NO.: 41) | |
| Exon 8 | single nuclease | 5′-ACATTACATTGCTGCTGAAGaGG | N/A |
| | | (SEQ. ID. NO.: 42) | |
| | paired nickase (5′) | 5′-TCTTGGCAACTGAGCGAATTtGG | N/A |
| | | (SEQ. ID. NO.: 43) | |
| | paired nickase (3′) | 5′-ACATTACATTGCTGCTGAAGaGG | N/A |
| | | (SEQ. ID. NO.: 44) | |
| Exon 9 | single nuclease | 5′-GAAGCTATTCAGCATGAATCaGG | 5′-GAAGCTATTCAGtATGAATCaGG |
| | | (SEQ. ID. NO.: 45) | (SEQ. ID. NO.: 88) |
| | paired nickase (5′) | 5′-AATAGCTTCACGAGTCTTAAaGG | N/A |
| | | (SEQ. ID. NO.: 46) | |
| | paired nickase (3′) | 5′-GAAGCTATTCAGCATGAATCaGG | N/A |
| | | (SEQ. ID. NO.: 47) | |
| Exon 10 | single nuclease | 5′-GGACATCAGTGATTCCGTGAgGG | N/A |
| | | (SEQ. ID. NO.: 48) | |
| | paired nickase (5′) | 5′-GGACATCAGTGATTCCGTGAgGG | N/A |
| | | (SEQ. ID. NO.: 49) | |
| | paired nickase (3′) | 5′-ATGTCCGTCCTTTGTATTCAaGG | N/A |
| | | (SEQ. ID. NO.: 50) | |
| Exon 11 | single nuclease | 5′-GATCTAGCTTCAGGACTCATtGG | 5′-GATCTAGCTTCAGGACTCATtGG |
| | | (SEQ. ID. NO.: 51) | (SEQ. ID. NO.: 89) |
| | paired nickase (5′) | 5′-AACGAAACTAGAGTAATAGCgGG | N/A |
| | | (SEQ. ID. NO.: 52) | |
| | paired nickase (3′) | 5′-GATCTAGCTTCAGGACTCATtGG | N/A |
| | | (SEQ. ID. NO.: 53) | |
| Exon 12 | single nuclease | 5′-CGCTTTCTCCCCAATCCAGCtGG | N/A |
| | | (SEQ. ID. NO.: 54) | |
| | paired nickase (5′) | 5′-AGCGTTGTATATTCTCTGTGaGG | N/A |
| | | (SEQ. ID. NO.: 55) | |
| | paired nickase (3′) | 5′-CGCTTTCTCCCCAATCCAGCtGG | N/A |
| | | (SEQ. ID. NO.: 56) | |
| Exon 13 | single nuclease | 5′-AGAAACTGTCTTCATGTCGAtGG | 5′-AGAAACTGTCTTCATGTCaAtGG |
| | | (SEQ. ID. NO.: 57) | (SEQ. ID. NO.: 90) |
| | paired nickase (5′) | 5′-ATAGACCATTTTGTGTTTGAaGG | 5′-ATAGACCATTTTGTGTTTGAaGG |
| | | (SEQ. ID. NO.: 58) | (SEQ. ID. NO.: 91) |
| | paired nickase (3′) | 5′-AGAAACTGTCTTCATGTCGAtGG | 5′-AGAAACTGTCTTCATGTCaAtGG |
| | | (SEQ. ID. NO.: 59) | (SEQ. ID. NO.: 92) |
| Exon 14 | single nuclease | 5′-ACACTATTTTATTGCTGCAGtGG | 5′-ACACTATTTcATTGCTGCAGtGG |
| | | (SEQ. ID. NO.: 60) | (SEQ. ID. NO.: 93) |
| | paired nickase (5′) | 5′-TTTTCTTTTGAAAGCTGCGGgGG | 5′-TTTTCTTTTGAAAGCTGCGGaGG |
| | | (SEQ. ID. NO.: 61) | (SEQ. ID. NO.: 94) |
| | paired nickase (3′) | 5′-ACACTATTTTATTGCTGCAGtGG | 5′-ACACTATTTcATTGCTGCAGtGG |
| | | (SEQ. ID. NO.: 62) | (SEQ. ID. NO.: 95) |
| Exon 15 | single nuclease | 5′-TCAACTTCTGCTCTTATATAtGG | 5′-TCAACTTCTGCTCTTATATAtGG |
| | | (SEQ. ID. NO.: 63) | (SEQ. ID. NO.: 96) |
| | paired nickase (5′) | 5′-ACGGTATAAGGGCTGAGTAAaGG | N/A |
| | | (SEQ. ID. NO.: 64) | |
| | paired nickase (3′) | 5′-AAATGAACATTTGGGACTCCtGG | N/A |
| | | (SEQ. ID. NO.: 65) | |
| Exon 16 | single nuclease | 5′-ATGAGTTTGACTGCAAAGCCtGG | 5′-ATGAGTTTGACTGCAAAGCCtGG |
| | | (SEQ. ID. NO.: 66) | (SEQ. ID. NO.: 97) |
| | paired nickase (5′) | 5′-CAGTCAAACTCATCTTTAGTgGG | 5′-CAGTCAAACTCATCTTTAGTgGG |
| | | (SEQ. ID. NO.: 67) | (SEQ. ID. NO.: 98) |
| | paired nickase (3′) | 5′-ATGAGTTTGACTGCAAAGCCtGG | 5′-ATGAGTTTGACTGCAAAGCCtGG |
| | | (SEQ. ID. NO.: 68) | (SEQ. ID. NO.: 99) |
| Exon 17 | single nuclease | 5′-GGCTCCCTGCAATATCCAGAtGG | 5′-aGCTCCCTGCAATgTCCAGAaGG |
| | | (SEQ. ID. NO.: 69) | (SEQ. ID. NO.: 100) |
| | paired nickase (5′) | 5′-TTCAGTGAAGTACCAGCTTTtGG | N/A |
| | | (SEQ. ID. NO.: 70) | |
| | paired nickase (3′) | 5′-GGCTCCCTGCAATATCCAGAtGG | N/A |
| | | (SEQ. ID. NO.: 71) | |
| Exon 18 | single nuclease | 5′-GTTCACTGTACGAAAAAAAGaGG | 5′-GTTCACTGTACGAAAAAAAGaGG |
| | | (SEQ. ID. NO.: 72) | (SEQ. ID. NO.: 101) |
| | paired nickase (5′) | 5′-GTCCACTGAAATGAATAGAAtGG | N/A |
| | | (SEQ. ID. NO.: 73) | |
| | paired nickase (3′) | 5′-GTTCACTGTACGAAAAAAAGaGG | N/A |
| | | (SEQ. ID. NO.: 74) | |
| Exon 19 | single nuclease | 5′-CAAAGCTGGAATTTGGCGGGtGG | N/A |
| | | (SEQ. ID. NO.: 75) | |
| | paired nickase (5′) | 5′-CGCCAAATTCCAGCTTTGGAtGG | N/A |
| | | (SEQ. ID. NO.: 76) | |
| | paired nickase (3′) | 5′-ATTGGCGAGCATCTACATGCtGG | N/A |
| | | (SEQ. ID. NO.: 77) | |
| Exon 20 | single nuclease | 5′-TGTCCAGAAGCCATTCCCAGgGG | N/A |
| | | (SEQ. ID. NO.: 78) | |
| | paired nickase (5′) | 5′-TGTCCAGAAGCCATTCCCAGgGG | N/A |
| | | (SEQ. ID. NO.: 79) | |
| | paired nickase (3′) | 5′-GATTTTCAGATTACAGCTTCaGG | N/A |
| | | (SEQ. ID. NO.: 80) | |
| Exon 21 | single nuclease | 5′-AATCAATGCCTGGAGCACCAaGG | 5′-AATCAATGCCTGGAGCACCAaGG |
| | | (SEQ. ID. NO.: 81) | (SEQ. ID. NO.: 102) |
| | paired nickase (5′) | 5′-TGATCCGGAATAATGAAGTCtGG | 5′-TGATCCGGAATAATGAAGTCtGG |
| | | (SEQ. ID. NO.: 82) | (SEQ. ID. NO.: 103) |
| | paired nickase (3′) | 5′-AATCAATGCCTGGAGCACCAaGG | 5′-AATCAATGCCTGGAGCACCAaGG |
| | | (SEQ. ID. NO.: 83) | (SEQ. ID. NO.: 104) |
| Exon 22 | single nuclease | 5′-AAGAAGTGGCAGACTTATCGaGG | N/A |
| | | (SEQ. ID. NO.: 84) | |
| | paired nickase (5′) | 5′-AGATAAACTGAGAGATGTAGaGG | N/A |
| | | (SEQ. ID. NO.: 85) | |
| | paired nickase (3′) | 5′-AAGAAGTGGCAGACTTATCGaGG | N/A |
| | | (SEQ. ID. NO.: 86) | |

Sequences contain the top 20 potential off-target sites computationally identified in the human genome for the previously mentioned CRIPSR binding sites in exons 1-22 are listed in tables 6-27, respectively below.

Top-Ranked Potential Off-Target Sites for sgRNAs in Human Genome

The top twenty potential off-target sites in the human genome (hg19 genome build) for single guide strands were located using an online tool (Hsu et al., Nature Biotechnology 2013). Mismatches to the intended binding sequence are shown in bold. The genomic region is annotated and the gene name given in parentheses.

TABLE 6
Targeting Exon 1

| Genome Coordinates | Sequence | Genomic Region |
|---|---|---|
| chrX: 154250739 | AGATACTACCTGGGTGCAGtGG | Exon Coding Sequence (F8) |
| | (SEQ. ID. NO.: 105) | |
| chr5: 65751749 | AAACACAACCTGGGTGCAGgGG | Intergenic |
| | (SEQ. ID. NO.: 106) | |
| chr9: 17600130 | AAAAAGTACCTGGGTGCAGaAG | Intron (SH3GL2) |
| | (SEQ. ID. NO.: 107) | |
| chr9: 100168533 | AGAAACTACATGGGTGCAGaGG | Intergenic |
| | (SEQ. ID. NO.: 108) | |
| chr21: 45748293 | GGCGACCACCTGGGTGCAGcAG | Intergenic |
| | (SEQ. ID. NO.: 109) | |
| chr2: 144598347 | ATTTACCAACTGGGTGCAGcAG | Intergenic |
| | (SEQ. ID. NO.: 110) | |
| chr3: 89701232 | ATTTACCATCTGGGTGCAGgGG | Intergenic |
| | (SEQ. ID. NO.: 111) | |
| chr10: 43493946 | AGATGCTTCCTGGGTGCAGcAG | Intergenic |
| | (SEQ. ID. NO.: 112) | |
| chr18: 37552785 | ACAAACTCCCTGGGTGCAGaGG | Intergenic |
| | (SEQ. ID. NO.: 113) | |
| chr7: 63413239 | ACACACTGCCTGGGTGCAGcAG | Intergenic |
| | (SEQ. ID. NO.: 114) | |
| chr7: 157859920 | GGAGACACCCTGGGTGCAGgAG | Intron (PTPRN2) |
| | (SEQ. ID. NO.: 115) | |
| chr22: 48920664 | AGGAACGCCCTGGGTGCAGaAG | Intron (FAM19A5) |
| | (SEQ. ID. NO.: 116) | |
| chr1: 153919242 | GGAAGCTACCTGGGTGCAGgGG | Promoter (DENND4B) |
| | (SEQ. ID. NO.: 117) | |
| chr11: 71136741 | AGATACCCTCTGGGTGCAGaAG | Intergenic |
| | (SEQ. ID. NO.: 118) | |
| chr2: 145627680 | AGATACCCTCTGGGTGCAGgAG | Intron (TEX41) |
| | (SEQ. ID. NO.: 119) | |
| chr2: 145629372 | AGATACCCTCTGGGTGCAGgAG | Intron (TEX41) |
| | (SEQ. ID. NO.: 120) | |
| chr4: 60481509 | AGATACTGCCTGGGTCCAGaGG | Intergenic |
| | (SEQ. ID. NO.: 121) | |
| chr6: 35192631 | AGATACTCCCTGGGTCCAGcAG | Intron (SCUBE3) |
| | (SEQ. ID. NO.: 122) | |
| chr10: 132278858 | GGATACTAGATGGGTGCAGaGG | Intergenic |
| | (SEQ. ID. NO.: 123) | |
| chr3: 86928921 | AGAGACTACAAGGGTGCAGtGG | Intergenic |
| | (SEQ. ID. NO.: 124) | |
| chr5: 61074999 | CAACACTACCTGGGTGCAAaAG | Intergenic |
| | (SEQ. ID. NO.: 125) | |

TABLE 7
Targeting Exon 2

| Genome Coordinates | Sequence | Genomic Region |
|---|---|---|
| chrX: 154227766 | TTTCAACATCGCTAAGCCAaGG | Exon Coding Sequence (F8) |
| | (SEQ. ID. NO.: 126) | |
| chr2: 134436424 | GAACAACATCGCTAAGCCAcAG | Intergenic |
| | (SEQ. ID. NO.: 127) | |
| chr17: 5583238 | TTTCATCATGGCTAAGCCAaGG | Intergenic |
| | (SEQ. ID. NO.: 128) | |
| chr4: 160223598 | TTTTAACATCTCTAAGCCAtAG | Intron (RAPGEF2) |
| | (SEQ. ID. NO.: 129) | |
| chr3: 164824288 | GTCAAACAACGCTAAGCCAaAG | Intergenic |
| | (SEQ. ID. NO.: 130) | |
| chr2: 183724846 | CTTCAAAATAGCTAAGCCAaGG | Intron (FRZB) |
| | (SEQ. ID. NO.: 131) | |
| chr3: 73371080 | TTCAAACATGGCTAAGCCAtGG | Intergenic |
| | (SEQ. ID. NO.: 132) | |
| chr8: 140582153 | GCTCAAAATGGCTAAGCCAaGG | Intergenic |
| | (SEQ. ID. NO.: 133) | |
| chrX: 142729463 | TTAGAATATTGCTAAGCCAgGG | Intergenic |
| | (SEQ. ID. NO.: 134) | |
| chr4: 47492384 | TTTTAAGATCCCTAAGCCAaGG | Intron (ATP10D) |
| | (SEQ. ID. NO.: 135) | |
| chr3: 77774351 | TTGCAACAACTCTAAGCCAgGG | Intergenic |
| | (SEQ. ID. NO.: 136) | |
| chr9: 107554384 | TGTCAATAACCCTAAGCCAtAG | Intron Near Splice Site (ABCA1) |
| | (SEQ. ID. NO.: 137) | |
| chr1: 7294804 | TCCCAAGATCGTTAAGCCAcAG | Intron (CAMTA1) |
| | (SEQ. ID. NO.: 138) | |
| chr5: 134348045 | TTCCATCATGGCTAAGCCAgAG | Intergenic |
| | (SEQ. ID. NO.: 139) | |
| chr9: 104470724 | TTGTAGCATTGCTAAGCCAtAG | Intergenic |
| | (SEQ. ID. NO.: 140) | |
| chr18: 70959070 | TAACAAAATCGCTAAGCTAaAG | Intron (GRIN3A) |
| | (SEQ. ID. NO.: 141) | |
| chr20: 33501453 | TTTCAGGATCTCTAAGCCAgGG | Intron Near Splice Site (ACSS2) |
| | (SEQ. ID. NO.: 142) | |
| chr15: 55955035 | TTTCAAAGTAGCTAAGCCAgAG | Intron (PRTG) |
| | (SEQ. ID. NO.: 143) | |
| chr2: 42120954 | TGCCACCATCACTAAGCCAgGG | Non-Coding Exon (LOC388942) |
| | (SEQ. ID. NO.: 144) | |
| chr2: 110379573 | TCTAAACCTGGCTAAGCCAaAG | Intergenic |
| | (SEQ. ID. NO.: 145) | |
| chr3: 189222172 | TTTCAACATGGCTTAGCCAgAG | Intergenic |
| | (SEQ. ID. NO.: 146) | |

TABLE 8
Targeting Exon 3

| Genome Coordinates | Sequence | Genomic Region |
|---|---|---|
| chrX: 154225260 | TGCTGTTGGTGTATCCTACtGG | Exon Coding Sequence (F8) |
| | (SEQ. ID. NO.: 147) | |
| chr8: 101315002 | ACCTGTTGGTCTATCCTACtAG | Intron (RNF19A) |
| | (SEQ. ID. NO.: 148) | |
| chr6: 11986802 | TGATGTTGATGTATCCTAAgGG | Intergenic |
| | (SEQ. ID. NO.: 149) | |
| chr18: 7788999 | AGCTGTTATTGTATCCTACcAG | Intron (PTPRM) |
| | (SEQ. ID. NO.: 150) | |
| chr7: 142177112 | CACTGTTGGTGCATCCTACaGG | Intron (TCRBV5S1A1T) |
| | (SEQ. ID. NO.: 151) | |
| chr11: 64781733 | TGCTCATGCTGTATCCTACcGG | Exon Coding Sequence (ARL2) |
| | (SEQ. ID. NO.: 152) | |
| chr7: 142120643 | CGCTGTTGTTGCATCCTACaGG | Intron (TCRBV5S1A1T) |
| | (SEQ. ID. NO.: 153) | |
| chr1: 173455250 | AGCAGTTGGTGTATCCTTCtAG | Intron (PRDX6) |
| | (SEQ. ID. NO.: 154) | |
| chr4: 92829594 | TTCTGTTGATGTATACTACtGG | Intergenic |
| | (SEQ. ID. NO.: 155) | |
| chr3: 25922674 | GGATGTTGATGTATCCTGCcAG | Intergenic |
| | (SEQ. ID. NO.: 156) | |
| chr8: 52992366 | TACTATTTCTGTATCCTACcAG | Intergenic |
| | (SEQ. ID. NO.: 157) | |
| chr6: 22351191 | TGGTGTTTGTTTATCCTACtGG | Intergenic |
| | (SEQ. ID. NO.: 158) | |
| chr16: 68592830 | GGCTGTGGGTGTTTCCTACaAG | Intron (ZFP90) |
| | (SEQ. ID. NO.: 159) | |
| chrX: 34758178 | TACATTTGGTGTATCCTAAgGG | Intergenic |
| | (SEQ. ID. NO.: 160) | |
| chr11: 43130254 | TGTTGTTGGAATATCCTACcAG | Intergenic |
| | (SEQ. ID. NO.: 161) | |
| chr1: 158097934 | TGCTCTTGTTGTATCCTAGgAG | Intergenic |
| | (SEQ. ID. NO.: 162) | |
| chr1: 36401755 | GGCTGTTCATGTATCCTAAcAG | Intron (AGO3) |
| | (SEQ. ID. NO.: 163) | |
| chr11: 41965586 | GGCTGCTGCTGCATCCTACcAG | Intergenic |
| | (SEQ. ID. NO.: 164) | |
| chr8: 105459008 | TGCAGATGGTGTATCCTTCaGG | Intron (DPYS) |
| | (SEQ. ID. NO.: 165) | |
| chr6: 154040707 | TGTTGCTGGTGTATACTACtAG | Intergenic |
| | (SEQ. ID. NO.: 166) | |
| chr1: 66031489 | ACCTGATGGTGTATCCTTCcAG | Intron (LEPR) |
| | (SEQ. ID. NO.: 167) | |

TABLE 9
Targeting Exon 4

| Genome Coordinates | Sequence | Genomic Region |
|---|---|---|
| chrX: 154221233 | ACTTGAATTCAGGCCTCATtGG | Exon Coding Sequence (F8) |
| | (SEQ. ID. NO.: 168) | |
| chr8: 139299124 | ATTTGTGTTCAGGCCTCATtGG | Intron (FAM135B) |
| | (SEQ. ID. NO.: 169) | |
| chr18: 53517971 | TCTTGAAATCAGGCCTCATgGG | Intergenic |
| | (SEQ. ID. NO.: 170) | |
| chr2: 133881897 | ACTTGATTTCAGGCCTCTTcAG | Intron (NCKAP5) |
| | (SEQ. ID. NO.: 171) | |
| chr10: 67974828 | ACTTGATTTCAGTCCTCATtGG | Intron (CTNNA3) |
| | (SEQ. ID. NO.: 172) | |
| chr10: 111641509 | ACTGGAATCCAGGCCTCTTtAG | Intron (XPNPEP1) |
| | (SEQ. ID. NO.: 173) | |
| chr15: 70549506 | AATGGGTTTCAGGCCTCATgGG | Intergenic |
| | (SEQ. ID. NO.: 174) | |
| chr4: 78272534 | ATGTGAATTCTGGCCTCATtGG | Intergenic |
| | (SEQ. ID. NO.: 175) | |
| chr6: 438167 | ACTGGACTTCAGGCCTCACcAG | Intergenic |
| | (SEQ. ID. NO.: 176) | |
| chr5: 154546093 | ATTTGAATTCAGGCCTGATaGG | Intergenic |
| | (SEQ. ID. NO.: 177) | |
| chr1: 201395287 | ACCAGAATCCAGGCCTCAGgAG | Intron (TNNI1) |
| | (SEQ. ID. NO.: 178) | |
| chr9: 129942145 | ACTTGAATCAAGGCCTCAAaGG | Intron (RALGPS1) |
| | (SEQ. ID. NO.: 179) | |
| chr9: 37521162 | ACTTGCCCTCAGGCCTCATcAG | Intron (FBXO10) |
| | (SEQ. ID. NO.: 180) | |
| chr4: 54822569 | ACAGGCACTCAGGCCTCATtAG | Intron (PDGFRA) |
| | (SEQ. ID. NO.: 181) | |
| chr5: 94218613 | TCTCAGATTCAGGCCTCATcAG | Intron (MCTP1) |
| | (SEQ. ID. NO.: 182) | |
| chr19: 16109453 | CCTTGGGTTGAGGCCTCATgGG | Intergenic |
| | (SEQ. ID. NO.: 183) | |
| chr8: 53120294 | AAATGAATTCAGGCCTCTTaAG | Intron (ST18) |
| | (SEQ. ID. NO.: 184) | |
| chr11: 126785415 | AGATGAATTCAGGCATCATaGG | Intron (KIRREL3) |
| | (SEQ. ID. NO.: 185) | |
| chr7: 146738774 | ATTTTATTTTAGGCCTCATaAG | Intron (CNTNAP2) |
| | (SEQ. ID. NO.: 186) | |
| chr7: 6731127 | ACCTGAATTCAGCCCTCATgAG | Exon Coding Sequence (ZNF12) |
| | (SEQ. ID. NO.: 187) | |
| chr18: 58966668 | ACTGAAATTCTGGCCTCATcAG | Intergenic |
| | (SEQ. ID. NO.: 188) | |

TABLE 10
Targeting Exon 5

| Genome Coordinates | Sequence | Genomic Region |
|---|---|---|
| chrX: 154215530 | AGTAGTATAAATTTGTGCAaGG | Exon Coding Sequence (F8) |
| | (SEQ. ID. NO.: 189) | |
| chr6: 110537589 | GGCAGTATTAATTTGTGCAgGG | Intron (CDC40) |
| | (SEQ. ID. NO.: 190) | |
| chr2: 177404495 | AAAAGAATAAATTTGTGCAaAG | Intergenic |
| | (SEQ. ID. NO.: 191) | |
| chr14: 43058612 | AGAAATTTAAATTTGTGCAaAG | Intergenic |
| | (SEQ. ID. NO.: 192) | |
| chr15: 61485533 | AGCAGTATAACTTTGTGCAgGG | Intron (RORA) |
| | (SEQ. ID. NO.: 193) | |
| chr10: 93110570 | GGTTGTATAATTTTGTGCAaGG | Non-coding Exon (LOC100188947) |
| | (SEQ. ID. NO.: 194) | |
| chr9: 129672140 | TGAAGTATAAGTTTGTGCAaAG | Intergenic |
| | (SEQ. ID. NO.: 195) | |
| chr2: 187591509 | ATTAGTATTAATTTGTGAAaGG | Intron (FAM171B) |
| | (SEQ. ID. NO.: 196) | |
| chr4: 78814146 | AGGACTAAAAATTTGTGCAaAG | Intron (MRPL1) |
| | (SEQ. ID. NO.: 197) | |
| chr12: 106567292 | AGTTGTATGAATTTGTGTAaAG | Intergenic |
| | (SEQ. ID. NO.: 198) | |
| chr18: 54908149 | AGTAGAAACAATTTGTGCAaAG | Intergenic |
| | (SEQ. ID. NO.: 199) | |
| chr4: 165991674 | AGCAGGATTAATTTGTGCAtGG | Intergenic |
| | (SEQ. ID. NO.: 200) | |
| chrX: 145115485 | AATAATATAGATTTGTGCAtAG | Intergenic |
| | (SEQ. ID. NO.: 201) | |
| chr9: 103735963 | TGAAGTAGAAATTTGTGCAtGG | Intergenic |
| | (SEQ. ID. NO.: 202) | |
| chr2: 25400266 | AGAGGAATCAATTTGTGCAgAG | Intergenic |
| | (SEQ. ID. NO.: 203) | |
| chr3: 176214435 | TTAAGTAGAAATTTGTGCAaAG | Intergenic |
| | (SEQ. ID. NO.: 204) | |
| chr5: 39747651 | AGAAGTCTACATTTGTGCAcAG | Intergenic |
| | (SEQ. ID. NO.: 205) | |
| chr11: 82871606 | GGGGTTATAAATTTGTGCAgAG | Intron (PCF11) |
| | (SEQ. ID. NO.: 206) | |
| chr19: 20791142 | CGTAATGTTAATTTGTGCAtAG | Intergenic |
| | (SEQ. ID. NO.: 207) | |
| chr1: 179850303 | AGTAGTTGAAATTTGTGCCaAG | Promoter (TOR1AIP1) |
| | (SEQ. ID. NO.: 208) | |
| chr9: 135854103 | AGAAGTATCTATTTGTGCAaAG | Exon 5′ UTR (GFI1B) |
| | (SEQ. ID. NO.: 209) | |

TABLE 11
Targeting Exon 6

| Genome Coordinates | Sequence | Genomic Region |
|---|---|---|
| chrX: 154212971 | AGTCAATGGTTATGTAAACaGG | Exon Coding Sequence (F8) |
| | (SEQ. ID. NO.: 210) | |
| chr2: 218967040 | AGTCAATAGTTATGTAAACcAG | Intergenic |
| | (SEQ. ID. NO.: 211) | |
| chr6: 107599653 | AGTGAATGGTTTTGTAAACtAG | Intron (PDSS2) |
| | (SEQ. ID. NO.: 212) | |
| chr9: 111061602 | AGGAAATGTTTATGTAAACcAG | Intergenic |
| | (SEQ. ID. NO.: 213) | |
| chr2: 70145337 | ATCCAAGGGTTATGTAAACcAG | Intron (MXD1) |
| | (SEQ. ID. NO.: 214) | |
| chr2: 179185240 | AATAAAGGGTTATGTAAACcAG | Intron (OSBPL6) |
| | (SEQ. ID. NO.: 215) | |
| chr2: 83865543 | CCTTAAAGGTTATGTAAACtGG | Intergenic |
| | (SEQ. ID. NO.: 216) | |
| chr7: 137752220 | AGCTAATGATTATGTAAACtAG | Intron (AKR1D1) |
| | (SEQ. ID. NO.: 217) | |
| chr6: 84118291 | AATCAATGTTCATGTAAACaGG | Intron (ME1) |
| | (SEQ. ID. NO.: 218) | |
| chr8: 101030343 | ACTCAAAGGTTATGTAATCaGG | Intron (RGS22) |
| | (SEQ. ID. NO.: 219) | |
| chr16: 49658902 | AGTAAAGGGTTTTGTAAACcAG | Intron (ZNF423) |
| | (SEQ. ID. NO.: 220) | |
| chr2: 144518454 | AGCTAATGGATATGTAAACtGG | Intron (ARHGAP15) |
| | (SEQ. ID. NO.: 221) | |
| chr22: 27359583 | TGAGTATGGTTATGTAAACaAG | Intergenic |
| | (SEQ. ID. NO.: 222) | |
| chr6: 75650424 | ATTCAAGGGCTATGTAAACaGG | Intergenic |
| | (SEQ. ID. NO.: 223) | |
| chr11: 46844386 | AGTCAATGTTTATATAAACaAG | Intron (CKAP5) |
| | (SEQ. ID. NO.: 224) | |
| chr3: 87666684 | AGCTAATCTTTATGTAAACtAG | Intergenic |
| | (SEQ. ID. NO.: 225) | |
| chr5: 117377148 | AGTTAATGTATATGTAAACgGG | Intron(LOC102467224) |
| | (SEQ. ID. NO.: 226) | |
| chr6: 88801506 | AGTCAAAGAATATGTAAACaGG | Intergenic |
| | (SEQ. ID. NO.: 227) | |
| chr3: 27607295 | AGTAAATGTTTATGTAAAAaAG | Intergenic |
| | (SEQ. ID. NO.: 228) | |
| chr6: 146115759 | AATGAATGATTATGTCAACtGG | Intron (LOC100507557) |
| | (SEQ. ID. NO.: 229) | |
| chr7: 26490738 | AGGCAATGATTTTGTAAACtAG | Intron (LOC441204) |
| | (SEQ. ID. NO.: 230) | |

TABLE 12
Targeting Exon 7

| Genome Coordinates | Sequence | Genomic Region |
|---|---|---|
| chrX: 154197646 | AAACACTCTTGATGGACCTtGG | Exon Coding Sequence (F8) |
| | (SEQ. ID. NO.: 231) | |
| chr1: 30609971 | GCATCCTCTTGATGGACCTgAG | Intergenic |
| | (SEQ. ID. NO.: 232) | |
| chr13: 44021944 | ATATACTCTTGATTGACCTcAG | Intron (ENOX1) |
| | (SEQ. ID. NO.: 233) | |
| chr15: 29524019 | AATTACTCTTTATGGACCTgAG | Intron (FAM189A1) |
| | (SEQ. ID. NO.: 234) | |
| chr9: 81224323 | CAACACACTTGATGGATCTtAG | Intergenic |
| | (SEQ. ID. NO.: 235) | |
| chr12: 1734560 | AAAGACTGTTTATGGACCTcAG | Intron (WNT5B) |
| | (SEQ. ID. NO.: 236) | |
| chr2: 151715442 | AAACACTCTTAATTGACCTtAG | Intergenic |
| | (SEQ. ID. NO.: 237) | |
| chr3: 100704459 | AACCACATTTGATGGACCAcAG | Intron (ABI3BP) |
| | (SEQ. ID. NO.: 238) | |
| chr15: 94791271 | TCACATTCTTGATGGCCCTaAG | Intron (MCTP2) |
| | (SEQ. ID. NO.: 239) | |
| chr1: 173103354 | AGACATTCTTGCTGGACCTgAG | Intergenic |
| | (SEQ. ID. NO.: 240) | |
| chr2: 5541938 | CAACACTGTTGATGGGCCTtGG | Intergenic |
| | (SEQ. ID. NO.: 241) | |
| chr9: 116815940 | CAATGCTCTTGGTGGACCTgAG | Exon 3′ UTR (ZNF618) |
| | (SEQ. ID. NO.: 242) | |
| chr12: 78013073 | AAATACTATTGATGGACATaAG | Intergenic |
| | (SEQ. ID. NO.: 243) | |
| chr8: 58242713 | AAACCCACTTGATGGACATtAG | Intergenic |
| | (SEQ. ID. NO.: 244) | |
| chr2: 80499580 | AAACACCACTGATGGTCCTtAG | Intron (CTNNA2) |
| | (SEQ. ID. NO.: 245) | |
| chr21: 30965875 | ACACACTCTTCATGGAGCTaGG | Intron (GRIK1) |
| | (SEQ. ID. NO.: 246) | |
| chr10: 130363988 | AAACACTCATGGTGGACATgAG | Intergenic |
| | (SEQ. ID. NO.: 247) | |
| chr1: 219054480 | AAAGAGTCTTGATAGACCTcGG | Intergenic |
| | (SEQ. ID. NO.: 248) | |
| chrX: 130574873 | AAAAAATTTTCATGGACCTcAG | Intron (IGSF1) |
| | (SEQ. ID. NO.: 249) | |
| chr3: 28891898 | TAACATTCTGCATGGACCTcAG | Intergenic |
| | (SEQ. ID. NO.: 250) | |
| chr18: 24094640 | AAACACTCCTCCTGGACCTaGG | Intron (KCTD1) |
| | (SEQ. ID. NO.: 251) | |

TABLE 13
Targeting Exon 8

| Genome Coordinates | Sequence | Genomic Region |
|---|---|---|
| chrX: 154194743 | CATTACATTGCTGCTGAAGaGG | Exon Coding Sequence (F8) |
| | (SEQ. ID. NO.: 252) | |
| chr4: 164547061 | CAATACATTGCTGCTGAATaGG | Intron (MARCH1) |
| | (SEQ. ID. NO.: 253) | |
| chr12: 88212345 | CTCTACATTGCTGCTGAAGcAG | Intergenic |
| | (SEQ. ID. NO.: 254) | |
| chr13: 58393603 | AATTATATTGCTGCTGAAGcAG | Intergenic |
| | (SEQ. ID. NO.: 255) | |
| chr11: 99963764 | CTGTATATTGCTGCTGAAGaGG | Intron (CNTN5) |
| | (SEQ. ID. NO.: 256) | |
| chr5: 147750887 | TATTACATTTCTGCTGAAGaAG | Intron (AK054753) |
| | (SEQ. ID. NO.: 257) | |
| chr3: 21956167 | CTGTACATTGCTGCTGAAAaGG | Intron (ZNF385D) |
| | (SEQ. ID. NO.: 258) | |
| chr8: 66325163 | TTCTACTTTGCTGCTGAAGaAG | Intergenic |
| | (SEQ. ID. NO.: 259) | |
| chr16: 23845478 | GGAGACATTGCTGCTGAAGtAG | Intergenic |
| | (SEQ. ID. NO.: 260) | |
| chr20: 25398809 | TTTCACATGGCTGCTGAAGaAG | Exon Coding Sequence (GINS1) |
| | (SEQ. ID. NO.: 261) | |
| chr7: 108238812 | TTTTACTTAGCTGCTGAAGaAG | Intergenic |
| | (SEQ. ID. NO.: 262) | |
| chr1: 170584156 | CTCCACATAGCTGCTGAAGgAG | Intergenic |
| | (SEQ. ID. NO.: 263) | |
| chr8: 100545059 | CAGTAAATTTCTGCTGAAGaAG | Intron (VPS13B) |
| | (SEQ. ID. NO.: 264) | |
| chr1: 188904130 | CATTCCATTGCTGCTGAAAtAG | Intergenic |
| | (SEQ. ID. NO.: 265) | |
| chr2: 186625904 | CAGTACTATGCTGCTGAAGgAG | Intron (FSIP2) |
| | (SEQ. ID. NO.: 266) | |
| chr5: 121271455 | CAACAAATAGCTGCTGAAGtAG | Intergenic |
| | (SEQ. ID. NO.: 267) | |
| chr18: 52247498 | AAAAACAGTGCTGCTGAAGgAG | Intergenic |
| | (SEQ. ID. NO.: 268) | |
| chr2: 45531502 | TAATTCTTTGCTGCTGAAGcAG | Intergenic |
| | (SEQ. ID. NO.: 269) | |
| chrX: 17770070 | CATTACATGGCTTCTGAAGaGG | Exon Coding Sequence (SCML1) |
| | (SEQ. ID. NO.: 270) | |
| chr2: 183371692 | CAGTACACAGCTGCTGAAGgAG | Intron (PDE1A) |
| | (SEQ. ID. NO.: 271) | |
| chr5: 90418188 | GATGACTTTTCTGCTGAAGgAG | Intron (GPR98) |
| | (SEQ. ID. NO.: 272) | |

TABLE 14
Targeting Exon 9

| Genome Coordinates | Sequence | Genomic Region |
|---|---|---|
| chrX: 154194290 | AACATATTCAGCATGAATTaAG | Exon Coding Sequence (F8) |
| | (SEQ. ID. NO.: 273) | |
| chr5: 44822900 | ACTTTATTCAGCATGAATCcAG | Intergenic |
| | (SEQ. ID. NO.: 274) | |
| chr6: 29094659 | AACATATTCAGCATGAATTaAG | Intergenic |
| | (SEQ. ID. NO.: 275) | |
| chr1: 15533155 | CTGATACTCAGCATGAATCaGG | Intron (TMEM51) |
| | (SEQ. ID. NO.: 276) | |
| chr10: 28683220 | ATGCAATTCTGCATGAATCtAG | Intergenic |
| | (SEQ. ID. NO.: 277) | |
| chr13: 27072101 | AAGATAACCAGCATGAATCaAG | Intergenic |
| | (SEQ. ID. NO.: 278) | |
| chr7: 83366196 | TAACTACACAGCATGAATCtGG | Intergenic |
| | (SEQ. ID. NO.: 279) | |
| chrX: 23428625 | ACACAATTCAGCATGAATCcGG | Intergenic |
| | (SEQ. ID. NO.: 280) | |
| chr10: 23364900 | AAGTTAGGAAGCATGAATCaGG | Intergenic |
| | (SEQ. ID. NO.: 281) | |
| chr5: 154769061 | AAACTATTCTTCATGAATCcAG | Intergenic |
| | (SEQ. ID. NO.: 282) | |
| chr1: 171760953 | GATCTAGTCATCATGAATCcAG | Intron (METTL13) |
| | (SEQ. ID. NO.: 283) | |
| chr13: 38900409 | AAACTAATCAGCATGAATAaAG | Intergenic |
| | (SEQ. ID. NO.: 284) | |
| chr3: 172881404 | AAGTTACTCAGCATGAATGtAG | Intergenic |
| | (SEQ. ID. NO.: 285) | |
| chr1: 236579905 | ATACTATTCAGCATGAATAaGG | Intron (EDARADD) |
| | (SEQ. ID. NO.: 286) | |
| chr16: 66359299 | CATCTAATCAGCATGTATCaGG | Intergenic |
| | (SEQ. ID. NO.: 287) | |
| chr14: 84181421 | AAGATGTTCTGCATGAATCtAG | Intergenic |
| | (SEQ. ID. NO.: 288) | |
| chr20: 13599375 | GAGCTTTAAAGCATGAATCaAG | Intron (TASP1) |
| | (SEQ. ID. NO.: 289) | |
| chr6: 5495962 | AAGATAATTAGCATGGATCaAG | Intron (FARS2) |
| | (SEQ. ID. NO.: 290) | |
| chr4: 181976718 | ATGCAGTTGAGCATGAATCtGG | Intergenic |
| | (SEQ. ID. NO.: 291) | |
| chr22: 25541937 | ATGGTATTCAGCATTAATCcAG | Intron (KIAA1671) |
| | (SEQ. ID. NO.: 292) | |
| chr19: 48634379 | AAGATCTTCAGCAGGAATCaGG | Exon Coding Sequence (LIG1) |
| | (SEQ. ID. NO.: 293) | |

TABLE 15
Targeting Exon 10

| Genome Coordinates | Sequence | Genomic Region |
|---|---|---|
| chrX: 154189379 | GACATCAGTGATTCCGTGAgGG | Exon Coding Sequence (F8) |
| | (SEQ. ID. NO.: 294) | |
| chr8: 1138530 | GGCGTCTGAGATTCCGTGAgGG | Intergenic |
| | (SEQ. ID. NO.: 295) | |
| chr2: 131289600 | GAAGTCATTGATTCCGTGAcAG | Intergenic |
| | (SEQ. ID. NO.: 296) | |
| chr2: 131346282 | GAAGTCATTGATTCCGTGAcAG | Intergenic |
| | (SEQ. ID. NO.: 297) | |
| chr18: 32629196 | GCCCTCTGTGATTCCCTGAgAG | Intron (MAPRE2) |
| | (SEQ. ID. NO.: 298) | |
| chr16: 86333722 | TCCATCTGTGAGTCCGTGAcAG | Intergenic |
| | (SEQ. ID. NO.: 299) | |
| chr10: 14078561 | AAAATCAGTGATTCCGTCAtGG | Intron (FRMD4A) |
| | (SEQ. ID. NO.: 300) | |
| chr17: 77497084 | GAGATTAGGGCTTCCGTGAaGG | Intron (RBFOX3) |
| | (SEQ. ID. NO.: 301) | |
| chr17: 77598354 | GAGATTAGGGCTTCCGTGAaGG | Intergenic |
| | (SEQ. ID. NO.: 302) | |
| chr6: 106596870 | TAGACCAGTGCTTCCGTGAgGG | Intergenic |
| | (SEQ. ID. NO.: 303) | |
| chrX: 82789988 | GCCATTAGTGATTCCTTGAaAG | Intergenic |
| | (SEQ. ID. NO.: 304) | |
| chrY: 16304327 | GACCTCAGTGATTCCATCAaAG | Intergenic |
| | (SEQ. ID. NO.: 305) | |
| chr8: 120276922 | GCCATCAGACATTCCGTGCaAG | Intergenic |
| | (SEQ. ID. NO.: 306) | |
| chr13: 80232725 | GACATCAGTGATGCCCTGAgGG | Intergenic |
| | (SEQ. ID. NO.: 307) | |
| chr10: 80878062 | GACCACAGAGATTCCTTGAtGG | Intron (ZMIZ1) |
| | (SEQ. ID. NO.: 308) | |
| chr2: 2966966 | GGCGTCAGTGGTTCCATGAaGG | Intron (AK095310) |
| | (SEQ. ID. NO.: 309) | |
| chr12: 119778660 | GTAATCAGTGATTCCATGCaGG | Intron (CCDC60) |
| | (SEQ. ID. NO.: 310) | |
| chr4: 2967154 | GAAATCAGCAATTCCGTAAgAG | Exon Coding Sequence (GRK4) |
| | (SEQ. ID. NO.: 311) | |
| chr12: 46200577 | GACACCAGTCATTCCGTGCtGG | Intron (ARID2) |
| | (SEQ. ID. NO.: 312) | |
| chr9: 86513993 | GGCATTAGTTATTCCCTGAtAG | Intron (KIF27) |
| | (SEQ. ID. NO.: 313) | |
| chr6: 26642811 | GAGTTCTGTGATACCGTGAaAG | Intron (ZNF322) |
| | (SEQ. ID. NO.: 314) | |

TABLE 16
Targeting Exon 11:

| Genome Coordinates | Sequence | Genomic Region |
|---|---|---|
| chrX: 154185280 | ATCTAGCTTCAGGACTCATtGG | Exon Coding Sequence (F8) |
| | (SEQ. ID. NO.: 315) | |
| chr16: 23190364 | ATTTATCTTCAGGACTCATgAG | Intergenic |
| | (SEQ. ID. NO.: 316) | |
| chr3: 186577494 | ATGCAGATTCAGGACTCATgGG | Intergenic |
| | (SEQ. ID. NO.: 317) | |
| chrX: 150674237 | ATTGAGTTTCAGGACTCATtGG | Intergenic |
| | (SEQ. ID. NO.: 318) | |
| chr2: 221884896 | ATCGGGCTCCAGGACTCATtGG | Intergenic |
| | (SEQ. ID. NO.: 319) | |
| chr10: 70243847 | ATCAAATTTCAGGACTCATtAG | Intron (SLC25A16) |
| | (SEQ. ID. NO.: 320) | |
| chr3: 148927976 | ATATTGCCTCAGGACTCATcGG | Exon Coding Sequence (CP) |
| | (SEQ. ID. NO.: 321) | |
| chr3: 179383328 | GTCTAACTTCATGACTCATcAG | Intron (USP13) |
| | (SEQ. ID. NO.: 322) | |
| chr2: 21468146 | AACTAACTTCAAGACTCATtGG | Intergenic |
| | (SEQ. ID. NO.: 323) | |
| chr6: 3455403 | CTTTAGCTACAGGACTCAGaGG | Intron (SLC22A23) |
| | (SEQ. ID. NO.: 324) | |
| chr2: 121527930 | GCCCAGCTTCAGGACCCATaGG | Intron (GLI2) |
| | (SEQ. ID. NO.: 325) | |
| chr1: 244407318 | TTCTTTGTTCAGGACTCATgGG | Intergenic |
| | (SEQ. ID. NO.: 326) | |
| chrX: 131818829 | TTCTTTGTTCAGGACTCATgGG | Intron (HS6ST2) |
| | (SEQ. ID. NO.: 327) | |
| chr2: 16363229 | ATCCACCTTCAGGACTCAGaGG | Intergenic |
| | (SEQ. ID. NO.: 328) | |
| chr6: 19171840 | ATCTAGATTCAAGACTCACtGG | Intron (AK097585) |
| | (SEQ. ID. NO.: 329) | |
| chr2: 20736595 | AGCCAGCTCCAGGACTCCTtGG | Intergenic |
| | (SEQ. ID. NO.: 330) | |
| chr6: 130923353 | ACCTAGGATCAGGACTCAGtGG | Intergenic |
| | (SEQ. ID. NO.: 331) | |
| chr9: 5363091 | CTCTAGGTTTTGGACTCATtGG | Intron (PLGRKT) |
| | (SEQ. ID. NO.: 332) | |
| chr14: 77583105 | ATCTGGCTTCTGGACTCAAtGG | Exon 3′ UTR (KIAA1737) |
| | (SEQ. ID. NO.: 333) | |
| chr12: 60244386 | ATAGAACTTCATGACTCATtAG | Intergenic |
| | (SEQ. ID. NO.: 334) | |
| chr5: 15918957 | AGTTAGCTTTAGGACTCAAgAG | Intron (FBXL7) |
| | (SEQ. ID. NO.: 335) | |

TABLE 17
Targeting Exon 12:

| Genome Coordinates | Sequence | Genomic Region |
|---|---|---|
| chrX: 154182213 | GCTTTCTCCCCAATCCAGCtGG | Exon Coding Sequence (F8) |
| | (SEQ. ID. NO.: 336) | |
| chr15: 79094755 | TCTGTCTCCCCAATCCAGGaGG | Intron (ADAMTS7) |
| | (SEQ. ID. NO.: 337) | |
| chr2: 235670611 | AATCTCTCCCCAATCCAGCaGG | Intergenic |
| | (SEQ. ID. NO.: 338) | |
| chr17: 43743770 | GCAGTTTCCCCAATCCAGCaGG | Intron (CRHR1) |
| | (SEQ. ID. NO.: 339) | |
| chrX: 68443853 | GACTTTTCCCCAATCCAGCaGG | Intergenic |
| | (SEQ. ID. NO.: 340) | |
| chr1: 165087672 | GCTTTCTCCTCAATCCAGGgAG | Intergenic |
| | (SEQ. ID. NO.: 341) | |
| chr17: 25876995 | CCATTCTCCCCAAACCAGCaGG | Intron (KSR1) |
| | (SEQ. ID. NO.: 342) | |
| chr2: 29518182 | TTTTTCTCCTCAATCCAGCaAG | Intron (ALK) |
| | (SEQ. ID. NO.: 343) | |
| chr22: 36723218 | GATCTCTCCACAATCCAGCtGG | Intron (MYH9) |
| | (SEQ. ID. NO.: 344) | |
| chr3: 184449552 | GCTTTCTCCCAAATCCAGAaAG | Intergenic |
| | (SEQ. ID. NO.: 345) | |
| chr8: 37532822 | GCTTTCATCCCAATCCAGGtGG | Intergenic |
| | (SEQ. ID. NO.: 346) | |
| chr2: 31030850 | TCTTTCTGCCCCATCCAGCaAG | Promoter (CAPN13) |
| | (SEQ. ID. NO.: 347) | |
| chr3: 6486747 | GCTATCTCACCCATCCAGCaGG | Intergenic |
| | (SEQ. ID. NO.: 348) | |
| chr11: 65297618 | ACTTCCTGCCCAATCCAGCcAG | Intron (SCYL1) |
| | (SEQ. ID. NO.: 349) | |
| chr11: 21451235 | GCTTTGTCATCAATCCAGCcAG | Intron (NELL1) |
| | (SEQ. ID. NO.: 350) | |
| chr4: 14748843 | CCTCTTTCCCAAATCCAGCaAG | Intron (MGC4836) |
| | (SEQ. ID. NO.: 351) | |
| chr2: 70941601 | GCCTCCTCCTCAATCCAGCcAG | Intron (ADD2) |
| | (SEQ. ID. NO.: 352) | |
| chr1: 171768046 | ACTTTCCTCACAATCCAGCaAG | Promoter (METTL13) |
| | (SEQ. ID. NO.: 353) | |
| chr7: 150731340 | TCTGTCTCCCCATTCCAGCtGG | Intron Near Splice Site (ABCB8) |
| | (SEQ. ID. NO.: 354) | |
| chr11: 62521856 | TCCTTCTACCTAATCCAGCaGG | Promoter (ZBTB3) |
| | (SEQ. ID. NO.: 355) | |
| chr19: 6904138 | GCTTTCATCCCAATCCAGAaGG | Exon Coding Sequence (EMR1) |
| | (SEQ. ID. NO.: 356) | |

TABLE 18
Targeting Exon 13:

| Genome Coordinates | Sequence | Genomic Region |
|---|---|---|
| chrX: 154175981 | GAAACTGTCTTCATGTCGAtGG | Exon Coding Sequence (F8) |
| | (SEQ. ID. NO.: 357) | |
| chr21: 34095440 | GACTCTGTCTTTATGTCGAtAG | Intron (SYNJ1) |
| | (SEQ. ID. NO.: 358) | |
| chrX: 83459827 | GAATCTTTCTTCATGTCCAaAG | Intergenic |
| | (SEQ. ID. NO.: 359) | |
| chr12: 14664172 | GGTACTTTCTTCATGTCGTaAG | Intron Near Splice Site (PLBD1) |
| | (SEQ. ID. NO.: 360) | |
| chr5: 53912853 | GAGACCTCCTTCATGTCGAaGG | Intergenic |
| | (SEQ. ID. NO.: 361) | |
| chr18: 72831123 | ACAACTCTCTTCATGTCTAaAG | Intergenic |
| | (SEQ. ID. NO.: 362) | |
| chr2: 165858924 | GAAACTATATTCATGTTGAaAG | Intergenic |
| | (SEQ. ID. NO.: 363) | |
| chr2: 50691597 | GAGACTGTATTCATGTCAAcAG | Intron (NRXN1) |
| | (SEQ. ID. NO.: 364) | |
| chr3: 177604193 | AAGACTGTTTTCATGTCAAgGG | Intron (AK056252) |
| | (SEQ. ID. NO.: 365) | |
| chr18: 75861775 | GAAACCGCCTTCATGTCCAaAG | Intergenic |
| | (SEQ. ID. NO.: 366) | |
| chr10: 21473461 | GAACCTGGCTTCATGGCGAtGG | Intergenic |
| | (SEQ. ID. NO.: 367) | |
| chr2: 91925133 | GAAGCTGTCTTCACGTCGCcAG | Intergenic |
| | (SEQ. ID. NO.: 368) | |
| chr6: 45450917 | GAAACTGTCTTCATGTTTAaGG | Intron (RUNX2) |
| | (SEQ. ID. NO.: 369) | |
| chr11: 8149451 | GTTACTATCTTCATGTTGAaAG | Intron (RIC3) |
| | (SEQ. ID. NO.: 370) | |
| chr5: 76255097 | GATACTTCCTTCATGTCAAaAG | Intron (CRHBP) |
| | (SEQ. ID. NO.: 371) | |
| chr16: 67002407 | GTGAATGTCTTCATGTCCAtGG | Intron (CES3) |
| | (SEQ. ID. NO.: 372) | |
| chrX: 9685009 | GATTGTGTCTTCATGTCCAcGG | Exon 3′ UTR (TBL1X) |
| | (SEQ. ID. NO.: 373) | |
| chr5: 4907531 | GGGACTGTCTGCATGCCGAcAG | Intergenic |
| | (SEQ. ID. NO.: 374) | |
| chr9: 81530191 | GACACTATCATCATGTCCAgGG | Intergenic |
| | (SEQ. ID. NO.: 375) | |
| chr3: 71439196 | CAAACTGTGTGCATGGCGAaGG | Intron (FOXP1) |
| | (SEQ. ID. NO.: 376) | |
| chr8: 81486615 | GAAACTGTAATCATGTCCAaGG | Intergenic |
| | (SEQ. ID. NO.: 377) | |

TABLE 19
Targeting Exon 14:

| Genome Coordinates | Sequence | Genomic Region |
|---|---|---|
| chrX: 154156897 | CACTATTTTATTGCTGCAGtGG | Exon Coding Sequence (F8) |
| | (SEQ. ID. NO.: 378) | |
| chr1: 30562288 | AACTATTTTATTGCTGCAAgAG | Intergenic |
| | (SEQ. ID. NO.: 379) | |
| chrX: 136566499 | CACCATTTTATTGCTGCAAaGG | Intergenic |
| | (SEQ. ID. NO.: 380) | |
| chr2: 190687632 | AAATATTTTGTTGCTGCAGcAG | Intron (PMS1) |
| | (SEQ. ID. NO.: 381) | |
| chr12: 70464237 | GAATATTTTATTGCTGCAAaAG | Intergenic |
| | (SEQ. ID. NO.: 382) | |
| chr15: 101020010 | GATTTTTTTATTGCTGCAGaAG | Intron (CERS3) |
| | (SEQ. ID. NO.: 383) | |
| chr15: 29992687 | CGCTGCTTTATTGCTGCAGaGG | Exon 3′ UTR (TJP1) |
| | (SEQ. ID. NO.: 384) | |
| chr3: 44601871 | AGCCACTTTATTGCTGCAGaAG | Intron (ZKSCAN7) |
| | (SEQ. ID. NO.: 385) | |
| chr22: 45864978 | AAATATTCTATTGCTGCAGcAG | Intergenic |
| | (SEQ. ID. NO.: 386) | |
| chr16: 52103653 | CAGAAATTCATTGCTGCAGgGG | Intron (C16orf97) |
| | (SEQ. ID. NO.: 387) | |
| chr1: 120881376 | CACCAGCTCATTGCTGCAGcAG | Intergenic |
| | (SEQ. ID. NO.: 388) | |
| chr1: 149424437 | CACCAGCTCATTGCTGCAGcAG | Intergenic |
| | (SEQ. ID. NO.: 389) | |
| chr12: 25277057 | GGTTATTCTATTGCTGCAGaAG | Intron (CASC1) |
| | (SEQ. ID. NO.: 390) | |
| chr10: 112904390 | AACTATTAGATTGCTGCAGaAG | Intergenic |
| | (SEQ. ID. NO.: 391) | |
| chr8: 70050560 | AAAGCTTTTATTGCTGCAGgAG | Intergenic |
| | (SEQ. ID. NO.: 392) | |
| chr8: 28231898 | AACTTTCTGATTGCTGCAGaAG | Intron (ZNF395) |
| | (SEQ. ID. NO.: 393) | |
| chr4: 91416984 | TTCTATTGCATTGCTGCAGgGG | Intron (CCSER1) |
| | (SEQ. ID. NO.: 394) | |
| chr2: 200633700 | CCGTATTAGATTGCTGCAGgAG | Intron (FTCDNL1) |
| | (SEQ. ID. NO.: 395) | |
| chr10: 59130250 | GCTTATTTTAGTGCTGCAGaAG | Intergenic |
| | (SEQ. ID. NO.: 396) | |
| chr17: 46350296 | ACATATTTTAGTGCTGCAGaAG | Intron (SKAP1) |
| | (SEQ. ID. NO.: 397) | |
| chr17: 70509338 | CACCATCTGTTTGCTGCAGcAG | Intron (LINC00673) |
| | (SEQ. ID. NO.: 398) | |

TABLE 20
Targeting Exon 15:

| Genome Coordinates | Sequence | Genomic Region |
|---|---|---|
| chrX: 154134707 | CAACTTCTGCTCTTATATAtGG | Exon Coding Sequence (F8) |
| | (SEQ. ID. NO.: 399) | |
| chr1: 218213257 | TAACTTCTGCTCTTATATCtAG | Intergenic |
| | (SEQ. ID. NO.: 400) | |
| chr9: 118248735 | CCACTTCTTCTCTTATATAcAG | Intergenic |
| | (SEQ. ID. NO.: 401) | |
| chr21: 19995903 | CAACTTGTGGTCTTATATAaAG | Intron (BC028044) |
| | (SEQ. ID. NO.: 402) | |
| chr6: 107914478 | CAGCTTCTGCTCTGATATAgGG | Intron (SOBP) |
| | (SEQ. ID. NO.: 403) | |
| chr6: 62756536 | CATTTTCTCCTCTTATATAaAG | Intron (KHDRBS2) |
| | (SEQ. ID. NO.: 404) | |
| chr1: 86987590 | CAACTTCTGTTCTTATATTtAG | Intergenic |
| | (SEQ. ID. NO.: 405) | |
| chr5: 164293350 | GAACTCCTGCTCTTATATAaGG | Intergenic |
| | (SEQ. ID. NO.: 406) | |
| chr3: 81865056 | CAACTTTTGCTCTTATATCaGG | Intergenic |
| | (SEQ. ID. NO.: 407) | |
| chr14: 79923464 | AAGATTCTGCTCTTATATAcAG | Intron (NRXN3) |
| | (SEQ. ID. NO.: 408) | |
| chr1: 52942388 | CATCTTGTACTCTTATATAtAG | Intron (ZCCHC11) |
| | (SEQ. ID. NO.: 409) | |
| chr14: 79314602 | GATCTTCTTCTCTTATATAgAG | Intron (NRXN3) |
| | (SEQ. ID. NO.: 410) | |
| chr1: 60518851 | CTAGTTTTTCTCTTATATAtAG | Intron (C1orf87) |
| | (SEQ. ID. NO.: 411) | |
| chr5: 26555643 | CAATTTGTGCTATTATATAcAG | Intergenic |
| | (SEQ. ID. NO.: 412) | |
| chr3: 183366063 | CAACTCATTCTCTTATATAtAG | Intron (KLHL24) |
| | (SEQ. ID. NO.: 413) | |
| chr9: 11538499 | CAAACTCTGATCTTATATAcAG | Intergenic |
| | (SEQ. ID. NO.: 414) | |
| chr4: 125027842 | AATCTTCTGATCTTATATAcAG | Intergenic |
| | (SEQ. ID. NO.: 415) | |
| chr7: 104902183 | CACCTTATGATCTTATATAtAG | Intron (SRPK2) |
| | (SEQ. ID. NO.: 416) | |
| chr4: 153730320 | AACCTTCCTCTCTTATATAgGG | Intron (ARFIP1) |
| | (SEQ. ID. NO.: 417) | |
| chr4: 166631085 | CAACCTCTGCTCTTAAATAgGG | Intergenic |
| | (SEQ. ID. NO.: 418) | |
| chr21: 18261294 | CACATTATGTTCTTATATAcAG | Intergenic |
| | (SEQ. ID. NO.: 419) | |

TABLE 21
Targeting Exon 16

| Genome Coordinates | Sequence | Genomic Region |
|---|---|---|
| chrX: 154133109 | TGAGTTTGACTGCAAAGCCtGG | Exon Coding Sequence (F8) |
| | (SEQ. ID. NO.: 420) | |
| chr2: 139083398 | TGATTGTGACTGCAAAGCCaGG | Intergenic |
| | (SEQ. ID. NO.: 421) | |
| chr4: 25019737 | TGAATGTGACTGCAAAGCCaAG | Exon Coding Sequence (LGI2) |
| | (SEQ. ID. NO.: 422) | |
| chr6: 109849332 | TGTGTTTAACTGCAAAGCCtGG | Intron (AK9) |
| | (SEQ. ID. NO.: 423) | |
| chr16: 64396489 | TTAGTCTGTCTGCAAAGCCtGG | Intergenic |
| | (SEQ. ID. NO.: 424) | |
| chr17: 17656377 | AGAGTTTGTCTCCAAAGCCaGG | Intron (RAI1) |
| | (SEQ. ID. NO.: 425) | |
| chr14: 80073468 | TGTTTTTGACTGCAAAGTCcAG | Intron (NRXN3) |
| | (SEQ. ID. NO.: 426) | |
| chr10: 23138453 | TAACTCAGACTGCAAAGCCaAG | Intergenic |
| | (SEQ. ID. NO.: 427) | |
| chr3: 68884768 | AAATTTTCACTGCAAAGCCcAG | Intron (FAM19A4) |
| | (SEQ. ID. NO.: 428) | |
| chr6: 143221421 | TGAGTATGGCTGCAAAGCAcAG | Intron (HIVEP2) |
| | (SEQ. ID. NO.: 429) | |
| chr5: 166979670 | TTGGCTTGTCTGCAAAGCCtGG | Intron (TENM2) |
| | (SEQ. ID. NO.: 430) | |
| chr4: 119920889 | TGATTTATCCTGCAAAGCCcAG | Intron (SYNPO2) |
| | (SEQ. ID. NO.: 431) | |
| chr15: 67172416 | GGGGTTTGACTGCAAAGCAgGG | Intergenic |
| | (SEQ. ID. NO.: 432) | |
| chr4: 148319629 | TCTTTTTGACTGCAAAGCTtAG | Intergenic |
| | (SEQ. ID. NO.: 433) | |
| chr4: 6970950 | TGAGTTTGTATGCAAAGCTtAG | Intron (TBC1D14) |
| | (SEQ. ID. NO.: 434) | |
| chr15: 45981291 | TGAGTTTGACTACAAAGCAgAG | Exon Coding Sequence (SQRDL) |
| | (SEQ. ID. NO.: 435) | |
| chr10: 71833193 | TCTCTTTGACTGCAAGGCCcAG | Intron (H2AFY2) |
| | (SEQ. ID. NO.: 436) | |
| chr5: 94591207 | TGAGTGGCACTGCAAAGCCaGG | Intron (MCTP1) |
| | (SEQ. ID. NO.: 437) | |
| chr20: 44873266 | TCTGTTTGACTCCAAAGCCcAG | Intron (CDH22) |
| | (SEQ. ID. NO.: 438) | |
| chr4: 62575894 | AGGCTTTGACTCCAAAGCCtGG | Intron (LPHN3) |
| | (SEQ. ID. NO.: 439) | |
| chr10: 19019007 | ACACTTTGACTTCAAAGCCtAG | Intergenic |
| | (SEQ. ID. NO.: 440) | |

TABLE 22
Targeting Exon 17

| Genome Coordinates | Sequence | Genomic Region |
|---|---|---|
| chrX: 154132606 | GCTCCCTGCAATATCCAGAtGG | Exon Coding Sequence (F8) |
| | (SEQ. ID. NO.: 441) | |
| chr12: 24549232 | ATTCCCTGCTATATCCAGAcGG | Intron (SOX5) |
| | (SEQ. ID. NO.: 442) | |
| chr5: 172088015 | GCTTCCCGCCATATCCAGAgGG | Intron (NEURL1B) |
| | (SEQ. ID. NO.: 443) | |
| chr10: 131845370 | GCTCCTGCCAATATCCAGAtGG | Intergenic |
| | (SEQ. ID. NO.: 444) | |
| chr5: 12139743 | ATTCCTAGCAATATCCAGAaAG | Intergenic |
| | (SEQ. ID. NO.: 445) | |
| chr15: 79497121 | GAACCAAGCAATATCCAGAgAG | Intron (LOC729911) |
| | (SEQ. ID. NO.: 446) | |
| chr15: 89285594 | GCTCCCTGCTATAGCCAGAcAG | Intergenic |
| | (SEQ. ID. NO.: 447) | |
| chr3: 13261374 | GCTGCCCACAATATCCAGAgAG | Intergenic |
| | (SEQ. ID. NO.: 448) | |
| chr4: 136894615 | GCTGCCGTCAATATCCAGAtAG | Intergenic |
| | (SEQ. ID. NO.: 449) | |
| chr2: 82342655 | GAACTCTGCAATATCCAGAtGG | Intergenic |
| | (SEQ. ID. NO.: 450) | |
| chrX: 128176291 | GCCCCCAGCAGTATCCAGAgAG | Intergenic |
| | (SEQ. ID. NO.: 451) | |
| chr1: 242952956 | GGACCCCGCAGTATCCAGAaGG | Intergenic |
| | (SEQ. ID. NO.: 452) | |
| chr10: 132576153 | GCTCCCAGCGATATCCAGGcGG | Intergenic |
| | (SEQ. ID. NO.: 453) | |
| chr4: 84717722 | GCATCCTGGAATATCCAGGtGG | Exon 3′ UTR (BC005018) |
| | (SEQ. ID. NO.: 454) | |
| chr17: 41807353 | CCGTCCTGCAAGATCCAGAtGG | Intergenic |
| | (SEQ. ID. NO.: 455) | |
| chr11: 44681497 | GCTTCCTGCCATATCCACAgGG | Intergenic |
| | (SEQ. ID. NO.: 456) | |
| chr7: 45574162 | TCTGACTACAATATCCAGAaAG | Intergenic |
| | (SEQ. ID. NO.: 457) | |
| chrX: 9405488 | TCTGACTACAATATCCAGAaAG | Intergenic |
| | (SEQ. ID. NO.: 458) | |
| chr10: 28642879 | GATCCCTTCCATATCCAGAaGG | Intergenic |
| | (SEQ. ID. NO.: 459) | |
| chr10: 90582741 | TCTCCGTGCAATATCCAGTgAG | Exon Coding Sequence (ANKRD22) |
| | (SEQ. ID. NO.: 460) | |
| chr1: 66491441 | ATTCTCTGCAATATCCAGCaAG | Intron (PDE4B) |
| | (SEQ. ID. NO.: 461) | |

TABLE 23
Targeting Exon 18:

| Genome Coordinates | Sequence | Genomic Region |
|---|---|---|
| chrX: 154132213 | TTCACTGTACGAAAAAAAGaGG | Exon Coding Sequence (F8) |
| | (SEQ. ID. NO.: 462) | |
| chr14: 51721622 | TTCACTGTGTGAAAAAAAGaAG | Exon 3′ UTR (TMX1) |
| | (SEQ. ID. NO.: 463) | |
| chr11: 23782919 | TTCACTGTTCCAAAAAAAGcAG | Intergenic |
| | (SEQ. ID. NO.: 464) | |
| chr10: 46229849 | TTCACATTAAGAAAAAAAGtAG | Intron (FAM21C) |
| | (SEQ. ID. NO.: 465) | |
| chr10: 51834846 | TTCACATTAAGAAAAAAAGtAG | Intron (FAM21A) |
| | (SEQ. ID. NO.: 466) | |
| chr2: 137923513 | TTCACATTAAGAAAAAAAGtAG | Intron (THSD7B) |
| | (SEQ. ID. NO.: 467) | |
| chr11: 28118088 | TTAACTCTAAGAAAAAAAGtAG | Intron (KIF18A) |
| | (SEQ. ID. NO.: 468) | |
| chr16: 14360256 | CTCACTTTATGAAAAAAAGgAG | Exon 3′ UTR (MKL2) |
| | (SEQ. ID. NO.: 469) | |
| chr18: 43382979 | TTCTCTATAGGAAAAAAAGgAG | Intergenic |
| | (SEQ. ID. NO.: 470) | |
| chrY: 7642466 | ATCACTTTAGGAAAAAAAGtGG | Intron (BC041884) |
| | (SEQ. ID. NO.: 471) | |
| chr4: 34490208 | TTAAGTGTACAAAAAAAAGgAG | Intergenic |
| | (SEQ. ID. NO.: 472) | |
| chr1: 58066637 | TCCACTGTAAGAAAAAAACaAG | Intron (DAB1) |
| | (SEQ. ID. NO.: 473) | |
| chr8: 94494323 | TCCCCTTTAGGAAAAAAAGcAG | Intron (LINC00535) |
| | (SEQ. ID. NO.: 474) | |
| chr2: 39972530 | TAGATTGTTCGAAAAAAAGaAG | Intron (THUMPD2) |
| | (SEQ. ID. NO.: 475) | |
| chr8: 70711498 | TTCACTGTATGAAAAGAAGaAG | Intron (SLCO5A1) |
| | (SEQ. ID. NO.: 476) | |
| chr1: 187113355 | TGCACTGTCCAAAAAAAAGaGG | Intergenic |
| | (SEQ. ID. NO.: 477) | |
| chr9: 113908333 | TTCACCCTACCAAAAAAAGtAG | Intergenic |
| | (SEQ. ID. NO.: 478) | |
| chr1: 222971317 | TTAACTGAAAGAAAAAAAGaGG | Intergenic |
| | (SEQ. ID. NO.: 479) | |
| chr5: 72092843 | TTGATTGTAAGAAAAAAAGtAG | Intergenic |
| | (SEQ. ID. NO.: 480) | |
| chr6: 102369780 | TTCAGTTTAAGAAAAAAAGcAG | Intron (GRIK2) |
| | (SEQ. ID. NO.: 481) | |
| chr3: 172742167 | ATCAATTTAAGAAAAAAAGaAG | Intron (SPATA16) |
| | (SEQ. ID. NO.: 482) | |

TABLE 24
Targeting Exon 19:

| Genome Coordinates | Sequence | Genomic Region |
|---|---|---|
| chrX: 154130388 | AAAGCTGGAATTTGGCGGGtGG | Exon Coding Sequence (F8) |
| | (SEQ. ID. NO.: 483) | |
| chr12: 57619554 | GAGGCTGGGATTTGGCGGGaGG | Exon Coding Sequence (NXPH4) |
| | (SEQ. ID. NO.: 484) | |
| chr7: 16597415 | AAAGCAGGAATTTGGCTGGtAG | Intron (LRRC72) |
| | (SEQ. ID. NO.: 485) | |
| chr1: 24818199 | AATCCTGGAATTTGGGGGGaGG | Intergenic |
| | (SEQ. ID. NO.: 486) | |
| chr22: 20200714 | AATGGTGGACTTTGGCGGGcGG | Intergenic |
| | (SEQ. ID. NO.: 487) | |
| chr13: 19691015 | GAGGCTGGACTTTGGCGGGtGG | Intergenic |
| | (SEQ. ID. NO.: 488) | |
| chr3: 197212576 | AAAACTGGGGTTTGGCGGGgGG | Intergenic |
| | (SEQ. ID. NO.: 489) | |
| chr16: 55151321 | AGGGCTGGCATTTGGCGGCaAG | Intergenic |
| | (SEQ. ID. NO.: 490) | |
| chr14: 78922207 | AAGTCTGGAATTTGGAGGGaGG | Intron (NRXN3) |
| | (SEQ. ID. NO.: 491) | |
| chr3: 193584475 | GAGGCTGGAATTTGGGGGGaGG | Intergenic |
| | (SEQ. ID. NO.: 492) | |
| chr5: 172092691 | GAGGCTGGAATTTGGGGGGaGG | Intron (NEURL1B) |
| | (SEQ. ID. NO.: 493) | |
| chr7: 64699779 | GAGGCTGGAATTTGGAGGGtGG | Intron (LOC441242) |
| | (SEQ. ID. NO.: 494) | |
| chr3: 20178832 | AGTCCTGGAATTTGGTGGGtAG | Intron (KAT2B) |
| | (SEQ. ID. NO.: 495) | |
| chr11: 105498469 | AGAGCTGGCATTTGGTGGGaGG | Intron (GRIA4) |
| | (SEQ. ID. NO.: 496) | |
| chr1: 154307590 | CAAGCTGGCATGTGGCGGGcAG | Intron (ATP8B2) |
| | (SEQ. ID. NO.: 497) | |
| chr17: 39777661 | CAAGCTGGGATCTGGCGGGtGG | Intron (KRT17) |
| | (SEQ. ID. NO.: 498) | |
| chr3: 9976636 | AGAGCAGAGATTTGGCGGGgAG | Intron Near Splice Site (CRELD1) |
| | (SEQ. ID. NO.: 499) | |
| chr5: 179358898 | AGATCTGGGATATGGCGGGaAG | Intergenic |
| | (SEQ. ID. NO.: 500) | |
| chr10: 48053919 | AAAGGTAGACTTTGGCGGGtAG | Intergenic |
| | (SEQ. ID. NO.: 501) | |
| chr10: 51999210 | AAAGGTAGACTTTGGCGGGtAG | Intron (ASAH2) |
| | (SEQ. ID. NO.: 502) | |
| chr16: 80598041 | AAAGCTGGAGTTTTGCGGGgAG | Intergenic |
| | (SEQ. ID. NO.: 503) | |

TABLE 25
Targeting Exon 20:

| Genome Coordinates | Sequence | Genomic Region |
|---|---|---|
| chrX: 154129683 | GTCCAGAAGCCATTCCCAGgGG | Exon Coding Sequence (F8) |
| | (SEQ. ID. NO.: 504) | |
| chr1: 43418299 | GTGCAGAAGCTATTCCCAGaGG | Intron (SLC2A1) |
| | (SEQ. ID. NO.: 505) | |
| chr19: 54867935 | GTCCAGGAGTCATTCCCAGgGG | Intron near Splice Site (LAIR1) |
| | (SEQ. ID. NO.: 506) | |
| chr4: 103462838 | ATCCAGAAGCCATTCCCACaGG | Intron (NFKB1) |
| | (SEQ. ID. NO.: 507) | |
| chr10: 75596575 | GCCAAGCAGCCATTCCCAGcAG | Intron (CAMK2G) |
| | (SEQ. ID. NO.: 508) | |
| chr1: 205910828 | GCCCAGCACCCATTCCCAGcAG | Intron (SLC26A9) |
| | (SEQ. ID. NO.: 509) | |
| chr1: 242583642 | TACCAGAAACCATTCCCAGcAG | Intron (PLD5) |
| | (SEQ. ID. NO.: 510) | |
| chr11: 113292618 | GTGCAGAAGCCATTCTCAGaGG | Intron (DRD2) |
| | (SEQ. ID. NO.: 511) | |
| chr4: 130365596 | GTCAAGAAGCCATTCTCAGaAG | Intergenic |
| | (SEQ. ID. NO.: 512) | |
| chr15: 97265743 | GCCCAGTAGCCTTTCCCAGgGG | Intergenic |
| | (SEQ. ID. NO.: 513) | |
| chr14: 38982693 | GTACTGAAGACATTCCCAGtAG | Intergenic |
| | (SEQ. ID. NO.: 514) | |
| chr17: 18377324 | CACCACAATCCATTCCCAGtGG | Intergenic |
| | (SEQ. ID. NO.: 515) | |
| chr17: 20373596 | CACCACAATCCATTCCCAGtGG | Intergenic |
| | (SEQ. ID. NO.: 516) | |
| chr17: 20604998 | CACCACAATCCATTCCCAGtGG | Intergenic |
| | (SEQ. ID. NO.: 517) | |
| chr12: 33507303 | GCCCATCACCCATTCCCAGcAG | Intergenic |
| | (SEQ. ID. NO.: 518) | |
| chr3: 126469354 | ATCCTGAAGCAATTCCCAGgAG | Intron (CHCHD6) |
| | (SEQ. ID. NO.: 519) | |
| chr6: 64203707 | CTTCAGAAGTCATTCCCAGgGG | Intergenic |
| | (SEQ. ID. NO.: 520) | |
| chr1: 74488374 | GACAAGAAGTCATTCCCAGtGG | Intergenic |
| | (SEQ. ID. NO.: 521) | |
| chr3: 38643456 | GCACAGAAGGCATTCCCAGgGG | Intron (SCN5A) |
| | (SEQ. ID. NO.: 522) | |
| chr1: 60451879 | GCCTGGAATCCATTCCCAGcAG | Intergenic |
| | (SEQ. ID. NO.: 523) | |
| chr10: 103753785 | GGGCTGAACCCATTCCCAGcAG | Intron (C10orf76) |
| | (SEQ. ID. NO.: 524) | |

TABLE 26
Targeting Exon 21

| Genome Coordinates | Sequence | Genomic Region |
|---|---|---|
| chrX: 154128160 | ATCAATGCCTGGAGCACCAaGG | Exon Coding Sequence (F8) |
| | (SEQ. ID. NO.: 525) | |
| chr3: 42547401 | ATCTACCCCTGGAGCACCAgGG | Intron (VIPR1) |
| | (SEQ. ID. NO.: 526) | |
| chr8: 128417948 | ATCTAATCCTGGAGCACCAaGG | Intron (DQ515898) |
| | (SEQ. ID. NO.: 527) | |
| chr12: 123621690 | TTCATTTCCTGGAGCACCAaAG | Intron (PITPNM2) |
| | (SEQ. ID. NO.: 528) | |
| chr16: 78686450 | AGAAATACCTGGAGCACCAgAG | Intron (WWOX) |
| | (SEQ. ID. NO.: 529) | |
| chr9: 108348273 | GTAAATGCCTGCAGCACCAtGG | Intron (FKTN) |
| | (SEQ. ID. NO.: 530) | |
| chr17: 44477088 | ACCAAAGCCTAGAGCACCAcAG | Intron (NSFP1) |
| | (SEQ. ID. NO.: 531) | |
| chr17: 44694678 | ACCAAAGCCTAGAGCACCAcAG | Intron (NSF) |
| | (SEQ. ID. NO.: 532) | |
| chr1: 111905632 | ATCGTTCCCTGGAGCACCAtAG | Intergenic |
| | (SEQ. ID. NO.: 533) | |
| chr1: 71470495 | AACAATGCCTGGATCACCAcAG | Intron (PTGER3) |
| | (SEQ. ID. NO.: 534) | |
| chr2: 207920140 | GTCTTTTCCTGGAGCACCAgAG | Intergenic |
| | (SEQ. ID. NO.: 535) | |
| chr17: 58128153 | AATCATGGCTGGAGCACCAgAG | Intron (HEATR6) |
| | (SEQ. ID. NO.: 536) | |
| chr1: 22917503 | GTCCATGCCTGGACCACCAcAG | Intron (EPHA8) |
| | (SEQ. ID. NO.: 537) | |
| chr3: 140814185 | GTCGCTGCCTGGAGCACCAtGG | Intron (SPSB4) |
| | (SEQ. ID. NO.: 538) | |
| chr1: 15137393 | GGCACTGCCTGGAGCACCAtGG | Intron (KAZN) |
| | (SEQ. ID. NO.: 539) | |
| chr16: 88812827 | AGCCCTGCCTGGAGCACCAgGG | Intron (PIEZO1) |
| | (SEQ. ID. NO.: 540) | |
| chr6: 43014827 | ATCAGTTCCTGGAGCACCTgGG | Exon Coding Sequence (CUL7) |
| | (SEQ. ID. NO.: 541) | |
| chr22: 18437396 | AACCATGCCTGGAACACCAtGG | Intron (MICAL3) |
| | (SEQ. ID. NO.: 542) | |
| chr15: 25425129 | ATCAAATCCTGGAGCCCCAgGG | Intron (SNURF-SNRPN) |
| | (SEQ. ID. NO.: 543) | |
| chr8: 144363328 | GGCAATGCCTGGAGCAACAaAG | Intergenic |
| | (SEQ. ID. NO.: 544) | |
| chr6: 141226784 | ATGAGTGCCTGAAGCACCAaGG | Intergenic |
| | (SEQ. ID. NO.: 545) | |

TABLE 27
Targeting Exon 22:

| Genome Coordinates | Sequence | Genomic Region |
|---|---|---|
| chX: 154124374 (target) | AGAAGTGGCAGACTTATCGaGG | Exon Coding Sequence (F8) |
| | (SEQ. ID. NO.: 546) | |
| chr21: 42038990 | AGAAGCAGCAGACTTATCCaGG | Intron (DSCAM) |
| | (SEQ. ID. NO.: 547) | |
| chr12: 69990980 | GGAAGTTGCAAACTTATCGaGG | Exon Coding Sequence (CCT2) |
| | (SEQ. ID. NO.: 548) | |
| chr7: 110964978 | GGATGTGGCAGACTTATCTtAG | Intron (IMMPL2) |
| | (SEQ. ID. NO.: 549) | |
| chr8: 42174378 | CTGAGTGGCAGGCTTATCGgGG | Exon Coding Sequence (IKBKB) |
| | (SEQ. ID. NO.: 550) | |
| chr3: 57930763 | AGAACAGGCAGACTTATCTtAG | Intergenic |
| | (SEQ. ID. NO.: 551) | |
| chr1: 52997435 | AGAAGAGGCATACTTATCTgAG | Intron (ZCCHC11) |
| | (SEQ. ID. NO.: 552) | |
| chr15: 27460224 | GAAACTGGCAGACTTATCTaGG | Intron (GABRG3) |
| | (SEQ. ID. NO.: 553) | |
| chr2: 102965996 | AGAAGTGGCAGAGTTATCCtGG | Intron (IL1RL1) |
| | (SEQ. ID. NO.: 554) | |
| chr20: 2306018 | AGGAGTGGCTGACTTATCTaAG | Intron (TGM3) |
| | (SEQ. ID. NO.: 555) | |
| chr8: 92580265 | AAAAATGGTAGACTTATCAaAG | Intergenic |
| | (SEQ. ID. NO.: 556) | |
| chr13: 113875149 | AGAAGTCGCAGGCTTATGGgAG | Intron (CUL4A) |
| | (SEQ. ID. NO.: 557) | |
| chr18: 30300891 | AGAAGAGGAAGACTTATGGaAG | Intron (KLHL14) |
| | (SEQ. ID. NO.: 558) | |
| chr2: 135308659 | AGTGCTGGCAGACTTATTGcAG | Intron (TMEM163) |
| | (SEQ. ID. NO.: 559) | |
| chr11: 133197425 | AGGAGGGGCAGATTTATCGaAG | Intron (OPCML) |
| | (SEQ. ID. NO.: 560) | |
| chr12: 102978261 | AGAAGTAGAAAACTTATCAtAG | Intergenic |
| | (SEQ. ID. NO.: 561) | |
| chr3: 30382779 | AGCAGTGGCAGACATATTGaAG | Intergenic |
| | (SEQ. ID. NO.: 562) | |
| chr6: 118027061 | AGAAGTGGATGACTTATTGcAG | Intron (NUS1) |
| | (SEQ. ID. NO.: 563) | |
| chr9: 117888881 | GCAAGTGGCAGGCTTATCTgGG | Intron (LOC101928748) |
| | (SEQ. ID. NO.: 564) | |
| chr2: 51293036 | GCAAGTGGCAGACTTTTCCaAG | Intergenic |
| | (SEQ. ID. NO.: 565) | |
| chr21: 36105270 | AAGAGTGGCAGACTTCTCAtGG | Non-coding Exon (LINC00160) |
| | (SEQ. ID. NO.: 566) | |

Sequences listed in Table 28 contain identified binding sites for TALENs within exons 1-22 respectively. If a similar sequence existed in the homologous exon in the canine genome (canFam3 genome build), that corresponding binding site is shown with any mismatches in lowercase red; if insufficient homology to permit a reasonable possibility of the TALENs being able to cleave the canine exon, the site is listed as “N/A”.

TABLE 28

| FVIII Gene | Genome Editing | Genomic Target of TALEN | Target of TALEN in Dogs |
|---|---|---|---|
| (Region) | Position | (DNA Sequence) | (DNA Sequence) |
| Exon 1 | 5′ Half-Site | 5′-TGGAACTGTCATGGGAC | N/A |
| | | (SEQ. ID. NO.: 569) | |
| | 3′ Half-Site | 5′-TCCACAGGCAGCTCACCGAG | N/A |
| | | (SEQ. ID. NO.: 570) | |
| Exon 2 | 5′ Half-Site | 5′-TCTGTTTGTAGAATTCACGG | N/A |
| | | (SEQ. ID. NO.: 571) | |
| | 3′ Half-Site | 5′-TGGCCTTGGCTTAGCGAT | N/A |
| | | (SEQ. ID. NO.: 572) | |
| Exon 3 | 5′ Half-Site | 5′-TACACTTAAGAACATGGCT | N/A |
| | | (SEQ. ID. NO.: 573) | |
| | 3′ Half-Site | 5′-TACACCAACAGCATGAAGAC | N/A |
| | | (SEQ. ID. NO.: 574) | |
| Exon 4 | 5′ Half-Site | 5′-TGTGCCTTACCTACTCATATCT | N/A |
| | | (SEQ. ID. NO.: 575) | |
| | 3′ Half-Site | 5′-TGAATTCAAGTCTTTTACCAG | N/A |
| | | (SEQ. ID. NO.: 576) | |
| Exon 5 | 5′ Half-Site | 5′-TCTGGCCAAGGAAAAGACACAGAC | 5′- |
| | | (SEQ. ID. NO.: 577) | TCTGGCCAAaGAAAgGACACAGAC |
| | | | (SEQ. ID. NO.: 613) |
| | 3′ Half-Site | 5′-TTCATCAAATACAGCAAAAAGTAG | 5′- |
| | | (SEQ. ID. NO.: 578) | TTCATCAAATACAGCAAAAAGTAG |
| | | | (SEQ. ID. NO.: 614) |
| Exon 6 | 5′ Half-Site | 5′-TGCTGCATCTGCTCGGG | N/A |
| | | (SEQ. ID. NO.: 579) | |
| | 3′ Half-Site | 5′-TTTACATAACCATTGACTGTGT | N/A |
| | | (SEQ. ID. NO.: 580) | |
| Exon 7 | 5′ Half-Site | 5′-TCTCGCCAATAACTTTCC | N/A |
| | | (SEQ. ID. NO.: 581) | |
| | 3′ Half-Site | 5′-TGTCCAAGGTCCATCAAGAG | N/A |
| | | (SEQ. ID. NO.: 582) | |
| Exon 8 | 5′ Half-Site | 5′-TCAGTTGCCAAGAAGCATCCTAA | 5′-TCAGTTGCCAAGAAGCATCCTAA |
| | | (SEQ. ID. NO.: 583) | (SEQ. ID. NO.: 615) |
| | 3′ Half-Site | 5′-TCCTCCTCTTCAGCAGCAATGT | 5′-TCCTCCTCcTCAGCAGCAATaT |
| | | (SEQ. ID. NO.: 584) | (SEQ. ID. NO.: 616) |
| Exon 9 | 5′ Half-Site | 5′-TTCAGCATGAATCAGGAA | N/A |
| | | (SEQ. ID. NO.: 585) | |
| | 3′ Half-Site | 5′-TCTCCAACTTCCCCATAA | N/A |
| | | (SEQ. ID. NO.: 586) | |
| Exon 10 | 5′ Half-Site | 5′-TATAACATCTACCCTCACGG | N/A |
| | | (SEQ. ID. NO.: 587) | |
| | 3′ Half-Site | 5′-TCTCCTTGAATACAAAGGAC | N/A |
| | | (SEQ. ID. NO.: 588) | |
| Exon 11 | 5′ Half-Site | 5′-TCTAGCTTCAGGACTCAT | 5′-TCTAGCTTCAGGACTCAT |
| | | (SEQ. ID. NO.: 589) | (SEQ. ID. NO.: 617) |
| | 3′ Half-Site | 5′-TCTACAGATTCTTTGTAGCAG | 5′-TCTACAGATTCTTTGTAGCAG |
| | | (SEQ. ID. NO.: 590) | (SEQ. ID. NO.: 618) |
| Exon 12 | 5′ Half-Site | 5′-TCACAGAGAATATACAACG | N/A |
| | | (SEQ. ID. NO.: 591) | |
| | 3′ Half-Site | 5′-TCCTCAAGCTGCACTCCAGCT | N/A |
| | | (SEQ. ID. NO.: 592) | |
| Exon 13 | 5′ Half-Site | 5′-TGTCTTCTTCTCTGGAT | 5′-TGTCTTCTTCTCTGGAT |
| | | (SEQ. ID. NO.: 593) | (SEQ. ID. NO.: 619) |
| | 3′ Half-Site | 5′-TGTGTCTTCATAGACCATTTT | 5′-TGTGTCTTCATAGACCATTTT |
| | | (SEQ. ID. NO.: 604) | (SEQ. ID. NO.: 620) |
| Exon 14 | 5′ Half-Site | 5′-TCAAAAGAAAACACGACACTATTT | 5′- |
| | | (SEQ. ID. NO.: 595) | TCAAAAGAAAACACGACACTATTT |
| | | | (SEQ. ID. NO.: 621) |
| | 3′ Half-Site | 5′-TCATCCCATAATCCCAGAGCCTCT | 5′- |
| | | (SEQ. ID. NO.: 596) | TCATCCCATAATCCCAGAGaCgCT |
| | | | (SEQ. ID. NO.: 622) |
| Exon 15 | 5′ Half-Site | 5′-TCAGCCCTTATACCGTGGAG | 5′-TCAGCCCTTATACCGTGGAG |
| | | (SEQ. ID. NO.: 597) | (SEQ. ID. NO.: 623) |
| | 3′ Half-Site | 5′-TATGGCCCCAGGAGTCCCAA | 5′-TATGGCCCCAaGAGTCCCAA |
| | | (SEQ. ID. NO.: 598) | (SEQ. ID. NO.: 624) |
| Exon 16 | 5′ Half-Site | 5′-TATGGCACCCACTAAAGATGAG | 5′-TATGGCACCCACTAAAGATGAG |
| | | (SEQ. ID. NO.: 599) | (SEQ. ID. NO.: 625) |
| | 3′ Half-Site | 5′-TCAGAGAAATAAGCCCAG | 5′-TCAGAaAAATAAGCCCAG |
| | | (SEQ. ID. NO.: 600) | (SEQ. ID. NO.: 626) |
| Exon 17 | 5′ Half-Site | 5′-TCTTTGATGAGACCAAA | N/A |
| | | (SEQ. ID. NO.: 601) | |
| | 3′ Half-Site | 5′-TCTTTCCATATTTTCAG | N/A |
| | | (SEQ. ID. NO.: 602) | |
| Exon 18 | 5′ Half-Site | 5′-TCTATTCATTTCAGTGGAC | N/A |
| | | (SEQ. ID. NO.: 603) | |
| | 3′ Half-Site | 5′-TATACTCCTCTTTTTTTCG | N/A |
| | | (SEQ. ID. NO.: 604) | |
| Exon 19 | 5′ Half-Site | 5′-TGTTACCATCCAAAGCT | N/A |
| | | (SEQ. ID. NO.: 605) | |
| | 3′ Half-Site | 5′-TGCTCGCCAATAAGGCATTCC | N/A |
| | | (SEQ. ID. NO.: 606) | |
| Exon 20 | 5′ Half-Site | 5′-TCCCCTGGGAATGGCTTCTGG | N/A |
| | | (SEQ. ID. NO.: 607) | |
| | 3′ Half-Site | 5′-TGTCCTGAAGCTGTAATCTGAA | N/A |
| | | (SEQ. ID. NO.: 608) | |
| Exon 21 | 5′ Half-Site | 5′-TGGGCCCCAAAGCTGGCCAG | 5′-TGGGCCCCAAAGCTGGCCAG |
| | | (SEQ. ID. NO.: 609) | (SEQ. ID. NO.: 627) |
| | 3′ Half-Site | 5′-TGCTCCAGGCATTGATTGAT | 5′-TGCTCCAGGCATTGATTGAT |
| | | (SEQ. ID. NO.: 610) | (SEQ. ID. NO.: 628) |
| Exon 22 | 5′ Half-Site | 5′-TCTACATCTCTCAGTTTAT | N/A |
| | | (SEQ. ID. NO.: 611) | |
| | 3′ Half-Site | 5′-TCTGCCACTTCTTCCCATCAAG | N/A |
| | | (SEQ. ID. NO.: 612) | |

Sequences listed in Tables 29-50 below contain the top 20 potential off-target sites computationally identified in the human genome for the previously mentioned TALEN binding sites in exons 1-22, respectively. Off-target analysis was performed using the PROGNOS algorithm (Fine et al., Nucleic Acids Research 2013) “TALEN v2.0” on the hg19 build of the human genome. The top 20 potential off-target sites are given for each TALEN pair. Homodimers were allowed in the search and spacing between the TALENs of 10-30 bp. The right half-site is listed as the sequence on the same strand as the left half-site; the right half-site is therefore listed in the reverse anti-sense orientation to the sequence which is bound by the TALEN. Left and right half-sites are given as the 5′ (left) and 3′ (right) binding sites on the positive strand of the chromosome; the “left” and “right” annotation may therefore differ from the annotation for TALENs designed to genes on the negative strand of chromosomes. Mismatches to the intended binding sequence are depicted in lowercase letters.

TABLE 29
Targeting Exon 1:

| Genome Coordinates | Left Half-Site | Right Half-Site | Genomic Region |
|---|---|---|---|
| chrX: 154250691 | TCCACAGGCAGCTCACCGAG | GTCCCATGACAGTTCCA | Exon (F8) |
| | (SEQ. ID. NO.: 629) | (SEQ. ID. NO.: 650) | |
| chr14: 45095676 | TGGAACTcTCATGGaAC | GagCaATGACtGTTCCA | Intergenic |
| | (SEQ. ID. NO.: 630) | (SEQ. ID. NO.: 651) | |
| chr6: 26839581 | aGGAgCTGTCAgtcaAC | GTCtCATGACAGTTaCA | Intron (GUSBP4 |
| | (SEQ. ID. NO.: 631) | (SEQ. ID. NO.: 652) | |
| chr10: 45462110 | TGGAACTGTCATGGtgC | CTCaGaGAGtTGCCTGgttA | Intron (RASSF4) |
| | (SEQ. ID. NO.: 632) | (SEQ. ID. NO.: 653) | |
| chr11: 101870316 | TGaAACTGTCATatGAC | tgCCCATGACtccTCCA | Exon (KIAA1377) |
| | (SEQ. ID. NO.: 633) | (SEQ. ID. NO.: 654) | |
| chr15: 20414578 | TGaAgCTGTCATGaaAC | cTtCCATtAtAGTTttA | Intergenic |
| | (SEQ. ID. NO.: 634) | (SEQ. ID. NO.: 655) | |
| chr16: 33444315 | TaaAACTaTaATGGaAg | GTttCATGACAGcTtCA | Intergenic |
| | (SEQ. ID. NO.: 635) | (SEQ. ID. NO.: 656) | |
| chr5: 61534127 | TGaAgCTGTCATGaaAC | cTtCCATtAtAGTTttA | Intergenic |
| | (SEQ. ID. NO.: 636) | (SEQ. ID. NO.: 657) | |
| chr7: 44551672 | TGGAcCcagCATGGGgC | GTtCCtTGACAtTTCCA | Intergenic |
| | (SEQ. ID. NO.: 637) | (SEQ. ID. NO.: 658) | |
| chr1: 165095506 | TGGAACTGTCATGtGAg | GTtCCATGgCAGaTaCt | Intergenic |
| | (SEQ. ID. NO.: 638) | (SEQ. ID. NO.: 659) | |
| chrX: 15724565 | TaGgACTGTCcTGaGcC | GgCtCAgGACAGTcCCA | Intergenic |
| | (SEQ. ID. NO.: 639) | (SEQ. ID. NO.: 660) | |
| chr7: 67809648 | TaGAACTaTCATGGGAa | GgCttcTGAgAcTTCCA | Intergenic |
| | (SEQ. ID. NO.: 640) | (SEQ. ID. NO.: 661) | |
| chr6: 13204828 | TGGcAtTGTCATGGaAC | GTCCtAgGtagGTTCCA | Intron (PHACTR1) |
| | (SEQ. ID. NO.: 641) | (SEQ. ID. NO.: 662) | |
| chr2: 37743218 | TGaAACccTCATGaGcC | GTCCtATGAgAtTTCtA | Intergenic |
| | (SEQ. ID. NO.: 642) | (SEQ. ID. NO.: 663) | |
| chr10: 78301531 | TGtAAaTGTCATGGaAC | GTCtCATttCAGTgtaA | Intron (C10orf11) |
| | (SEQ. ID. NO.: 643) | (SEQ. ID. NO.: 664) | |
| chrX: 106781486 | TGGAAaTGTCATaGaAC | cTCCatTGACAGaTCtt | Intergenic |
| | (SEQ. ID. NO.: 644) | (SEQ. ID. NO.: 665) | |
| chr12: 70809983 | TaGgtCTGTCtTGGGtC | GctCCATGtCAGTTtCA | Intron (KCNMB4) |
| | (SEQ. ID. NO.: 645) | (SEQ. ID. NO.: 666) | |
| chr11: 46818282 | TatAACTGTCAaGaGAC | GTCCaATttCAGTcCaA | Intron (CKAP5) |
| | (SEQ. ID. NO.: 646) | (SEQ. ID. NO.: 667) | |
| chr3: 30945924 | TGGAgCTGaaAaGcaAC | GTCtCcTGACAGcTCCA | Intergenic |
| | (SEQ. ID. NO.: 647) | (SEQ. ID. NO.: 668) | |
| chr9: 13642916 | TaGAACTaaCATaaaAC | GTgtCATtAtAGTTgCA | Intergenic |
| | (SEQ. ID. NO.: 648) | (SEQ. ID. NO.: 669) | |
| chr14: 27743308 | TaGAAaTaTCcTGGGAt | aTtgCATGAtAGTTCCA | Intergenic |
| | (SEQ. ID. NO.: 649) | (SEQ. ID. NO.: 670) | |

TABLE 30
Targeting Exon 2:

| Genome Coordinates | Left Half-Site | Right Half-Site | Genomic Region |
|---|---|---|---|
| chrX: 154227764 | TGGCCTTGGCTTAGCGAT | CCGTGAATTCTACAAACAGA | Exon (F8) |
| | (SEQ. ID. NO.: 671) | (SEQ. ID. NO.: 692) | |
| chr12: 51122429 | TGGaCTTGGCTTcGCGcT | ATgGaaAAGCCAAGGagA | Exon (DIP2B) |
| | (SEQ. ID. NO.: 672) | (SEQ. ID. NO.: 693) | |
| chr14: 83666273 | TaGCCTTGGCTTAGaaAa | cTgGCTAAGCaAAGataA | Intergenic |
| | (SEQ. ID. NO.: 673) | (SEQ. ID. NO.: 694) | |
| chr15: 99285268 | gGaaCTTGaCTTAGCccT | cctGCTAAGCCAAGGCtA | Intron (IGF1R) |
| | (SEQ. ID. NO.: 674) | (SEQ. ID. NO.: 695) | |
| chr15: 29750773 | TGcCCTgGaCTTgGaGgT | AgaGaTAAGCCAAGGtCA | Intron (FAM189A1) |
| | (SEQ. ID. NO.: 675) | (SEQ. ID. NO.: 696) | |
| chr20: 59053322 | TGGCCTTGGtTTAGaaAa | AgCGaTAAGgaAAGGttA | Intergenic |
| | (SEQ. ID. NO.: 676) | (SEQ. ID. NO.: 697) | |
| chr1: 163956121 | TCTaTTTGTAGAATTactaG | tTgGtTAAGCCAAttCCA | Intergenic |
| | (SEQ. ID. NO.: 677) | (SEQ. ID. NO.: 698) | |
| chr2: 123622749 | TCTtTTTGTAaAAaTgACGa | ATtcCgAAGCCAAGGatA | Intergenic |
| | (SEQ. ID. NO.: 678) | (SEQ. ID. NO.: 699) | |
| chr12: 92444873 | TGtCCaTGGCcTgGgGgT | ATCttgAAGCCAAGGCtA | Intron (LOC256021) |
| | (SEQ. ID. NO.: 679) | (SEQ. ID. NO.: 700) | |
| chr14: 86193436 | caGCCTTGGCTTgtgGAT | tTtaCTAAGaCAAGGCCA | Intergenic |
| | (SEQ. ID. NO.: 680) | (SEQ. ID. NO.: 701) | |
| chr8: 1184501 | TGaCCTctcCTTAaCcAT | ATttCTAAaCtAAGGtCA | Intergenic |
| | (SEQ. ID. NO.: 681) | (SEQ. ID. NO.: 702) | |
| chr4: 60350711 | TGGCaaTGcCTTAGaaAT | ATtGCTAAGtCAAatCaA | Intergenic |
| | (SEQ. ID. NO.: 682) | (SEQ. ID. NO.: 703) | |
| chr2: 109270631 | TttCCTTGGCTTAGtGAT | ATtGCTAActCAAtcaCA | Promoter (LIMS1) |
| | (SEQ. ID. NO.: 683) | (SEQ. ID. NO.: 704) | |
| chr2: 110655405 | TttCCTTGGCTTAGtGAT | ATtGCTAActCAAtcaCA | Promoter (LIMS3-LOC440895) |
| | (SEQ. ID. NO.: 684) | (SEQ. ID. NO.: 705) | |
| chr2: 111231206 | TGtgaTTGagTTAGCaAT | ATCaCTAAGCCAAGGaaA | Promoter (LIMS3-LOC440895) |
| | (SEQ. ID. NO.: 685) | (SEQ. ID. NO.: 706) | |
| chr7: 105518314 | ctGCCcTGGCTgAaCcAT | ATCGCTAAGCCAgtGttA | Intergenic |
| | (SEQ. ID. NO.: 686) | (SEQ. ID. NO.: 707) | |
| chrX: 12453009 | TtGCaTTtaCTcAGCcAT | ATCttTtAGCCAAtGCCA | Intron (FRMPD4) |
| | (SEQ. ID. NO.: 687) | (SEQ. ID. NO.: 708) | |
| chr9: 133831225 | TGGCCTgaGCTTtGgGgT | ActGCTAAGaCAAGcCCA | Intergenic |
| | (SEQ. ID. NO.: 688) | (SEQ. ID. NO.: 709) | |
| chr7: 27778567 | TgTGcTTaTAaAATTCACtG | CaGTtAtTTCTACtAcCAGA | Promoter (TAX1BP1) |
| | (SEQ. ID. NO.: 689) | (SEQ. ID. NO.: 710) | |
| chr8: 22054601 | TaGggcTGGCTTgGCGAg | gTaGCTAAGtCAAGGCtA | Intron (BMP1) |
| | (SEQ. ID. NO.: 690) | (SEQ. ID. NO.: 711) | |
| chr6: 102761808 | TGGCagTaGCTctGCcAT | AattCTAAGCtAAGGCCA | Intergenic |
| | (SEQ. ID. NO.: 691) | (SEQ. ID. NO.: 712) | |

TABLE 31
Targeting Exon 3:

| Genome Coordinates | Left Half-Site | Right Half-Site | Genomic Region |
|---|---|---|---|
| chrX: 154225270 | TACACCAACAGCATGAAGAC | AGCCATGTTCTTAAGTGTA | Exon (F8) |
| | (SEQ. ID. NO.: 713) | (SEQ. ID. NO.: 734) | |
| chr2: 175647194 | aACAaTcAgGctCATGGCa | AGCCATGTTtTTAAGaGTA | Intergenic |
| | (SEQ. ID. NO.: 714) | (SEQ. ID. NO.: 735) | |
| chr4: 164801896 | TAtACTTAAaAACATaGCT | AGtgATtTTtTTcAaTGaA | Intron (MARCH1) |
| | (SEQ. ID. NO.: 715) | (SEQ. ID. NO.: 736) | |
| chr3: 1591042 | TACAtTTAAaAACATGtCT | AGCtATcTTaTTcAtTtTA | Intergenic |
| | (SEQ. ID. NO.: 716) | (SEQ. ID. NO.: 737) | |
| chr21: 39750804 | TACgCTgcAGAgCtgGGCa | AGaCATtTTtTTAAGTGTA | Intron (ERG) |
| | (SEQ. ID. NO.: 717) | (SEQ. ID. NO.: 738) | |
| chrX: 46478957 | TACACaTAAcAACATGGCT | AGCCAgacaCTaAAaTaTA | Intron (SLC9A7) |
| | (SEQ. ID. NO.: 718) | (SEQ. ID. NO.: 739) | |
| chrX: 99327213 | aAtcCTTAAGAACATGaCT | AtCCtTGTTCTTAtGTtcA | Intergenic |
| | (SEQ. ID. NO.: 719) | (SEQ. ID. NO.: 740) | |
| chr8: 103196820 | cACACTgAAGAcCATGGCT | GTCTTCATcaTGTTaGTGTc | Intergenic |
| | (SEQ. ID. NO.: 720) | (SEQ. ID. NO.: 741) | |
| chr9: 76364644 | TAgACTTAAtcAtgTaGCT | gGCtATGTTCTTAAGTGTc | Intergenic |
| | (SEQ. ID. NO.: 721) | (SEQ. ID. NO.: 742) | |
| chr8: 19520723 | TACACTTgtGAAgATGGaT | AGgCtTGTaCTTAAtTGTA | Intron (CSGALNACT1) |
| | (SEQ. ID. NO.: 722) | (SEQ. ID. NO.: 743) | |
| chr1: 7465386 | TACACTTAgaAAaAaaGCT | GTtTgttTGCTGTTGtTGTt | Intron (CAMTA1) |
| | (SEQ. ID. NO.: 723) | (SEQ. ID. NO.: 744) | |
| chrX: 151388800 | TACACTTAtGtgttTGGCT | AtCCATGTTgTTgAGTGTA | Intron (GABRA3) |
| | (SEQ. ID. NO.: 724) | (SEQ. ID. NO.: 745) | |
| chr8: 52110351 | aACACTTAAaAACAgGGCT | AtCtATtTaCTaAAtTGTt | Intergenic |
| | (SEQ. ID. NO.: 725) | (SEQ. ID. NO.: 746) | |
| chr11: 42440454 | aACAaaTAAtAtCATcaCT | AtCtATGTTCTTAAGTcTA | Intergenic |
| | (SEQ. ID. NO.: 726) | (SEQ. ID. NO.: 747) | |
| chr2: 74468885 | cgCACaaAAaAACATGGaT | AGgCATGTTtTTAAGTGgg | Intron (SLC4A5) |
| | (SEQ. ID. NO.: 727) | (SEQ. ID. NO.: 748) | |
| chr6: 82600824 | cACAtTTgAGAACATGGCT | GctTTCAgtCTGgTGGTtTA | Intergenic |
| | (SEQ. ID. NO.: 728) | (SEQ. ID. NO.: 749) | |
| chr2: 65094538 | TgCACTTAAaAAtATGaCa | AGCacaGTgCTTAAGTGcA | Intergenic |
| | (SEQ. ID. NO.: 729) | (SEQ. ID. NO.: 750) | |
| chrX: 87497023 | TACACTgAAGAgaATGGag | AGCaATGTTtTTAAGTGat | Intergenic |
| | (SEQ. ID. NO.: 730) | (SEQ. ID. NO.: 751) | |
| chr13: 74882688 | TtCAtTgAAGAAaAaaGCT | aTtTTtATGCTGTTGGaGTA | Intergenic |
| | (SEQ. ID. NO.: 731) | (SEQ. ID. NO.: 752) | |
| chr21: 25077810 | TACAtTTAAGcAtATGGCT | tGCttTagTCTTAAtTGTA | Intergenic |
| | (SEQ. ID. NO.: 732) | (SEQ. ID. NO.: 753) | |
| chr10: 92935297 | TACcCcTgtGAACATGGaa | tGCttTGTTCTTAAaTGTA | Intron (PCGF5) |
| | (SEQ. ID. NO.: 733) | (SEQ. ID. NO.: 754) | |

TABLE 32
Targeting Exon 4:

| Genome Coordinates | Left Half-Site | Right Half-Site | Genomic Region |
|---|---|---|---|
| chrX: 154221245 | TGAATTCAAGTCTTTTACCAG | AGATATGAGTAGGTAAGGCACA | Exon (F8) |
| | (SEQ. ID. NO.: 755) | (SEQ. ID. NO.: 776) | |
| chr5: 166223644 | TGAATTCAAaTCTTTTtCCtG | tTGGaAAAAtcCcTtAATaCA | Intergenic |
| | (SEQ. ID. NO.: 756) | (SEQ. ID. NO.: 777) | |
| chr3: 48957213 | TGAtTTCtAGTtTTgTgCCAa | tTaGTAAAtGACcTGAATTCA | Promoter (C3orf71) |
| | (SEQ. ID. NO.: 757) | (SEQ. ID. NO.: 778) | |
| chr1: 14460511 | TGAcaTtAAGaCaTTTAaCAG | CTGGgAAAAGAagTGgATTCA | Intergenic |
| | (SEQ. ID. NO.: 758) | (SEQ. ID. NO.: 779) | |
| chr8: 26674607 | gaAAggCAAGcCaTaTACtAG | CTGaTAAAtGACTTGtATTCA | Intron (ADRA1A) |
| | (SEQ. ID. NO.: 759) | (SEQ. ID. NO.: 780) | |
| chr15: 41366843 | TGcATaCAAtTCcTTTACCAa | CTGaTAAAcaAtTTtAATTtA | Intron (INO80) |
| | (SEQ. ID. NO.: 760) | (SEQ. ID. NO.: 781) | |
| chr6: 134930070 | TaAAgTCActTCcTTTACgAc | aTGGTtgAtGACTTGAATTCA | Intergenic |
| | (SEQ. ID. NO.: 761) | (SEQ. ID. NO.: 782) | |
| chr6: 121097474 | TGAATcCAAaaCTTTTACCtG | CTGGgttAAtACaTttATTtA | Intergenic |
| | (SEQ. ID. NO.: 762) | (SEQ. ID. NO.: 783) | |
| chr11: 49119615 | gGAATTaAAGTCcTTcACata | tTGGTtAcAGACTTGAAgTCA | Intergenic |
| | (SEQ. ID. NO.: 763) | (SEQ. ID. NO.: 784) | |
| chr1: 74307557 | gGAATTCAAtTCaaTaACaAG | tgGGcAAAAGACcTGAATTgA | Intergenic |
| | (SEQ. ID. NO.: 764) | (SEQ. ID. NO.: 785) | |
| chr18: 38466162 | TGtATTCAAGTCcTTaAaaAG | tTGGTtAAAattTTGAAcTCA | Intergenic |
| | (SEQ. ID. NO.: 765) | (SEQ. ID. NO.: 786) | |
| chr20: 45113912 | atAATTCtAGTCTTaggaCAG | CTGGgAAAAGttTgGAATTtA | Intergenic |
| | (SEQ. ID. NO.: 766) | (SEQ. ID. NO.: 787) | |
| chr5: 26641542 | TGAATTCcttcCTTgTACCAt | tgGaTtAAAGACTTGAATgCA | Intergenic |
| | (SEQ. ID. NO.: 767) | (SEQ. ID. NO.: 788) | |
| chr3: 160034110 | TGAAagCAAaTCTTTccCCAG | CTGGTcAAtGcCTTGctTgCA | Intron (IFT80) |
| | (SEQ. ID. NO.: 768) | (SEQ. ID. NO.: 789) | |
| chr2: 241783612 | TGAcTTCAAGTCTTTaAaCAa | aTcagAAAAtctTTGAATcCA | Intergenic |
| | (SEQ. ID. NO.: 769) | (SEQ. ID. NO.: 790) | |
| chr6: 123852751 | gGTcaCTaAtCTACTCtTATCT | AGATATGAacAGGTAAGGCACt | Intron (TRDN) |
| | (SEQ. ID. NO.: 770) | (SEQ. ID. NO.: 791) | |
| chr2: 89343189 | TGAATTCAAcTCTTTagaCAG | gTaaggAAAGctTTGAATTCA | Intergenic |
| | (SEQ. ID. NO.: 771) | (SEQ. ID. NO.: 792) | |
| chr2: 90195655 | TGAATTCAAagCTTTccttAc | CTGtctAAAGAgTTGAATTCA | Intergenic |
| | (SEQ. ID. NO.: 772) | (SEQ. ID. NO.: 793) | |
| chr8: 13349868 | TGAAaTtgAaTCTgaTtCCAG | tTtGTcAAAGACTTGtATTtA | Intron (DLC1) |
| | (SEQ. ID. NO.: 773) | (SEQ. ID. NO.: 794) | |
| chrY: 4231090 | TGAATTCAAtTCTTcagCCAG | tcaGaAAAAtctTTGAATcCA | Intergenic |
| | (SEQ. ID. NO.: 774) | (SEQ. ID. NO.: 795) | |
| chrX: 90035974 | TGAATTCAAtTCTTcagCCAG | tcaGaAAAAtctTTGAATcCA | Intergenic |
| | (SEQ. ID. NO.: 775) | (SEQ. ID. NO.: 796) | |

TABLE 33
Targeting Exon 5:

| Genome Coordinates | Left Half-Site | Right Half-Site | Genomic Region |
|---|---|---|---|
| chrX: 154215513 | TTCATCAAATACAGCAAAAAGTAG | GTCTGTGTCTTTTCCTTGGCCAGA | Exon (F8) |
| | (SEQ. ID. NO.: 797) | (SEQ. ID. NO.: 818) | |
| chr8: 65938903 | TCTaGCCAAGccAgAGgCACtGAC | GgCTcTGTCTTTTCCTctGCCAcA | Intergenic |
| | (SEQ. ID. NO.: 798) | (SEQ. ID. NO.: 819) | |
| chr1: 26774318 | TTCAaCAAcaACAaCAAAAAagca | cTCTGTGcCaTgTaCTTGGCCAGA | Intron (DHDDS) |
| | (SEQ. ID. NO.: 799) | (SEQ. ID. NO.: 820) | |
| chr10: 102225665 | cTCAcCAAgcAttGCAtAAAGctG | CTACTTTTaGgTGTATTTtATGAA | Intron (WNT8B) |
| | (SEQ. ID. NO.: 800) | (SEQ. ID. NO.: 821) | |
| chr7: 14755743 | TTCATCAAcTcCAGgAAAAAcaAc | GTaTaTGTgTTTTCacTGGaCAGA | Intron (DGKB) |
| | (SEQ. ID. NO.: 801) | (SEQ. ID. NO.: 822) | |
| chr8: 124089292 | TTCATaAtATcaAGtAAtAcGTga | GTtTGgGTtTTTTtCTTtGaCAGA | Intron (WDR67) |
| | (SEQ. ID. NO.: 802) | (SEQ. ID. NO.: 823) | |
| chr6: 70049288 | TCTGGCCAtGacAgAtAaACgctC | aTACTTTTTGCTGTgTTTGATtcA | Exon (BAI3) |
| | (SEQ. ID. NO.: 803) | (SEQ. ID. NO.: 824) | |
| chr17: 37764808 | TCaaaCCAAGGgAAAGACAgAGAa | GTCTGTGcCTcTgCaTgGGCgtGt | Promoter |
| | (SEQ. ID. NO.: 804) | (SEQ. ID. NO.: 825) | (NEUROD2) |
| chr2: 92285124 | TCTtGCCAcaaAAAAtACACAGAa | CTACgTTgTGaTGTgTTTacTcAA | Intergenic |
| | (SEQ. ID. NO.: 805) | (SEQ. ID. NO.: 826) | |
| chr11: 80679047 | TTaATaAAgTgaAaCtAAAAGTAa | GTCTGTaTgTTTTatTTtGCtAGA | Intergenic |
| | (SEQ. ID. NO.: 806) | (SEQ. ID. NO.: 827) | |
| chr7: 49746821 | TCaGaCCAAGccAgAGgtgCAcAC | GgCTtTGTCaTTTCCTTGGCCtGt | Intergenic |
| | (SEQ. ID. NO.: 807) | (SEQ. ID. NO.: 828) | |
| chr2: 92283421 | TCTGGCCAcaaAAActACACAGAa | CTACgTTgTGaTGTgTTTacTcAA | Intergenic |
| | (SEQ. ID. NO.: 808) | (SEQ. ID. NO.: 829) | |
| chr6: 53622618 | TCcacCCAAGGAAtAGgCAgAGAg | CTAaTcTTTGCTGTATTTtATtgA | Intergenic |
| | (SEQ. ID. NO.: 809) | (SEQ. ID. NO.: 830) | |
| chr7: 64186025 | gcCAaCAgcaACAGCAAcAAaaAG | GTtTtTGTCTTTTttTTaGaCAGA | Intergenic |
| | (SEQ. ID. NO.: 810) | (SEQ. ID. NO.: 831) | |
| chr8: 76622826 | TCatGaaAAatAAAAGAaACAGta | GTtTtTtTtTTTTCtTgGGaCAGA | Intergenic |
| | (SEQ. ID. NO.: 811) | (SEQ. ID. NO.: 832) | |
| chr13: 27818295 | TCTGtCCAAaaAAAAaAaAaAaAa | gTttTgTTTcCTGaATTTGATaAA | Intergenic |
| | (SEQ. ID. NO.: 812) | (SEQ. ID. NO.: 833) | |
| chr18: 68100701 | TCaGGCCAAtaAAAAacaACAaAC | tgcCTTTTTttTtTtTTTttTGAA | Intergenic |
| | (SEQ. ID. NO.: 813) | (SEQ. ID. NO.: 834) | |
| chr5: 72817667 | TCTaGCaAAGaAAAAtAaACAaAa | tTaTtTtTCTTTTttTTttCCAGc | Intergenic |
| | (SEQ. ID. NO.: 814) | (SEQ. ID. NO.: 835) | |
| chr15: 43320939 | TCaaaCaAAaaAAAAaAaACAaAC | aTaTaTaTaTaTTCCTTGGCCgGA | Intron (UBR1) |
| | (SEQ. ID. NO.: 815) | (SEQ. ID. NO.: 836) | |
| chr4: 12953588 | TaCATaAAAcACAaCAAgAAaTAG | tTACTTacattTGTATTTGAaGAt | Intergenic |
| | (SEQ. ID. NO.: 816) | (SEQ. ID. NO.: 837) | |
| chr22: 49683417 | TCTGGCaAAaGgAtAGcCACAGAt | tTgTGTtTCTTTTtCcTGGgCAtg | Intergenic |
| | (SEQ. ID. NO.: 817) | (SEQ. ID. NO.: 838) | |

TABLE 34
Targeting Exon 6:

| Genome Coordinates | Left Half-Site | Right Half-Site | Genomic Region |
|---|---|---|---|
| chrX: 154212976 | TTTACATAACCATTGACTGTGT | CCCGAGCAGATGCAGCA | Exon (F8) |
| | (SEQ. ID. NO.: 839) | (SEQ. ID. NO.: 860) | |
| chr3: 140445224 | TGCTGCATtaGCTCaGa | CCaGAGCAGAgGCAGCt | Intergenic |
| | (SEQ. ID. NO.: 840) | (SEQ. ID. NO.: 861) | |
| chr8: 56002214 | TaCTGCATCTtCTCtGG | CtgGAGtAGgcGCtGCA | Intergenic |
| | (SEQ. ID. NO.: 841) | (SEQ. ID. NO.: 862) | |
| chr12: 49424040 | gGtgGCATCTGCTCttG | CCCGgGCAGAgGCAGCA | Exon (MLL2) |
| | (SEQ. ID. NO.: 842) | (SEQ. ID. NO.: 863) | |
| chr1: 70622888 | TtCTaCtTCTGCTttaG | tCtGtGtAGATGCAGCA | Intron (LRRC40) |
| | (SEQ. ID. NO.: 843) | (SEQ. ID. NO.: 864) | |
| chr4: 184357162 | TtCTGCcTCTGCTCGaG | ttttAcaAGATGCAGCA | Intergenic |
| | (SEQ. ID. NO.: 844) | (SEQ. ID. NO.: 865) | |
| chr5: 172342828 | TGCaGCcTCTGCTCaGa | CCtGAGCtGggGttGCA | Intron (ERGIC1) |
| | (SEQ. ID. NO.: 845) | (SEQ. ID. NO.: 866) | |
| chr6: 115061184 | TGtTaCAcCTGCTCtGG | gCtGAGCAtATGCAGgA | Intergenic |
| | (SEQ. ID. NO.: 846) | (SEQ. ID. NO.: 867) | |
| chr12: 39726775 | TGaTGCATCTGtTtcGa | CCtGAGCAGgTGCAtCA | Exon (KIF21A) |
| | (SEQ. ID. NO.: 847) | (SEQ. ID. NO.: 868) | |
| chr7: 88799625 | TTTACcTAACCAaTGAaaGTGT | CCtttGtAGATGCAGaA | Intron (ZNF804B) |
| | (SEQ. ID. NO.: 848) | (SEQ. ID. NO.: 869) | |
| chr20: 17949040 | TGCTGCAgCaaCTCGGG | CtCGAGCAGggGCcGCc | Exon (SNX5) |
| | (SEQ. ID. NO.: 849) | (SEQ. ID. NO.: 870) | |
| chr1: 189751560 | TttTcCATCaGCTCaGa | CCtGAGCAGcTtCAGCA | Intergenic |
| | (SEQ. ID. NO.: 850) | (SEQ. ID. NO.: 871) | |
| chr21: 42907464 | TGCcaCATCaGCTCtGG | CCaGAGCAGcaGgAGCA | Intergenic |
| | (SEQ. ID. NO.: 851) | (SEQ. ID. NO.: 872) | |
| chr5: 2548607 | TGCTGCcTCTGCcttca | CatGAGCAGgTGCAGCA | Intergenic |
| | (SEQ. ID. NO.: 852) | (SEQ. ID. NO.: 873) | |
| chr8: 19923395 | TtCTaCATCTGCTCaGa | tCCtgGgAagTGCAGCA | Intergenic |
| | (SEQ. ID. NO.: 853) | (SEQ. ID. NO.: 874) | |
| chr6: 15883284 | TGCTGtcTCTGCTCaGG | CCtGAGCgGAaGCAGag | Intergenic |
| | (SEQ. ID. NO.: 854) | (SEQ. ID. NO.: 875) | |
| chr17: 81092958 | TGCaGCcTCTGCTCcaG | tCCcAGgAGATGtAGaA | Intergenic |
| | (SEQ. ID. NO.: 855) | (SEQ. ID. NO.: 876) | |
| chrX: 153711226 | TGCTGCATCTaCTCctG | CCCGgGCAGATctAttg | Intergenic |
| | (SEQ. ID. NO.: 856) | (SEQ. ID. NO.: 877) | |
| chr1: 3370563 | TGCaGCcTCTGCcCGGG | tCCcAGCAGgcGgAGCA | Promoter |
| | (SEQ. ID. NO.: 857) | (SEQ. ID. NO.: 878) | (ARHGEF16) |
| chr17: 58495805 | TaCTGCATCTtCTCaGa | CaaaAGCAGtTtCAaCA | Intergenic |
| | (SEQ. ID. NO.: 858) | (SEQ. ID. NO.: 879) | |
| chr5: 169541385 | TGtTGCATCaGCTCGGG | CCtGAtCAGcgaCAGCc | Intergenic |
| | (SEQ. ID. NO.: 859) | (SEQ. ID. NO.: 880) | |

TABLE 35
Targeting Exon 7:

| Genome Coordinates | Left Half-Site | Right Half-Site | Genomic Region |
|---|---|---|---|
| chrX: 154197644 | TGTCCAAGGTCCATCAAGAG | GGAAAGTTATTGGCGAGA | Exon (F8) |
| | (SEQ. ID. NO.: 881) | (SEQ. ID. NO.: 902) | |
| chr2: 18105031 | TGTCaAAaaTCaATCAAaAa | tTaTTGATtGAttTTtGACA | Intron (KCNS3) |
| | (SEQ. ID. NO.: 882) | (SEQ. ID. NO.: 903) | |
| chr7: 26500117 | TGTCCAAaGTCCATtttGAG | tTtTTcATGGACacTGGgCA | Intron (LOC441204) |
| | (SEQ. ID. NO.: 883) | (SEQ. ID. NO.: 904) | |
| chr4: 27239786 | TGTCacAGGTCCtTaAAGAG | atAAAGTTATTGGgGtGA | Intergenic |
| | (SEQ. ID. NO.: 884) | (SEQ. ID. NO.: 905) | |
| chr4: 27428400 | TCTtaCCAATcACTTTCt | GGAAAGgcAgTGGtGAGA | Intergenic |
| | (SEQ. ID. NO.: 885) | (SEQ. ID. NO.: 906) | |
| chrX: 79810036 | TGTCCAAaGTCacTtgAGAG | GGAAAGTTgTTtGaGAGt | Intergenic |
| | (SEQ. ID. NO.: 886) | (SEQ. ID. NO.: 907) | |
| chr1: 172943650 | TaTCCAgacTCCATCcAcAG | tTaTgGAaGGAgtTTGGACA | Intergenic |
| | (SEQ. ID. NO.: 887) | (SEQ. ID. NO.: 908) | |
| chr18: 40289853 | aGTCCAAcaTCCAgCAAGAa | CTCTTGATtGAgCTTaGAac | Intergenic |
| | (SEQ. ID. NO.: 888) | (SEQ. ID. NO.: 909) | |
| chr17: 53122291 | TCTtttCAATAACTgTCC | CTaTTGATGGACaTTaGACt | Intron (STXBP4) |
| | (SEQ. ID. NO.: 889) | (SEQ. ID. NO.: 910) | |
| chr1: 184048225 | TCTgGCCAATAACcgTtC | CTCTTaATGatCtTTGGAtA | Intergenic |
| | (SEQ. ID. NO.: 890) | (SEQ. ID. NO.: 911) | |
| chr19: 32600353 | TGaCCctGaTCCATCcAGAG | GacAAGTTAgTGGCcAGA | Intergenic |
| | (SEQ. ID. NO.: 891) | (SEQ. ID. NO.: 912) | |
| chr3: 29286452 | TGcCaAAGagCCATCAAGAa | ttAAAGTTATgGGaaAGA | Intergenic |
| | (SEQ. ID. NO.: 892) | (SEQ. ID. NO.: 913) | |
| chrX: 145253799 | TGTCCAAGGTCCcaCAgttG | CTCTTGATGccCaTTGtAgA | Intergenic |
| | (SEQ. ID. NO.: 893) | (SEQ. ID. NO.: 914) | |
| chr9: 85073714 | TcctCAAGGgCaATCtAGAG | CTCTTGATtGtCtTgGGtCA | Intergenic |
| | (SEQ. ID. NO.: 894) | (SEQ. ID. NO.: 915) | |
| chr22: 25490404 | TGTCCAAGGcCCcTCAgcAG | GGgAAGTaAaaGGtGAGA | Intron (KIAA1671) |
| | (SEQ. ID. NO.: 895) | (SEQ. ID. NO.: 916) | |
| chr8: 61847049 | TCcaGagAcTAACTTTgC | CcCTTGATtGACCTaGGACA | Intergenic |
| | (SEQ. ID. NO.: 896) | (SEQ. ID. NO.: 917) | |
| chr4: 177996308 | TGTCCAgaGTCCAagAAaAa | CaCTTGAaGGAtggTGGAaA | Intergenic |
| | (SEQ. ID. NO.: 897) | (SEQ. ID. NO.: 918) | |
| chr2: 63471205 | TaTCaAAGGTCtcTCAAaAc | CTCTTGAattAttTTGGgCA | Intron (WDPCP) |
| | (SEQ. ID. NO.: 898) | (SEQ. ID. NO.: 919) | |
| chr14: 101569007 | TGTCCAcatTCCcTCcAGAG | CcCaTGATGGACCcaGccCA | Intergenic |
| | (SEQ. ID. NO.: 899) | (SEQ. ID. NO.: 920) | |
| chr2: 75005696 | ctTCCAAGGcCCAcagAGAG | CcCcTGATtGcCtTTGGAtA | Intergenic |
| | (SEQ. ID. NO.: 900) | (SEQ. ID. NO.: 921) | |
| chr18: 36812500 | TCTCtCCAATAACTgTga | tgCTTcATGtAtCTTGGcCA | Intron (LOC647946) |
| | (SEQ. ID. NO.: 901) | (SEQ. ID. NO.: 922) | |

TABLE 36
Targeting Exon 8:

| Genome Coordinates | Left Half-Site | Right Half-Site | Genomic Region |
|---|---|---|---|
| chrX: 154194740 | TCCTCCTCTTCAGCAGCAATGT | TTAGGATGCTTCTTGGCAACTGA | Exon (F8) |
| | (SEQ. ID. NO.: 923) | (SEQ. ID. NO.: 944) | |
| chr5: 33245024 | cCAGaTtCCAAGAgaCATCaTAA | ACATgGCaGCTGAAGAGGAtGt | Intergenic |
| | (SEQ. ID. NO.: 924) | (SEQ. ID. NO.: 945) | |
| chr3: 159590558 | TCCTCCTCaTCAGtAatAATGT | TTAGaATGtTcagTtGCAAtTGt | Intron (SCHP1) |
| | (SEQ. ID. NO.: 925) | (SEQ. ID. NO.: 946) | |
| chrY: 14031090 | TCAtTTtCaAtGgAtCATCCTAA | ACATgGagGagGAgGAGGAGGA | Intergenic |
| | (SEQ. ID. NO.: 926) | (SEQ. ID. NO.: 947) | |
| chr10: 83854828 | TCctTTtCCtgGAAGCtTtCTcA | TTtGGATGCTTtTgGGaAcCTGA | Intron (NRG3) |
| | (SEQ. ID. NO.: 927) | (SEQ. ID. NO.: 948) | |
| chr12: 86811646 | TCAaaaGCCAAaAAaCAagCaAA | TTAttATGCTcaTTtGCAAaTGA | Intron (MGAT4C) |
| | (SEQ. ID. NO.: 928) | (SEQ. ID. NO.: 949) | |
| chr6: 43379997 | TgAGaTaCCAttAcaCATCCTAg | AaAgTGCTGgTGAAGAtGtGGA | Intergenic |
| | (SEQ. ID. NO.: 929) | (SEQ. ID. NO.: 950) | |
| chr15: 60816292 | TCtgCCTCcTCccCAcCcATaT | TTAGGcTGCTTCTTGGCAcCTtc | Intron (RORA) |
| | (SEQ. ID. NO.: 930) | (SEQ. ID. NO.: 951) | |
| chr4: 104036767 | TtAaaaGCCAgGAAGCATCCTAA | ttATTGaTtaTGAAtgcGAGGA | Intron (CENPE) |
| | (SEQ. ID. NO.: 931) | (SEQ. ID. NO.: 952) | |
| chr2: 220922430 | aCAaTTcCacAGAAtCATCCaAA | aatGGATGCTcCTTGGCAtCaGA | Intergenic |
| | (SEQ. ID. NO.: 932) | (SEQ. ID. NO.: 953) | |
| chr6: 151256031 | TCAGcTaCCAAGAgaaATtCTAA | TTgGGAcatTTaTTtGCAcCTGg | Intron (MTHFD1L) |
| | (SEQ. ID. NO.: 933) | (SEQ. ID. NO.: 954) | |
| chr12: 14116257 | TCtcCCTCaTCAGCAGaAATGa | gCATgaCaGCTGtAGtGGAGGg | Intron (GRIN2B) |
| | (SEQ. ID. NO.: 934) | (SEQ. ID. NO.: 955) | |
| chr11: 41540671 | TttTCaTCTTCAtCtGtgATtT | caATTGCTGCTGAAGgtGAGGA | Intergenic |
| | (SEQ. ID. NO.: 935) | (SEQ. ID. NO.: 956) | |
| chr10: 607478 | TaCTCCTCTaaAaCcaCAATGg | acAGGATGgTTCTcaGCcACTGA | Intron (DIP2C) |
| | (SEQ. ID. NO.: 936) | (SEQ. ID. NO.: 957) | |
| chr18: 64076819 | TCAtTTaCCAAacAGaATtaTAA | gTAaGATGtTTCcTGatttCTGA | Intergenic |
| | (SEQ. ID. NO.: 937) | (SEQ. ID. NO.: 958) | |
| chr3: 159590555 | TCaTCCTCcTCAtCAGtAATaa | TTAGaATGtTcagTtGCAAtTGt | Intron (SCHIP1) |
| | (SEQ. ID. NO.: 938) | (SEQ. ID. NO.: 959) | |
| chr2: 25775417 | TCCcCaTCaTtAGCAGCAATGc | TcAGGtTtCcTtTTGcaAACaGA | Intron (DTNB) |
| | (SEQ. ID. NO.: 939) | (SEQ. ID. NO.: 960) | |
| chr5: 60672404 | aCCTCCaCTTCAGtAatAATGa | TTAGaATGtgTtaTGtCAttTGA | Intron (ZSWIM6) |
| | (SEQ. ID. NO.: 940) | (SEQ. ID. NO.: 961) | |
| chr2: 158235451 | TCAaaTGaCAtaAcaCATtCTAA | tCATTatTaCTGAAGtGGAGGt | Intergenic |
| | (SEQ. ID. NO.: 941) | (SEQ. ID. NO.: 962) | |
| chr11: 131914316 | TCtGagGCCAAaAAGaAaaaTAA | AtgTgtCTGtTcAAGAGGAGGA | Intron (NTM) |
| | (SEQ. ID. NO.: 942) | (SEQ. ID. NO.: 963) | |
| chrY: 3867095 | aCAGTTaCCAAaAAGCAaaaTAA | gCAagatgGCTGAAtAGGAaGA | Intergenic |
| | (SEQ. ID. NO.: 943) | (SEQ. ID. NO.: 964) | |

TABLE 37
Targeting Exon 9:

| Genome Coordinates | Left Half-Site | Right Half-Site | Genomic Region |
|---|---|---|---|
| chrX: 154194255 | TCTCCAACTTCCCCATAA | TTCCTGATTCATGCTGAA | Exon (F8) |
| | (SEQ. ID. NO.: 965) | (SEQ. ID. NO.: 986) | |
| chr4: 150672318 | TTCAGCtTaAcaCtGGAt | TTCCTGATTCcTGaTGAA | Intergenic |
| | (SEQ. ID. NO.: 966) | (SEQ. ID. NO.: 987) | |
| chr2: 89399484 | TgCAGCATagATCAGGgA | TcCCTGgTTtcTGCTGAA | Intergenic |
| | (SEQ. ID. NO.: 967) | (SEQ. ID. NO.: 988) | |
| chr5: 19372097 | TTCAtCATaAAgCtaaAA | TTCtTaATTaATGCTGAA | Intergenic |
| | (SEQ. ID. NO.: 968) | (SEQ. ID. NO.: 989) | |
| chr4: 56376997 | TTCAGaATGAAaCAGGAA | TTCCTGAgaCAaGaTGgg | Intron (CLOCK) |
| | (SEQ. ID. NO.: 969) | (SEQ. ID. NO.: 990) | |
| chr14: 98831622 | TtTCCtcCTTCCCCATAc | gTtCTGATTCATGaTGAA | Intergenic |
| | (SEQ. ID. NO.: 970) | (SEQ. ID. NO.: 991) | |
| chr20: 6216194 | TTCAGCATGAAgCAaGAA | TTCCTGAaaCATcaacAA | Intergenic |
| | (SEQ. ID. NO.: 971) | (SEQ. ID. NO.: 992) | |
| chr3: 76350178 | TTCAGCtTGAATtAGGAA | cTtgTGtTTaATGaTGAA | Intergenic |
| | (SEQ. ID. NO.: 972) | (SEQ. ID. NO.: 993) | |
| chr6: 79957598 | TTCAGCATaAATaAtaAA | TTCtTGtTTaATtCTcAA | Intergenic |
| | (SEQ. ID. NO.: 973) | (SEQ. ID. NO.: 994) | |
| chr5: 129714571 | TTCAcCATctATCtGaAA | TTtCTGAggCATGtTGAA | Intergenic |
| | (SEQ. ID. NO.: 974) | (SEQ. ID. NO.: 995) | |
| chr2: 183992955 | aTCAaCATGtAaCAGaAA | TTttTGATTCATGtaGgA | Intron (NUP35) |
| | (SEQ. ID. NO.: 975) | (SEQ. ID. NO.: 1656) | |
| chr11: 100927598 | TTCAatATGAtTaAGtAt | TTgaTGATTtATGCTGAA | Intron (PGR) |
| | (SEQ. ID. NO.: 976) | (SEQ. ID. NO.: 996) | |
| chr5: 118162509 | TgCAGCAgtAAaCAtGAA | TTtCTaATTCATGCTaAA | Intergenic |
| | (SEQ. ID. NO.: 977) | (SEQ. ID. NO.: 997) | |
| chr7: 136796091 | TgCAGCATaAATtAaGgA | aTCCTGggTCATGtTGAA | Intron |
| | (SEQ. ID. NO.: 978) | (SEQ. ID. NO.: 998) | (LOC349160) |
| chrX: 114442244 | TTCcaCATaAAaaAGGAc | TTCCTGtTgtAgGCTGAA | Intron (LRCH2) |
| | (SEQ. ID. NO.: 979) | (SEQ. ID. NO.: 999) | |
| chr17: 70147587 | TTaAaaATGAATCAaaAc | TTtCaGATcaATGCTGAA | Intergenic |
| | (SEQ. ID. NO.: 980) | (SEQ. ID. NO.: 1000) | |
| chr22: 17414552 | TgCAGCATGAATtAGGAg | TcCCTGgTTtcTGCTGAt | Intergenic |
| | (SEQ. ID. NO.: 981) | (SEQ. ID. NO.: 1001) | |
| chr1: 220485886 | TTCAGgAgaAATCgaGAA | TTCCTGATatATGtTGAg | Intergenic |
| | (SEQ. ID. NO.: 982) | (SEQ. ID. NO.: 1002) | |
| chr2: 89292060 | TgCAGCATagATCAGGAg | TcCCTGgTTttTGCTGAt | Intergenic |
| | (SEQ. ID. NO.: 983) | (SEQ. ID. NO.: 1003) | |
| chr2: 89309611 | TgCAGCATagATCAGGAg | TcCCTGgTTttTGCTGAt | Intergenic |
| | (SEQ. ID. NO.: 984) | (SEQ. ID. NO.: 1004) | |
| chr2: 90260070 | aTCAGCAaaAAcCAGGgA | cTCCTGATctATGCTGcA | Intergenic |
| | (SEQ. ID. NO.: 985) | (SEQ. ID. NO.: 1005) | |

TABLE 38
Targeting Exon 10:

| | | | Genomic |
|---|---|---|---|
| Genome Coordinates | Left Half-Site | Right Half-Site | Region |
| chrX: 154189360 | TCTCCTTGAATACAAAGGAC | CCGTGAGGGTAGATGTTATA | Exon (F8) |
| | (SEQ. ID. NO.: 1006) | (SEQ. ID. NO.: 1027) | |
| chr6: 129821493 | TgTCCTTaAAaACAAAGGAC | CttTGAGGtTAcATGTTAgA | Intron (LAMA2) |
| | (SEQ. ID. NO.: 1007) | (SEQ. ID. NO.: 1028) | |
| chr2: 147755789 | TtTCCTTGgATACAAAGaAC | aaaaTTTaTATgCAAGGAGg | Intergenic |
| | (SEQ. ID. NO.: 1008) | (SEQ. ID. NO.: 1029) | |
| chr15: 35542434 | TATAAgATaTACCCTaAtGG | tTCCTgTGTcTTCAAaGAGA | Intergenic |
| | (SEQ. ID. NO.: 1009) | (SEQ. ID. NO.: 1030) | |
| chrX: 106606342 | TCTCCcTGcATACAgAGatC | GTtCTTTGTATaagAGGAGg | Intergenic |
| | (SEQ. ID. NO.: 1010) | (SEQ. ID. NO.: 1031) | |
| chr11: 116391255 | TCTCCaaaAATAaAAAaGAa | GcCtaTTGTATTCcAGGAaA | Intergenic |
| | (SEQ. ID. NO.: 1011) | (SEQ. ID. NO.: 1032) | |
| chr4: 174370428 | TaTCtTcaAATtCAAAGGAC | aTCCTTTGTAgTCAAGGAtg | Intergenic |
| | (SEQ. ID. NO.: 1012) | (SEQ. ID. NO.: 1033) | |
| chrX: 48388946 | TgTCCTTGcATgCAAAatAC | cTCtTTTGTtTTtttGGAGA | Intergenic |
| | (SEQ. ID. NO.: 1013) | (SEQ. ID. NO.: 1034) | |
| chr1: 184030566 | TCTtaTTattTACAAAGagC | GTCtcTTtTATTgAAGGAGA | Intron (TSEN15) |
| | (SEQ. ID. NO.: 1014) | (SEQ. ID. NO.: 1035) | |
| chr8: 105838647 | aCatCTTaAATACAAAGaAC | GgCaTcTGTAaTCAAGtgGA | Intergenic |
| | (SEQ. ID. NO.: 1015) | (SEQ. ID. NO.: 1036) | |
| chr14: 60101345 | TCTCCaTaAATACAAAGGga | CaGaGgGGGaAaATtTTAcA | Intron (RTN1) |
| | (SEQ. ID. NO.: 1016) | (SEQ. ID. NO.: 1037) | |
| chr6: 32447046 | gCTCtTTGtgaACAAAGGcC | tTCCTTTGTATTtActGAGA | Intergenic |
| | (SEQ. ID. NO.: 1017) | (SEQ. ID. NO.: 1038) | |
| chr6_qbl_hap6: 3707956 | gCTCtTTGtgaACAAAGGcC | tTCCTTTGTATTtActGAGA | Intergenic |
| | (SEQ. ID. NO.: 1018) | (SEQ. ID. NO.: 1039) | |
| chr6_apd_hap1: 3761430 | gCTCtTTGtgaACAAAGGcC | tTCCTTTGTATTtActGAGA | Intergenic |
| | (SEQ. ID. NO.: 1019) | (SEQ. ID. NO.: 1040) | |
| chr6: 153043585 | TgTAAtATtTtCCCcCAaGc | GTatTTTGTATTCAAtGtGA | Exon (MYCT1) |
| | (SEQ. ID. NO.: 1020) | (SEQ. ID. NO.: 1041) | |
| chrX: 129578399 | TCaCCaTcAgTgCAAgaGAC | GgCtTTgGTATTaAAtGAGA | Intergenic |
| | (SEQ. ID. NO.: 1021) | (SEQ. ID. NO.: 1042) | |
| chr2: 237165553 | TCTCgTaGAAagCAAAGaAa | tTttTcTGTATTtAAaGAGA | Intron (ASB18) |
| | (SEQ. ID. NO.: 1022) | (SEQ. ID. NO.: 1043) | |
| chr14: 74504800 | TATcttATCTcCCCTaAtaG | GTCCTTTGTATTCAttGAaA | Intron (C14orf45) |
| | (SEQ. ID. NO.: 1023) | (SEQ. ID. NO.: 1044) | |
| chr14: 94651285 | TCTCCTgGggaAtgAAGGtC | GatacTTGTATTCAAGGAGA | Intron (PPP4R4) |
| | (SEQ. ID. NO.: 1024) | (SEQ. ID. NO.: 1045) | |
| chr14: 42051030 | TtTCCTaGtATACAAAaGAt | aTCtTTTGTATaCtAGGAaA | Intergenic |
| | (SEQ. ID. NO.: 1025) | (SEQ. ID. NO.: 1046) | |
| chr11: 31557496 | caTCCTTGgATACAgAGGgC | GattTTgGTATTCAtGGAGt | Intron (ELP4) |
| | (SEQ. ID. NO.: 1026) | (SEQ. ID. NO.: 1047) | |

TABLE 39
Targeting Exon 11:

| Genome Coordinates | Left Half-Site | Right Half-Site | Genomic Region |
|---|---|---|---|
| chrX: 154185248 | TCTACAGATTCTTTGTAGCAG | ATGAGTCCTGAAGCTAGA | Exon (F8) |
| | (SEQ. ID. NO.: 1048) | (SEQ. ID. NO.: 1069) | |
| chr8: 91254790 | TCTAGtTTCAGcAgTatT | ATGAGTCaTGAAGCTtGA | Intron (LINC00534) |
| | (SEQ. ID. NO.: 1049) | (SEQ. ID. NO.: 1070) | |
| chr2: 220340352 | TCcAtCTTCAGGACTCAc | AgGAGcCCTGAAGtTtGg | Intron (SPEG) |
| | (SEQ. ID. NO.: 1050) | (SEQ. ID. NO.: 1071) | |
| chr13: 65583211 | TtTACAGATgCTTTaTAGCAG | CTGgcAatAAacATCTGTAGA | Intergenic |
| | (SEQ. ID. NO.: 1051) | (SEQ. ID. NO.: 1072) | |
| chr8: 136213502 | cCTACAaATcCTTTGTgGCAG | ATGgGctCTGgAGCcAGA | Intergenic |
| | (SEQ. ID. NO.: 1052) | (SEQ. ID. NO.: 1073) | |
| chr4: 79545446 | TtcAcCTTCctGACTCAT | ATGAGTtCTGggGCTAGA | Intergenic |
| | (SEQ. ID. NO.: 1053) | (SEQ. ID. NO.: 1074) | |
| chr6: 105454604 | TCTcaCTTCAGGACcCAg | ATaAGTttTGAAGCagGA | Intron (LIN28B) |
| | (SEQ. ID. NO.: 1054) | (SEQ. ID. NO.: 1075) | |
| chr17: 50618031 | TCcAaCcTCAGaACTCAT | cTGAGTtCTGAgGtTgGg | Intergenic |
| | (SEQ. ID. NO.: 1055) | (SEQ. ID. NO.: 1076) | |
| chr21: 40482039 | TCTAaaaTCAGGACTCcT | gTGAtTgtTGAAGCcAGA | Intergenic |
| | (SEQ. ID. NO.: 1056) | (SEQ. ID. NO.: 1077) | |
| chr11: 132218577 | TCTcaCTTaAGGACTtAc | tTGAGTCCaGAAGtTtGA | Intergenic |
| | (SEQ. ID. NO.: 1057) | (SEQ. ID. NO.: 1078) | |
| chr2: 27385297 | TCTgtCTTCAGaAgTCcT | gTGAGTtCTGAAtCTgGA | Intergenic |
| | (SEQ. ID. NO.: 1058) | (SEQ. ID. NO.: 1079) | |
| chr14: 22481030 | TCTAcCTTCAGcACTCtg | tTttGTtCTGAAGCcAGA | Intergenic |
| | (SEQ. ID. NO.: 1059) | (SEQ. ID. NO.: 1080) | |
| chr3: 31348185 | TCTcGCaTCAaGACcCAT | tgGAGTtCaGAtGCTAaA | Intergenic |
| | (SEQ. ID. NO.: 1060) | (SEQ. ID. NO.: 1081) | |
| chr4: 87584049 | aCTACAGcTaCTTgGaAGCAG | tTGAGcCCaGAAGtTtGA | Intron (PTPN13) |
| | (SEQ. ID. NO.: 1061) | (SEQ. ID. NO.: 1082) | |
| chr4: 71281490 | TCaAaCTcCtGacCTCAT | tTGtTtCAAAtAATtTGTAtA | Intergenic |
| | (SEQ. ID. NO.: 1062) | (SEQ. ID. NO.: 1083) | |
| chr2: 108857249 | TCTctCTcCAGtACTCAT | ATGtGTgCTGtgGgTAGA | Intergenic |
| | (SEQ. ID. NO.: 1063) | (SEQ. ID. NO.: 1084) | |
| chrX: 47785928 | TgTAGCTTCtGtACTacT | ATaAGTCtTGAAGtcAGA | Intergenic |
| | (SEQ. ID. NO.: 10674) | (SEQ. ID. NO.: 1085) | |
| chr8: 79584265 | TCTtGCcTgAGGACTCAT | tgGgGaCtTGAAGtTAGA | Intron (ZC2HC1A) |
| | (SEQ. ID. NO.: 1065) | (SEQ. ID. NO.: 1086) | |
| chr1: 216023388 | TCaAGaTcCAGaACTCAa | ATaAGTaCTGAAGCTAtt | Intron (USH2A) |
| | (SEQ. ID. NO.: 1066) | (SEQ. ID. NO.: 1087) | |
| chr17: 50619873 | TaTAcaTaCAGaACTtAT | ATGAGTtCTGAgGtTAGg | Intergenic |
| | (SEQ. ID. NO.: 1067) | (SEQ. ID. NO.: 1088) | |
| chr13: 20930589 | aCTAGCTTCAttAtTCAT | ATtAGTCtTGAAGtatGA | Intergenic |
| | (SEQ. ID. NO.: 1068) | (SEQ. ID. NO.: 1089) | |

TABLE 40
Targeting Exon 12:

| Genome | | | |
|---|---|---|---|
| Coordinates | Left Half-Site | Right Half-Site | Genomic Region |
| chrX: 154182199 | TCCTCAAGCTGCACTCCAGCT | CGTTGTATATTCTCTGTGA | Exon (F8) |
| | (SEQ. ID. NO.: 1090) | (SEQ. ID. NO.: 1111) | |
| chr7: 156430074 | TCCaCAAGCTGgACTCCAaCT | atTTGaAcAcTtTCTGTGA | Intergenic |
| | (SEQ. ID. NO.: 1091) | (SEQ. ID. NO.: 1112) | |
| chr9: 43597045 | TCACAaAGAATAaACAACt | CtaTGTATATaaTCTtTtA | Intergenic |
| | (SEQ. ID. NO.: 1092) | (SEQ. ID. NO.: 1113) | |
| chr10: 899227 | TCcCAGtGAATATAaAAat | tGTTGTATATTtaaTGTGA | Intron (LARP4B) |
| | (SEQ. ID. NO.: 1093) | (SEQ. ID. NO.: 1114) | |
| chr5: 44595593 | TCAaAGtGgAaATACAACa | CtTTGTATATTtTCTtTtA | Intergenic |
| | (SEQ. ID. NO.: 1094) | (SEQ. ID. NO.: 1115) | |
| chr12: 13837730 | TCcCAGAGAAaATACcAaG | CGTTaTcTcTTtTtTGTGA | Intron (GRIN2B) |
| | (SEQ. ID. NO.: 1095) | (SEQ. ID. NO.: 1116) | |
| chr10: 85585731 | TCAtAGAaAATAagaAACt | tGTTGTATATTCTgTGTcA | Intergenic |
| | (SEQ. ID. NO.: 1096) | (SEQ. ID. NO.: 1117) | |
| chr10: 64580474 | TCcCAGAGgcTATAaAcCa | AaCTGttGTGaAGCTTGAGGA | Intergenic |
| | (SEQ. ID. NO.: 1097) | (SEQ. ID. NO.: 1118) | |
| chrX: 38783417 | TCCTCAAaCTGCtCTCCAaCa | CtTccTATtTgtTCTtTGA | Intergenic |
| | (SEQ. ID. NO.: 1098) | (SEQ. ID. NO.: 1119) | |
| chr2: 193570138 | TtACAtAGAATtTACAAta | CaTTGTAaATTCTaTGTGA | Intergenic |
| | (SEQ. ID. NO.: 1099) | (SEQ. ID. NO.: 1120) | |
| chr7: 110741635 | TaAtAcAGAATATACAtaG | tcTTGTATATTtcCTGTGA | Intron (IMMP2L) |
| | (SEQ. ID. NO.: 1100) | (SEQ. ID. NO.: 1121) | |
| chr3: 191344909 | TCcCAaAGAcTgTtCtAaG | gGTgtTATATTCTCTGTGA | Intergenic |
| | (SEQ. ID. NO.: 1101) | (SEQ. ID. NO.: 1122) | |
| chr9: 39389206 | TaAaAGAttATATACAtaG | ttTTGTtTATTCTtTGTGA | Intergenic |
| | (SEQ. ID. NO.: 1102) | (SEQ. ID. NO.: 1123) | |
| chr9: 39918509 | TaAaAGAttATATACAtaG | ttTTGTtTATTCTtTGTGA | Intergenic |
| | (SEQ. ID. NO.: 1103) | (SEQ. ID. NO.: 1124) | |
| chr9: 40733954 | TaAaAGAttATATACAtaG | ttTTGTtTATTCTtTGTGA | Intergenic |
| | (SEQ. ID. NO.: 1104) | (SEQ. ID. NO.: 1125) | |
| chr9: 41293775 | TCACAaAGAATAaACAAaa | CtaTGTATATaaTCTtTtA | Intergenic |
| | (SEQ. ID. NO.: 1105) | (SEQ. ID. NO.: 1126) | |
| chr9: 65476200 | TCACAaAGAATAaACAAaa | CtaTGTATATaaTCTtTtA | Intergenic |
| | (SEQ. ID. NO.: 1106) | (SEQ. ID. NO.: 1127) | |
| chrX: 50790890 | gCACAGActATAggCAgCc | CaTgGTATATTCTtTGTGA | Intergenic |
| | (SEQ. ID. NO.: 1107) | (SEQ. ID. NO.: 1128) | |
| chr5: 5141262 | TCCcCAAcCTttcCTCCttCT | CGTTGctTATTCTCaGTGA | Intron (ADAMTS16) |
| | (SEQ. ID. NO.: 1108) | (SEQ. ID. NO.: 1129) | |
| chrX: 22329605 | TCAaAtgGAgTAaACAACt | CtTTGTAcATTtTCTGTGt | Intron |
| | (SEQ. ID. NO.: 1109) | (SEQ. ID. NO.: 1130) | (LOC100873065) |
| chr7: 105616909 | TCACAGAGcATATACtcCa | ttTaGTATATTCaCaGTcA | Intron (CDHR3) |
| | (SEQ. ID. NO.: 1110) | (SEQ. ID. NO.: 1131) | |

TABLE 41
Targeting Exon 13:

| Genome Coordinates | Left Half-Site | Right Half-Site | Genomic Region |
|---|---|---|---|
| chrX: 154176028 | TGTGTCTTCATAGACCATTTT | ATCCAGAGAAGAAGACA | Exon (F8) |
| | (SEQ. ID. NO.: 1132) | (SEQ. ID. NO.: 1153) | |
| chr19: 31555212 | TaTCTTCTTCTCTGGAT | cTCCAtgGAAGAAaAaA | Intergenic |
| | (SEQ. ID. NO.: 1133) | (SEQ. ID. NO.: 1154) | |
| chr11: 98185196 | TaTcTCTTaATAGcCCATTTT | ATaCAGAGAAGAAaACA | Intergenic |
| | (SEQ. ID. NO.: 1134) | (SEQ. ID. NO.: 1155) | |
| chr9: 126179092 | TGTGTCTTtATgGAaCAacTa | ATtCAGAGAAtAAGACA | Intron (DENND1A) |
| | (SEQ. ID. NO.: 1135) | (SEQ. ID. NO.: 1156) | |
| chr1: 197582736 | aGTtcTCaTCcCTGtAT | cTCCAGAGAAGAAGACA | Intron (DENND1B) |
| | (SEQ. ID. NO.: 1136) | (SEQ. ID. NO.: 1157) | |
| chr9: 25886338 | TtTtTaCTTCTCaGaAT | ATtCAGAGAAGcAGAtA | Intergenic |
| | (SEQ. ID. NO.: 1137) | (SEQ. ID. NO.: 1158) | |
| chr16: 65046771 | TGcCTTCTTCTCTGaAT | cTCtAGAccAaAAGtCA | Intron (CDH11) |
| | (SEQ. ID. NO.: 1138) | (SEQ. ID. NO.: 1159) | |
| chr6: 37769405 | TGaGTCTTCATAGAaCATTTT | AgCtgGAagAGAAGACc | Intergenic |
| | (SEQ. ID. NO.: 1139) | (SEQ. ID. NO.: 1160) | |
| chr4: 53116406 | TGgCTTCTgCTCTGtgT | AgCCAGAGAtGAAGtCA | Intergenic |
| | (SEQ. ID. NO.: 1140) | (SEQ. ID. NO.: 1161) | |
| chr10: 117955396 | acTaaaCTTCTCTGaAT | AgCCAGAGAtGAAGACA | Intron (GFRA1) |
| | (SEQ. ID. NO.: 1141) | (SEQ. ID. NO.: 1162) | |
| chr4: 157999316 | TaTaTTCTTaTaTGGAg | AAggTGGTtTATGAAGACACA | Intron (GLRB) |
| | (SEQ. ID. NO.: 1142) | (SEQ. ID. NO.: 1163) | |
| chr4: 172676113 | TGTCaTCTTCTCTGtAT | tTtaAGAGAAaAAtACt | Intergenic |
| | (SEQ. ID. NO.: 1143) | (SEQ. ID. NO.: 1164) | |
| chr7: 70692951 | TGcCTTCTTCcCTGGAT | cgatAGAGgAGgAGACA | Intron (WBSCR17) |
| | (SEQ. ID. NO.: 1144) | (SEQ. ID. NO.: 1165) | |
| chr1: 153460499 | TGTCTTCTTCTCTGtcT | ATCtAGAGAAtggGAgt | Intergenic |
| | (SEQ. ID. NO.: 1145) | (SEQ. ID. NO.: 1166) | |
| chr17: 55521352 | gGTCaTCaTCTtTGGtT | AgCCAGgGAAGAAGACA | Intron (MSI2) |
| | (SEQ. ID. NO.: 1146) | (SEQ. ID. NO.: 1167) | |
| chr15: 37159972 | TGTtTTCTTCTCTGcAT | tAAATaaTCTATGAtGAgAtA | Intron (LOC145845) |
| | (SEQ. ID. NO.: 1147) | (SEQ. ID. NO.: 1168) | |
| chr10: 81475753 | TcTCTTCTTCTCTGtAT | AggCAtAGAtGAtGgCA | Intergenic |
| | (SEQ. ID. NO.: 1148) | (SEQ. ID. NO.: 1169) | |
| chr10: 88997979 | TcTCTTCTTCTCTGtAT | AggCAtAGAtGAtGgCA | Intergenic |
| | (SEQ. ID. NO.: 1149) | (SEQ. ID. NO.: 1170) | |
| chr10: 89259535 | TGcCaTCaTCTaTGccT | ATaCAGAGAAGAAGAgA | Intergenic |
| | (SEQ. ID. NO.: 1150) | (SEQ. ID. NO.: 1171) | |
| chr2: 12846210 | ctTCTTCTTCTCTGaAT | ATatAtAGAAGAAtAtA | Intergenic |
| | (SEQ. ID. NO.: 1151) | (SEQ. ID. NO.: 1172) | |
| chr13: 107009889 | TGTCTcCcaCTCTGctg | ATaCAGAGAAGAAGgCA | Intergenic |
| | (SEQ. ID. NO.: 1152) | (SEQ. ID. NO.: 1173) | |

TABLE 42
Targeting Exon 14:

| Genome Coordinates | Left Half-Site | Right Half-Site | Genomic Region |
|---|---|---|---|
| chrX: 154156874 | TCATCCCATAATCCCAGAGCCTCT | AAATAGTGTCGTGTTTTCTTTTGA | Exon (F8) |
| | (SEQ. ID. NO.: 1174) | (SEQ. ID. NO.: 1195) | |
| chr6: 17669261 | TgAAAAaAAAAaAaaACACTATTa | AAATAcctTttTtTTTTtTTTTGA | Intron (NUP153) |
| | (SEQ. ID. NO.: 1175) | (SEQ. ID. NO.: 1196) | |
| chr11: 12730893 | TaAAAAaAAAAaACcAgAaTAaTT | ttATAGTtTtGTtTcTTtTTTTGA | Intron (TEAD1) |
| | (SEQ. ID. NO.: 1176) | (SEQ. ID. NO.: 1197) | |
| chr11: 68651384 | TCAAAAaAAAcCAaaACACTtaTT | AAtTAaTtTtaTtTaTTtTTTTGA | Intergenic |
| | (SEQ. ID. NO.: 1177) | (SEQ. ID. NO.: 1198) | |
| chr5: 132729450 | TagAAAGgAgACAaGggtCTAgTT | AGAaGCTCTGtGAgTtTGGGATGA | Intron (FSTL4) |
| | (SEQ. ID. NO.: 1178) | (SEQ. ID. NO.: 1199) | |
| chr5: 102197872 | TCAAAAaAAAAaAaaAaAaaAaTT | AcATAtTGTCtTtTTTTtTTTTaA | Intergenic |
| | (SEQ. ID. NO.: 1179) | (SEQ. ID. NO.: 1200) | |
| chr6: 150020193 | TCAAAAaAAAAaAaGgCACTATcT | AGtaGgTtaGGGtTTcTGaaATGA | Intron (LATS1) |
| | (SEQ. ID. NO.: 1180) | (SEQ. ID. NO.: 1201) | |
| chr8: 102067589 | TCAgAAaAtAAtAtGACACTtTTg | AAATttTGTCaTGTTTgCTTTaGA | Intron (FLJ42969) |
| | (SEQ. ID. NO.: 1181) | (SEQ. ID. NO.: 1202) | |
| chr5: 96436598 | aaAAAAaAAAAaAaaAgAaTATaT | AAtTAGTGTtGTcTTTTCcTgTGA | Intron (LIX1) |
| | (SEQ. ID. NO.: 1182) | (SEQ. ID. NO.: 1203) | |
| chr22: 31430439 | TCAAAAaAAAAaAaGcCcCTgTcc | AtATAtTtTttTtTTTTtTTTTGA | Intergenic |
| | (SEQ. ID. NO.: 1183) | (SEQ. ID. NO.: 1204) | |
| chr5: 96436600 | aaAAAAaAAAAaAaGAataTATaT | AAtTAGTGTtGTcTTTTCcTgTGA | Intron (LIX1) |
| | (SEQ. ID. NO.: 1184) | (SEQ. ID. NO.: 1205) | |
| chr8: 129874245 | TtAAAAGAAAcagCGACACTATTT | AtAaAaTagCaTtTTcTCTTcTGA | Intergenic |
| | (SEQ. ID. NO.: 1185) | (SEQ. ID. NO.: 1206) | |
| chr8: 76048195 | TaAcAcagAAtCACctCACTATaT | tAATAGTtTttTtTTTTtTTTTGA | Intergenic |
| | (SEQ. ID. NO.: 1186) | (SEQ. ID. NO.: 1207) | |
| chr3: 167630709 | TtAAAAaAAAAaAaaAgcCTATTT | AAATtGTGaCaTcTTTTtTTTTaA | Intron (LOC646168) |
| | (SEQ. ID. NO.: 1187) | (SEQ. ID. NO.: 1208) | |
| chr17: 79330592 | TCAAAAaAAAAaAaaAaAtTATTT | tttTttTGTttTGTTTTgTTTTGt | Intergenic |
| | (SEQ. ID. NO.: 1188) | (SEQ. ID. NO.: 1209) | |
| chr7: 56511801 | aaAAAAGAAAACtgGtgtCaATTT | AAAaAGTGTCGgGTTTTtTTTTtt | Intron (LOC650226) |
| | (SEQ. ID. NO.: 1189) | (SEQ. ID. NO.: 1210) | |
| chrX: 108947147 | TaAAAAaAAAAaAattCACTATgT | AAATAtTGTgGgGTTTTtTTgTtg | Intron (ACSL4) |
| | (SEQ. ID. NO.: 1190) | (SEQ. ID. NO.: 1211) | |
| chr12: 123230886 | TCAAtAaAAAtaAaaAtAaaATTT | tAATAGTaTttTtTTTTtTTTTGA | Intergenic |
| | (SEQ. ID. NO.: 1191) | (SEQ. ID. NO.: 1212) | |
| chr3: 163374286 | TaAAccaAAAACtCaACAaTcaTT | AAATAtgGTtGgtTTgTtTTTTGA | Intergenic |
| | (SEQ. ID. NO.: 1192) | (SEQ. ID. NO.: 1213) | |
| chr12: 9357687 | TCAAAAaAAAACAaaACAaagTTT | gAAaAGTcTttTcTTTTtTaTTtA | Intron (PZP) |
| | (SEQ. ID. NO.: 1193) | (SEQ. ID. NO.: 1214) | |
| chr2: 188514899 | TCAAAAGtAAAaAgtAaACTATTT | tAATAGTGagGTaaTTTCTTTatA | Intergenic |
| | (SEQ. ID. NO.: 1194) | (SEQ. ID. NO.: 1215) | |

TABLE 43
Targeting Exon 15:

| Genome Coordinates | Left Half-Site | Right Half-Site | Genomic Region |
|---|---|---|---|
| chrX: 154134726 | TATGGCCCCAGGAGTCCCAA | CTCCACGGTATAAGGGCTGA | Exon (F8) |
| | (SEQ. ID. NO.: 1216) | (SEQ. ID. NO.: 1237) | |
| chr1: 43805061 | TATGGCCCCAGagaTCCCAA | tcCCACGGTcatAcaGCTGA | Exon (MPL) |
| | (SEQ. ID. NO.: 1217) | (SEQ. ID. NO.: 1238) | |
| chr17: 48220703 | TATaGCCCCcatgGTCaCcA | CTtCAgGGcATAgGGGCTGA | Intron (PPP1R9B) |
| | (SEQ. ID. NO.: 1218) | (SEQ. ID. NO.: 1239) | |
| chr6: 10659136 | TCAatCCTTATgCCaaGGAG | TctGGtCTCCTGtGGtCAcA | Intergenic |
| | (SEQ. ID. NO.: 1219) | (SEQ. ID. NO.: 1240) | |
| chr4: 138564864 | TATGaCCCaAaGAaaCCaAA | tTCtAtGtTAaAAGtGaTGA | Intergenic |
| | (SEQ. ID. NO.: 1220) | (SEQ. ID. NO.: 1241) | |
| chr1: 242357075 | TgTGaCCCCAGGAGTCatAA | CTtCAaGGgcTAtGGGagGA | Intron (PLD5) |
| | (SEQ. ID. NO.: 1221) | (SEQ. ID. NO.: 1242) | |
| chr20: 53898975 | TCAaCCCTaATtCCtTaGAG | CTCtAgGGgATAAGGctTcA | Intergenic |
| | (SEQ. ID. NO.: 1222) | (SEQ. ID. NO.: 1243) | |
| chr16: 10915221 | TcTGaCCCtAaGAaTCaCcA | TTGGGgtTCCTGGaGtCATg | Intergenic |
| | (SEQ. ID. NO.: 1223) | (SEQ. ID. NO.: 1244) | |
| chr10: 134224399 | TgTGGCCCCAGGgGcCCaAc | agGGGACTttTGGGGgCgTA | Intron (PWWP2B) |
| | (SEQ. ID. NO.: 1224) | (SEQ. ID. NO.: 1245) | |
| chrX: 17609569 | TaAGCCCTTATAatGgGtAG | tTCCAtGGTATttGGtaTGA | Intron (NHS) |
| | (SEQ. ID. NO.: 1225) | (SEQ. ID. NO.: 1246) | |
| chr12: 4412126 | TggGcCCCaAGGAGTCCCAc | TTGGGAaTCtTGGaGCCtaA | Exon (CCND2) |
| | (SEQ. ID. NO.: 1226) | (SEQ. ID. NO.: 1247) | |
| chr22: 48089574 | TgTGGgCCCAGGAGTCaCgA | CcCCAgGGTATcAGGGtgGc | Intergenic |
| | (SEQ. ID. NO.: 1227) | (SEQ. ID. NO.: 1248) | |
| chr17: 1538247 | TgTGGCCCCAGGAagCCCAg | TTGGGgCTCtgGccGaCAgA | Exon (SCARF1) |
| | (SEQ. ID. NO.: 1228) | (SEQ. ID. NO.: 1249) | |
| chr19: 35657806 | TAccaCCCCAGcAGTCaCAA | tggCAgGGaAcAAGGGCTGA | Intron (FXYD5) |
| | (SEQ. ID. NO.: 1229) | (SEQ. ID. NO.: 1250) | |
| chr1: 158375793 | TcTaGCtCCAtaAGTCCCtA | TTGGGtCTCtTGGGatCtgA | Intergenic |
| | (SEQ. ID. NO.: 1230) | (SEQ. ID. NO.: 1251) | |
| chr14: 99426061 | TCAGCaCTTATcCaGTGGAc | TTGGGACaCCaGaGaaCAcA | Intergenic |
| | (SEQ. ID. NO.: 1231) | (SEQ. ID. NO.: 1252) | |
| chr1: 34177797 | cATcaCaCCAGGAtTCCCAA | TgGGGtCcCCTGGGGtCAgg | Intron (CSMD2) |
| | (SEQ. ID. NO.: 1232) | (SEQ. ID. NO.: 1253) | |
| chr13: 19522623 | cCAcCCCcccTACaGgGGAG | TgGGcACTCCTGGGcCCATA | Intergenic |
| | (SEQ. ID. NO.: 1233) | (SEQ. ID. NO.: 1254) | |
| chr11: 17783271 | TcTGGCCCCAtGgaTCCCAA | caGaGcCTCCTGGGGCacaA | Intron (KCNC1) |
| | (SEQ. ID. NO.: 1234) | (SEQ. ID. NO.: 1255) | |
| chr14: 71921590 | TCtGCCCTTtTACtGTGGAG | acGGGACaCCTGatGtCAcA | Intergenic |
| | (SEQ. ID. NO.: 1235) | (SEQ. ID. NO.: 1256) | |
| chr10: 132968471 | TCAGCCaTTccACCGTGGAa | acGGctCTCCgGGGGCCAct | Intron (TCERG1L) |
| | (SEQ. ID. NO.: 1236) | (SEQ. ID. NO.: 1257) | |

TABLE 44
Targeting Exon 16:

| Genome Coordinates | Left Half-Site | Right Half-Site | Genomic Region |
|---|---|---|---|
| chrX: 154133096 | TCAGAGAAATAAGCCCAG | CTCATCTTTAGTGGGTGCCATA | Exon (F8) |
| | (SEQ. ID. NO.: 1258) | (SEQ. ID. NO.: 1279) | |
| chr7: 25537263 | TCtGtGcAATAAtCtCAG | CTGtGCTTATTTaTtTGA | Intergenic |
| | (SEQ. ID. NO.: 1259) | (SEQ. ID. NO.: 1280) | |
| chr1: 85241221 | TaAaAaAAAaAAGCCCAG | CTGGGCTTtcTTCTggGA | Intergenic |
| | (SEQ. ID. NO.: 1260) | (SEQ. ID. NO.: 1281) | |
| chr17: 49365434 | TCcaAGAAAcAAaCCCAa | CaGGtgTTAcTTCTCTGA | Exon (UTP18) |
| | (SEQ. ID. NO.: 1261) | (SEQ. ID. NO.: 1282) | |
| chr10: 15407376 | TATGaCAtCaACTAAAGATGcG | agGGGCTTAaTTCcCaGA | Intron (FAM171A1) |
| | (SEQ. ID. NO.: 1262) | (SEQ. ID. NO.: 1283) | |
| chr6: 66455619 | cCAGAcAgAgAAcCCCAG | CTGGGtTTATTgCaCTGA | Intergenic |
| | (SEQ. ID. NO.: 1263) | (SEQ. ID. NO.: 1284) | |
| chr2: 168339348 | TCAaAaAAgaAAGCCaAG | CTGtGCTTATaTCTCTcA | Intergenic |
| | (SEQ. ID. NO.: 1264) | (SEQ. ID. NO.: 1285) | |
| chr8: 3275497 | TCAGtGAcATAAGCCCAG | CTGtGCTTgTTaaaaTGA | Intron (CSMD1) |
| | (SEQ. ID. NO.: 1265) | (SEQ. ID. NO.: 1286) | |
| chr1: 172577364 | TCAtAGtAATAAaCagAG | tTGtGtTTATTTCTCTaA | Intron (SUCO) |
| | (SEQ. ID. NO.: 1266) | (SEQ. ID. NO.: 1287) | |
| chr9: 131943933 | gaAGgGgAATAgGCCCAa | CTGGcCTTATTTCTCTGt | Intergenic |
| | (SEQ. ID. NO.: 1267) | (SEQ. ID. NO.: 1288) | |
| chr14: 30487657 | TCAtAGAAATAtGCCCAa | CTGaGCTcATgggTtTGA | Intergenic |
| | (SEQ. ID. NO.: 1268) | (SEQ. ID. NO.: 1289) | |
| chr3: 82950355 | aCAtAtAAATAAGaaCAt | CTtGGCTTATTTtaCTGA | Intergenic |
| | (SEQ. ID. NO.: 1269) | (SEQ. ID. NO.: 1290) | |
| chr22: 40341367 | TCAGAGAAATgAGCCCct | tcGGctTTAaTcCTCTGA | Intron (GRAP2) |
| | (SEQ. ID. NO.: 1270) | (SEQ. ID. NO.: 1291) | |
| chr20: 19686090 | TtgGAaAAATAAtCCCAG | taGGGCTTATTTgctTGA | Intron (SLC24A3) |
| | (SEQ. ID. NO.: 1271) | (SEQ. ID. NO.: 1292) | |
| chr4: 20811976 | TCAGAGAcAatAtCaaAG | gTGGGtTTATTTgTCTGA | Intron (KCNIP4) |
| | (SEQ. ID. NO.: 1272) | (SEQ. ID. NO.: 1293) | |
| chrX: 97284124 | TCAGgGcAATcAGCCCAG | CTGGGgTTtcTTgTCTGg | Intergenic |
| | (SEQ. ID. NO.: 1273) | (SEQ. ID. NO.: 1294) | |
| chr18: 41220996 | TCAaAtgAATAAGaCaAt | tTGGttTTgTTTCTCTGA | Intergenic |
| | (SEQ. ID. NO.: 1274) | (SEQ. ID. NO.: 1295) | |
| chrY: 19504648 | TCAGgaAAAaAAtCCCAG | CTtGttTTATTctcCTGA | Intergenic |
| | (SEQ. ID. NO.: 1275) | (SEQ. ID. NO.: 1296) | |
| chr6: 11989807 | TCAtAtAAATgAGCtCAt | CTtGGCTTcTTTCaCTGA | Intergenic |
| | (SEQ. ID. NO.: 1276) | (SEQ. ID. NO.: 1297) | |
| chr11: 100111323 | TaAaAttAATgAGCCCAG | tTtGGCTTATTTCcaTGA | Intron (CNTN5) |
| | (SEQ. ID. NO.: 1277) | (SEQ. ID. NO.: 1298) | |
| chr13: 26279732 | agAGAGAAAaAgGCCgAG | tTGGGtTTATTTtTCTaA | Intron (ATP8A2) |
| | (SEQ. ID. NO.: 1278) | (SEQ. ID. NO.: 1299) | |

TABLE 45
Targeting Exon 17:

| Genome Coordinates | Left Half-Site | Right Half-Site | Genomic Region |
|---|---|---|---|
| chrX: 154132638 | TCTTTCCATATTTTCAG | TTTGGTCTCATCAAAGA | Exon (F8) |
| | (SEQ. ID. NO.: 1300) | (SEQ. ID. NO.: 1321) | |
| chr11: 86435291 | aCTTTCCATAgTTTCAG | CTGAAAATATtGAAtGA | Intergenic |
| | (SEQ. ID. NO.: 1301) | (SEQ. ID. NO.: 1322) | |
| chr17: 191390 | TCTTaGAgGAcACCAAA | TTTGGTgTCATCtAAGA | Intron (RPH3AL) |
| | (SEQ. ID. NO.: 1302) | (SEQ. ID. NO.: 1323) | |
| chrX: 16807199 | TCTaTCCtTtTTTTCAG | tTGAAAATATtGAAAGA | Intron (TXLNG) |
| | (SEQ. ID. NO.: 1303) | (SEQ. ID. NO.: 1324) | |
| chrX: 4909433 | TtTTTCCATATTTTCAG | TcaGtTtTCtTCAAAGA | Intergenic |
| | (SEQ. ID. NO.: 1304) | (SEQ. ID. NO.: 1325) | |
| chr15: 98192520 | TCTTTCCAcATTTTCAG | CTGAAAATATtaAAtaA | Intergenic |
| | (SEQ. ID. NO.: 1305) | (SEQ. ID. NO.: 1326) | |
| chr3: 65632758 | TCTTTGAaaAGACCAAA | CTGAcAAcAgGGAAAaA | Intron (MAGI1) |
| | (SEQ. ID. NO.: 1306) | (SEQ. ID. NO.: 1327) | |
| chrX: 81782933 | TCaTTtaATATTTTtgG | CTGAAAATgTGGAAAGA | Intergenic |
| | (SEQ. ID. NO.: 1307) | (SEQ. ID. NO.: 1328) | |
| chr20: 48433923 | TCTTTaATGAtACCAAA | TTaGGTCTttTCAgAaA | Intron (SLC9A8) |
| | (SEQ. ID. NO.: 1308) | (SEQ. ID. NO.: 1329) | |
| chr8: 84366161 | TCaTTtCATATTTTCAG | CTGAAAtTgTGGAAAGt | Intergenic |
| | (SEQ. ID. NO.: 1309) | (SEQ. ID. NO.: 1657) | |
| chr1: 93406669 | atTTTGATaAGAtCAAA | TTTGGTgTCATCtAAGA | Intron (FAM69A) |
| | (SEQ. ID. NO.: 1310) | (SEQ. ID. NO.: 1330) | |
| chr3: 23702529 | TaTTTGATttaAtCAAA | TTTGGTtTCATgAAAGA | Intergenic |
| | (SEQ. ID. NO.: 1311) | (SEQ. ID. NO.: 1331) | |
| chr4: 127360864 | TCTTTCCAcATTcTCtG | gTTGGTtTCATCcAAGA | Intergenic |
| | (SEQ. ID. NO.: 1312) | (SEQ. ID. NO.: 1332) | |
| chr9: 10862420 | TtTTaGAaGAaAaCAAA | TTTGGTgTCAgCAAAGA | Intergenic |
| | (SEQ. ID. NO.: 1313) | (SEQ. ID. NO.: 1333) | |
| chr2: 30136701 | TCTcTCCATATTcTCca | CTGAAAATAcaGAAAGA | Intron (ALK) |
| | (SEQ. ID. NO.: 1314) | (SEQ. ID. NO.: 1334) | |
| chr2: 8966383 | TtTTTaATaAtcCCAAA | TTgGGgCTCATtAAAGA | Intron (KIDINS220) |
| | (SEQ. ID. NO.: 1315) | (SEQ. ID. NO.: 1335) | |
| chr10: 106620765 | TCcTgGgTGAGACCcAA | TcTGGTtTCATCAAgGA | Intron (SORCS3) |
| | (SEQ. ID. NO.: 1316) | (SEQ. ID. NO.: 1336) | |
| chrX: 108769761 | TaTTTGATGAGACCAAc | aTGAgAATATaGcAAGA | Intergenic |
| | (SEQ. ID. NO.: 1317) | (SEQ. ID. NO.: 1337) | |
| chr1: 111227475 | TCaTTtaATATTTTCAG | CTGAAAtTATGGAAAGc | Intergenic |
| | (SEQ. ID. NO.: 1318) | (SEQ. ID. NO.: 1338) | |
| chr3: 114347859 | TCTTTGATGAaAaCcAA | TTTGtTtTCAcaAAtGA | Intron (ZBTB20) |
| | (SEQ. ID. NO.: 1319) | (SEQ. ID. NO.: 1339) | |
| chr6: 24241996 | TCTTTCCATATTTTaAt | taGAAtATATGaAtAGA | Intron (DCDC2) |
| | (SEQ. ID. NO.: 1320) | (SEQ. ID. NO.: 1340) | |

TABLE 46
Targeting Exon 18:

| Genome Coordinates | Left Half-Site | Right Half-Site | Genomic Region |
|---|---|---|---|
| chrX: 154132208 | TATACTCCTCTTTTTTTCG | GTCCACTGAAATGAATAGA | Exon (F8) |
| | (SEQ. ID. NO.: 1341) | (SEQ. ID. NO.: 1362) | |
| chr3: 89963270 | TCTATTCATTaCtGTttAC | GTCCAtTGAAtTGcATAaA | Intergenic |
| | (SEQ. ID. NO.: 1342) | (SEQ. ID. NO.: 1363) | |
| chr13: 71330234 | TtTATTCATTTCAtTGaAa | GTCtAtTtAAATaAAgAGA | Intergenic |
| | (SEQ. ID. NO.: 1343) | (SEQ. ID. NO.: 1364) | |
| chr7: 52504835 | TCTATaCATTTCAGaacAC | GcaCACTaAAAaGAAcAGA | Intergenic |
| | (SEQ. ID. NO.: 1344) | (SEQ. ID. NO.: 1365) | |
| chr7: 93233952 | aATACTCCTCcTTcTTTtt | aTaCACTGAAATGgATAGA | Intergenic |
| | (SEQ. ID. NO.: 1345) | (SEQ. ID. NO.: 1366) | |
| chr20: 8957392 | TATAaaCgTtTaTTTTTCt | GTtaACTGAAATGAcTAGA | Intergenic |
| | (SEQ. ID. NO.: 1346) | (SEQ. ID. NO.: 1367) | |
| chr2: 55547229 | TATACTtCTCTTTTgTTCa | tGAAAAAAtGtGtAcTAgA | Intron (CCDC88A) |
| | (SEQ. ID. NO.: 1347) | (SEQ. ID. NO.: 1368) | |
| chr6: 55916123 | cATACTCCTCTTaTTTTCa | tgCCACTGAAATGAcTttt | Intergenic |
| | (SEQ. ID. NO.: 1348) | (SEQ. ID. NO.: 1369) | |
| chr8: 93952422 | TCTATcCATgTCAaaGaAC | GTCttCTcAAATGtAcAGA | Intron (TRIQK) |
| | (SEQ. ID. NO.: 1349) | (SEQ. ID. NO.: 1370) | |
| chr14: 61101496 | TCTATcCATTTCtGTGtAC | tGcAAAtAAaAGtAGTATt | Intergenic |
| | (SEQ. ID. NO.: 1350) | (SEQ. ID. NO.: 1371) | |
| chr11: 33381162 | TATACTtCTaTTTTTTTat | aGAAAAAgAGAGtAGTAcA | Intergenic |
| | (SEQ. ID. NO.: 1351) | (SEQ. ID. NO.: 1372) | |
| chr6: 84078984 | TCTATTacTgaCAcTGaAC | GTCtACTGAAgTGAActGA | Intron (ME1) |
| | (SEQ. ID. NO.: 1352) | (SEQ. ID. NO.: 1373) | |
| chr11: 123025415 | aATcCcCCTCaTTTTTctG | tTCCACTGAAATGAtTAtA | Intron (CLMP) |
| | (SEQ. ID. NO.: 1353) | (SEQ. ID. NO.: 1374) | |
| chr1: 58698828 | TAatCaCCTCTTTTTcTCc | GTatAtTGAAATGtAgAGA | Intron (DAB1) |
| | (SEQ. ID. NO.: 1354) | (SEQ. ID. NO.: 1375) | |
| chr13: 90438048 | TCTATTaATaTCAGTaaAC | GgCCAaTGAAAcaAATgGc | Intergenic |
| | (SEQ. ID. NO.: 1355) | (SEQ. ID. NO.: 1376) | |
| chr3: 20841157 | TCTtccCATTTCtGTGaAa | GTtaAaTGgAATGAATAGA | Intergenic |
| | (SEQ. ID. NO.: 1356) | (SEQ. ID. NO.: 1377) | |
| chr5: 22000977 | TCTATTaAaaTCAaTaGAC | GTttACTtAcATtAtTAGA | Intron (CDH12) |
| | (SEQ. ID. NO.: 1357) | (SEQ. ID. NO.: 1378) | |
| chr5: 69306485 | TCTATTaAaaTCAaTaGAC | GTttACTtAcATtAtTAGA | Intergenic |
| | (SEQ. ID. NO.: 1358) | (SEQ. ID. NO.: 1379) | |
| chr5: 70181567 | TCTATTaAaaTCAaTaGAC | GTttACTtAcATtAtTAGA | Intergenic |
| | (SEQ. ID. NO.: 1359) | (SEQ. ID. NO.: 1380) | |
| chr3: 62322281 | aCTATaCATTTCAaTaGtC | tTCCACTGtAATtAgTAtA | Intergenic |
| | (SEQ. ID. NO.: 1360) | (SEQ. ID. NO.: 1381) | |
| chr1: 239837471 | TtaAaTtATTTCcGTGGAa | GTCCACaGAtATGAATAtA | Intron (CHRM3) |
| | (SEQ. ID. NO.: 1361) | (SEQ. ID. NO.: 1382) | |

TABLE 47
Targeting Exon 19:

| Genome Coordinates | Left Half-Site | Right Half-Site | Genomic Region |
|---|---|---|---|
| chrX: 154130370 | TGCTCGCCAATAAGGCATTCC | AGCTTTGGATGGTAACA | Dmn (F8) |
| | (SEQ. ID. NO.: 1383) | (SEQ. ID. NO.: 1404) | |
| chr4: 53352906 | TGTgACCATCCAAgGCT | AGCaTTGGAgGGgAACA | Intergenic |
| | (SEQ. ID. NO.: 1384) | (SEQ. ID. NO.: 1405) | |
| chr21: 36529769 | TGTTcCCAcCCAAAtCT | AGaTTTGGgTGGggACA | Intergenic |
| | (SEQ. ID. NO.: 1385) | (SEQ. ID. NO.: 1406) | |
| chr9: 76182583 | aaTTACaAaCaAAAGCc | tGCTTTtGATGGTAAtA | Intergenic |
| | (SEQ. ID. NO.: 1386) | (SEQ. ID. NO.: 1407) | |
| chr3: 81470457 | TGTTACttTgCAAAtgc | AatTTTGGATGGTAACA | Intergenic |
| | (SEQ. ID. NO.: 1387) | (SEQ. ID. NO.: 1408) | |
| chr1: 203239036 | TGTTACCAgCCAAAcCT | AGggaTGGAgGGTtgCA | Intergenic |
| | (SEQ. ID. NO.: 1388) | (SEQ. ID. NO.: 1409) | |
| chr3: 65643349 | TGTTtCCtTtaAAAtCT | AGCTTTGtcTGGTAACA | Intron (MAGI1) |
| | (SEQ. ID. NO.: 1389) | (SEQ. ID. NO.: 1410) | |
| chr2: 52456162 | TaTTgCCtTCatcAGCT | AGCTTTGGAaGGTAtCA | Intergenic |
| | (SEQ. ID. NO.: 1390) | (SEQ. ID. NO.: 1411) | |
| chr4: 150055809 | TtTcACCATCCAAAtCT | AttgTTGGgTGGTAAgA | Intergenic |
| | (SEQ. ID. NO.: 1391) | (SEQ. ID. NO.: 1412) | |
| chr11: 43851516 | TacTACCATaCAAAGCT | tGgaTTGGATGtTcACA | Intron (HSD17B12) |
| | (SEQ. ID. NO.: 1392) | (SEQ. ID. NO.: 1413) | |
| chr7: 114250318 | TaTTACtgTCtAtAtCT | AGCTTTGaATGGTAAaA | Intron (FOXP2) |
| | (SEQ. ID. NO.: 1393) | (SEQ. ID. NO.: 1414) | |
| chr3: 167657104 | TGTgAaCATCCAAgGCT | AGCTcTtGATGGTcACt | Intergenic |
| | (SEQ. ID. NO.: 1394) | (SEQ. ID. NO.: 1415) | |
| chrX: 149844333 | TGgTgCCtaCCAcAcCT | AGCTTTGGATGGTcAgA | Intergenic |
| | (SEQ. ID. NO.: 1395) | (SEQ. ID. NO.: 1416) | |
| chr9: 29156612 | TGaTAaCtTCCAAgaCT | gtCTTTGGAaGGTAACA | Intron (UNGO2) |
| | (SEQ. ID. NO.: 1396) | (SEQ. ID. NO.: 1417) | |
| chr4: 70236889 | TaTTACCATCaAAAtCa | AGCTTTtGtaGGTAAtg | Intergenic |
| | (SEQ. ID. NO.: 1397) | (SEQ. ID. NO.: 1418) | |
| chr3: 151160745 | aaTTcCaAcCCAAAGgT | AGCcTTGGATGGTAACc | Exon (IGSF10) |
| | (SEQ. ID. NO.: 1398) | (SEQ. ID. NO.: 1419) | |
| chr13: 35431619 | TtTTACCcTCCAAAcCc | AGCTTTGGAaaaTAACA | Intergenic |
| | (SEQ. ID. NO.: 1399) | (SEQ. ID. NO.: 1420) | |
| chr4: 29377428 | TGTTAaaATCCtAAtCc | AcCTTTGGATGGTAAtt | Intergenic |
| | (SEQ. ID. NO.: 1400) | (SEQ. ID. NO.: 1421) | |
| chr13: 62451673 | TGTTcCCAcCCAAAtCT | AGagTTGGAgGGaAgtA | Intergenic |
| | (SEQ. ID. NO.: 1401) | (SEQ. ID. NO.: 1422) | |
| chr12: 95616056 | TtTTcCCATttAgAtCT | AttTTTGtATGGTAACA | Intron (VEZT) |
| | (SEQ. ID. NO.: 1402) | (SEQ. ID. NO.: 1423) | |
| chr18: 28761651 | TagaACCATCCAAAaCT | AGaTTTGcATGtTtAaA | Intergenic |
| | (SEQ. ID. NO.: 1403) | (SEQ. ID. NO.: 1424) | |

TABLE 48
Targeting Exon 20:

| Genome Coordinates | Left Half-Site | Right Half-Site | Genomic Region |
|---|---|---|---|
| chrX: 154129651 | TGTCCTGAAGCTGTAATCTGAA | CCAGAAGCCATTCCCAGGGGA | Exon (F8) |
| | (SEQ. ID. NO.: 1425) | (SEQ. ID. NO.: 1446) | |
| chr8: 31295960 | TCCCCTaGGAcTGaCTTCaGa | CCAGActCtATTgCCAtGtGg | Intergenic |
| | (SEQ. ID. NO.: 1426) | (SEQ. ID. NO.: 1447) | |
| chr2: 165151202 | aaTCCaGAAGCaGTAAcCaGtA | CgtGAAtCCtTTCCCAGGGGA | Intergenic |
| | (SEQ. ID. NO.: 1427) | (SEQ. ID. NO.: 1448) | |
| chr15: 66216735 | TCCCCaGGGAATGGgaTCTGG | ACAGggGtCtcTCCCAGtGGt | Intron (MEGF11) |
| | (SEQ. ID. NO.: 1428) | (SEQ. ID. NO.: 1658) | |
| chr14: 97246034 | TgCCaTGGGAtTtGCTTCTGc | CCAGAAGCagTcttCAGGGGA | Intergenic |
| | (SEQ. ID. NO.: 1429) | (SEQ. ID. NO.: 1449) | |
| chr1: 17425225 | TCCaCTGaaAtgacCTTCTGG | CCtGtAGtCATgCCCAtGGGA | Intron (PADI2) |
| | (SEQ. ID. NO.: 1430) | (SEQ. ID. NO.: 1450) | |
| chr19: 11752845 | TCCCCTGGGAcactCagCTtt | CCAGAttCCATTCCttGGGGA | Intergenic |
| | (SEQ. ID. NO.: 1431) | (SEQ. ID. NO.: 1451) | |
| chr6: 165113924 | TCCCtTGGcAATtGCTTCTct | CCccAttCCATTCaCAGGGGA | Intergenic |
| | (SEQ. ID. NO.: 1432) | (SEQ. ID. NO.: 1452) | |
| chr3: 18310932 | TtCCCTGattATaGCTTtctG | CCAGAAGaCATTtCaAGGaGA | Intergenic |
| | (SEQ. ID. NO.: 1433) | (SEQ. ID. NO.: 1453) | |
| chr16: 54478454 | TCtCCaGaGAgaGGCTTCTaG | CCtGAtGtCcTTCCtttGGGA | Intergenic |
| | (SEQ. ID. NO.: 1434) | (SEQ. ID. NO.: 1454) | |
| chr2: 100885233 | TCCtCaGtcAATGGCTTCTGG | atgGAAaCCAgTCCaAGGGaA | Intergenic |
| | (SEQ. ID. NO.: 1435) | (SEQ. ID. NO.: 1455) | |
| chr6: 160576093 | TgCtCTtGGgATGtCTTCTGG | taAGAAtCCATTCCtAGGatA | Intron (SLC22A1) |
| | (SEQ. ID. NO.: 1436) | (SEQ. ID. NO.: 1456) | |
| chr1: 888254 | TaCCCTGGccATGGCcTCaGG | agAGAgGCCcTcCCCtGGGGA | Intron (NOC2L) |
| | (SEQ. ID. NO.: 1437) | (SEQ. ID. NO.: 1457) | |
| chr11: 24688064 | TCCatTGaaAATaGCTcCTGa | gCAGgAGCtATTCtCAGacGA | Intron (LUZP2) |
| | (SEQ. ID. NO.: 1438) | (SEQ. ID. NO.: 1458) | |
| chr3: 188747522 | TCCCtTGtGAATGGCTTggtG | aCcGtAGtCATTCCCAtGaGA | Intergenic |
| | (SEQ. ID. NO.: 1439) | (SEQ. ID. NO.: 1459) | |
| chr10: 74502577 | TcTCCTGAAGaTGTAATtaGAg | CCtGAgGtgATTtCtAGGGGg | Intron (MCU) |
| | (SEQ. ID. NO.: 1440) | (SEQ. ID. NO.: 14670) | |
| chrX: 28644076 | TCCaCaGaGAATaGtTTaTGc | CttGtAcCCATTCCatGGGGA | Intron (IL1RAPL1) |
| | (SEQ. ID. NO.: 1441) | (SEQ. ID. NO.: 1461) | |
| chr2: 167140954 | cGTCCTtAcGCTGTcATCaGAA | gCAGAAGCtgTcCattGGGGA | Intron (SCN9A) |
| | (SEQ. ID. NO.: 1442) | (SEQ. ID. NO.: 1462) | |
| chr10: 3095266 | gCaCCTtGaAATGGgcaCTGG | CCgGAAGCCATTCCaAatGGA | Intergenic |
| | (SEQ. ID. NO.: 1443) | (SEQ. ID. NO.: 1463) | |
| chr5: 73250307 | TCCCCTGGGAActGCTgaTGG | CCAGAAGggATggtaAaGGGA | Intergenic |
| | (SEQ. ID. NO.: 1444) | (SEQ. ID. NO.: 1464) | |
| chr1: 145822030 | TCaCCTGGGAATaGtaTCTaG | CaAGAAGaaAacaCtAGaGGA | Intron (GPR89A) |
| | (SEQ. ID. NO.: 1445) | (SEQ. ID. NO.: 1465) | |

TABLE 49
Targeting Exon 21:

| Genome Coordinates | Left Half-Site | Right Half-Site | Genomic Region |
|---|---|---|---|
| chrX: 154128167 | TGCTCCAGGCATTGATTGAT | CTGGCCAGCTTTGGGGCCCA | Exon (F8) |
| | (SEQ. ID. NO.: 1466) | (SEQ. ID. NO.: 1487) | |
| chr10: 123955374 | TGGtCCCacAgGCTGGCCAG | CTGGaCAGCTcTGGGcCCCA | Intron (TACC2) |
| | (SEQ. ID. NO.: 1467) | (SEQ. ID. NO.: 1488) | |
| chr6: 73606839 | TaCTCCAGGCATaGAagGAg | tTGGaCcaCTTTGGGGCCCA | Intron (KCNQ5) |
| | (SEQ. ID. NO.: 1468) | (SEQ. ID. NO.: 1489) | |
| chr15: 87990891 | aGaGCCCCAtAtCTccCaAG | ATCAgTCAtTGtCTGGAGCA | Intergenic |
| | (SEQ. ID. NO.: 1469) | (SEQ. ID. NO.: 1490) | |
| chr13: 104866433 | TGCTtCAGaCAcTGATTGAg | aTtGCCAcaTTTGGGGCCCA | Intergenic |
| | (SEQ. ID. NO.: 1470) | (SEQ. ID. NO.: 1491) | |
| chr21: 44889451 | TGGtCCCCAAAcCTGGCCAa | CTGGaCAGaTgccaGGgCCA | Intron (LINC00313) |
| | (SEQ. ID. NO.: 1471) | (SEQ. ID. NO.: 1492) | |
| chr15: 72922848 | gGaGgCCCAAAcgTGGCCtt | CTaGCCAGCTcTGGGGCCCA | Intergenic |
| | (SEQ. ID. NO.: 1472) | (SEQ. ID. NO.: 1493) | |
| chr8: 20252698 | TGCTCattGCAcTGgTgGAT | CTGGCaAGCTTTGGGGtCtg | Intergenic |
| | (SEQ. ID. NO.: 1473) | (SEQ. ID. NO.: 1494) | |
| chr18: 32975516 | TGtGgCCCAtAGCTGGCCAG | CTGGCCAGCTaTGGGttttc | Intergenic |
| | (SEQ. ID. NO.: 1474) | (SEQ. ID. NO.: 1495) | |
| chr16: 989379 | TGcGCCaCAAAGCTGGCCAc | AgCAATaAAaaCCaGGAaCA | Intron (LMF1) |
| | (SEQ. ID. NO.: 1475) | (SEQ. ID. NO.: 1496) | |
| chr20: 44515651 | TGGGCCCCAggcCTGGgCAG | CTGctCAGCTTTctGGCtCA | Exon (SPATA25) |
| | (SEQ. ID. NO.: 1476) | (SEQ. ID. NO.: 1497) | |
| chr2: 240861687 | TaGGCaCCtcAGCTGGCCAa | CTGGgCAGCcTgGGaGCCCt | Intergenic |
| | (SEQ. ID. NO.: 1477) | (SEQ. ID. NO.: 1498) | |
| chr9: 132364724 | TGaGCCaCtgAGCTGGCCAG | cTtAtTCctTGtCTGGAGaA | Intergenic |
| | (SEQ. ID. NO.: 1478) | (SEQ. ID. NO.: 1499) | |
| chr1: 151341446 | TGGtCtaCtgAGCTGGCaAG | tTGtgCAGCTTTGGGGCCCg | Intron (SELENBP1) |
| | (SEQ. ID. NO.: 1479) | (SEQ. ID. NO.: 1500) | |
| chr12: 1996302 | TGGaCCCCcAAGaTGGCCAt | CaGaaCAGCTTTGGaGCtag | Intron (CACNA2D4) |
| | (SEQ. ID. NO.: 1480) | (SEQ. ID. NO.: 1501) | |
| chr16: 68354549 | TGCTgCAGagATTtgTTtAT | tTGGCCAGaTTTGGGGgCCt | Intron (PRMT7) |
| | (SEQ. ID. NO.: 1481) | (SEQ. ID. NO.: 1502) | |
| chr3: 64099060 | TGGGgCCCcAgcCTGGCCAc | tTGGgtAcCTTgGGGGCCCA | Intron (PRICKLE2) |
| | (SEQ. ID. NO.: 1482) | (SEQ. ID. NO.: 1503) | |
| chr12: 133199141 | TGGtCCCCAcAGCcaGCCAG | CTGcCCAGgcTgGGaGtgCA | Intergenic |
| | (SEQ. ID. NO.: 1483) | (SEQ. ID. NO.: 1504) | |
| chr12: 53741716 | TaaGaaCCAAAGCTaatCAG | tTcttCAGtTTTGtGGCCCA | Intergenic |
| | (SEQ. ID. NO.: 1484) | (SEQ. ID. NO.: 1505) | |
| chr16: 3006381 | TGGGgCCCAAAtgaaGCCAG | CctGCCAGCcTTGGGGtCCt | Intergenic |
| | (SEQ. ID. NO.: 1485) | (SEQ. ID. NO.: 1506) | |
| chr5: 53389184 | aGcaCCCCAAAcCTGGCCtG | tTGGgCAGCaTTtGGcCCCA | Intron (ARL15) |
| | (SEQ. ID. NO.: 1486) | (SEQ. ID. NO.: 1507) | |

TABLE 50
Targeting Exon 22:

| Genome Coordinates | Left Half-Site | Right Half-Site | Genomic Region |
|---|---|---|---|
| chrX: 154124384 | TCTGCCACTTCTTCCCATCAAG | ATAAACTGAGAGATGTAGA | Exon (F8) |
| | (SEQ. ID. NO.: 1508) | (SEQ. ID. NO.: 1529) | |
| chr17: 55200444 | TCaACATCTgTCAGacgAT | ATAAAaTGAGAGtTGTAGc | Intergenic |
| | (SEQ. ID. NO.: 1509) | (SEQ. ID. NO.: 1530) | |
| chr7: 149959793 | TCTACATCTaaCAtTTTAT | ATAAAtgGAaAacTGgAGA | Intron (ACTR3C) |
| | (SEQ. ID. NO.: 1510) | (SEQ. ID. NO.: 1531) | |
| chr3: 182164176 | TgcACATCTCTCAcTTTAa | AaAAgCTGAGAGAgGTtGA | Intergenic |
| | (SEQ. ID. NO.: 1511) | (SEQ. ID. NO.: 1532) | |
| chr8: 85206496 | TgTgCtTaTCTaAGTacAT | gcAAAtTGAGAGATGTAGA | Intron (RALYL) |
| | (SEQ. ID. NO.: 1512) | (SEQ. ID. NO.: 1533) | |
| chr1: 107949372 | TtTACATCTaTCAGTTTAT | AaAAACTGAGctAcagAGg | Mtron (NTNG1) |
| | (SEQ. ID. NO.: 1513) | (SEQ. ID. NO.: 1534) | |
| chr3: 150421949 | TCTtCgTCTCTCAGcTTAT | CTTGggtGGAgGAAGTGGCttc | Promoter (FAM194A) |
| | (SEQ. ID. NO.: 1514) | (SEQ. ID. NO.: 1535) | |
| chr8: 22075977 | gCTcCATCTCaaAaaTaAT | ATAAAaTGAtAGATGcAGA | Intergenic |
| | (SEQ. ID. NO.: 1515) | (SEQ. ID. NO.: 1536) | |
| chr5: 56152387 | TaTACATtTCTCAtTTTAT | tTtAgtcGtGAGATGgAGA | Intron (MAP3K1) |
| | (SEQ. ID. NO.: 1516) | (SEQ. ID. NO.: 1537) | |
| chrX: 147805582 | TtgGCCACTTCTTCCCATCccG | tTAAcCTGAaAcATGgAGA | Intron (AFF2) |
| | (SEQ. ID. NO.: 1517) | (SEQ. ID. NO.: 1538) | |
| chr3: 59243225 | aCgAtATCaCTatGTTTAc | ATAAtCTGAGAGtTGTAtA | Intergenic |
| | (SEQ. ID. NO.: 1518) | (SEQ. ID. NO.: 1539) | |
| chr15: 88546432 | TCTAgATCTaaCtGacaAT | ATAAACTGgGAGgcGTAGA | Intron (NTRK3) |
| | (SEQ. ID. NO.: 1519) | (SEQ. ID. NO.: 1540) | |
| chr3: 101738660 | TCTAgATCTCTCAGgTTAa | caActCTGtGAGATGaAGA | Intergenic |
| | (SEQ. ID. NO.: 1520) | (SEQ. ID. NO.: 1541) | |
| chr15: 64473144 | TCTAgtTCTCTCAGTTTAT | ATAgACTtAGtGcTGatGt | Intron (CSNK1G1) |
| | (SEQ. ID. NO.: 1521) | (SEQ. ID. NO.: 1542) | |
| chr15: 96928325 | agTACATCTtTtAaTTTAT | CcTGATGGGAAGAAtTaGaAGA | Intergenic |
| | (SEQ. ID. NO.: 1522) | (SEQ. ID. NO.: 1543) | |
| chr11: 85386305 | cCatCcTCaCTaAGTTTAa | tTAAAgTGAGAGATGTAtA | Intergenic |
| | (SEQ. ID. NO.: 1523) | (SEQ. ID. NO.: 1544) | |
| chr5: 117743942 | TCTcCATCTggCAaTTgAg | cTAAACTGgaAGATGTAGA | Intergenic |
| | (SEQ. ID. NO.: 1524) | (SEQ. ID. NO.: 1545) | |
| chr1: 5052686 | TaTACATtTCTCAGTTgAT | CTTGtTctGAcGAtGctGCAGA | Intergenic |
| | (SEQ. ID. NO.: 1525) | (SEQ. ID. NO.: 1546) | |
| chr6: 9920117 | caTACATCTCTCAcTTTAT | tTAAACTtAGtGAgGaAGg | Intergenic |
| | (SEQ. ID. NO.: 1526) | (SEQ. ID. NO.: 1547) | |
| chr1: 159052090 | TCTcCATgTCTCAGTTTgT | ATAgACTaAGtGActTAtA | Intergenic |
| | (SEQ. ID. NO.: 1527) | (SEQ. ID. NO.: 1548) | |
| chr20: 25560526 | TCTACAaaTgTaAaaTTcT | AaAAACTGAGAGATtTtGA | Intron (NINL) |
| | (SEQ. ID. NO.: 1528) | (SEQ. ID. NO.: 1549) | |

In all exons 1-22, favorable sites were able to be located for TALENs, Cas9-nuclease, Cas9 paired-nickase, and dCas9 RNA-guided FokI Nucleases (RFNs). These sites met guidelines established for predicting high on-target activity (using the SAPTA algorithm for TALENs and avoiding stretches of pyrimidines in the PAM-proximal region of the target). These sites also met guidelines established for being relatively unique throughout the genome and having no high-scoring predicted off-target sites. Analysis of TALEN sites using PROGNOS yielded no sites generating warnings as scoring substantially similar to the designated target site. Analysis of Cas9-nuclease off-target sites found in almost all cases that no sites existed with fewer than two mismatches to the target sequence; furthermore, sites with few mismatches typically had mismatches in disruptive regions such as the PAM, or the 12 bp PAM-proximal ‘seed region’. Cas9-nickases and RFNs have been shown to have very low off-target activity approaching the detection limit of deep-sequencing assays (Ran & Hsu et al. Cell 2013, Tsai S Q et al. Nature Biotech 2014).

Taken together, this example identified the sequences to repair the F8 gene at the 3′ end of any exon 1-22 for TALENs, Cas9-nucleases, Cas9-nickases, or RFNs; by using the abovementioned selected target sites. High on-target activity allows efficacious clinical repair of HA and low off-target activity ensures the safety of the proposed therapy.

### Example 4

### Homologous Repair Vehicles for Repair at Different Exon-Intron Junctions

Repair at different exon-intron junctions throughout the FVIII gene employ methodology similar to example 3 described above, the repair vehicles used however are different for each junction. This example describes various repair vehicles.

All repair vehicles contain the same basic components: a left homology arm corresponding to the genomic sequence 5′ of the relevant nuclease cut site, a cDNA sequence comprising the downstream protein coding sequence of FVIII, a polyadenylation signal (such as the human growth hormone polyadenylation signal, or the bovine growth hormone polyadenylation signal, or other signals well known in the art), and a right homology arm corresponding the genomic sequence 3′ of the relevant nuclease cut site. The cDNA optionally contains several synonymous SNPs to aid experimental validation that productive repair has occurred. Further, the cDNA in different repair vehicles may contain non-synonymous SNPs in order to be a haplotypic match for different patients.

For example, a vehicle designed for repair at exon 22 consists of a left homology arm comprising the 5′ portion of exon 22 and possibly continuing into the 3′ portion of intron 21, a cDNA containing exons 23-26, and a right homology arm comprising a portion of the 5′ region of intron 22; such a repair vehicle is detailed in the sequence in Table 51 below.

TABLE 51
TTAAGGATCTCAGTCTAATAAGGAAAGCAGAAAAGCAAAGCAACCTTATA
ATATGGTGCAATAATTTGCTATAATGAAGTTATATACAAAGTGAAGTAGA
AGCATAGAAGAAGCAGCACTAAATTTGTCTGGGTGAGTCAGAGAAGGCTA
ACCAGGAAAAATAGTTTCTGAACTAACACTTGAAGGAGGTGTAGCAGTTC
ATCACTGACAGTGATGTTGGGGTGGGTCTGGTTTCAGGAGAGGGGAGGAA
ATTGGCTTTGGTCTGAGGCTGAGGTGTGGGCAAAGCATTAGCTTATGTGG
GTCCATTAGCTTATGTGAGTCCACAAAAGGTGTGTGTGTGTTTGTGTGTA
TGTGTGTGTGTGTGTGTGTGTGTGTGTGTGTACGAAATGGGGGCTCAATG
ATTTGGTAGTGGTTTGGTTTGTCAAGAAGCAGGCTGGGAACTCAATAAGC
ATCTTTCCATTCATTTCTACTGTGTATCCCACAGCTTCACACACACATGC
ACATTTCAACATTGGTGACTGCTTCACTTGCACACCTAAGGTAATGATGG
ACACACCTGTAGCAATGTAGATTCTTCCTAAGCTAATAATTAGTTTCAGG
AGGTAGCACATACATTTAAAAATAGGTTAAAATAAAGTGTTATTTTAATT
GGTAGGTGGATCTGTTGGCACCAATGATTATTCACGGCATCAAGACCCAG
GGTGCCCGTCAGAAGTTCTCCAGTCTCTATATCTCTCAGTTTATCATCAT
GTATAGTCTCGACGGCAAGAAGTGGCAGACGTACCGAGGAAATTCCAGTG
GAACCTTAATGgtcttctttggcaatgtggattcatctgggataaaacac
aatatttttaaccctccaattattgctcgatacatccgtttgcacccaac
tcattatagcattcgcagcactcttcgcatggagttgatgggctgtgatt
taaatagttgcagcatgccattgggaatggagagtaaagcaatatcagat
gcacagattactgcttcatcctactttaccaatatgtttgccacctggtc
tccttcaaaagctcgacttcacctccaagggaggagtaatgcctggagac
ctcaggtgaataatccaaaagagtggctgcaagtggacttccagaagaca
atgaaagtcacaggagtaactactcagggagtaaaatctctgcttaccag
catgtatgtgaaggagttcctcatctccagcagtcaagatggccatcagt
ggactctcttttttcagaatggcaaagtaaaggtttttcagggaaatcaa
gactccttcacacctgtggtgaactctctagacccaccgttactgactcg
ctaccttcgaattcacccccagagttgggtgcaccagattgccctgagga
tggaggttctgggctgcgaggcacaggacctctactgagaattcCTAGAG
CTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGG
GAAGAgAATAGCAGGCATGCTGGGGAGTATGTAATTAGTCATTTAAAGGG
AATGCCTGAATACTTTAAAGAATTTTGGCAGATTTCAGATATTGGACAAA
CACTCTTAGCTTCCACAAACTTAATTCCAAAAAATAATTTTTCACTTATG
AGCAATAGAGTTATTACGGACATATCAGCAAAAATGTAGTAGTGTCAAGG
CTCATAGATGATAGAAATGAAGAGATGCTGTATTGATAGAAATATGTGAT
TCAGGACTGTGTGGATTGATGATTGTGAGCTTGCTTATGGATATCCTAGG
TTTGAGGTTATAGTAGGACAATCAGGTTGAAATGTCCAGCAGGCAGTAGG
TGAAAGACAAGTTTAGGGGGCAAAACCATGGATGGAGATGAAGATTCATG
ACTTCCACATAAAAGGATGGGTGAAACTTTGGGAATTGATGAATTCTCTA
GAGGTGAGCTCAAGACCCTTAAAGGCTTAAAACCTCAGCGTTATTGTCTA
CTCTTCCCTCATTTTTATGCCCACAAATCTGGTCAATCCTTTATTTGCAA
TGCCTCTCACATCTCTTTCTTCTGTTTCCATTTATACCGCTGTTGCCACA
GCCCAGGGTCCCATCACCTCACACTTGATCTATTGTATTACATTCCTAAC
TAGTCTTCCCCCGTTTCTAATCTGTTCTCCGATAAAAGCTGCACATCATT
TTCAGGATAATCATCAGTCGCCTGCCTAAAACTTTTCAATGTCTTCCCAT
TGTCTTTAGAATAAAGTTCAAAGTCTTCAAATGACCCCAAGCAAGATAAC
TTTTGTTTGCCCCTTTAGATCCATTTT
(SEQ. ID. NO.: 1550)

Another example is a repair vehicle designed for repair at exon 21 which consists of a left homology arm comprising the 5′ portion of exon 21 and possibly continuing into the 3′ portion of intron 20, a cDNA containing exons 22-26, and a right homology arm comprising a portion of the 5′ region of intron 21; such a repair vehicle is detailed in Table 52 below.

TABLE 52
GCCCTTTACAGAAAAAGTTTGCCAACCTATGTTGTTGTGAGGTAAAAAAA
AATCCTCTTGAAAAGGAGGCGTGAGAGTTTTACACCAAAATAGTAACATT
TTTCACTAGGTGGAAGGGTTACATTTTAAAATGTCTTTTATTTGTATTTT
TACTAATTTTTACTTTTCATTTTCTGATTTTTCTACAATGAACATACATT
GCGTAATAAATAATAGGCGGGGCACGTTGGCTCATGCCTCCCAGCACTTT
GCAAGGCTGAGGCAAGCAGATCACCTGAGGTCAGGAGTTCAAGACCAGCC
TGGCCAACATGGTGAAACTCCGTCTCTACTAAAAATACAAAAATTAGTCG
GGCATGGTGGTACGCGATTGTAGTCCCAGCTACCTAGGAGACTGAGGCAG
GAGAATTGCTTGAACTCAGGAGGTGGAGGTTGCAGTGAGCCAAGATCATG
CCATTGCACTCCAGCCTGGGTGACAAAGCAAGACTCCATCTCAAAAAAAG
AAAGAAAAGAAGAAATAATATTATTATTTGGTAGTGTTGGTAACAAATTG
CAGTATCAGCTAGTTAGAGGTGCTAACAATTAACAAAATTATAAATTTTA
GAAAATAAAATGGACAACAAGGATAAGCAATATCCTTAGATAGTAATTGA
TACTGGTATGCCATAAAGCCTTTATGTTTTTCTCTATTTTCACCACAGCT
TAGATTAACCTTTCTCAAGACAATAATTTTATTCTCAAGTGTCTAGGACT
AACCCAGCTGAATTTAATCTCTGTTTCTTTACTTGGGCAAAGGACAGTGG
GCCCCAAAGCTGGCCAGACTTCACTACTCTGGATCAATCAATGCATGGTC
TACCAAGGAGCCCTTTTCTTGGATCAAGGTgtggatctgttggcaccaat
gattattcacggcatcaagacccagggtgcccgtcagaagttctccagcc
tctacatctctcagtttatcatcatgtatagtcttgatgggaagaagtgg
cagacttatcgaggaaattccactggaaccttaatggtcttctttggcaa
tgtggattcatctgggataaaacacaatatttttaaccctccaattattg
ctcgatacatccgtttgcacccaactcattatagcattcgcagcactctt
cgcatggagttgatgggctgtgatttaaatagttgcagcatgccattggg
aatggagagtaaagcaatatcagatgcacagattactgcttcatcctact
ttaccaatatgtttgccacctggtctccttcaaaagctcgacttcacctc
caagggaggagtaatgcctggagacctcaggtgaataatccaaaagagtg
gctgcaagtggacttccagaagacaatgaaagtcacaggagtaactactc
agggagtaaaatctctgcttaccagcatgtatgtgaaggagttcctcatc
tccagcagtcaagatggccatcagtggactctcttttttcagaatggcaa
agtaaaggtttttcagggaaatcaagactccttcacacctgtggtgaact
ctctagacccaccgttactgactcgctaccttcgaattcacccccagagt
tgggtgcaccagattgccctgaggatggaggttctgggctgcgaggcaca
ggacctctactgagaattcCTAGAGCTCGCTGATCAGCCTCGACTGTGCC
TTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGG
GCAGGACAGCAAGGGGGAGGATTGGGAAGAgAATAGCAGGCATGCTGGGG
ATAGAAAATGTAATCAATGATGGGAAATGTATCACATTCAATCAATTGCA
TTACTTATTCCTCTTGCAAGCTCAAAGGATTCTATGAATATGAGAAAACT
AAAGAACAGAATGCCTTAATGATTTGTACAAAAGCAGTCATGAACAAAGA
GATATGGGGATAGAATTGAGTATATTGATATGTCCTGTTTCTGTATTTTA
GTCCTTCTACTGGGATTAGAACATCTGAATATTTTCTATAATATTGAACT
CGTCATCTCTCAAGACAGTATATGTTATTATTAGATGCTTCCAACTGCCC
ACGTGTCCTTAAGTACTCCAATCCCCTTTATTTTAACATAAAACAAATGG
TTCACAAATGCAAACCACATGTGTACTTTTACATTTTCTGTAGCCACGTT
TTCAAAAATGTGAAATTCACTTTAATAATACATTTTATTTAACTCAACAT
ATCTGAAAATACTATCATTTCAACATATGATCAATGAGGCCCCTTCAAAG
ACAGACAGATGGAAACTCTTGGGTCTCTTCCATGCCTCACAAAAGCTGAG
GGCAGCTTGGAAGTGCCTGCTCAGCCTCTCCACCTAAACATAAGGCTAGA
TGCCTTCTAGAAGCCCAAACAGGAAATGGAGAAAACATTTTGGTTTCCAT
CTTTGCAAATAGCATGTCTATTAATGCCACAGCATTGTTTTGTAGACACT
GCCAATTTTGACTCAATCTGAGCTGCTGTTCACTAATCCCTAAGTATTTT
TTGTTGGTTTGTGCTTCTGCCAAACAA
(SEQ. ID. NO.: 1551)

For repair at exons 1-13, the cDNA may contain the well-described B-domain-deleted version of exon 14 rather than the full length exon. For example, a vehicle designed for repair at exon 1 would consist of a left homology arm comprising the 5′ portion of exon 1 and possibly continuing into the promoter region of FVIII, a cDNA containing exons 2-26 or a cDNA comprising exons 2-13, the B-domain-deleted exon 14, and exons 15-26, and a right homology arm comprising a portion of the 5′ region of intron 1; such a repair vehicle for the full cDNA is detailed in Table 53 below and the B-domain-deleted alternative is detailed in Table 54 below.

TABLE 53
CTGAGAAGAGGAGTGACAGGACTCGCTTTATAGTTTTAAATTATAACTAT
AAATTATAGTTTTTAAAACAATAGTTGCCTAACCTCATGTTATATGTAAA
ACTACAGTTTTAAAAACTATAAATTCCTCATACTGGCAGCAGTGTGAGGG
GCAAGGGCAAAAGCAGAGAGACTAACAGGTTGCTGGTTACTCTTGCTAGT
GCAAGTGAATTCTAGAATCTTCGACAACATCCAGAACTTCTCTTGCTGCT
GCCACTCAGGAAGAGGGTTGGAGTAGGCTAGGAATAGGAGCACAAATTAA
AGCTCCTGTTCACTTTGACTTCTCCATCCCTCTCCTCCTTTCCTTAAAGG
TTCTGATTAAAGCAGACTTATGCCCCTACTGCTCTCAGAAGTGAATGGGT
TAAGTTTAGCAGCCTCCCTTTTGCTACTTCAGTTCTTCCTGTGGCTGCTT
CCCACTGATAAAAAGGAAGCAATCCTATCGGTTACTGCTTAGTGCTGAGC
ACATCCAGTGGGTAAAGTTCCTTAAAATGCTCTGCAAAGAAATTGGGACT
TTTCATTAAATCAGAAATTTTACTTTTTTCCCCTCCTGGGAGCTAAAGAT
ATTTTAGAGAAGAATTAACCTTTTGCTTCTCCAGTTGAACATTTGTAGCA
ATAAGTCATGCAAATAGAGCTCTCCACCTGCTTCTTTCTGTGCCTTTTGC
GATTCTGCTTTAGTGCCACCAGAAGATACTACCTGGGTGCAGTGGAACTG
TCATGGGACTATATGCAAAGTGATCTCGGTGAGCTGCCTGTGGACGCAAG
atttcctcctagagtgccaaaatcttttccattcaacacctcagtcgtgt
acaaaaagactctgtttgtagaattcacggatcaccttttcaacatcgct
aagccaaggccaccctggatgggtctgctaggtcctaccatccaggctga
ggtttatgatacagtggtcattacacttaagaacatggcttcccatcctg
tcagtcttcatgctgttggtgtatcctactggaaagcttctgagggagct
gaatatgatgatcagaccagtcaaagggagaaagaagatgataaagtctt
ccctggtggaagccatacatatgtctggcaggtcctgaaagagaatggtc
caatggcctctgacccactgtgccttacctactcatatctttctcatgtg
gacctggtaaaagacttgaattcaggcctcattggagccctactagtatg
tagagaagggagtctggccaaggaaaagacacagaccttgcacaaattta
tactactttttgctgtatttgatgaagggaaaagttggcactcagaaaca
aagaactccttgatgcaggatagggatgctgcatctgctcgggcctggcc
taaaatgcacacagtcaatggttatgtaaacaggtctctgccaggtctga
ttggatgccacaggaaatcagtctattggcatgtgattggaatgggcacc
actcctgaagtgcactcaatattcctcgaaggtcacacatttcttgtgag
gaaccatcgccaggcgtccttggaaatctcgccaataactttccttactg
ctcaaacactcttgatggaccttggacagtttctactgttttgtcatatc
tcttcccaccaacatgatggcatggaagcttatgtcaaagtagacagctg
tccagaggaaccccaactacgaatgaaaaataatgaagaagcggaagact
atgatgatgatcttactgattctgaaatggatgtggtcaggtttgatgat
gacaactctccttcctttatccaaattcgctcagttgccaagaagcatcc
taaaacttgggtacattacattgctgctgaagaggaggactgggactatg
ctcccttagtcctcgcccccgatgacagaagttataaaagtcaatatttg
aacaatggccctcagcggattggtaggaagtacaaaaaagtccgatttat
ggcatacacagatgaaacctttaagactcgtgaagctattcagcatgaat
caggaatcttgggacctttactttatggggaagttggagacacactgttg
attatatttaagaatcaagcaagcagaccatataacatctaccctcacgg
aatcactgatgtccgtcctttgtattcaaggagattaccaaaaggtgtaa
aacatttgaaggattttccaattctgccaggagaaatattcaaatataaa
tggacagtgactgtagaagatgggccaactaaatcagatcctcggtgcct
gacccgctattactctagtttcgttaatatggagagagatctagcttcag
gactcattggccctctcctcatctgctacaaagaatctgtagatcaaaga
ggaaaccagataatgtcagacaagaggaatgtcatcctgttttctgtatt
tgatgagaaccgaagctggtacctcacagagaatatacaacgctttctcc
ccaatccagctggagtgcagcttgaggatccagagttccaagcctccaac
atcatgcacagcatcaatggctatgtttttgatagtttgcagttgtcagt
ttgtttgcatgaggtggcatactggtacattctaagcattggagcacaga
ctgacttcctttctgtcttcttctctggatataccttcaaacacaaaatg
gtctatgaagacacactcaccctattcccattctcaggagaaactgtctt
catgtcgatggaaaacccaggtctatggattctggggtgccacaactcag
actttcggaacagaggcatgaccgccttactgaaggtttctagttgtgac
aagaacactggtgattattacgaggacagttatgaagatatttcagcata
cttgctgagtaaaaacaatgccattgaaccaagaagcttctcccagaatt
caagacaccctagcactaggcaaaagcaatttaatgccaccacaattcca
gaaaatgacatagagaagactgacccttggtttgcacacagaacacctat
gcctaaaatacaaaatgtctcctctagtgatttgttgatgctcttgcgac
agagtcctactccacatgggctatccttatctgatctccaagaagccaaa
tatgagactttttctgatgatccatcacctggagcaatagacagtaataa
cagcctgtctgaaatgacacacttcaggccacagctccatcacagtgggg
acatggtatttacccctgagtcaggcctccaattaagattaaatgagaaa
ctggggacaactgcagcaacagagttgaagaaacttgatttcaaagtttc
tagtacatcaaataatctgatttcaacaattccatcagacaatttggcag
caggtactgataatacaagttccttaggacccccaagtatgccagttcat
tatgatagtcaattagataccactctatttggcaaaaagtcatctcccct
tactgagtctggtggacctctgagcttgagtgaagaaaataatgattcaa
agttgttagaatcaggtttaatgaatagccaagaaagttcatggggaaaa
aatgtatcgtcaacagagagtggtaggttatttaaagggaaaagagctca
tggacctgctttgttgactaaagataatgccttattcaaagttagcatct
ctttgttaaagacaaacaaaacttccaataattcagcaactaatagaaag
actcacattgatggcccatcattattaattgagaatagtccatcagtctg
gcaaaatatattagaaagtgacactgagtttaaaaaagtgacacctttga
ttcatgacagaatgcttatggacaaaaatgctacagctttgaggctaaat
catatgtcaaataaaactacttcatcaaaaaacatggaaatggtccaaca
gaaaaaagagggccccattccaccagatgcacaaaatccagatatgtcgt
tctttaagatgctattcttgccagaatcagcaaggtggatacaaaggact
catggaaagaactctctgaactctgggcaaggccccagtccaaagcaatt
agtatccttaggaccagaaaaatctgtggaaggtcagaatttcttgtctg
agaaaaacaaagtggtagtaggaaagggtgaatttacaaaggacgtagga
ctcaaagagatggtttttccaagcagcagaaacctatttcttactaactt
ggataatttacatgaaaataatacacacaatcaagaaaaaaaaattcagg
aagaaatagaaaagaaggaaacattaatccaagagaatgtagttttgcct
cagatacatacagtgactggcactaagaatttcatgaagaaccttttctt
actgagcactaggcaaaatgtagaaggttcatatgacggggcatatgctc
cagtacttcaagattttaggtcattaaatgattcaacaaatagaacaaag
aaacacacagctcatttctcaaaaaaaggggaggaagaaaacttggaagg
cttgggaaatcaaaccaagcaaattgtagagaaatatgcatgcaccacaa
ggatatctcctaatacaagccagcagaattttgtcacgcaacgtagtaag
agagctttgaaacaattcagactcccactagaagaaacagaacttgaaaa
aaggataattgtggatgacacctcaacccagtggtccaaaaacatgaaac
atttgaccccgagcaccctcacacagatagactacaatgagaaggagaaa
ggggccattactcagtctcccttatcagattgccttacgaggagtcatag
catccctcaagcaaatagatctccattacccattgcaaaggtatcatcat
ttccatctattagacctatatatctgaccagggtcctattccaagacaac
tcttctcatcttccagcagcatcttatagaaagaaagattctggggtcca
agaaagcagtcatttcttacaaggagccaaaaaaaataacctttctttag
ccattctaaccttggagatgactggtgatcaaagagaggttggctccctg
gggacaagtgccacaaattcagtcacatacaagaaagttgagaacactgt
tctcccgaaaccagacttgcccaaaacatctggcaaagttgaattgcttc
caaaagttcacatttatcagaaggacctattccctacggaaactagcaat
gggtctcctggccatctggatctcgtggaagggagccttcttcagggaac
agagggagcgattaagtggaatgaagcaaacagacctggaaaagttccct
ttctgagagtagcaacagaaagctctgcaaagactccctccaagctattg
gatcctcttgcttgggataaccactatggtactcagataccaaaagaaga
gtggaaatcccaagagaagtcaccagaaaaaacagcttttaagaaaaagg
ataccattttgtccctgaacgcttgtgaaagcaatcatgcaatagcagca
ataaatgagggacaaaataagcccgaaatagaagtcacctgggcaaagca
aggtaggactgaaaggctgtgctctcaaaacccaccagtcttgaaacgcc
atcaacgggaaataactcgtactactcttcagtcagatcaagaggaaatt
gactatgatgataccatatcagttgaaatgaagaaggaagattttgacat
ttatgatgaggatgaaaatcagagcccccgcagctttcaaaagaaaacac
gacactattttattgctgcagtggagaggctctgggattatgggatgagt
agctccccacatgttctaagaaacagggctcagagtggcagtgtccctca
gttcaagaaagttgttttccaggaatttactgatggctcctttactcagc
ccttataccgtggagaactaaatgaacatttgggactcctggggccatat
ataagagcagaagttgaagataatatcatggtaactttcagaaatcaggc
ctctcgtccctattccttctattctagccttatttcttatgaggaagatc
agaggcaaggagcagaacctagaaaaaactttgtcaagcctaatgaaacc
aaaacttacttttggaaagtgcaacatcatatggcacccactaaagatga
gtttgactgcaaagcctgggcttatttctctgatgttgacctggaaaaag
atgtgcactcaggcctgattggaccccttctggtctgccacactaacaca
ctgaaccctgctcatgggagacaagtgacagtacaggaatttgctctgtt
tttcaccatctttgatgagaccaaaagctggtacttcactgaaaatatgg
aaagaaactgcagggctccctgcaatatccagatggaagatcccactttt
aaagagaattatcgcttccatgcaatcaatggctacataatggatacact
acctggcttagtaatggctcaggatcaaaggattcgatggtatctgctca
gcatgggcagcaatgaaaacatccattctattcatttcagtggacatgtg
ttcactgtacgaaaaaaagaggagtataaaatggcactgtacaatctcta
tccaggtgtttttgagacagtggaaatgttaccatccaaagctggaattt
ggcgggtggaatgccttattggcgagcatctacatgctgggatgagcaca
ctttttctggtgtacagcaataagtgtcagactcccctgggaatggcttc
tggacacattagagattttcagattacagcttcaggacaatatggacagt
gggccccaaagctggccagacttcattattccggatcaatcaatgcctgg
agcaccaaggagcccttttcttggatcaaggtggatctgttggcaccaat
gattattcacggcatcaagacccagggtgcccgtcagaagttctccagcc
tctacatctctcagtttatcatcatgtatagtcttgatgggaagaagtgg
cagacttatcgaggaaattccactggaaccttaatggtcttctttggcaa
tgtggattcatctgggataaaacacaatatttttaaccctccaattattg
ctcgatacatccgtttgcacccaactcattatagcattcgcagcactctt
cgcatggagttgatgggctgtgatttaaatagttgcagcatgccattggg
aatggagagtaaagcaatatcagatgcacagattactgcttcatcctact
ttaccaatatgtttgccacctggtctccttcaaaagctcgacttcacctc
caagggaggagtaatgcctggagacctcaggtgaataatccaaaagagtg
gctgcaagtggacttccagaagacaatgaaagtcacaggagtaactactc
agggagtaaaatctctgcttaccagcatgtatgtgaaggagttcctcatc
tccagcagtcaagatggccatcagtggactctcttttttcagaatggcaa
agtaaaggtttttcagggaaatcaagactccttcacacctgtggtgaact
ctctagacccaccgttactgactcgctaccttcgaattcacccccagagt
tgggtgcaccagattgccctgaggatggaggttctgggctgcgaggcaca
ggacctctactgagaattcCTAGAGCTCGCTGATCAGCCTCGACTGTGCC
TTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGG
GCAGGACAGCAAGGGGGAGGATTGGGAAGAGAATAGCAGGCATGCTGGGG
AGTAAAGGCATGTCCTGTAGGGTCTGATCGGGGCCAGGATTGTGGGGATG
TAAGTCTGCTTGGAGGAAGGTGCAGACATCGGGTTAGGATGGTTGTGATG
CTACCTGGGCCCCAAAGAAACATTTCTGGGTAAGGTGTGCACACATCTGT
GTTATTAGCAGAAATGCTAACTGCCAATTCTTTTCATAGGTCTGACCTAT
TTGTTGATATTTTGTTCTGTTTTGTCCATTGCTTCTCTTCGTCATATGCT
GCTCCTCCAGAATCTAGAGACTGGAGTAGAGGGAGGGTGAAGGGACAAAG
ACAAAACTTCCCTCTGCCTGCCCAAGCTTCCATAGAGAGAATCAAGGCAA
TGAAATCCAATCAATATCACACACAAGTTTCATGTCTGGTTCTCTTGTGT
GTACATGCAATGTGTGTTTTTATAATATCTTTTCCTACTTTGGGTGTAAG
GATAATATGAGCCTTGAGTTCAGAAGCTTTTCGTGTTTTGGGGGTTCTGG
TGCATTTAGGCAGAGTATTAAATAACTTTATCAATATTGTCTATGGTCAT
CAGTTGATTCAGATTTTTCTACCTCTTCTTCAGTAAATATTGGTATATTT
TGGTCTATACTTTCATAGAAAGCAATCTACTGTCCCTAGATTTGATAATG
TATTGGTATCAAGTTATGTAAGAGTCTCCTGTGATTTTGTTAAACTGTTC
TGTGTCTGTAGTTATATTTTCTTTTTCATTCCTTATGTTGTATATGTTCT
CTTCCTCTCTTTTAAAAATAATATTTCCAGGAGTTTTCTTGATTTTAT
TGG
(SEQ. ID. NO.: 1552)

TABLE 54
CTGAGAAGAGGAGTGACAGGACTCGCTTTATAGTTTTAAATTATAACTAT
AAATTATAGTTTTTAAAACAATAGTTGCCTAACCTCATGTTATATGTAAA
ACTACAGTTTTAAAAACTATAAATTCCTCATACTGGCAGCAGTGTGAGGG
GCAAGGGCAAAAGCAGAGAGACTAACAGGTTGCTGGTTACTCTTGCTAGT
GCAAGTGAATTCTAGAATCTTCGACAACATCCAGAACTTCTCTTGCTGCT
GCCACTCAGGAAGAGGGTTGGAGTAGGCTAGGAATAGGAGCACAAATTAA
AGCTCCTGTTCACTTTGACTTCTCCATCCCTCTCCTCCTTTCCTTAAAGG
TTCTGATTAAAGCAGACTTATGCCCCTACTGCTCTCAGAAGTGAATGGGT
TAAGTTTAGCAGCCTCCCTTTTGCTACTTCAGTTCTTCCTGTGGCTGCTT
CCCACTGATAAAAAGGAAGCAATCCTATCGGTTACTGCTTAGTGCTGAGC
ACATCCAGTGGGTAAAGTTCCTTAAAATGCTCTGCAAAGAAATTGGGACT
TTTCATTAAATCAGAAATTTTACTTTTTTCCCCTCCTGGGAGCTAAAGAT
ATTTTAGAGAAGAATTAACCTTTTGCTTCTCCAGTTGAACATTTGTAGCA
ATAAGTCATGCAAATAGAGCTCTCCACCTGCTTCTTTCTGTGCCTTTTGC
GATTCTGCTTTAGTGCCACCAGAAGATACTACCTGGGTGCAGTGGAACTG
TCATGGGACTATATGCAAAGTGATCTCGGTGAGCTGCCTGTGGACGCAAG
atttcctcctagagtgccaaaatcttttccattcaacacctcagtcgtgt
acaaaaagactctgtttgtagaattcacggatcaccttttcaacatcgct
aagccaaggccaccctggatgggtctgctaggtcctaccatccaggctga
ggtttatgatacagtggtcattacacttaagaacatggcttcccatcctg
tcagtcttcatgctgttggtgtatcctactggaaagcttctgagggagct
gaatatgatgatcagaccagtcaaagggagaaagaagatgataaagtctt
ccctggtggaagccatacatatgtctggcaggtcctgaaagagaatggtc
caatggcctctgacccactgtgccttacctactcatatctttctcatgtg
gacctggtaaaagacttgaattcaggcctcattggagccctactagtatg
tagagaagggagtctggccaaggaaaagacacagaccttgcacaaattta
tactactttttgctgtatttgatgaagggaaaagttggcactcagaaaca
aagaactccttgatgcaggatagggatgctgcatctgctcgggcctggcc
taaaatgcacacagtcaatggttatgtaaacaggtctctgccaggtctga
ttggatgccacaggaaatcagtctattggcatgtgattggaatgggcacc
actcctgaagtgcactcaatattcctcgaaggtcacacatttcttgtgag
gaaccatcgccaggcgtccttggaaatctcgccaataactttccttactg
ctcaaacactcttgatggaccttggacagtttctactgttttgtcatatc
tcttcccaccaacatgatggcatggaagcttatgtcaaagtagacagctg
tccagaggaaccccaactacgaatgaaaaataatgaagaagcggaagact
atgatgatgatcttactgattctgaaatggatgtggtcaggtttgatgat
gacaactctccttcctttatccaaattcgctcagttgccaagaagcatcc
taaaacttgggtacattacattgctgctgaagaggaggactgggactatg
ctcccttagtcctcgcccccgatgacagaagttataaaagtcaatatttg
aacaatggccctcagcggattggtaggaagtacaaaaaagtccgatttat
ggcatacacagatgaaacctttaagactcgtgaagctattcagcatgaat
caggaatcttgggacctttactttatggggaagttggagacacactgttg
attatatttaagaatcaagcaagcagaccatataacatctaccctcacgg
aatcactgatgtccgtcctttgtattcaaggagattaccaaaaggtgtaa
aacatttgaaggattttccaattctgccaggagaaatattcaaatataaa
tggacagtgactgtagaagatgggccaactaaatcagatcctcggtgcct
gacccgctattactctagtttcgttaatatggagagagatctagcttcag
gactcattggccctctcctcatctgctacaaagaatctgtagatcaaaga
ggaaaccagataatgtcagacaagaggaatgtcatcctgttttctgtatt
tgatgagaaccgaagctggtacctcacagagaatatacaacgctttctcc
ccaatccagctggagtgcagcttgaggatccagagttccaagcctccaac
atcatgcacagcatcaatggctatgtttttgatagtttgcagttgtcagt
ttgtttgcatgaggtggcatactggtacattctaagcattggagcacaga
ctgacttcctttctgtcttcttctctggatataccttcaaacacaaaatg
gtctatgaagacacactcaccctattcccattctcaggagaaactgtctt
catgtcgatggaaaacccaggtctatggattctggggtgccacaactcag
actttcggaacagaggcatgaccgccttactgaaggtttctagttgtgac
aagaacactggtgattattacgaggacagttatgaagatatttcagcata
cttgctgagtaaaaacaatgccattgaaccaagaagcttctcccagaatt
caagacaccctagccaaaacccaccagtcttgaaacgccatcaacgggaa
ataactcgtactactcttcagtcagatcaagaggaaattgactatgatga
taccatatcagttgaaatgaagaaggaagattttgacatttatgatgagg
atgaaaatcagagcccccgcagctttcaaaagaaaacacgacactatttt
attgctgcagtggagaggctctgggattatgggatgagtagctccccaca
tgttctaagaaacagggctcagagtggcagtgtccctcagttcaagaaag
ttgttttccaggaatttactgatggctcctttactcagcccttataccgt
ggagaactaaatgaacatttgggactcctggggccatatataagagcaga
agttgaagataatatcatggtaactttcagaaatcaggcctctcgtccct
attccttctattctagccttatttcttatgaggaagatcagaggcaagga
gcagaacctagaaaaaactttgtcaagcctaatgaaaccaaaacttactt
ttggaaagtgcaacatcatatggcacccactaaagatgagtttgactgca
aagcctgggcttatttctctgatgttgacctggaaaaagatgtgcactca
ggcctgattggaccccttctggtctgccacactaacacactgaaccctgc
tcatgggagacaagtgacagtacaggaatttgctctgtttttcaccatct
ttgatgagaccaaaagctggtacttcactgaaaatatggaaagaaactgc
agggctccctgcaatatccagatggaagatcccacttttaaagagaatta
tcgcttccatgcaatcaatggctacataatggatacactacctggcttag
taatggctcaggatcaaaggattcgatggtatctgctcagcatgggcagc
aatgaaaacatccattctattcatttcagtggacatgtgttcactgtacg
aaaaaaagaggagtataaaatggcactgtacaatctctatccaggtgttt
ttgagacagtggaaatgttaccatccaaagctggaatttggcgggtggaa
tgccttattggcgagcatctacatgctgggatgagcacactttttctggt
gtacagcaataagtgtcagactcccctgggaatggcttctggacacatta
gagattttcagattacagcttcaggacaatatggacagtgggccccaaag
ctggccagacttcattattccggatcaatcaatgcctggagcaccaagga
gcccttttcttggatcaaggtggatctgttggcaccaatgattattcacg
gcatcaagacccagggtgcccgtcagaagttctccagcctctacatctct
cagtttatcatcatgtatagtcttgatgggaagaagtggcagacttatcg
aggaaattccactggaaccttaatggtcttctttggcaatgtggattcat
ctgggataaaacacaatatttttaaccctccaattattgctcgatacatc
cgtttgcacccaactcattatagcattcgcagcactcttcgcatggagtt
gatgggctgtgatttaaatagttgcagcatgccattgggaatggagagta
aagcaatatcagatgcacagattactgcttcatcctactttaccaatatg
tttgccacctggtctccttcaaaagctcgacttcacctccaagggaggag
taatgcctggagacctcaggtgaataatccaaaagagtggctgcaagtgg
acttccagaagacaatgaaagtcacaggagtaactactcagggagtaaaa
tctctgcttaccagcatgtatgtgaaggagttcctcatctccagcagtca
agatggccatcagtggactctcttttttcagaatggcaaagtaaaggttt
ttcagggaaatcaagactccttcacacctgtggtgaactctctagaccca
ccgttactgactcgctaccttcgaattcacccccagagttgggtgcacca
gattgccctgaggatggaggttctgggctgcgaggcacaggacctctact
gagaattcCTAGAGCTCGCTGATCAGCCTCGACTGTGCCTTCTAGTTGCC
AGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTG
TCTGAGTAGGTGTCATTCTATTCTGGGGGGTGGGGTGGGGCAGGACAGCA
AGGGGGAGGATTGGGAAGAgAATAGCAGGCATGCTGGGGAGTAAAGGCAT
GTCCTGTAGGGTCTGATCGGGGCCAGGATTGTGGGGATGTAAGTCTGCTT
GGAGGAAGGTGCAGACATCGGGTTAGGATGGTTGTGATGCTACCTGGGCC
CCAAAGAAACATTTCTGGGTAAGGTGTGCACACATCTGTGTTATTAGCAG
AAATGCTAACTGCCAATTCTTTTCATAGGTCTGACCTATTTGTTGATATT
TTGTTCTGTTTTGTCCATTGCTTCTCTTCGTCATATGCTGCTCCTCCAGA
ATCTAGAGACTGGAGTAGAGGGAGGGTGAAGGGACAAAGACAAAACTTCC
CTCTGCCTGCCCAAGCTTCCATAGAGAGAATCAAGGCAATGAAATCCAAT
CAATATCACACACAAGTTTCATGTCTGGTTCTCTTGTGTGTACATGCAAT
GTGTGTTTTTATAATATCTTTTCCTACTTTGGGTGTAAGGATAATATGAG
CCTTGAGTTCAGAAGCTTTTCGTGTTTTGGGGGTTCTGGTGCATTTAGGC
AGAGTATTAAATAACTTTATCAATATTGTCTATGGTCATCAGTTGATTCA
GATTTTTCTACCTCTTCTTCAGTAAATATTGGTATATTTTGGTCTATACT
TTCATAGAAAGCAATCTACTGTCCCTAGATTTGATAATGTATTGGTATCA
AGTTATGTAAGAGTCTCCTGTGATTTTGTTAAACTGTTCTGTGTCTGTAG
TTATATTTTCTTTTTCATTCCTTATGTTGTATATGTTCTCTTCCTCTCTT
TTAAAAATAATATTTCCAGGAGTTTTCTTGATTTTATTGG
(SEQ. ID. NO.: 1553)

### Example 5

### Paired CRISPRs for Repair at Different Exon-Intron Junctions

Because mutations causing Hemophilia A occur throughout the FVIII gene, different repair strategies may be employed at different exon-intron junctions in order to allow the use of repair vehicles which correct a wider range of patient mutations. All gene repairs employ the methodology described above use a nuclease to induce a double-strand break near the 3′ end of an exon, thereby allowing homologous recombination to incorporate a therapeutic repair vehicle encoding the cDNA for the downstream exons of the gene into the genome in order to be operably linked to the 3′ end of that exon. In this example we describe a method using paired CRISPR nickases discussed by Ran F A, Hsu P D et al., in Cell 2013, incorporated herein by reference in order to induce double strand breaks. As well as paired CRISPRs using a Cas9 fused to the Fok1 domain (also known as RNA-guided Fok1 nucleases, “RFNs”) described by Tsai S Q et al. in Nature Biotechnology 2014, incorporated herein by reference.

To choose paired CRISPR nickase target sites in exons 1-22, several considerations were taken into account. The ˜100 bp of the 3′ end of each exon (hg19 human genome build) were searched for CRISPR/Cas9 binding sites using an online algorithm described by Hsu et al. in Nature Biotechnology 2013, incorporated herein by reference. Binding sites that function as paired nickases (using the D10A Cas9 mutant) were chosen by adding the consideration that they be orientated to create 5′ overhangs and be spaced apart within the recommended range for good activity as disclosed in Shen B, et al., Nature Methods 2014, incorporated herein by reference. Pairs of single guide RNAs (sgRNAs) were chosen based the proximity of the cleavage site to the 3′ end of the exon, and guidelines for increasing the likelihood of high on-target activity as described by Wang T et al. in Science 2014, incorporated herein by reference. Final consideration was given to choosing individual sgRNAs which each had low potential for off-target activity throughout the human genome, as assessed by the online computational tool described by Hsu et al in Nature Biotechnology 2013, incorporated herein by reference.

Sequences listed in Table 55 below contain identified binding sites for paired CRISPR nickases within exons 1-22 respectively.

TABLE 55

| FVIII Gene | Genome Editing | Genomic Target of SG/PG RNAs |
|---|---|---|
| (Region) | (Desired Activity) | (DNA Sequence) |
| Exon 1 | paired nickase (5′) | 5′-CACTAAAGCAGAATCGCAAAaGG |
| | | (SEQ. ID. NO.: 1554) |
| | paired nickase (3′) | 5′-AAGATACTACCTGGGTGCAGtGG |
| | | (SEQ. ID. NO.: 1555) |
| Exon 2 | paired nickase (5′) | 5′-AGTCTTTTTGTACACGACTGaGG |
| | | (SEQ. ID. NO.: 1556) |
| | paired nickase (3′) | 5′-TTTTCAACATCGCTAAGCCAaGG |
| | | (SEQ. ID. NO.: 1557) |
| Exon 3 | paired nickase (5′) | 5′-CAGCATGAAGACTGACAGGAtGG |
| | | (SEQ. ID. NO.: 1558) |
| | paired nickase (3′) | 5′-ATGCTGTTGGTGTATCCTACtGG |
| | | (SEQ. ID. NO.: 1559) |
| Exon 4 | paired nickase (5′) | 5′-TATGAGTAGGTAAGGCACAGtGG |
| | | (SEQ. ID. NO.: 1561) |
| | paired nickase (3′) | 5′-GACTTGAATTCAGGCCTCATtGG |
| | | (SEQ. ID. NO.: 1562) |
| Exon 5 | paired nickase (5′) | 5′-AAGTAGTATAAATTTGTGCAaGG |
| | | (SEQ. ID. NO.: 1563) |
| | paired nickase (3′) | 5′-CTTTTTGCTGTATTTGATGAaGG |
| | | (SEQ. ID. NO.: 1564) |
| Exon 6 | paired nickase (5′) | 5′-GACTGTGTGCATTTTAGGCCaGG |
| | | (SEQ. ID. NO.: 1565) |
| | paired nickase (3′) | 5′-CAGTCAATGGTTATGTAAACaGG |
| | | (SEQ. ID. NO.: 1566) |
| Exon 7 | paired nickase (5′) | 5′-GCGAGATTTCCAAGGACGCCtGG |
| | | (SEQ. ID. NO.: 1567) |
| | paired nickase (3′) | 5′-CAAACACTCTTGATGGACCTtGG |
| | | (SEQ. ID. NO.: 1568) |
| Exon 8 | paired nickase (5′) | 5′-TCTTGGCAACTGAGCGAATTtGG |
| | | (SEQ. ID. NO.: 1569) |
| | paired nickase (3′) | 5′-ACATTACATTGCTGCTGAAGaGG |
| | | (SEQ. ID. NO.: 1570) |
| Exon 9 | paired nickase (5′) | 5′-AATAGCTTCACGAGTCTTAAaGG |
| | | (SEQ. ID. NO.: 1571) |
| | paired nickase (3′) | 5′-GAAGCTATTCAGCATGAATCaGG |
| | | (SEQ. ID. NO.: 1572) |
| Exon 10 | paired nickase (5′) | 5′-GGACATCAGTGATTCCGTGAgGG |
| | | (SEQ. ID. NO.: 1573) |
| | paired nickase (3′) | 5′-ATGTCCGTCCTTTGTATTCAaGG |
| | | (SEQ. ID. NO.: 1574) |
| Exon 11 | paired nickase (5′) | 5′-AACGAAACTAGAGTAATAGCgGG |
| | | (SEQ. ID. NO.: 1575) |
| | paired nickase (3′) | 5′-GATCTAGCTTCAGGACTCATtGG |
| | | (SEQ. ID. NO.: 1576) |
| Exon 12 | paired nickase (5′) | 5′-AGCGTTGTATATTCTCTGTGaGG |
| | | (SEQ. ID. NO.: 1577) |
| | paired nickase (3′) | 5′-CGCTTTCTCCCCAATCCAGCtGG |
| | | (SEQ. ID. NO.: 1578) |
| Exon 13 | paired nickase (5′) | 5′-ATAGACCATTTTGTGTTTGAaGG |
| | | (SEQ. ID. NO.: 1579) |
| | paired nickase (3′) | 5′-AGAAACTGTCTTCATGTCGAtGG |
| | | (SEQ. ID. NO.: 1580) |
| Exon 14 | paired nickase (5′) | 5′-TTTTCTTTTGAAAGCTGCGGgGG |
| | | (SEQ. ID. NO.: 1581) |
| | paired nickase (3′) | 5′-ACACTATTTTATTGCTGCAGtGG |
| | | (SEQ. ID. NO.: 1582) |
| Exon 15 | paired nickase (5′) | 5′-ACGGTATAAGGGCTGAGTAAaGG |
| | | (SEQ. ID. NO.: 1583) |
| | paired nickase (3′) | 5′-AAATGAACATTTGGGACTCCtGG |
| | | (SEQ. ID. NO.: 1584) |
| Exon 16 | paired nickase (5′) | 5′-CAGTCAAACTCATCTTTAGTgGG |
| | | (SEQ. ID. NO.: 1585) |
| | paired nickase (3′) | 5′-ATGAGTTTGACTGCAAAGCCtGG |
| | | (SEQ. ID. NO.: 1586) |
| Exon 17 | paired nickase (5′) | 5′-TTCAGTGAAGTACCAGCTTTtGG |
| | | (SEQ. ID. NO.: 1587) |
| | paired nickase (3′) | 5′-GGCTCCCTGCAATATCCAGAtGG |
| | | (SEQ. ID. NO.: 1588) |
| Exon 18 | paired nickase (5′) | 5′-GTCCACTGAAATGAATAGAAtGG |
| | | (SEQ. ID. NO.: 1589) |
| | paired nickase (3′) | 5′-GTTCACTGTACGAAAAAAAGaGG |
| | | (SEQ. ID. NO.: 1590) |
| Exon 19 | paired nickase (5′) | 5′-CGCCAAATTCCAGCTTTGGAtGG |
| | | (SEQ. ID. NO.: 1591) |
| | paired nickase (3′) | 5′-ATTGGCGAGCATCTACATGCtGG |
| | | (SEQ. ID. NO.: 1592) |
| Exon 20 | paired nickase (5′) | 5′-TGTCCAGAAGCCATTCCCAGgGG |
| | | (SEQ. ID. NO.: 1593) |
| | paired nickase (3′) | 5′-GATTTTCAGATTACAGCTTCaGG |
| | | (SEQ. ID. NO.: 1594) |
| Exon 21 | paired nickase (5′) | 5′-TGATCCGGAATAATGAAGTCtGG |
| | | (SEQ. ID. NO.: 1595) |
| | paired nickase (3′) | 5′-AATCAATGCCTGGAGCACCAaGG |
| | | (SEQ. ID. NO.: 1596) |
| Exon 22 | paired nickase (5′) | 5′-AGATAAACTGAGAGAGTAGAGG |
| | | (SEQ. ID. NO.: 1597) |
| | paired nickase (3′) | 5′-AAGAAGTGGCAGACTTATCGaGG |
| | | (SEQ. ID. NO.: 1598) |

The spacing requirements between the sgRNAs differ between paired CRISPR nickases and RFNs, but the other considerations regarding on-target and off-target activity remain the same and were taken into account when searching for RFN target sites in exons 1-22.

The ˜140 bp of the 3′ end of each exon (hg19 human genome build) was searched for RFN binding sites matching the spacing distances using the ZiFiT targeter disclosed in Tsai S Q et al. Nature Biotech 2014, incorporated herein by reference. For some exons, there was no targetable sequence matching the PAM orientation and spacing requirements of the RFN system. Sequences in table 56 below contain identified binding sites for RFNs within exons 1-22 respectively.

TABLE 56

| | Genome | |
|---|---|---|
| FVIII Gene | Editing | Genomic Target of RFN |
| (Region) | Position | (DNA Sequence) |
| Exon 1 | 5′ Half-Site | 5′-GCACCCAGGTAGTATCTTCtGG |
| | | (SEQ. ID. NO.: 1599) |
| | 3′ Half-Site | 5′-ACTATATGCAAAGTGATCTcGG |
| | | (SEQ. ID. NO.: 1600) |
| Exon 2 | 5′ Half-Site | No Compatible Sites |
| | 3′ Half-Site | No Compatible Sites |
| Exon 3 | 5′ Half-Site | No Compatible Sites |
| | 3′ Half-Site | No Compatible Sites |
| Exon 4 | 5′ Half-Site | 5′-ACATGAGAAAGATATGAGTaGG |
| | | (SEQ. ID. NO.: 1601) |
| | 3′ Half-Site | 5′-ACTTGAATTCAGGCCTCATtGG |
| | | (SEQ. ID. NO.: 1602) |
| Exon 5 | 5′ Half-Site | 5′-AAGGTCTGTGTCTTTTCCTtGG |
| | | (SEQ. ID. NO.: 1603) |
| | 3′ Half-Site | 5′-TTTTTGCTGTATTTGATGAaGG |
| | | (SEQ. ID. NO.: 1604) |
| Exon 6 | 5′ Half-Site | 5′-TTTTCCCTGATGAGAGAGAaGG |
| | | (SEQ. ID. NO.: 1605) |
| | 3′ Half-Site | 5′-ACAAAGAACTCCTTGATGCaGG |
| | | (SEQ. ID. NO.: 1606) |
| Exon 7 | 5′ Half-Site | 5′-GTTATTGGCGAGATTTCCAaGG |
| | | (SEQ. ID. NO.: 1607) |
| | 3′ Half-Site | 5′-AAACACTCTTGATGGACCTtGG |
| | | (SEQ. ID. NO.: 1608) |
| Exon 8 | 5′ Half-Site | No Compatible Sites |
| | 3′ Half-Site | No Compatible Sites |
| Exon 9 | 5′ Half-Site | 5′-ATAGCTTCACGAGTCTTAAaGG |
| | | (SEQ. ID. NO.: 1609) |
| | 3′ Half-Site | 5′-TCTTGGGACCTTTACTTTAtGG |
| | | (SEQ. ID. NO.: 1610) |
| Exon 10 | 5′ Half-Site | No Compatible Sites |
| | 3′ Half-Site | No Compatible Sites |
| Exon 11 | 5′ Half-Site | 5′-ACGAAACTAGAGTAATAGCgGG |
| | | (SEQ. ID. NO.: 1611) |
| | 3′ Half-Site | 5′-ATCTAGCTTCAGGACTCATtGG |
| | | (SEQ. ID. NO.: 1612) |
| Exon 12 | 5′ Half-Site | No Compatible Sites |
| | 3′ Half-Site | No Compatible Sites |
| Exon 13 | 5′ Half-Site | No Compatible Sites |
| | 3′ Half-Site | No Compatible Sites |
| Exon 14 | 5′ Half-Site | 5′-TGTTTTCTTTTGAAAGCTGcGG |
| | | (SEQ. ID. NO.: 1613) |
| | 3′ Half-Site | 5′-GCTGCAGTGGAGAGGCTCTgGG |
| | | (SEQ. ID. NO.: 1614) |
| Exon 15 | 5′ Half-Site | No Compatible Sites |
| | 3′ Half-Site | No Compatible Sites |
| Exon 16 | 5′ Half-Site | 5′-AGTCAAACTCATCTTTAGTgGG |
| | | (SEQ. ID. NO.: 1615) |
| | 3′ Half-Site | 5′-TATTTCTCTGATGTTGACCtGG |
| | | (SEQ. ID. NO.: 1616) |
| Exon 17 | 5′ Half-Site | 5′-CTTTTGGTCTCATCAAAGAtGG |
| | | (SEQ. ID. NO.: 1617) |
| | 3′ Half-Site | 5′-AATATGGAAAGAAACTGCAgGG |
| | | (SEQ. ID. NO.: 1618) |
| Exon 18 | 5′ Half-Site | No Compatible Sites |
| | 3′ Half-Site | No Compatible Sites |
| Exon 19 | 5′ Half-Site | 5′-GCCAAATTCCAGCTTTGGAtGG |
| | | (SEQ. ID. NO.: 1619) |
| | 3′ Half-Site | 5′-TTGGCGAGCATCTACATGCtGG |
| | | (SEQ. ID. NO.: 1620) |
| Exon 20 | 5′ Half-Site | 5′-TGTCCAGAAGCCATTCCCAgGG |
| | | (SEQ. ID. NO.: 1621) |
| | 3′ Half-Site | 5′-TTACAGCTTCAGGACAATAtGG |
| | | (SEQ. ID. NO.: 1622) |
| Exon 21 | 5′ Half-Site | 5′-GATCCGGAATAATGAAGTCtGG |
| | | (SEQ. ID. NO.: 1623) |
| | 3′ Half-Site | 5′-CACCAAGGAGCCCTTTTCTtGG |
| | | (SEQ. ID. NO.: 1624) |
| Exon 22 | 5′ Half-Site | 5′-AGGCTGGAGAACTTCTGACgGG |
| | | (SEQ. ID. NO.: 1625) |
| | 3′ Half-Site | 5′-TCATCATGTATAGTCTTGAtGG |
| | | (SEQ. ID. NO.: 1626) |

### Example 6

### Additional Methods and Examples for FVIII Gene Repair in Cells

Purifying CRISPR/Cas9 Plasmids and Repair Plasmids (DNA-RS)

A protocol for preparing CRISPR/Cas9 plasmids (DNA-SE) and repair plasmids (DNA-RS) using endotoxin-free methods is described in the following example. For this protocol, a Qiagen EndoFree Plasmid Maxi Kit is used. The Qiagen EndoFree Plasmid Maxi Kit and its contents are stored at room temperature. Once RNAse and LyseBlue are added to Buffer P1 from the kit, this buffer is stored at 4° C. The kit also requires 100% ethanol and isopropanol (2-propanol).

According to this protocol, at Day 1, a 1 mL seed culture of Escherichia coli (E. coli) in Luria Broth (LB) and appropriate antibiotic is prepared and placed on a shaker at 37° C. Whether an antibiotic is appropriate is dependent on the antibiotic resistance gene that is present in the plasmid that is being prepared and purified. For example, such an antibiotic may be ampicillin, kanamycin, or other antibiotics. Approximately 5 hours from when the seed culture is prepared, the seed culture is then used to inoculate a 100 mL LB culture and the suspension is left shaking overnight (or for at least about 8 hours) at 37° C.

At day 2, the 100 mL culture is transferred into 2×50 mL conical tubes and spun for 10 min at 4000 g; the supernatant is dumped out. The resulting cell pellet can be stored at −20° C. for an indefinite period of time. During the spin, Buffer P3 is placed on ice. Following the spin and removal of the supernatant, 10 mL of Buffer P1 are added to the first 50 mL tube of each prep. This solution is then vortexed to resuspend the pelleted cells. The resuspended mixture is poured a second tube and vortexed to resuspend. Next, 10 mL of Buffer P2 are added and the suspension is inverted 6× to mix (until mixture is homogenously blue). This suspension is incubated for 3 min at room temperature. Next, 10 mL of Buffer P3 is added to each tube, and each tube is inverted ˜10×.

Next, the suspensions are centrifuged for 5 minutes at 4000 g. During the spin, a fresh 50 mL tube is labeled for each abovementioned prep. A cap is screwed onto a filter cartridge and placed in the fresh 50 mL tube. After the spin, a p1000 pipette tip is used to hold back debris while pouring the liquid from the spun suspension into the cartridge. The suspension is then incubated for 10 minutes at room temperature in the cartridge. Next, the cartridge is uncapped and a plunger is used to push the liquid into the 50 mL tube; the cartridge/plunger is trashed following this step. Next, 2.5 mL of Buffer ER is added to each tube, and each tube is inverted 10× until the liquid becomes cloudy. The suspension is incubated on ice for 30 minutes. During the incubation, Qiagen-Tip-500 tubes are labeled and placed in a clamp draining into a 1000 mL beaker. 10 mL of Buffer QBT is added to Qiagen-Tips to equilibrate the system. After the 30 minute incubation, the prep mixture is poured into the respectively labeled Qiagen-tips. Buffer QC is used to wash the tips.

Next, the Qiagen-Tip-Tubes are placed into 50 mL tubes capable of withstanding spins @ 15000 g. 15 mL of Buffer QN is added to the Qiagen-Tip-Tubes and centrifuged at 4° C. to allow the DNA to elute from the Qiagen-Tip-Tubes as the buffer QN drains through. The eluted DNA can be stored at 4° C. overnight.

Next, 10.5 mL of Isopropanol is added and the suspension is inverted 10× to mix. The samples are then centrifuged at 15000 g for 10 min at 4° C.; The DNA will be present as a pellet. After the supernatant is dumped out, 5 mL of 70% Ethanol (EtOH) is added to the pelleted DNA. The samples are centrifuged at 15000 g for 10 min at 4° C. Then, the supernatant is decanted using a p1000 pipette. The tube is then left to air-dry for 10 min. Next, 150 uL of Tris EDTA buffer (TE) is added. Isolated plasmid concentration is then determined.

In the example described, four CRISPR plasmids were prepared using these methods, each in triplicate, in addition to the preparation of a pGFP plasmid in duplicate. These procedures yielded the results shown in Table 57:

TABLE 57
Concentration of isolated CRISPR and pGFP plasmid preps

| Sample # | [DNA] | Unit | A260 | A280 | 260/280 | 260/230 |
|---|---|---|---|---|---|---|
| pH0007-1 | 273.7 | ng/μl | 5.475 | 2.881 | 1.9 | 2.28 |
| pH0007-2 | 262.8 | ng/μl | 5.257 | 2.771 | 1.9 | 2.26 |
| pH0007-3 | 350 | ng/μl | 7 | 3.688 | 1.9 | 2.27 |
| pH0009-1 | 328.1 | ng/μl | 6.561 | 3.462 | 1.9 | 2.26 |
| pH0009-2 | 345 | ng/μl | 6.901 | 3.637 | 1.9 | 2.27 |
| pH0009-3 | 274.9 | ng/μl | 5.499 | 2.909 | 1.89 | 2.19 |
| pH0011-1 | 320.4 | ng/μl | 6.408 | 3.378 | 1.9 | 2.26 |
| pH0011-2 | 295.2 | ng/μl | 5.905 | 3.122 | 1.89 | 2.25 |
| pH0011-3 | 328 | ng/μl | 6.559 | 3.469 | 1.89 | 2.27 |
| pH0013-1 | 323.3 | ng/μl | 6.466 | 3.388 | 1.91 | 2.27 |
| pH0013-2 | 311 | ng/μl | 6.22 | 3.274 | 1.9 | 2.22 |
| pH0013-3 | 306.7 | ng/μl | 6.135 | 3.23 | 1.9 | 2.28 |
| pGFP-1 | 273.8 | ng/μl | 5.477 | 2.877 | 1.9 | 2.28 |
| pGFP-2 | 341.9 | ng/μl | 6.838 | 3.623 | 1.89 | 2.2 |

Nucleofection Conditions and Methods

A protocol for nucleofection is described in the following example. The protocol described uses 20 uL Nucleovette Strips (Lonza). The number of cells recommended for this technique is 200,000 cells per condition or sample. The maximum mass of DNA used in this technique is ˜1000 ng. It is recommended that a significantly greater amount of repair plasmid be used compared to the CRISPR/Cas9 plasmid as this minimizes the likelihood of off-target effects while maximizing the likelihood of homologous recombination. Typically a ratio of 4:1 repair plasmid:CRISPR/Cas9 plasmid is used.

To facilitate all of the analyses involved with these methods, the following reaction conditions are recommended. First, for the “experimental” condition, 200 ng of CRISPR/Cas9 plasmid (DNA-SE), 800 ng of repair plasmid (DNA-RS), and 40 ng of MaxGFP plasmid are used for transfection. Second, for the “no repair plasmid” control condition (also suitable for T7 Endonuclease (T7E1) analysis), 200 ng of CRISPR/Cas9 plasmid (DNA-SE), 800 ng of stuffer plasmid (pUC19), and 40 ng of MaxGFP plasmid are used for transfection. Third, for the “no CRISPR plasmid” condition, 200 ng of stuffer plasmid (pUC19), 800 ng of repair plasmid (DNA-RS), and 40 ng of MaxGFP plasmid are used for transfection. Fourth, for the “GFP alone” condition, 1000 ng of stuffer plasmid (pUC19) and 40 ng of MaxGFP plasmid are used for transfection.

For the method, first, 500 ul of media is added to the required number of wells in a 24 well plate. This is pre-warmed in an incubator set to 37° C., 5% CO₂. Next, 1 μg of total DNA in minimum of 2 μl is used. Next, the DNA is setup into a new strip tubes.

Next, the cells are prepared for nucleofection. 200,000 cells per nucleofection reaction are preferred. 1.2× of master mix of cells is prepared to account for cell loss during media aspiration and pipetting errors. Next, the cells are pelleted by centrifugation at 300×g for 5 minutes. Next, if the Nucleocuvette strip kit is used, a nucleofection solution provided with kit is used. All of the supplement is added to Nucleofector solution; 20 μl of the combined buffer is required per nucleofection.

Next, during the spin a plate is labeled. The media is then aspirated from the cells and the cells are resuspended in 1.1× Nucleofector buffer (22 ul per nucleofection—352 uL/16 nucleofections, 374 uL/17 reactions). Next, 20 ul of cell suspension (approx. 200,000 cells) is aliquoted to DNA solutions. Next, the Nucleocuvette strip is placed in the 4D Nucleofector X-module and the corresponding program is selected. Next, the cuvette is allowed to incubate for 10 minute following shocking of the cells. Next, 50 ul of media from 24 well plate is added to the Nucleocuvette. All of the cell/media mix from the cuvette is then added to the 24 well plate and incubated at 37° C. for 72 hours.

Protocol for QuickExtract Method for gDNA Extraction

A protocol for gDNA extraction is described in the following example. This method allows for the extraction of genomic DNA (gDNA) from live cell samples using QuickExtract™ DNA Extraction Solution (Epicentre). First, about 100,000 cells are pelleted by centrifugation. Then 80 μL of the QuickExtract solution is added to the cells and the suspension is transferred to a thermocycler tube. The suspension is then vortexed. The suspension is then run in a thermocycler for 15 min at 65° C. and 8 min at 98° C.; The solution can then be stored at −20° C. and freeze/thawed for at least 40 times. Next, ˜1 μL of this solution is used as the genomic DNA template per 50 μL of PCR reaction.

Protocol for T7E1 Assay

A protocol for a T7E1 assay is described in the following example. According to the protocol, 35 cycles of PCR is used on isolated gDNA to amplify a target locus at the exon22/intron22 boundary using T7E1 primers that flank this boundary. The forward primer has a sequence of 5′-GGTAATGATGGACACACCTGTAGC-3′ (SEQ. ID. NO.: 1627) and the reverse primer has a sequence of 5′-GGTTTTGCCCCCTAAACTTGTC-3′ (SEQ. ID. NO.: 1628) and PCR with these primers results in amplicons of 623 nucleotides in length. The PCR amplicons are then purified using Wizard SV Gel and PCR Clean-up System (Promega) according to manufacturer's instructions.

Next, 200 ng of purified PCR product is placed in 1×NEBuffer 2 (New England Biolabs, Buffer 2, a component of the T7 Endonuclease 1 kit that is available from New England Biolabs) in a total volume of 18 uL. Next, the suspension is vortexed and centrifuged. Next, the samples are placed in a thermocycler programmed with the following protocol: A) 95° C. for 5 min; B) 95-25° C. in −1° C./s steps; C) hold at 4° C.

10 units of T7 Endonuclease 1 is are added to the hybridized PCR products in a 2 uL volume of 1×NEBuffer 2 (for a final reaction volume of 20 uL). Note that for each sample, a side-by-side negative control (no T7E1 enzyme control) is prepared, wherein 2 uL volume of 1×NEBuffer is used in the absence of the enzyme. Next, the suspensions are vortexed and centrifuged. The suspensions are then incubated at 37° C. for 30 minutes. Following incubation, the samples are placed on ice and stop solution is added to them. The stop solution is prepared by adding 2.45 uL 0.5M EDTA to 4.49 uL 6× loading dye for each reaction (6.94 uL volume per reaction, resulting in a final concentration of 45 mM EDTA and 1× loading dye).

Next, the samples by agarose gel electrophoresis. The gel image can be quantified with ImageJ using the following procedure: 1) the image is inverted; 2) the background is subtracted (set to 30 pixels, check light background box); 3) rectangles are drawn about the middle of a gel lane, avoid the “smiling” on the end of the gel lanes; 4) in the analyze gel lane, “select first lane” option is selected; 5) subsequent lanes are selected; 6) Quantitative analysis is performed (fraction cleaved=area cleaved/area of all); 7) Calculate % gene modification with the following equation:

% gene modification=100×(1−(1−fraction cleaved)^{1/2})

### Protocol for Restriction Fragment Length Polymorphism (RFLP) Assay

A protocol for a RFLP assay is described in the following example. According to the protocol, 35 cycles of PCR is used on gDNA to amplify a target locus at the exon22/intron22 boundary using RFLP primers that flank this boundary. The forward primer has a sequence of 5′-GTTAGGTGACTCAAATGGGTTCAC-3′ (SEQ. ID. NO.: 1629) and the reverse primer has a sequence of 5′-GAACAAGAAGCAGGGTAGAGAAGC-3′ (SEQ. ID. NO.: 1630) and PCR with these primers results in amplicons of 1667 nucleotides in length. The PCR amplicons are purified using Wizard SV Gel and PCR Clean-up System (Promega) according to manufacturer's instructions.

Next, a mixture with 20 μL reaction with 0.5 μL (5 U) of restriction enzyme, 2 uL reaction buffer (provided in the enzyme kit), and then 17.5 μL of the cleaned PCR reaction is prepared. This mixture is then incubated at 37° C. for 1 hour. Next, the samples are analyzed the samples by agarose gel electrophoresis. The gel image is then quantified with ImageJ using the following procedure: 1) the image is inverted; 2) the background is subtracted (set to 30 pixels, check light background box); 3) rectangles are drawn about the middle of a gel lane, avoid the “smiling” on the end of the gel lanes; 4) in the analyze gel lane, “select first lane” option is selected; 5) subsequent lanes are selected; 6) Quantitative analysis is performed (fraction cleaved=area cleaved/area of all); 7) Calculation of % homologous recombination with the following equation:

% HR=(cut band)/(cut band+uncut band)

### Protocol for PCR Amplification at Gene Repair Site

A protocol for PCR amplification at a gene repair site is described in the following example. According to the protocol, as a first qualitative approach, PCR with RFLP primers is performed to examine the presence of a band distinct from the main band. The primers and procedures in this method are the same as those described above in the section entitled “Protocol for Restriction Fragment Length Polymorphism (RFLP) Assay.” The main (uncut) band is expected to be about 1.7 kb in size, wherease the cut band is expected to be about 1.0 kb in size.

In a second qualitative approach according to this protocol, a reverse RFLP primer (with sequence 5′-GAACAAGAAGCAGGGTAGAGAAGC-3′) (SEQ. ID. NO.: 1631) that anneals within exon 22 is paired with a primer that anneals within the gene repair site (with sequence 5′-AAGATGGCCATCAGTGGACTCTC-3′) (SEQ. ID. NO.: 1632) is used. This PCR will only form a product of about 1.3 kb in size if there is successful gene correction.

Following analysis of the results from the PCR analyses described above, clonal colonies are grown out. This is done either through limiting dilution of the cells or by FACS sorting of single cells into a 96-well plate. With either method, initially plate 1 cell into ˜50 uL of media. Then after 1 week add ˜150 uL of new media to the wells. After about a second week, or when there are >10,000 cells, use the QuickExtract protocol to isolate gDNA. Proceed to perform the same two PCRs described above—the 2nd PCR method will demonstrate if there is at least monoallelic gene correction, the first PCR (with the RFLP primers) will demonstrate if there is biallelic correction (because all of the PCR product will be at a different band size) and also serve as a positive control to determine that the QuickExtract for that sample is a viable PCR template.

### Protocol for Gene Repair in FVIII

A protocol for gene repair in FVIII is described in the following example. According to the protocol, seed cell cultures were prepared 2 days before transfection, with a final target density of 800,000 cells/mL on the day of transfection. Next, CRISPR/Cas9 plasmids (DNA-SE) and repair plasmids (DNA-RS) were prepared as indicated above in the protocol for endotoxin-free plasmid maxiprep. Next, the transfection setup details for nucleofection, such as plasmid concentrations and volumes, cell concentrations and volumes were determined as discussed above in the protocol for nucleofection conditions and methods. Next, nucleofection was performed, followed by culturing the cells for 72 hours as discussed above in the protocol for nucleofection conditions and methods.

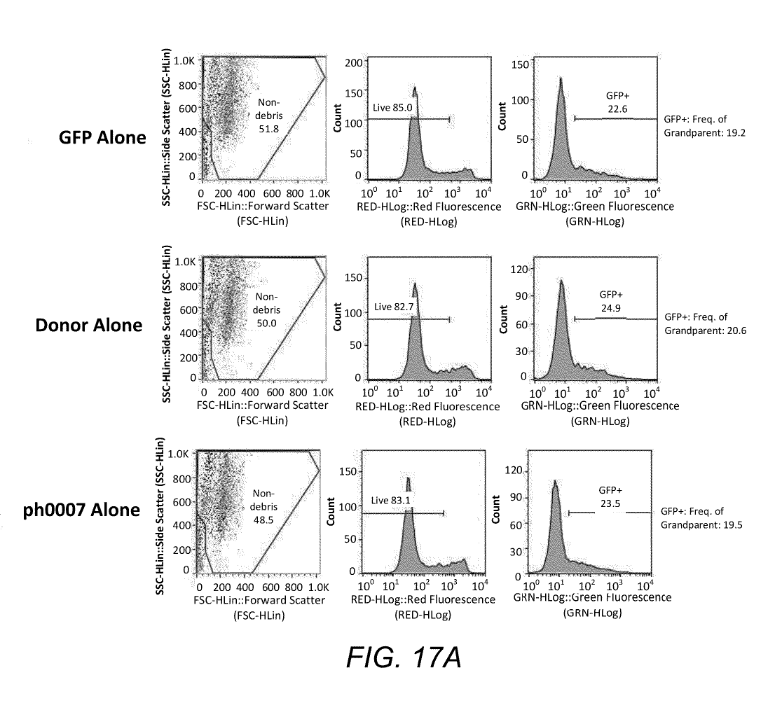

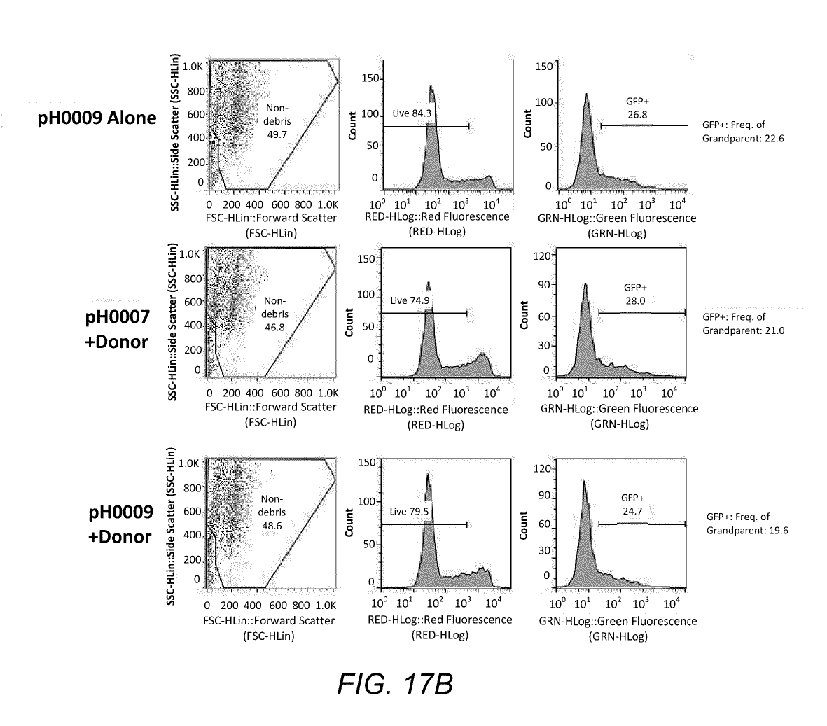

Flow cytometry analysis was used to determine % viability and % GFP+ cells in each sample on one quarter of the cells collected from the nucleofection step. Results using the CRISPR/Cas9 plasmids pH0007 and pH0009 as well as a repair plasmid (labeled “donor”) are shown in FIGS. 17A-B. In FIGS. 17A-B, the left-most graph for each sample displays the FSC/SSC characteristics of the population and allows for gating on non-debris in the sample; the center graph for each sample displays in histogram format the distribution of live cells in the sample as evidenced by inclusion of propidium iodide which enters only dead cells and yields a red fluorescence; and the right-most graph for each sample displays in histogram format the distribution of cells that have been successfully transfected as evidenced by green fluorescence that is due to the presence of GFP. As can be seen from the results, the percentages for each parameter are similar across all samples, with a range for each parameter of 46.8-51.8% (non-debris), 74.9-85.0% (Live), and 22.6-26.8% (GFP+). Thus the rates of successful transfection do not differ substantially as a function of the plasmid used.

In this example, gDNA from one quarter of the cells from the nucleofection event was isolated following the protocol for gDNA extraction described above. The gDNA was then analyzed using the following protocols described above: 1) protocol for T7 E1 assay; 2) protocol for RFLP assay; and 3) protocol for PCR amplification at gene repair site.

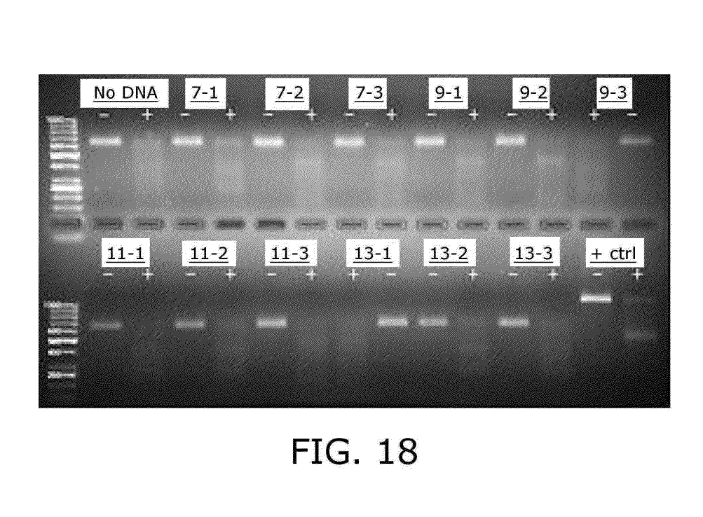

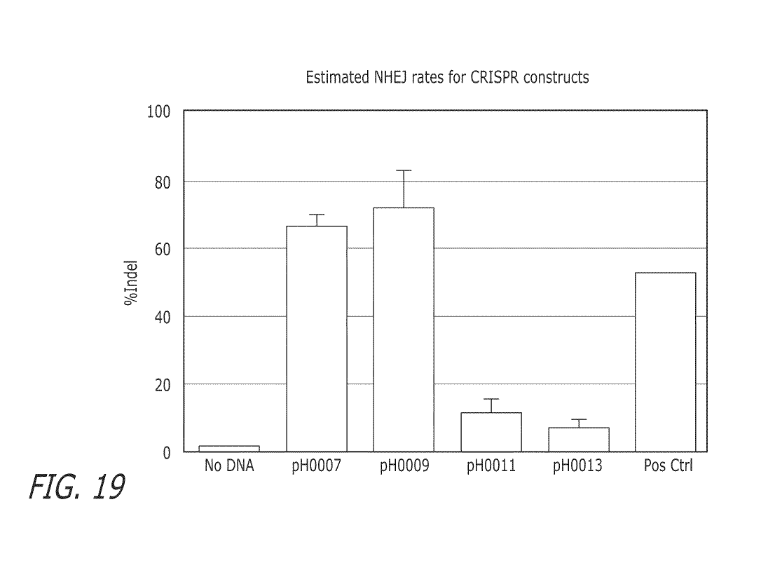

Results from the analysis following the T7E1 assay are shown in FIG. 18 and in FIG. 19. FIG. 18 and FIG. 19 show results from using CRISPR/Cas9 plasmids pH0007, pH0009, pH0011, and pH0013. FIG. 18 shows an image from an agarose gel electrophoresis assay. In FIG. 18 the samples names are abbreviated such that the three pH0007 are listed as 7-1, 7-2, and 7-3, and this pattern is continued for pH0009, pH0011, and pH0013. A negative control (No DNA) and positive control (+ ctrl) in the analysis. For each sample there are two lanes: one labeled at the top of the lane with a “+” which sample contained the T7E1 enzyme, and a second labeled with a “−” which sample contained no T7E1 enzyme. In the absence of T7E1, no nuclease activity is present and there is a single band present in the lane. In the presence of T7E1, some cleavage occurs resulting in a second smaller band that appears. This qualitative data demonstrates that pH0007 and pH0009 yield the better result than pH0011 and pH0013 as there is a greater relative abundance of the smaller band in those samples. This is quantified in FIG. 19. FIG. 19 shows the calculated values for percent gene modification by NHEJ (non-homologous end joining), demonstrating that pH0007 and pH0009 cause indel formation at the target site at a rate of 66% and 72% respectively, and that both of these yield statistically significantly superior rates of indel formation compared to pH0011 and pH0013. This statistical significance is evidenced by the error bars which display the standard error of the mean for each sample.

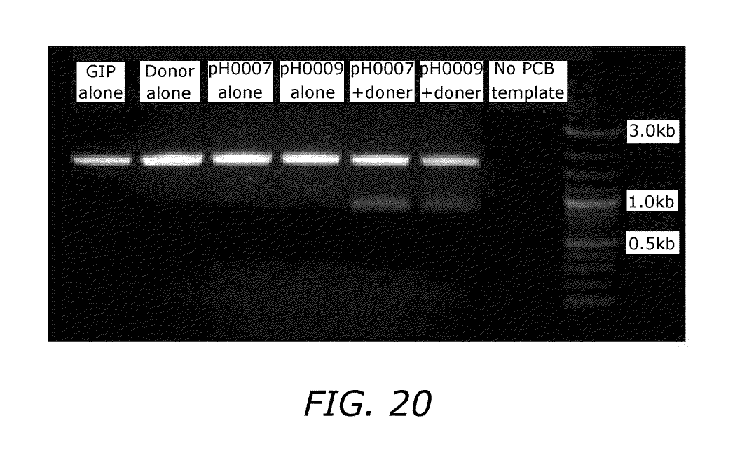

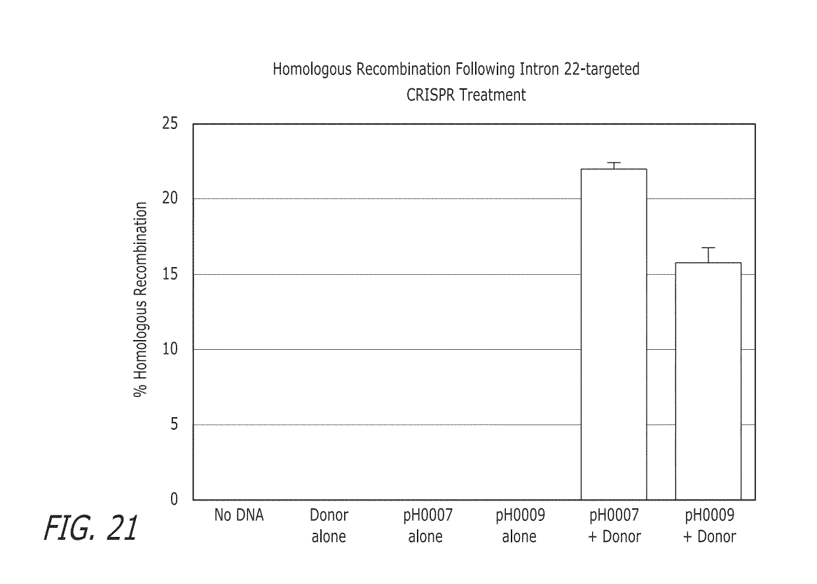

Results from the analysis following the RFLP assay are shown in FIG. 20 and FIG. 21. FIG. 20 and FIG. 21 show results from using CRISPR/Cas9 plasmids pH0007, pH0009, as well as a repair plasmid (labeled “Donor”). FIG. 20 shows an image from an agarose gel electrophoresis assay. In FIG. 20 displays the results of a simple and standard RFLP assay demonstrating that only in those samples that contain the donor plasmid along with either pH0007 or pH0009 is there a smaller band which indicates restriction digestion, the presence of the restriction site and thus successful recombination in those samples. In the other control samples, no such smaller band is seen. FIG. 21 shows the calculated values for percent gene modification by following Intron 22-targeted CRISPR treatment. As can be seen from the data, homologous recombination occurs only in those samples that were transfected with the donor plasmid and pH0007 or pH0009 at a rate of 22% and 16% respectively. The control samples that were transfected with only donor plasmid, only pH0007, only pH0009, or none of the three show a rate of homologous recombination of 0% for each sample.

Next, cells were cloned out either by limiting serial dilution or single-cell FACS. Clones were cultured until the clonal colonies reach cell numbers of ?20,000. gDNA from ?10,000 cells of each clonal culture using was then extracted. PCR was used to amplify across the repair site, using as template each of the extracted gDNA samples from the clonal cultures. Next, sanger sequencing methods were used to sequence the repair-site PCR amplicons. Next, the DNA sequence immediately upstream (about 25 bases), immediately downstream (about 25 bases), and across the repair was analyzed.

Clones not displaying the desired or expected integration events were eliminated. Next, it was determined if any DNA sequence modifications have been made at sites in the genome that have been predicted by algorithm to be the top 20 potential off-target sites in the genome. Clonal cultures for which DNA sequence modifications have been made at off-target sites in the genome we eliminated.

Remaining clones were cultured out until clonal colonies reach cell numbers of ≧1×10⁶. mRNA was extracted from ≧100,000 cells of each clonal culture; mRNA was also extracted from ≧100,000 cells of the parent culture (in which no gene repair has been performed).

Quantitative reverse-transcription PCR (qRT-PCR) primers were designed for the detection of: a) Transcription of the F8 gene, targeting an exonic site 5′ of the gene repair site; b) Transcription of the F8 gene, targeting an exonic site 3′ of the gene repair site; c) Transcription of the F8 gene, targeting a sequence that is unique to the gene repair site itself, that furthermore overlaps the junction of (i) the gene repair site and (ii) an endogenous, non-repaired exonic site 5′ of the gene repair site. This amplified product should only be detected in cells that have been correctly repaired; and d) Transcription of house-keeping genes that can be used for normalization of F8 gene transcription, including at least the genes for beta-actin (ACTB), gamma-tubulin (TUBG1), and RNA polymerase II (POLR2A).

Using qRT-PCR methods, transcription of the F8 gene using the mRNA extracted from each clonal culture and the parent culture was analyzed; yielded a quantitative value for each sample analyzed (ΔCₜ value).

The transcription of the F8 gene across all samples was compared. Clonal cultures that exhibit the highest ΔCₜ values for transcription of F8 when measured using qRT-PCR primers targeting the gene repair site itself were further isolated. These cells were cultured until the clonal colonies reach cell numbers of ≧5×10⁷

Next, ≧5×10⁷ cells from each culture were removed and pelleted. Cell lysate from the cell pellets was collected. A modified enzyme-linked immunosorbent assay (mELISA) was then used to detect the presence of FVIII protein in both the culture medium and the whole cell lysates from each culture. This yielded a quantitative value for each sample analyzed in units of nanograms of FVIII protein per cell number (ng/5×10⁷ cells). FVIII protein secretion across all samples was compared. The culture yielding the highest secretion of FVIII protein was chosen to proceed for therapeutic purposes.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications can be made without departing from the spirit and scope of the present disclosure. Accordingly, other embodiments are within the scope of the following claims.

1. A method for repairing one or more mutations in a Factor VIII gene (F8 gene) sequence of a subject, the method comprising
introducing into a cell of the subject one or more polynucleotides encoding a DNA scission enzyme (DNA-SE) and one or more repair vehicles (RVs) containing at least a cDNA-repair sequence (RS) flanked by an upstream flanking sequence (uFS) and a downstream flanking sequence (dFS) to form a DNA donor within each of the one or more repair vehicles (RVs),
wherein
the DNA-SE is selected to be capable of targeting a portion of the F8 gene of the subject and to create a first break in one strand of the F8 gene and a second break in the other strand of the F8 gene for subsequent repair by the cDNA-RS,
the cDNA-RS comprises a repaired version of the F8 gene sequence of the subject comprising the one or more mutations within a cDNA sequence encoding for a truncated Factor VIII, and
the upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene,
and wherein
introducing into a cell of the subject one or more polynucleotides encoding a DNA scission enzyme (DNA-SE) and one or more repair vehicles (RVs) is performed to allow insertion of the cDNA-RS through homologous recombination of the upstream flanking sequence (uFS) and the downstream flanking sequence (dFS) with the F8 gene of the subject (sF8) to provide a repaired F8 gene (rF8), the repaired F8 gene (rF8) upon expression forming a functional FVIII conferring improved coagulation functionality to the FVIII protein encoded by the sF8.

2. The method of claim 1, wherein the one or more mutations of Factor VIII gene of the subject result in a mutated Factor VIII gene comprise at least one Factor VIII functional coding sequence upstream to at least one Factor VIII non-functional coding sequence, the first break and the second break define a DNA-SE target site located upstream of a non-functional coding sequence to be repaired and the cDNA-RS is configured in the one or more repair vehicles to be in frame with the Factor VIII functional coding sequence upstream the DNA-SE target site.

3. The method of claim 2, wherein the DNA-SE target site is located about 50 bp to about 100 bp upstream from a 5′ end of the Factor VIII non-functional coding sequence to be repaired.

4. The method of claim 2, wherein the upstream flanking sequence (uFS) is homologous to a genomic nucleic acid sequence of at least 200 bp from the DNA-SE target site and the downstream flanking sequence (dFS) is homologous to a genomic nucleic acid sequences of at least 200 bp downstream of the DNA-SE target site.

5. The method of claim 2, wherein the DNA-SE target site is adjacent to a 3′ end of the Factor VIII functional coding sequence.

6. The method of claim 5, wherein the 3′ end of the functional coding sequence is a 3′ end of a Factor VIII exon.

7. The method of claim 2, wherein the one or more mutations comprise a replacement of one or more wild type nucleotide residues within an exon of the Factor VIII gene with one or more mutated nucleotide residues, the Factor VIII non-functional sequence is formed by the one or more mutated residues and the repaired version of the Factor VIII non-functional coding sequence is formed by the one or more mutated residues replaced by the one or more wild type nucleotide residues.

8. The method of claim 2, wherein the one or more mutations comprise an insertion of one or more nucleotide residues within an exon of the Factor VIII gene, the Factor VIII non-functional sequence is formed by the one or more inserted nucleotide residues and the repaired version of the Factor VIII non-functional coding sequence is formed by at least two nucleotide residues adjacent to a 5′ and 3′ end of the one or more inserted nucleotide residues.

9. The method of claim 2, wherein the one or more mutations comprise a deletion of one or more wild type nucleotide residues of at least one exon of the Factor VIII gene, the Factor VIII non-functional sequence is formed by one or more nucleotide residues downstream the one or more nucleotide residue deleted from the at least one exons, and the repaired version of the Factor VIII non-functional coding sequence comprises the one or more wild type nucleotide residues deleted from the at least one exon of Factor VIII.

10. The method of claim 2, wherein the one or more mutations comprise an intron 22 inversion, the Factor VIII functional coding sequence comprises exons 1 to 22 of the Factor VIII gene, the non-functional coding sequence comprises exons 23 to 24 of the Factor VIII gene and a repaired version of the Factor VIII non-functional coding sequence comprises exons 23 to 26 of the Factor VIII gene.

11. The method of claim 2, wherein the upstream flanking sequence (uFS) is homologous to a genomic nucleic acid sequence of at least about 400 bp from the DNA-SE target site and the downstream flanking sequence (dFS) is homologous to a genomic nucleic acid sequences of at least about 400 bp downstream of the DNA-SE target site.

12. The method of claim 2, wherein the upstream flanking sequence (uFS) is homologous to a genomic nucleic acid sequence of at least about 400-800 bp from the DNA-SE target site and the downstream flanking sequence (dFS) is homologous to a genomic nucleic acid sequences of at least about 400-800 bp downstream of the DNA-SE target site.

13. The method of claim 2, wherein the uFS is homologous to a genomic nucleic acid sequence of at least about 800-3000 bp from the DNA-SE target site and the dFS is homologous to a genomic nucleic acid sequences of at least about 800-3000 bp downstream of the DNA-SE target site.

14. The method of claim 2, wherein the cDNA repair sequence (cDNA-RS) encodes for one or more repaired Factor VIII non-functional sequences consisting essentially of the amino acid sequence encoded by exons 1, 2, 3, 4, 5, 6, 7, 8, 9, 10, 11, 12, 13, 14, 15, 16, 17, 18, 19, 20, 21, 22, 23, 24, 25, 26, or an in frame portion or combination thereof.

15. The method of claim 1, wherein the cDNA repair sequence (cDNA-RS) is in an editing cassette further comprising a polyadenylation site located at a 3′ end of the cDNA repair sequence (cDNA-RS), the editing cassette flanked by the upstream flanking sequence (uFS) and the downstream flanking sequence (dFS).

16. The method of claim 15, wherein the editing cassette further comprises a splice acceptor operatively linked to the cDNA repair sequence (cDNA-RS).

17. The method of claim 1, wherein the one or more mutations cause hemophilia A in the subject and the repair results in treatment of the hemophilia A in the subject

18. The method of claim 1, wherein the repaired version of the Factor VIII non-functional coding sequence comprises Factor VIII exons of a replacement FVIII protein product and the repair results in inducing immune tolerance to the FVIII replacement product.

19. A system for repairing one or more mutations in a Factor VIII gene (F8 gene) sequence of a subject, the system comprising
one or more polynucleotides encoding a DNA scission enzyme (DNA-SE) and one or more repair vehicles (RVs) containing at least a cDNA-repair sequence (RS) flanked by an upstream flanking sequence (uFS) and a downstream flanking sequence (dFS) to form a DNA donor within each of the one or more repair vehicles (RVs),
wherein
the DNA-SE is selected to be capable of targeting a portion of the F8 gene of the subject and to create a first break in one strand of the F8 gene and a second break in the other strand of the F8 gene for subsequent repair by the cDNA-RS,
the cDNA-RS comprises a repaired version of the F8 gene sequence of the subject comprising the one or more mutations within a cDNA sequence encoding for a truncated Factor VIII, and
the upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene,
and wherein, the DNA scission enzyme (DNA-SE), and the DNA donor are selected and configured so that upon insertion of the cDNA-RS through homologous recombination of the upstream flanking sequence (uFS) and the downstream flanking sequence (dFS) of the DNA donor sequence with the subject's F8 gene (sF8) a repaired F8 gene (rF8) is provided, the repaired F8 gene (rF8) upon expression forms functional FVIII that confers improved coagulation functionality to the FVIII protein encoded by the sF8 without the repair.

20. The system of claim 19, wherein the one or more nucleic acids encoding a DNA scission enzyme (DNA-SE) encode for a DNA-SE selected from the group consisting of zinc finder nuclease (ZFN), transcription activator-like effector nuclease (TALEN), cluster regularly interspaced short palindromic repeats (CRISPR)-associated (Cas) nuclease, CRISPR-Paired Nickase (CRISPR-PN), and CRISPR-RNA-guided Fok1 nucleases (CRISPR-RFN).

21. The system of claim 19, wherein the cDNA-RS encodes a truncated Factor VIII polypeptide consisting essentially of the amino acid sequence encoded by each of exons 1, 2, 3, 4, 5, 6, 7, 8, 9, 10, 11, 12, 13, 14, 15, 16, 17, 18, 19, 20, 21, 22, 23, 24, 25, 26 of a F8 gene or an in frame combination thereof.

22. A cDNA configured to be used as a cDNA-repair sequence (RS) for repairing one or more mutations in a Factor VIII gene (F8 gene) sequence of a subject, wherein the cDNA encodes a truncated Factor VIII polypeptide consisting essentially of the amino acid sequence encoded by each of exons 1, 2, 3, 4, 5, 6, 7, 8, 9, 10, 11, 12, 13, 14, 15, 16, 17, 18, 19, 20, 21, 22, 23, 24, 25, 26 of a F8 gene or an in frame combination thereof.

23. The cDNA of claim 22 wherein the each of the exons has a sequence of a corresponding exon in the F8 gene of the subject.

24. A repair vehicle (RV) configured to be used for repairing one or more mutations in a Factor VIII gene (F8 gene) sequence of a subject in combination with a DNA scission enzyme (DNA-SE) selected to target a portion of the F8 gene of the subject and to create a first break in one strand of the F8 gene and a second break in the other strand of the F8 gene,
the repair vehicle comprising a cDNA-repair sequence (RS) comprising a repaired version of the F8 gene sequence of the subject comprising the one or more mutations within a cDNA sequence encoding for a truncated Factor VIII.
wherein the cDNA-RS is flanked by an upstream flanking sequence (uFS) and a downstream flanking sequence (dFS) to form a DNA donor within the RV. The upstream flanking sequence (uFS) is homologous to a nucleic acid sequence upstream of the first break in the one strand of the F8 gene and the downstream flanking sequence (dFS) homologous to a nucleic acid sequences downstream of the second break in the other strand of the F8 gene.

25. A polynucleotide encoding a DNA scission enzyme (DNA-SE) configured for repairing one or more mutations in a Factor VIII gene (F8 gene) sequence of a subject, the DNA scission enzyme selected to be capable of targeting a portion of the F8 gene of the subject and to create a first break in one strand of the F8 gene and a second break in the other strand of the F8 gene for subsequent repair by a cDNA-RS flanked by an upstream flanking sequence (uFS) and a downstream flanking sequence (dFS) to form a DNA donor within each of the one or more repair vehicles (RVs),
the cDNA-RS comprising a repaired version of the F8 gene sequence of the subject comprising the one or more mutations within a cDNA sequence encoding for a truncated Factor VIII, and
the upstream flanking sequence (uFS) being homologous to a nucleic acid sequence upstream of the first break in the one strand of the F8 gene and the downstream flanking sequence (dFS) homologous to a nucleic acid sequences downstream of the second break in the other strand of the F8 gene.

26. A cell comprising the one or more repair vehicles (RVs) of claim 24 and one or more polynucleotide encoding the DNA scission enzyme (DNA-SE).

27. A composition for repairing one or more mutations in a Factor VIII gene (F8 gene) sequence of a subject, the composition comprising one or more repair vehicles (RVs) according to claim 24 and one or more polynucleotides encoding the DNA scission enzyme (DNA-SE), together with a suitable excipient.

28. A pharmaceutical composition for treatment of hemophilia in a subject, the composition comprising the one or more repair vehicles (RVs) according to claim 24 and one or more polynucleotides encoding the DNA scission enzyme (DNA-SE), together with a pharmaceutically acceptable excipient.


SEQUENCE LISTING (1659 sequences)

SEQ ID NO: 1 - DNA, 50 bp, Homo sapiens
tactatgggatgagttgcagatggcaagtaagacactggggagattaaat

SEQ ID NO: 2 - DNA, 49 bp, Homo sapiens
tggaaccttaatggtatgtaattagtcatttaaagggaatgcctgaata

SEQ ID NO: 3 - DNA, 44 bp, Homo sapiens
ttagtattatagtttctcagattatcaccagtgatactatggga

SEQ ID NO: 4 - protein, 544 aa, Artificial Sequence
  synthetic construct
LTPDQVVAIASNHGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHG

SEQ ID NO: 5 - DNA, 1632 bp, Artificial Sequence
  synthetic construct
ctgactccggaccaagtggtggctatcgccagcaaccacggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatggg

SEQ ID NO: 6 - protein, 544 aa, Artificial Sequence
  synthetic construct
LTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHG

SEQ ID NO: 7 - DNA, 1632 bp, Artificial Sequence
  synthetic construct
ctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatggg

SEQ ID NO: 8 - protein, 544 aa, Artificial Sequence
  synthetic construct
LTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNHGGKQALETVQRLLPVLCQDHG

SEQ ID NO: 9 - DNA, 1632 bp, Artificial Sequence
  synthetic construct
ctgactccggaccaagtggtggctatcgccagcaacatcggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatggg

SEQ ID NO: 10 - protein, 544 aa, Artificial Sequence
  synthetic construct
LTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASNGGGKQALETVQRLLPVLCQDHG

SEQ ID NO: 11 - DNA, 1632 bp, Artificial Sequence
  synthetic construct
ctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagccacgatggcggcaagcaagcgctcgaaacggtgcagcggctgttgccggtgctgtgccaggaccatgggctgactccggaccaagtggtggctatcgccagcaacatcggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatgggctgactccggaccaagtggtggctatcgccagcaaccacggcggcaagcaagcgctcgaaacggtgcagcggctgttgccggtgctgtgccaggaccatgggctgactccggaccaagtggtggctatcgccagcaacggtggcggcaagcaagcgctcgaaacggtgcagcggctgttgccggtgctgtgccaggaccatggg

SEQ ID NO: 12 - DNA, 7753 bp, Artificial Sequence
  synthetic construct
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagagcagcactaaatttgtctgggtgagtcagagaaggctaaccaggaaaaatagtttctgaactaacacttgaaggaggtgtagcagttcatcactgacagtgatgttggggtgggtctggtttcaggagaggggaggaaattggctttggtctgaggctgaggtgtgggcaaagcattagcttatgtgggtccattagcttatgtgagtccacaaaaggtgtgtgtgtgtttgtgtgtatgtgtgtgtgtgtgtgtgtgtgtgtgtgtgtacgaaatgggggctcaatgatttggtagtggtttggtttgtcaagaagcaggctgggaactcaataagcatctttccattcatttctactgtgtatcccacagcttcacacacacatgcacatttcaacattggtgactgcttcacttgcacacctaaggtaatgatggacacacctgtagcaatgtagattcttcctaagctaataattagtttcaggaggtagcacatacatttaaaaataggttaaaataaagtgttattttaattggtaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactagccgcggtgaagcttgatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgcctgaatactttaaagaattttggcagatttcagatattggacaaacactcttagcttccacaaacttaattccaaaaaataatttttcacttatgagcaatagagttattacggacatatcagcaaaaatgtagtagtgtcaaggctcatagatgatagaaatgaagagatgctgtattgatagaaatatgtgattcaggactgtgtggattgatgattgtgagcttgcttatggatatcctaggtttgaggttatagtaggacaatcaggttgaaatgtccagcaggcagtaggtgaaagacaagtttagggggcaaaaccatggatggagatgaagattcatgacttccacataaaaggatgggtgaaactttgggaattgatgaattctctagaggtgagctcaagacccttaaaggcttaaaacctcagcgttattgtctactcttccctcatttttatgcccacaaatctggtcaatcctttatttgcaatgcctctcacatctctttcttctgtttccatttataccgctgttgccacagcccagggtcccatcacctcacacttgatctattgtattacattcctaactagtcttcccccgtttctaatctgttctccgataaaagctgcacatcattttcaggataatcatcagtcgcctgcctaaaacttttcaatgtcttcccattgtaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - DNA, 11418 bp, Artificial Sequence
  synthetic construct
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgccatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactagccgcggtgaagcttgatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttttgtggggatgtaagtctgcttggaggaaggtgcagacatcgggttaggatggttgtgatgctacctgggccccaaagaaacatttctgggtaaggtgtgcacacatctgtgttattagcagaaatgctaactgccaattcttttcataggtctgacctatttgttgatattttgttctgttttgtccattgcttctcttcgtcatatgctgctcctccagaatctagagactggagtagagggagggtgaagggacaaagacaaaacttccctctgcctgcccaagcttccatagagagaatcaaggcaatgaaatccaatcaatatcacacacaagtttcatgtctggttctcttgtgtgtacatgcaatgtgtgtttttataatatcttttcctactttgggtgtaaggataatatgagccttgagttcagaagcttttcgtgttttgggggttctggtgcatttaggcagagtattaaataactttatcaatattgtctatggtcatcagttgattcagatttttctacctcttcttcagtaaatattggtatattttggtctatactttcatagaaagcaatctactgtccctagatttgataatgtattggtatcaagttatgtaagagtctcctgtgattttgttaaactgttctgtgtcaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagctcgagcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 7755 bp, Artificial Sequence
  synthetic construct
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgaggcttccaggtcgtaggtagattcaaagattttctgattggcaattggttgaaagagttaagttattgtgtaaagacttagaatcaatagagaggaacatcggggttaagataaggggttgtgaagaccaaggttctgtcatgcagatgaagcctccaggtagcaggcttcagagagaatagattgtaaatgtttcttatcagacttaaagagtctgttctatcagtctgaaggtctgtgttgatgttaatgctggtcagatttttctgaattccaaaagggaggcgggtataataaggcatatttgatccccactttcccatcatggcctgaacgtttttcaggttaactttggaaggccctttgccgaaaggaggggggattagaattttattttgggtttacaggatggtaatactctgttcccaccctcctaactagtatctttattaaaccttccacaaattatcctaatttccatgttttctgttccttgctggatcccttgtttcatacagtaattggtgctagaagaaaccccaggaaacagattttcaaaatgcaattctaaggttatgttgctaatatattcaagaaacacagagataacatatttgccaaggaagaaaatgagcagttagggaatccatgacatgtgttagtattatagtttctcagattatcaccagtgatactatgggaggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactagccgcggtgaagcttgatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttggagattaaatgacagtggcatttagtcactgtggcaacaaacgtagcattacctgattgtagagtggtctgtcttcttacagccctagagggcatacacatggaaaaagaaatgaaatgttatgaatatatacaaaataagaacactgatgaacatacatggaaaatcaggatgcatgcatagagcttttgaggaatactccgtatcctgtggttgtaggcagatacgacttaggggctgagcataagttgcagagctgcagtgacaattaaatgcttaactccaccagatctattatgctgtggtaagagtaccggtgggaaggagtgaaactctgaggcctgagatggaggcatttaggcagacatggatgaggctgagaattgcaaacctccaaattcccctgaacctcctttgcctgaggaggcaaccactccccagtctctgaagcagtcatccctcttttgtgtaaaagcctttcagtggctataactgagataggtgcctcacaaaccagtgactattctccttagagaccctgtttggacactacgaaagccaggcgagtcacagaaaatgacagcagatcacaaatttaatcaggtggtgatgccaaaaaacaattgcaattccagatatcatatctctgttgaagcaaatttacacagccccaggcacctgatatggaagtattgacctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 11359 bp, Artificial Sequence
  synthetic construct
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaaggtaaaggcatgtcctgtagggtctgatcggggccaggattgtggggatgtaagtctgcttggaggaaggtgcagacatcgggttaggatggttgtgatgctacctgggccccaaagaaacatttctgggtaaggtgtgcacacatctgtgttattagcagaaatgctaactgccaattcttttcataggtctgacctatttgttgatattttgttctgttttgtccattgcttctcttcgtcatatgctgctcctccaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactagccgcggtgaagcttgatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttagggacaaagacaaaacttccctctgcctgcccaagcttccatagagagaatcaaggcaatgaaatccaatcaatatcacacacaagtttcatgtctggttctcttgtgtgtacatgcaatgtgtgtttttataatatcttttcctactttgggtgtaaggataatatgagccttgagttcagaagcttttcgtgttttgggggttctggtgcatttaggcagagtattaaataactttatcaatattgtctatggtcatcagttgattcagatttttctacctcttcttcagtaaatattggtatattttggtctatactttcatagaaagcaatctactgtccctagatttgataatgtattggtatcaagttatgtaagagtctcctgtgattttgttaaactgttctgtgtctgtagttatattttctttttcattccttatgttgtatatgttctcttcctctcttttaaaaataatatttccaggagttttcttgattttattggtcttgtcaagaattttcttttggtttgatttatcaatctcttttttctttctgttgcatcagtttctgcttctactttcattgatttattccttccttctaatttcctttggttcattttgttgttagatttttgcttcttgagttgaatgctgaaatcatttattttatttttttgtcttctttaaataaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagctcgagcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17 - DNA, 1114 bp, Homo sapiens
gtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaag

SEQ ID NO: 18 - DNA, 6909 bp, Homo sapiens
tttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 19 - DNA, 1109 bp, Homo sapiens
ggggccaggattgtggggatgtaagtctgcttggaggaaggtgcagacatcgggttaggatggttgtgatgctacctgggccccaaagaaacatttctgggtaaggtgtgcacacatctgtgttattagcagaaatgctaactgccaattcttttcataggtctgacctatttgttgatattttgttctgttttgtccattgcttctcttcgtcatatgctgctcctccagaatctagagactggagtagagggagggtgaagggacaaagacaaaacttccctctgcctgcccaagcttccatagagagaatcaaggcaatgaaatccaatcaatatcacacacaagtttcatgtctggttctcttgtgtgtacatgcaatgtgtgtttttataatatcttttcctactttgggtgtaaggataatatgagccttgagttcagaagcttttcgtgttttgggggttctggtgcatttaggcagagtattaaataactttatcaatattgtctatggtcatcagttgattcagatttttctacctcttcttcagtaaatattggtatattttggtctatactttcatagaaagcaatctactgtccctagatttgataatgtattggtatcaagttatgtaagagtctcctgtgattttgttaaactgttctgtgtctgtagttatattttctttttcattccttatgttgtatatgttctcttcctctcttttaaaaataatatttccaggagttttcttgattttattggtcttgtcaagaattttcttttggtttgatttatcaatctcttttttctttctgttgcatcagtttctgcttctactttcattgatttattccttccttctaatttcctttggttcattttgttgttagatttttgcttcttgagttgaatgctgaaatcatttattttatttttttgtcttctttaaatgtgtattataaagatttaaatataatacatagattgtggctgtgtaaacattaaatgtggtcatgttgtacatactttatattctttttggttctttctgtttggctccccaccctctttccacatcagtccccttctcccccacctc

SEQ ID NO: 20 - DNA, 23 bp, homo sapiens
aagatactacctgggtgcagtgg

SEQ ID NO: 21 - DNA, 23 bp, homo sapiens
cactaaagcagaatcgcaaaagg

SEQ ID NO: 22 - DNA, 23 bp, homo sapiens
aagatactacctgggtgcagtgg

SEQ ID NO: 23 - DNA, 23 bp, homo sapiens
ttttcaacatcgctaagccaagg

SEQ ID NO: 24 - DNA, 23 bp, homo sapiens
agtctttttgtacacgactgagg

SEQ ID NO: 25 - DNA, 23 bp, homo sapiens
ttttcaacatcgctaagccaagg

SEQ ID NO: 26 - DNA, 23 bp, homo sapiens
atgctgttggtgtatcctactgg

SEQ ID NO: 27 - DNA, 23 bp, homo sapiens
cagcatgaagactgacaggatgg

SEQ ID NO: 28 - DNA, 23 bp, homo sapiens
atgctgttggtgtatcctactgg

SEQ ID NO: 29 - DNA, 23 bp, homo sapiens
gacttgaattcaggcctcattgg

SEQ ID NO: 30 - DNA, 23 bp, homo sapiens
tatgagtaggtaaggcacagtgg

SEQ ID NO: 31 - DNA, 23 bp, homo sapiens
gacttgaattcaggcctcattgg

SEQ ID NO: 32 - DNA, 23 bp, homo sapiens
aagtagtataaatttgtgcaagg

SEQ ID NO: 33 - DNA, 23 bp, homo sapiens
aagtagtataaatttgtgcaagg

SEQ ID NO: 34 - DNA, 23 bp, homo sapiens
ctttttgctgtatttgatgaagg

SEQ ID NO: 35 - DNA, 23 bp, homo sapiens
ctttttgctgtatttgatgaagg

SEQ ID NO: 36 - DNA, 23 bp, homo sapiens
cagtcaatggttatgtaaacagg

SEQ ID NO: 37 - DNA, 23 bp, homo sapiens
gactgtgtgcattttaggccagg

SEQ ID NO: 38 - DNA, 23 bp, homo sapiens
cagtcaatggttatgtaaacagg

SEQ ID NO: 39 - DNA, 23 bp, homo sapiens
caaacactcttgatggaccttgg

SEQ ID NO: 40 - DNA, 23 bp, homo sapiens
gcgagatttccaaggacgcctgg

SEQ ID NO: 41 - DNA, 23 bp, homo sapiens
caaacactcttgatggaccttgg

SEQ ID NO: 42 - DNA, 23 bp, homo sapiens
acattacattgctgctgaagagg

SEQ ID NO: 43 - DNA, 23 bp, homo sapiens
tcttggcaactgagcgaatttgg

SEQ ID NO: 44 - DNA, 23 bp, homo sapiens
acattacattgctgctgaagagg

SEQ ID NO: 45 - DNA, 23 bp, homo sapiens
gaagctattcagcatgaatcagg

SEQ ID NO: 46 - DNA, 23 bp, homo sapiens
aatagcttcacgagtcttaaagg

SEQ ID NO: 47 - DNA, 23 bp, homo sapiens
gaagctattcagcatgaatcagg

SEQ ID NO: 48 - DNA, 23 bp, homo sapiens
ggacatcagtgattccgtgaggg

SEQ ID NO: 49 - DNA, 23 bp, homo sapiens
ggacatcagtgattccgtgaggg

SEQ ID NO: 50 - DNA, 23 bp, homo sapiens
atgtccgtcctttgtattcaagg

SEQ ID NO: 51 - DNA, 23 bp, homo sapiens
gatctagcttcaggactcattgg

SEQ ID NO: 52 - DNA, 23 bp, homo sapiens
aacgaaactagagtaatagcggg

SEQ ID NO: 53 - DNA, 23 bp, homo sapiens
gatctagcttcaggactcattgg

SEQ ID NO: 54 - DNA, 23 bp, homo sapiens
cgctttctccccaatccagctgg

SEQ ID NO: 55 - DNA, 23 bp, homo sapiens
agcgttgtatattctctgtgagg

SEQ ID NO: 56 - DNA, 23 bp, homo sapiens
cgctttctccccaatccagctgg

SEQ ID NO: 57 - DNA, 23 bp, homo sapiens
agaaactgtcttcatgtcgatgg

SEQ ID NO: 58 - DNA, 23 bp, homo sapiens
atagaccattttgtgtttgaagg

SEQ ID NO: 59 - DNA, 23 bp, homo sapiens
agaaactgtcttcatgtcgatgg

SEQ ID NO: 60 - DNA, 23 bp, homo sapiens
acactattttattgctgcagtgg

SEQ ID NO: 61 - DNA, 23 bp, homo sapiens
ttttcttttgaaagctgcggggg

SEQ ID NO: 62 - DNA, 23 bp, homo sapiens
acactattttattgctgcagtgg

SEQ ID NO: 63 - DNA, 23 bp, homo sapiens
tcaacttctgctcttatatatgg

SEQ ID NO: 64 - DNA, 23 bp, homo sapiens
acggtataagggctgagtaaagg

SEQ ID NO: 65 - DNA, 23 bp, homo sapiens
acggtataagggctgagtaaagg

SEQ ID NO: 66 - DNA, 23 bp, homo sapiens
atgagtttgactgcaaagcctgg

SEQ ID NO: 67 - DNA, 23 bp, homo sapiens
cagtcaaactcatctttagtggg

SEQ ID NO: 68 - DNA, 23 bp, homo sapiens
atgagtttgactgcaaagcctgg

SEQ ID NO: 69 - DNA, 23 bp, homo sapiens
ggctccctgcaatatccagatgg

SEQ ID NO: 70 - DNA, 23 bp, homo sapiens
ttcagtgaagtaccagcttttgg

SEQ ID NO: 71 - DNA, 23 bp, homo sapiens
ggctccctgcaatatccagatgg

SEQ ID NO: 72 - DNA, 23 bp, homo sapiens
gttcactgtacgaaaaaaagagg

SEQ ID NO: 73 - DNA, 23 bp, homo sapiens
gtccactgaaatgaatagaatgg

SEQ ID NO: 74 - DNA, 23 bp, homo sapiens
gttcactgtacgaaaaaaagagg

SEQ ID NO: 75 - DNA, 23 bp, homo sapiens
caaagctggaatttggcgggtgg

SEQ ID NO: 76 - DNA, 23 bp, homo sapiens
cgccaaattccagctttggatgg

SEQ ID NO: 77 - DNA, 23 bp, homo sapiens
attggcgagcatctacatgctgg

SEQ ID NO: 78 - DNA, 23 bp, homo sapiens
tgtccagaagccattcccagggg

SEQ ID NO: 79 - DNA, 23 bp, homo sapiens
tgtccagaagccattcccagggg

SEQ ID NO: 80 - DNA, 23 bp, homo sapiens
gattttcagattacagcttcagg

SEQ ID NO: 81 - DNA, 23 bp, homo sapiens
aatcaatgcctggagcaccaagg

SEQ ID NO: 82 - DNA, 23 bp, homo sapiens
tgatccggaataatgaagtctgg

SEQ ID NO: 83 - DNA, 23 bp, homo sapiens
aatcaatgcctggagcaccaagg

SEQ ID NO: 84 - DNA, 23 bp, homo sapiens
aagaagtggcagacttatcgagg

SEQ ID NO: 85 - DNA, 23 bp, homo sapiens
agataaactgagagatgtagagg

SEQ ID NO: 86 - DNA, 23 bp, homo sapiens
aagaagtggcagacttatcgagg

SEQ ID NO: 87 - DNA, 23 bp, canis familiaris
ccatcaatggctatgtaaacagg

SEQ ID NO: 88 - DNA, 23 bp, canis familiaris
gaagctattcagtatgaatcagg

SEQ ID NO: 89 - DNA, 23 bp, canis familiaris
gatctagcttcaggactcattgg

SEQ ID NO: 90 - DNA, 23 bp, canis familiaris
agaaactgtcttcatgtcaatgg

SEQ ID NO: 91 - DNA, 23 bp, canis familiaris
atagaccattttgtgtttgaagg

SEQ ID NO: 92 - DNA, 23 bp, canis familiaris
agaaactgtcttcatgtcaatgg

SEQ ID NO: 93 - DNA, 23 bp, canis familiaris
acactatttcattgctgcagtgg

SEQ ID NO: 94 - DNA, 23 bp, canis familiaris
ttttcttttgaaagctgcggagg

SEQ ID NO: 95 - DNA, 23 bp, canis familiaris
acactatttcattgctgcagtgg

SEQ ID NO: 96 - DNA, 23 bp, canis familiaris
tcaacttctgctcttatatatgg

SEQ ID NO: 97 - DNA, 23 bp, canis familiaris
atgagtttgactgcaaagcctgg

SEQ ID NO: 98 - DNA, 23 bp, canis familiaris
cagtcaaactcatctttagtggg

SEQ ID NO: 99 - DNA, 23 bp, canis familiaris
atgagtttgactgcaaagcctgg

SEQ ID NO: 100 - DNA, 23 bp, canis familiaris
agctccctgcaatgtccagaagg

SEQ ID NO: 101 - DNA, 23 bp, canis familiaris
gttcactgtacgaaaaaaagagg

SEQ ID NO: 102 - DNA, 23 bp, canis familiaris
aatcaatgcctggagcaccaagg

SEQ ID NO: 103 - DNA, 23 bp, canis familiaris
tgatccggaataatgaagtctgg

SEQ ID NO: 104 - DNA, 23 bp, homo sapiens
aatcaatgcctggagcaccaagg

SEQ ID NO: 105 - DNA, 22 bp, homo sapiens
agatactacctgggtgcagtgg

SEQ ID NO: 106 - DNA, 22 bp, homo sapiens
aaacacaacctgggtgcagggg

SEQ ID NO: 107 - DNA, 22 bp, homo sapiens
aaaaagtacctgggtgcagaag

SEQ ID NO: 108 - DNA, 22 bp, homo sapiens
agaaactacatgggtgcagagg

SEQ ID NO: 109 - DNA, 22 bp, homo sapiens
ggcgaccacctgggtgcagcag

SEQ ID NO: 110 - DNA, 22 bp, homo sapiens
atttaccaactgggtgcagcag

SEQ ID NO: 111 - DNA, 22 bp, homo sapiens
atttaccatctgggtgcagggg

SEQ ID NO: 112 - DNA, 22 bp, homo sapiens
agatgcttcctgggtgcagcag

SEQ ID NO: 113 - DNA, 22 bp, homo sapiens
acaaactccctgggtgcagagg

SEQ ID NO: 114 - DNA, 22 bp, homo sapiens
acacactgcctgggtgcagcag

SEQ ID NO: 115 - DNA, 22 bp, homo sapiens
ggagacaccctgggtgcaggag

SEQ ID NO: 116 - DNA, 22 bp, homo sapiens
aggaacgccctgggtgcagaag

SEQ ID NO: 117 - DNA, 22 bp, homo sapiens
ggaagctacctgggtgcagggg

SEQ ID NO: 118 - DNA, 22 bp, homo sapiens
agataccctctgggtgcagaag

SEQ ID NO: 119 - DNA, 22 bp, homo sapiens
agataccctctgggtgcaggag

SEQ ID NO: 120 - DNA, 22 bp, homo sapiens
agataccctctgggtgcaggag

SEQ ID NO: 121 - DNA, 22 bp, homo sapiens
agatactgcctgggtccagagg

SEQ ID NO: 122 - DNA, 22 bp, homo sapiens
agatactccctgggtccagcag

SEQ ID NO: 123 - DNA, 22 bp, homo sapiens
ggatactagatgggtgcagagg

SEQ ID NO: 124 - DNA, 22 bp, homo sapiens
agagactacaagggtgcagtgg

SEQ ID NO: 125 - DNA, 22 bp, homo sapiens
caacactacctgggtgcaaaag

SEQ ID NO: 126 - DNA, 22 bp, homo sapiens
tttcaacatcgctaagccaagg

SEQ ID NO: 127 - DNA, 22 bp, homo sapiens
gaacaacatcgctaagccacag

SEQ ID NO: 128 - DNA, 22 bp, homo sapiens
tttcatcatggctaagccaagg

SEQ ID NO: 129 - DNA, 22 bp, homo sapiens
ttttaacatctctaagccatag

SEQ ID NO: 130 - DNA, 22 bp, homo sapiens
gtcaaacaacgctaagccaaag

SEQ ID NO: 131 - DNA, 22 bp, homo sapiens
cttcaaaatagctaagccaagg

SEQ ID NO: 132 - DNA, 22 bp, homo sapiens
ttcaaacatggctaagccatgg

SEQ ID NO: 133 - DNA, 22 bp, homo sapiens
gctcaaaatggctaagccaagg

SEQ ID NO: 134 - DNA, 22 bp, homo sapiens
ttagaatattgctaagccaggg

SEQ ID NO: 135 - DNA, 22 bp, homo sapiens
ttttaagatccctaagccaagg

SEQ ID NO: 136 - DNA, 22 bp, homo sapiens
ttgcaacaactctaagccaggg

SEQ ID NO: 137 - DNA, 22 bp, homo sapiens
tgtcaataaccctaagccatag

SEQ ID NO: 138 - DNA, 22 bp, homo sapiens
tcccaagatcgttaagccacag

SEQ ID NO: 139 - DNA, 22 bp, homo sapiens
ttccatcatggctaagccagag

SEQ ID NO: 140 - DNA, 22 bp, homo sapiens
ttgtagcattgctaagccatag

SEQ ID NO: 141 - DNA, 22 bp, homo sapiens
taacaaaatcgctaagctaaag

SEQ ID NO: 142 - DNA, 22 bp, homo sapiens
tttcaggatctctaagccaggg

SEQ ID NO: 143 - DNA, 22 bp, homo sapiens
tttcaaagtagctaagccagag

SEQ ID NO: 144 - DNA, 22 bp, homo sapiens
tgccaccatcactaagccaggg

SEQ ID NO: 145 - DNA, 22 bp, homo sapiens
tctaaacctggctaagccaaag

SEQ ID NO: 146 - DNA, 22 bp, homo sapiens
tttcaacatggcttagccagag

SEQ ID NO: 147 - DNA, 22 bp, homo sapiens
tgctgttggtgtatcctactgg

SEQ ID NO: 148 - DNA, 22 bp, homo sapiens
acctgttggtctatcctactag

SEQ ID NO: 149 - DNA, 22 bp, homo sapiens
tgatgttgatgtatcctaaggg

SEQ ID NO: 150 - DNA, 22 bp, homo sapiens
agctgttattgtatcctaccag

SEQ ID NO: 151 - DNA, 22 bp, homo sapiens
cactgttggtgcatcctacagg

SEQ ID NO: 152 - DNA, 22 bp, homo sapiens
tgctcatgctgtatcctaccgg

SEQ ID NO: 153 - DNA, 22 bp, homo sapiens
cgctgttgttgcatcctacagg

SEQ ID NO: 154 - DNA, 22 bp, homo sapiens
agcagttggtgtatccttctag

SEQ ID NO: 155 - DNA, 22 bp, homo sapiens
ttctgttgatgtatactactgg

SEQ ID NO: 156 - DNA, 22 bp, homo sapiens
ggatgttgatgtatcctgccag

SEQ ID NO: 157 - DNA, 22 bp, homo sapiens
tactatttctgtatcctaccag

SEQ ID NO: 158 - DNA, 22 bp, homo sapiens
tggtgtttgtttatcctactgg

SEQ ID NO: 159 - DNA, 22 bp, homo sapiens
ggctgtgggtgtttcctacaag

SEQ ID NO: 160 - DNA, 22 bp, homo sapiens
tacatttggtgtatcctaaggg

SEQ ID NO: 161 - DNA, 22 bp, homo sapiens
tgttgttggaatatcctaccag

SEQ ID NO: 162 - DNA, 22 bp, homo sapiens
tgctcttgttgtatcctaggag

SEQ ID NO: 163 - DNA, 22 bp, homo sapiens
ggctgttcatgtatcctaacag

SEQ ID NO: 164 - DNA, 22 bp, homo sapiens
ggctgctgctgcatcctaccag

SEQ ID NO: 165 - DNA, 22 bp, homo sapiens
tgcagatggtgtatccttcagg

SEQ ID NO: 166 - DNA, 22 bp, homo sapiens
tgttgctggtgtatactactag

SEQ ID NO: 167 - DNA, 22 bp, homo sapiens
acctgatggtgtatccttccag

SEQ ID NO: 168 - DNA, 22 bp, homo sapiens
acttgaattcaggcctcattgg

SEQ ID NO: 169 - DNA, 22 bp, homo sapiens
atttgtgttcaggcctcattgg

SEQ ID NO: 170 - DNA, 22 bp, homo sapiens
tcttgaaatcaggcctcatggg

SEQ ID NO: 171 - DNA, 22 bp, homo sapiens
acttgatttcaggcctcttcag

SEQ ID NO: 172 - DNA, 22 bp, homo sapiens
acttgatttcagtcctcattgg

SEQ ID NO: 173 - DNA, 22 bp, homo sapiens
actggaatccaggcctctttag

SEQ ID NO: 174 - DNA, 22 bp, homo sapiens
aatgggtttcaggcctcatggg

SEQ ID NO: 175 - DNA, 22 bp, homo sapiens
atgtgaattctggcctcattgg

SEQ ID NO: 176 - DNA, 22 bp, homo sapiens
actggacttcaggcctcaccag

SEQ ID NO: 177 - DNA, 22 bp, homo sapiens
atttgaattcaggcctgatagg

SEQ ID NO: 178 - DNA, 22 bp, homo sapiens
accagaatccaggcctcaggag

SEQ ID NO: 179 - DNA, 22 bp, homo sapiens
acttgaatcaaggcctcaaagg

SEQ ID NO: 180 - DNA, 22 bp, homo sapiens
acttgccctcaggcctcatcag

SEQ ID NO: 181 - DNA, 22 bp, homo sapiens
acaggcactcaggcctcattag

SEQ ID NO: 182 - DNA, 22 bp, homo sapiens
tctcagattcaggcctcatcag

SEQ ID NO: 183 - DNA, 22 bp, homo sapiens
ccttgggttgaggcctcatggg

SEQ ID NO: 184 - DNA, 22 bp, homo sapiens
aaatgaattcaggcctcttaag

SEQ ID NO: 185 - DNA, 22 bp, homo sapiens
agatgaattcaggcatcatagg

SEQ ID NO: 186 - DNA, 22 bp, homo sapiens
attttattttaggcctcataag

SEQ ID NO: 187 - DNA, 22 bp, homo sapiens
acctgaattcagccctcatgag

SEQ ID NO: 188 - DNA, 22 bp, homo sapiens
actgaaattctggcctcatcag

SEQ ID NO: 189 - DNA, 22 bp, homo sapiens
agtagtataaatttgtgcaagg

SEQ ID NO: 190 - DNA, 22 bp, homo sapiens
ggcagtattaatttgtgcaggg

SEQ ID NO: 191 - DNA, 22 bp, homo sapiens
aaaagaataaatttgtgcaaag

SEQ ID NO: 192 - DNA, 22 bp, homo sapiens
agaaatttaaatttgtgcaaag

SEQ ID NO: 193 - DNA, 22 bp, homo sapiens
agcagtataactttgtgcaggg

SEQ ID NO: 194 - DNA, 22 bp, homo sapiens
ggttgtataattttgtgcaagg

SEQ ID NO: 195 - DNA, 22 bp, homo sapiens
tgaagtataagtttgtgcaaag

SEQ ID NO: 196 - DNA, 22 bp, homo sapiens
attagtattaatttgtgaaagg

SEQ ID NO: 197 - DNA, 22 bp, homo sapiens
aggactaaaaatttgtgcaaag

SEQ ID NO: 198 - DNA, 22 bp, homo sapiens
agttgtatgaatttgtgtaaag

SEQ ID NO: 199 - DNA, 22 bp, homo sapiens
agtagaaacaatttgtgcaaag

SEQ ID NO: 200 - DNA, 22 bp, homo sapiens
agcaggattaatttgtgcatgg

SEQ ID NO: 201 - DNA, 22 bp, homo sapiens
aataatatagatttgtgcatag

SEQ ID NO: 202 - DNA, 22 bp, homo sapiens
tgaagtagaaatttgtgcatgg

SEQ ID NO: 203 - DNA, 22 bp, homo sapiens
agaggaatcaatttgtgcagag

SEQ ID NO: 204 - DNA, 22 bp, homo sapiens
ttaagtagaaatttgtgcaaag

SEQ ID NO: 205 - DNA, 22 bp, homo sapiens
agaagtctacatttgtgcacag

SEQ ID NO: 206 - DNA, 22 bp, homo sapiens
ggggttataaatttgtgcagag

SEQ ID NO: 207 - DNA, 22 bp, homo sapiens
cgtaatgttaatttgtgcatag

SEQ ID NO: 208 - DNA, 22 bp, homo sapiens
agtagttgaaatttgtgccaag

SEQ ID NO: 209 - DNA, 22 bp, homo sapiens
agaagtatctatttgtgcaaag

SEQ ID NO: 210 - DNA, 22 bp, homo sapiens
agtcaatggttatgtaaacagg

SEQ ID NO: 211 - DNA, 22 bp, homo sapiens
agtcaatagttatgtaaaccag

SEQ ID NO: 212 - DNA, 22 bp, homo sapiens
agtgaatggttttgtaaactag

SEQ ID NO: 213 - DNA, 22 bp, homo sapiens
aggaaatgtttatgtaaaccag

SEQ ID NO: 214 - DNA, 22 bp, homo sapiens
atccaagggttatgtaaaccag

SEQ ID NO: 215 - DNA, 22 bp, homo sapiens
aataaagggttatgtaaaccag

SEQ ID NO: 216 - DNA, 22 bp, homo sapiens
ccttaaaggttatgtaaactgg

SEQ ID NO: 217 - DNA, 22 bp, homo sapiens
agctaatgattatgtaaactag

SEQ ID NO: 218 - DNA, 22 bp, homo sapiens
aatcaatgttcatgtaaacagg

SEQ ID NO: 219 - DNA, 22 bp, homo sapiens
actcaaaggttatgtaatcagg

SEQ ID NO: 220 - DNA, 22 bp, homo sapiens
agtaaagggttttgtaaaccag

SEQ ID NO: 221 - DNA, 22 bp, homo sapiens
agctaatggatatgtaaactgg

SEQ ID NO: 222 - DNA, 22 bp, homo sapiens
tgagtatggttatgtaaacaag

SEQ ID NO: 223 - DNA, 22 bp, homo sapiens
attcaagggctatgtaaacagg

SEQ ID NO: 224 - DNA, 22 bp, homo sapiens
agtcaatgtttatataaacaag

SEQ ID NO: 225 - DNA, 22 bp, homo sapiens
agctaatctttatgtaaactag

SEQ ID NO: 226 - DNA, 22 bp, homo sapiens
agttaatgtatatgtaaacggg

SEQ ID NO: 227 - DNA, 22 bp, homo sapiens
agtcaaagaatatgtaaacagg

SEQ ID NO: 228 - DNA, 22 bp, homo sapiens
agtaaatgtttatgtaaaaaag

SEQ ID NO: 229 - DNA, 22 bp, homo sapiens
aatgaatgattatgtcaactgg

SEQ ID NO: 230 - DNA, 22 bp, homo sapiens
aggcaatgattttgtaaactag

SEQ ID NO: 231 - DNA, 22 bp, homo sapiens
aaacactcttgatggaccttgg

SEQ ID NO: 232 - DNA, 22 bp, homo sapiens
gcatcctcttgatggacctgag

SEQ ID NO: 233 - DNA, 22 bp, homo sapiens
atatactcttgattgacctcag

SEQ ID NO: 234 - DNA, 22 bp, homo sapiens
aattactctttatggacctgag

SEQ ID NO: 235 - DNA, 22 bp, homo sapiens
caacacacttgatggatcttag

SEQ ID NO: 236 - DNA, 22 bp, homo sapiens
aaagactgtttatggacctcag

SEQ ID NO: 237 - DNA, 22 bp, homo sapiens
aaacactcttaattgaccttag

SEQ ID NO: 238 - DNA, 22 bp, homo sapiens
aaccacatttgatggaccacag

SEQ ID NO: 239 - DNA, 22 bp, homo sapiens
tcacattcttgatggccctaag

SEQ ID NO: 240 - DNA, 22 bp, homo sapiens
agacattcttgctggacctgag

SEQ ID NO: 241 - DNA, 22 bp, homo sapiens
caacactgttgatgggccttgg

SEQ ID NO: 242 - DNA, 22 bp, homo sapiens
caatgctcttggtggacctgag

SEQ ID NO: 243 - DNA, 22 bp, homo sapiens
aaatactattgatggacataag

SEQ ID NO: 244 - DNA, 22 bp, homo sapiens
aaacccacttgatggacattag

SEQ ID NO: 245 - DNA, 22 bp, homo sapiens
aaacaccactgatggtccttag

SEQ ID NO: 246 - DNA, 22 bp, homo sapiens
acacactcttcatggagctagg

SEQ ID NO: 247 - DNA, 22 bp, homo sapiens
aaacactcatggtggacatgag

SEQ ID NO: 248 - DNA, 22 bp, homo sapiens
aaagagtcttgatagacctcgg

SEQ ID NO: 249 - DNA, 22 bp, homo sapiens
aaaaaattttcatggacctcag

SEQ ID NO: 250 - DNA, 22 bp, homo sapiens
taacattctgcatggacctcag

SEQ ID NO: 251 - DNA, 22 bp, homo sapiens
aaacactcctcctggacctagg

SEQ ID NO: 252 - DNA, 22 bp, homo sapiens
cattacattgctgctgaagagg

SEQ ID NO: 253 - DNA, 22 bp, homo sapiens
caatacattgctgctgaatagg

SEQ ID NO: 254 - DNA, 22 bp, homo sapiens
ctctacattgctgctgaagcag

SEQ ID NO: 255 - DNA, 22 bp, homo sapiens
aattatattgctgctgaagcag

SEQ ID NO: 256 - DNA, 22 bp, homo sapiens
ctgtatattgctgctgaagagg

SEQ ID NO: 257 - DNA, 22 bp, homo sapiens
tattacatttctgctgaagaag

SEQ ID NO: 258 - DNA, 22 bp, homo sapiens
ctgtacattgctgctgaaaagg

SEQ ID NO: 259 - DNA, 22 bp, homo sapiens
ttctactttgctgctgaagaag

SEQ ID NO: 260 - DNA, 22 bp, homo sapiens
ggagacattgctgctgaagtag

SEQ ID NO: 261 - DNA, 22 bp, homo sapiens
tttcacatggctgctgaagaag

SEQ ID NO: 262 - DNA, 22 bp, homo sapiens
ttttacttagctgctgaagaag

SEQ ID NO: 263 - DNA, 22 bp, homo sapiens
ctccacatagctgctgaaggag

SEQ ID NO: 264 - DNA, 22 bp, homo sapiens
cagtaaatttctgctgaagaag

SEQ ID NO: 265 - DNA, 22 bp, homo sapiens
cattccattgctgctgaaatag

SEQ ID NO: 266 - DNA, 22 bp, homo sapiens
cagtactatgctgctgaaggag

SEQ ID NO: 267 - DNA, 22 bp, homo sapiens
caacaaatagctgctgaagtag

SEQ ID NO: 268 - DNA, 22 bp, homo sapiens
aaaaacagtgctgctgaaggag

SEQ ID NO: 269 - DNA, 22 bp, homo sapiens
taattctttgctgctgaagcag

SEQ ID NO: 270 - DNA, 22 bp, homo sapiens
cattacatggcttctgaagagg

SEQ ID NO: 271 - DNA, 22 bp, homo sapiens
cagtacacagctgctgaaggag

SEQ ID NO: 272 - DNA, 22 bp, homo sapiens
gatgacttttctgctgaaggag

SEQ ID NO: 273 - DNA, 22 bp, homo sapiens
aagctattcagcatgaatcagg

SEQ ID NO: 274 - DNA, 22 bp, homo sapiens
actttattcagcatgaatccag

SEQ ID NO: 275 - DNA, 22 bp, homo sapiens
aacatattcagcatgaattaag

SEQ ID NO: 276 - DNA, 22 bp, homo sapiens
ctgatactcagcatgaatcagg

SEQ ID NO: 277 - DNA, 22 bp, homo sapiens
atgcaattctgcatgaatctag

SEQ ID NO: 278 - DNA, 22 bp, homo sapiens
aagataaccagcatgaatcaag

SEQ ID NO: 279 - DNA, 22 bp, homo sapiens
taactacacagcatgaatctgg

SEQ ID NO: 280 - DNA, 22 bp, homo sapiens
acacaattcagcatgaatccgg

SEQ ID NO: 281 - DNA, 22 bp, homo sapiens
aagttaggaagcatgaatcagg

SEQ ID NO: 282 - DNA, 22 bp, homo sapiens
aaactattcttcatgaatccag

SEQ ID NO: 283 - DNA, 22 bp, homo sapiens
gatctagtcatcatgaatccag

SEQ ID NO: 284 - DNA, 22 bp, homo sapiens
aaactaatcagcatgaataaag

SEQ ID NO: 285 - DNA, 22 bp, homo sapiens
aagttactcagcatgaatgtag

SEQ ID NO: 286 - DNA, 22 bp, homo sapiens
atactattcagcatgaataagg

SEQ ID NO: 287 - DNA, 22 bp, homo sapiens
catctaatcagcatgtatcagg

SEQ ID NO: 288 - DNA, 22 bp, homo sapiens
aagatgttctgcatgaatctag

SEQ ID NO: 289 - DNA, 22 bp, homo sapiens
gagctttaaagcatgaatcaag

SEQ ID NO: 290 - DNA, 22 bp, homo sapiens
aagataattagcatggatcaag

SEQ ID NO: 291 - DNA, 22 bp, homo sapiens
atgcagttgagcatgaatctgg

SEQ ID NO: 292 - DNA, 22 bp, homo sapiens
atggtattcagcattaatccag

SEQ ID NO: 293 - DNA, 22 bp, homo sapiens
aagatcttcagcaggaatcagg

SEQ ID NO: 294 - DNA, 22 bp, homo sapiens
gacatcagtgattccgtgaggg

SEQ ID NO: 295 - DNA, 22 bp, homo sapiens
ggcgtctgagattccgtgaggg

SEQ ID NO: 296 - DNA, 22 bp, homo sapiens
gaagtcattgattccgtgacag

SEQ ID NO: 297 - DNA, 22 bp, homo sapiens
gaagtcattgattccgtgacag

SEQ ID NO: 298 - DNA, 22 bp, homo sapiens
gccctctgtgattccctgagag

SEQ ID NO: 299 - DNA, 22 bp, homo sapiens
tccatctgtgagtccgtgacag

SEQ ID NO: 300 - DNA, 22 bp, homo sapiens
aaaatcagtgattccgtcatgg

SEQ ID NO: 301 - DNA, 22 bp, homo sapiens
gagattagggcttccgtgaagg

SEQ ID NO: 302 - DNA, 22 bp, homo sapiens
gagattagggcttccgtgaagg

SEQ ID NO: 303 - DNA, 22 bp, homo sapiens
tagaccagtgcttccgtgaggg

SEQ ID NO: 304 - DNA, 22 bp, homo sapiens
gccattagtgattccttgaaag

SEQ ID NO: 305 - DNA, 22 bp, homo sapiens
gacctcagtgattccatcaaag

SEQ ID NO: 306 - DNA, 22 bp, homo sapiens
gccatcagacattccgtgcaag

SEQ ID NO: 307 - DNA, 22 bp, homo sapiens
gacatcagtgatgccctgaggg

SEQ ID NO: 308 - DNA, 22 bp, homo sapiens
gaccacagagattccttgatgg

SEQ ID NO: 309 - DNA, 22 bp, homo sapiens
ggcgtcagtggttccatgaagg

SEQ ID NO: 310 - DNA, 22 bp, homo sapiens
gtaatcagtgattccatgcagg

SEQ ID NO: 311 - DNA, 22 bp, homo sapiens
gaaatcagcaattccgtaagag

SEQ ID NO: 312 - DNA, 22 bp, homo sapiens
gacaccagtcattccgtgctgg

SEQ ID NO: 313 - DNA, 22 bp, homo sapiens
ggcattagttattccctgatag

SEQ ID NO: 314 - DNA, 22 bp, homo sapiens
gagttctgtgataccgtgaaag

SEQ ID NO: 315 - DNA, 22 bp, homo sapiens
atctagcttcaggactcattgg

SEQ ID NO: 316 - DNA, 22 bp, homo sapiens
atttatcttcaggactcatgag

SEQ ID NO: 317 - DNA, 22 bp, homo sapiens
atgcagattcaggactcatggg

SEQ ID NO: 318 - DNA, 22 bp, homo sapiens
attgagtttcaggactcattgg

SEQ ID NO: 319 - DNA, 22 bp, homo sapiens
atcgggctccaggactcattgg

SEQ ID NO: 320 - DNA, 22 bp, homo sapiens
atcaaatttcaggactcattag

SEQ ID NO: 321 - DNA, 22 bp, homo sapiens
atattgcctcaggactcatcgg

SEQ ID NO: 322 - DNA, 22 bp, homo sapiens
gtctaacttcatgactcatcag

SEQ ID NO: 323 - DNA, 22 bp, homo sapiens
aactaacttcaagactcattgg

SEQ ID NO: 324 - DNA, 22 bp, homo sapiens
ctttagctacaggactcagagg

SEQ ID NO: 325 - DNA, 22 bp, homo sapiens
gcccagcttcaggacccatagg

SEQ ID NO: 326 - DNA, 22 bp, homo sapiens
ttctttgttcaggactcatggg

SEQ ID NO: 327 - DNA, 22 bp, homo sapiens
ttctttgttcaggactcatggg

SEQ ID NO: 328 - DNA, 22 bp, homo sapiens
atccaccttcaggactcagagg

SEQ ID NO: 329 - DNA, 22 bp, homo sapiens
atctagattcaagactcactgg

SEQ ID NO: 330 - DNA, 22 bp, homo sapiens
agccagctccaggactccttgg

SEQ ID NO: 331 - DNA, 22 bp, homo sapiens
acctaggatcaggactcagtgg

SEQ ID NO: 332 - DNA, 22 bp, homo sapiens
ctctaggttttggactcattgg

SEQ ID NO: 333 - DNA, 22 bp, homo sapiens
atctggcttctggactcaatgg

SEQ ID NO: 334 - DNA, 22 bp, homo sapiens
atagaacttcatgactcattag

SEQ ID NO: 335 - DNA, 22 bp, homo sapiens
agttagctttaggactcaagag

SEQ ID NO: 336 - DNA, 22 bp, homo sapiens
gctttctccccaatccagctgg

SEQ ID NO: 337 - DNA, 22 bp, homo sapiens
tctgtctccccaatccaggagg

SEQ ID NO: 338 - DNA, 22 bp, homo sapiens
aatctctccccaatccagcagg

SEQ ID NO: 339 - DNA, 22 bp, homo sapiens
gcagtttccccaatccagcagg

SEQ ID NO: 340 - DNA, 22 bp, homo sapiens
gacttttccccaatccagcagg

SEQ ID NO: 341 - DNA, 22 bp, homo sapiens
gctttctcctcaatccagggag

SEQ ID NO: 342 - DNA, 22 bp, homo sapiens
ccattctccccaaaccagcagg

SEQ ID NO: 343 - DNA, 22 bp, homo sapiens
tttttctcctcaatccagcaag

SEQ ID NO: 344 - DNA, 22 bp, homo sapiens
gatctctccacaatccagctgg

SEQ ID NO: 345 - DNA, 22 bp, homo sapiens
gctttctcccaaatccagaaag

SEQ ID NO: 346 - DNA, 22 bp, homo sapiens
gctttcatcccaatccaggtgg

SEQ ID NO: 347 - DNA, 22 bp, homo sapiens
tctttctgccccatccagcaag

SEQ ID NO: 348 - DNA, 22 bp, homo sapiens
gctatctcacccatccagcagg

SEQ ID NO: 349 - DNA, 22 bp, homo sapiens
acttcctgcccaatccagccag

SEQ ID NO: 350 - DNA, 22 bp, homo sapiens
gctttgtcatcaatccagccag

SEQ ID NO: 351 - DNA, 22 bp, homo sapiens
cctctttcccaaatccagcaag

SEQ ID NO: 352 - DNA, 22 bp, homo sapiens
gcctcctcctcaatccagccag

SEQ ID NO: 353 - DNA, 22 bp, homo sapiens
actttcctcacaatccagcaag

SEQ ID NO: 354 - DNA, 22 bp, homo sapiens
tctgtctccccattccagctgg

SEQ ID NO: 355 - DNA, 22 bp, homo sapiens
tccttctacctaatccagcagg

SEQ ID NO: 356 - DNA, 22 bp, homo sapiens
gctttcatcccaatccagaagg

SEQ ID NO: 357 - DNA, 22 bp, homo sapiens
gaaactgtcttcatgtcgatgg

SEQ ID NO: 358 - DNA, 22 bp, homo sapiens
gactctgtctttatgtcgatag

SEQ ID NO: 359 - DNA, 22 bp, homo sapiens
gaatctttcttcatgtccaaag

SEQ ID NO: 360 - DNA, 22 bp, homo sapiens
ggtactttcttcatgtcgtaag

SEQ ID NO: 361 - DNA, 22 bp, homo sapiens
gagacctccttcatgtcgaagg

SEQ ID NO: 362 - DNA, 22 bp, homo sapiens
acaactctcttcatgtctaaag

SEQ ID NO: 363 - DNA, 22 bp, homo sapiens
gaaactatattcatgttgaaag

SEQ ID NO: 364 - DNA, 22 bp, homo sapiens
gagactgtattcatgtcaacag

SEQ ID NO: 365 - DNA, 22 bp, homo sapiens
aagactgttttcatgtcaaggg

SEQ ID NO: 366 - DNA, 22 bp, homo sapiens
gaaaccgccttcatgtccaaag

SEQ ID NO: 367 - DNA, 22 bp, homo sapiens
gaacctggcttcatggcgatgg

SEQ ID NO: 368 - DNA, 22 bp, homo sapiens
gaagctgtcttcacgtcgccag

SEQ ID NO: 369 - DNA, 22 bp, homo sapiens
gaaactgtcttcatgtttaagg

SEQ ID NO: 370 - DNA, 22 bp, homo sapiens
gttactatcttcatgttgaaag

SEQ ID NO: 371 - DNA, 22 bp, homo sapiens
gatacttccttcatgtcaaaag

SEQ ID NO: 372 - DNA, 22 bp, homo sapiens
gtgaatgtcttcatgtccatgg

SEQ ID NO: 373 - DNA, 22 bp, homo sapiens
gattgtgtcttcatgtccacgg

SEQ ID NO: 374 - DNA, 22 bp, homo sapiens
gggactgtctgcatgccgacag

SEQ ID NO: 375 - DNA, 22 bp, homo sapiens
gacactatcatcatgtccaggg

SEQ ID NO: 376 - DNA, 22 bp, homo sapiens
caaactgtgtgcatggcgaagg

SEQ ID NO: 377 - DNA, 22 bp, homo sapiens
gaaactgtaatcatgtccaagg

SEQ ID NO: 378 - DNA, 22 bp, homo sapiens
cactattttattgctgcagtgg

SEQ ID NO: 379 - DNA, 22 bp, homo sapiens
aactattttattgctgcaagag

SEQ ID NO: 380 - DNA, 22 bp, homo sapiens
caccattttattgctgcaaagg

SEQ ID NO: 381 - DNA, 22 bp, homo sapiens
aaatattttgttgctgcagcag

SEQ ID NO: 382 - DNA, 22 bp, homo sapiens
gaatattttattgctgcaaaag

SEQ ID NO: 383 - DNA, 22 bp, homo sapiens
gatttttttattgctgcagaag

SEQ ID NO: 384 - DNA, 22 bp, homo sapiens
cgctgctttattgctgcagagg

SEQ ID NO: 385 - DNA, 22 bp, homo sapiens
agccactttattgctgcagaag

SEQ ID NO: 386 - DNA, 22 bp, homo sapiens
aaatattctattgctgcagcag

SEQ ID NO: 387 - DNA, 22 bp, homo sapiens
cagaaattcattgctgcagggg

SEQ ID NO: 388 - DNA, 22 bp, homo sapiens
caccagctcattgctgcagcag

SEQ ID NO: 389 - DNA, 22 bp, homo sapiens
caccagctcattgctgcagcag

SEQ ID NO: 390 - DNA, 22 bp, homo sapiens
ggttattctattgctgcagaag

SEQ ID NO: 391 - DNA, 22 bp, homo sapiens
aactattagattgctgcagaag

SEQ ID NO: 392 - DNA, 22 bp, homo sapiens
aaagcttttattgctgcaggag

SEQ ID NO: 393 - DNA, 22 bp, homo sapiens
aactttctgattgctgcagaag

SEQ ID NO: 394 - DNA, 22 bp, homo sapiens
ttctattgcattgctgcagggg

SEQ ID NO: 395 - DNA, 22 bp, homo sapiens
ccgtattagattgctgcaggag

SEQ ID NO: 396 - DNA, 22 bp, homo sapiens
gcttattttagtgctgcagaag

SEQ ID NO: 397 - DNA, 22 bp, homo sapiens
acatattttagtgctgcagaag

SEQ ID NO: 398 - DNA, 22 bp, homo sapiens
caccatctgtttgctgcagcag

SEQ ID NO: 399 - DNA, 22 bp, homo sapiens
caacttctgctcttatatatgg

SEQ ID NO: 400 - DNA, 22 bp, homo sapiens
taacttctgctcttatatctag

SEQ ID NO: 401 - DNA, 22 bp, homo sapiens
ccacttcttctcttatatacag

SEQ ID NO: 402 - DNA, 22 bp, homo sapiens
caacttgtggtcttatataaag

SEQ ID NO: 403 - DNA, 22 bp, homo sapiens
cagcttctgctctgatataggg

SEQ ID NO: 404 - DNA, 22 bp, homo sapiens
cattttctcctcttatataaag

SEQ ID NO: 405 - DNA, 22 bp, homo sapiens
caacttctgttcttatatttag

SEQ ID NO: 406 - DNA, 22 bp, homo sapiens
gaactcctgctcttatataagg

SEQ ID NO: 407 - DNA, 22 bp, homo sapiens
caacttttgctcttatatcagg

SEQ ID NO: 408 - DNA, 22 bp, homo sapiens
aagattctgctcttatatacag

SEQ ID NO: 409 - DNA, 22 bp, homo sapiens
catcttgtactcttatatatag

SEQ ID NO: 410 - DNA, 22 bp, homo sapiens
gatcttcttctcttatatagag

SEQ ID NO: 411 - DNA, 22 bp, homo sapiens
ctagtttttctcttatatatag

SEQ ID NO: 412 - DNA, 22 bp, homo sapiens
caatttgtgctattatatacag

SEQ ID NO: 413 - DNA, 22 bp, homo sapiens
caactcattctcttatatatag

SEQ ID NO: 414 - DNA, 22 bp, homo sapiens
caaactctgatcttatatacag

SEQ ID NO: 415 - DNA, 22 bp, homo sapiens
aatcttctgatcttatatacag

SEQ ID NO: 416 - DNA, 22 bp, homo sapiens
caccttatgatcttatatatag

SEQ ID NO: 417 - DNA, 22 bp, homo sapiens
aaccttcctctcttatataggg

SEQ ID NO: 418 - DNA, 22 bp, homo sapiens
caacctctgctcttaaataggg

SEQ ID NO: 419 - DNA, 22 bp, homo sapiens
cacattatgttcttatatacag

SEQ ID NO: 420 - DNA, 22 bp, homo sapiens
tgagtttgactgcaaagcctgg

SEQ ID NO: 421 - DNA, 22 bp, homo sapiens
tgattgtgactgcaaagccagg

SEQ ID NO: 422 - DNA, 22 bp, homo sapiens
tgaatgtgactgcaaagccaag

SEQ ID NO: 423 - DNA, 22 bp, homo sapiens
tgtgtttaactgcaaagcctgg

SEQ ID NO: 424 - DNA, 22 bp, homo sapiens
ttagtctgtctgcaaagcctgg

SEQ ID NO: 425 - DNA, 22 bp, homo sapiens
agagtttgtctccaaagccagg

SEQ ID NO: 426 - DNA, 22 bp, homo sapiens
tgtttttgactgcaaagtccag

SEQ ID NO: 427 - DNA, 22 bp, homo sapiens
taactcagactgcaaagccaag

SEQ ID NO: 428 - DNA, 22 bp, homo sapiens
aaattttcactgcaaagcccag

SEQ ID NO: 429 - DNA, 22 bp, homo sapiens
tgagtatggctgcaaagcacag

SEQ ID NO: 430 - DNA, 22 bp, homo sapiens
ttggcttgtctgcaaagcctgg

SEQ ID NO: 431 - DNA, 22 bp, homo sapiens
tgatttatcctgcaaagcccag

SEQ ID NO: 432 - DNA, 22 bp, homo sapiens
ggggtttgactgcaaagcaggg

SEQ ID NO: 433 - DNA, 22 bp, homo sapiens
tctttttgactgcaaagcttag

SEQ ID NO: 434 - DNA, 22 bp, homo sapiens
tgagtttgtatgcaaagcttag

SEQ ID NO: 435 - DNA, 22 bp, homo sapiens
tgagtttgactacaaagcagag

SEQ ID NO: 436 - DNA, 22 bp, homo sapiens
tctctttgactgcaaggcccag

SEQ ID NO: 437 - DNA, 22 bp, homo sapiens
tgagtggcactgcaaagccagg

SEQ ID NO: 438 - DNA, 22 bp, homo sapiens
tctgtttgactccaaagcccag

SEQ ID NO: 439 - DNA, 22 bp, homo sapiens
aggctttgactccaaagcctgg

SEQ ID NO: 440 - DNA, 22 bp, homo sapiens
acactttgacttcaaagcctag

SEQ ID NO: 441 - DNA, 22 bp, homo sapiens
gctccctgcaatatccagatgg

SEQ ID NO: 442 - DNA, 22 bp, homo sapiens
attccctgctatatccagacgg

SEQ ID NO: 443 - DNA, 22 bp, homo sapiens
gcttcccgccatatccagaggg

SEQ ID NO: 444 - DNA, 22 bp, homo sapiens
gctcctgccaatatccagatgg

SEQ ID NO: 445 - DNA, 22 bp, homo sapiens
attcctagcaatatccagaaag

SEQ ID NO: 446 - DNA, 22 bp, homo sapiens
gaaccaagcaatatccagagag

SEQ ID NO: 447 - DNA, 22 bp, homo sapiens
gctccctgctatagccagacag

SEQ ID NO: 448 - DNA, 22 bp, homo sapiens
gctgcccacaatatccagagag

SEQ ID NO: 449 - DNA, 22 bp, homo sapiens
gctgccgtcaatatccagatag

SEQ ID NO: 450 - DNA, 22 bp, homo sapiens
gaactctgcaatatccagatgg

SEQ ID NO: 451 - DNA, 22 bp, homo sapiens
gcccccagcagtatccagagag

SEQ ID NO: 452 - DNA, 22 bp, homo sapiens
ggaccccgcagtatccagaagg

SEQ ID NO: 453 - DNA, 22 bp, homo sapiens
gctcccagcgatatccaggcgg

SEQ ID NO: 454 - DNA, 22 bp, homo sapiens
gcatcctggaatatccaggtgg

SEQ ID NO: 455 - DNA, 22 bp, homo sapiens
ccgtcctgcaagatccagatgg

SEQ ID NO: 456 - DNA, 22 bp, homo sapiens
gcttcctgccatatccacaggg

SEQ ID NO: 457 - DNA, 22 bp, homo sapiens
tctgactacaatatccagaaag

SEQ ID NO: 458 - DNA, 22 bp, homo sapiens
tctgactacaatatccagaaag

SEQ ID NO: 459 - DNA, 22 bp, homo sapiens
gatcccttccatatccagaagg

SEQ ID NO: 460 - DNA, 22 bp, homo sapiens
tctccgtgcaatatccagtgag

SEQ ID NO: 461 - DNA, 22 bp, homo sapiens
attctctgcaatatccagcaag

SEQ ID NO: 462 - DNA, 22 bp, homo sapiens
ttcactgtacgaaaaaaagagg

SEQ ID NO: 463 - DNA, 22 bp, homo sapiens
ttcactgtgtgaaaaaaagaag

SEQ ID NO: 464 - DNA, 22 bp, homo sapiens
ttcactgttccaaaaaaagcag

SEQ ID NO: 465 - DNA, 22 bp, homo sapiens
ttcacattaagaaaaaaagtag

SEQ ID NO: 466 - DNA, 22 bp, homo sapiens
ttcacattaagaaaaaaagtag

SEQ ID NO: 467 - DNA, 22 bp, homo sapiens
ttcacattaagaaaaaaagtag

SEQ ID NO: 468 - DNA, 22 bp, homo sapiens
ttaactctaagaaaaaaagtag

SEQ ID NO: 469 - DNA, 22 bp, homo sapiens
ctcactttatgaaaaaaaggag

SEQ ID NO: 470 - DNA, 22 bp, homo sapiens
ttctctataggaaaaaaaggag

SEQ ID NO: 471 - DNA, 22 bp, homo sapiens
atcactttaggaaaaaaagtgg

SEQ ID NO: 472 - DNA, 22 bp, homo sapiens
ttaagtgtacaaaaaaaaggag

SEQ ID NO: 473 - DNA, 22 bp, homo sapiens
tccactgtaagaaaaaaacaag

SEQ ID NO: 474 - DNA, 22 bp, homo sapiens
tcccctttaggaaaaaaagcag

SEQ ID NO: 475 - DNA, 22 bp, homo sapiens
tagattgttcgaaaaaaagaag

SEQ ID NO: 476 - DNA, 22 bp, homo sapiens
ttcactgtatgaaaagaagaag

SEQ ID NO: 477 - DNA, 22 bp, homo sapiens
tgcactgtccaaaaaaaagagg

SEQ ID NO: 478 - DNA, 22 bp, homo sapiens
ttcaccctaccaaaaaaagtag

SEQ ID NO: 479 - DNA, 22 bp, homo sapiens
ttaactgaaagaaaaaaagagg

SEQ ID NO: 480 - DNA, 22 bp, homo sapiens
ttgattgtaagaaaaaaagtag

SEQ ID NO: 481 - DNA, 22 bp, homo sapiens
ttcagtttaagaaaaaaagcag

SEQ ID NO: 482 - DNA, 22 bp, homo sapiens
atcaatttaagaaaaaaagaag

SEQ ID NO: 483 - DNA, 22 bp, homo sapiens
aaagctggaatttggcgggtgg

SEQ ID NO: 484 - DNA, 22 bp, homo sapiens
gaggctgggatttggcgggagg

SEQ ID NO: 485 - DNA, 22 bp, homo sapiens
aaagcaggaatttggctggtag

SEQ ID NO: 486 - DNA, 22 bp, homo sapiens
aatcctggaatttggggggagg

SEQ ID NO: 487 - DNA, 22 bp, homo sapiens
aatggtggactttggcgggcgg

SEQ ID NO: 488 - DNA, 22 bp, homo sapiens
gaggctggactttggcgggtgg

SEQ ID NO: 489 - DNA, 22 bp, homo sapiens
aaaactggggtttggcgggggg

SEQ ID NO: 490 - DNA, 22 bp, homo sapiens
agggctggcatttggcggcaag

SEQ ID NO: 491 - DNA, 22 bp, homo sapiens
aagtctggaatttggagggagg

SEQ ID NO: 492 - DNA, 22 bp, homo sapiens
gaggctggaatttggggggagg

SEQ ID NO: 493 - DNA, 22 bp, homo sapiens
gaggctggaatttggggggagg

SEQ ID NO: 494 - DNA, 22 bp, homo sapiens
gaggctggaatttggagggtgg

SEQ ID NO: 495 - DNA, 22 bp, homo sapiens
agtcctggaatttggtgggtag

SEQ ID NO: 496 - DNA, 22 bp, homo sapiens
agagctggcatttggtgggagg

SEQ ID NO: 497 - DNA, 22 bp, homo sapiens
caagctggcatgtggcgggcag

SEQ ID NO: 498 - DNA, 22 bp, homo sapiens
caagctgggatctggcgggtgg

SEQ ID NO: 499 - DNA, 22 bp, homo sapiens
agagcagagatttggcggggag

SEQ ID NO: 500 - DNA, 22 bp, homo sapiens
agatctgggatatggcgggaag

SEQ ID NO: 501 - DNA, 22 bp, homo sapiens
aaaggtagactttggcgggtag

SEQ ID NO: 502 - DNA, 22 bp, homo sapiens
aaaggtagactttggcgggtag

SEQ ID NO: 503 - DNA, 22 bp, homo sapiens
aaagctggagttttgcggggag

SEQ ID NO: 504 - DNA, 22 bp, homo sapiens
gtccagaagccattcccagggg

SEQ ID NO: 505 - DNA, 22 bp, homo sapiens
gtgcagaagctattcccagagg

SEQ ID NO: 506 - DNA, 22 bp, homo sapiens
gtccaggagtcattcccagggg

SEQ ID NO: 507 - DNA, 22 bp, homo sapiens
atccagaagccattcccacagg

SEQ ID NO: 508 - DNA, 22 bp, homo sapiens
gccaagcagccattcccagcag

SEQ ID NO: 509 - DNA, 22 bp, homo sapiens
gcccagcacccattcccagcag

SEQ ID NO: 510 - DNA, 22 bp, homo sapiens
taccagaaaccattcccagcag

SEQ ID NO: 511 - DNA, 22 bp, homo sapiens
gtgcagaagccattctcagagg

SEQ ID NO: 512 - DNA, 22 bp, homo sapiens
gtcaagaagccattctcagaag

SEQ ID NO: 513 - DNA, 22 bp, homo sapiens
gcccagtagcctttcccagggg

SEQ ID NO: 514 - DNA, 22 bp, homo sapiens
gtactgaagacattcccagtag

SEQ ID NO: 515 - DNA, 22 bp, homo sapiens
caccacaatccattcccagtgg

SEQ ID NO: 516 - DNA, 22 bp, homo sapiens
caccacaatccattcccagtgg

SEQ ID NO: 517 - DNA, 22 bp, homo sapiens
caccacaatccattcccagtgg

SEQ ID NO: 518 - DNA, 22 bp, homo sapiens
gcccatcacccattcccagcag

SEQ ID NO: 519 - DNA, 22 bp, homo sapiens
atcctgaagcaattcccaggag

SEQ ID NO: 520 - DNA, 22 bp, homo sapiens
cttcagaagtcattcccagggg

SEQ ID NO: 521 - DNA, 22 bp, homo sapiens
gacaagaagtcattcccagtgg

SEQ ID NO: 522 - DNA, 22 bp, homo sapiens
gcacagaaggcattcccagggg

SEQ ID NO: 523 - DNA, 22 bp, homo sapiens
gcctggaatccattcccagcag

SEQ ID NO: 524 - DNA, 22 bp, homo sapiens
gggctgaacccattcccagcag

SEQ ID NO: 525 - DNA, 22 bp, homo sapiens
atcaatgcctggagcaccaagg

SEQ ID NO: 526 - DNA, 22 bp, homo sapiens
atctacccctggagcaccaggg

SEQ ID NO: 527 - DNA, 22 bp, homo sapiens
atctaatcctggagcaccaagg

SEQ ID NO: 528 - DNA, 22 bp, homo sapiens
ttcatttcctggagcaccaaag

SEQ ID NO: 529 - DNA, 22 bp, homo sapiens
agaaatacctggagcaccagag

SEQ ID NO: 530 - DNA, 22 bp, homo sapiens
gtaaatgcctgcagcaccatgg

SEQ ID NO: 531 - DNA, 22 bp, homo sapiens
accaaagcctagagcaccacag

SEQ ID NO: 532 - DNA, 22 bp, homo sapiens
accaaagcctagagcaccacag

SEQ ID NO: 533 - DNA, 22 bp, homo sapiens
atcgttccctggagcaccatag

SEQ ID NO: 534 - DNA, 22 bp, homo sapiens
aacaatgcctggatcaccacag

SEQ ID NO: 535 - DNA, 22 bp, homo sapiens
gtcttttcctggagcaccagag

SEQ ID NO: 536 - DNA, 22 bp, homo sapiens
aatcatggctggagcaccagag

SEQ ID NO: 537 - DNA, 22 bp, homo sapiens
gtccatgcctggaccaccacag

SEQ ID NO: 538 - DNA, 22 bp, homo sapiens
gtcgctgcctggagcaccatgg

SEQ ID NO: 539 - DNA, 22 bp, homo sapiens
ggcactgcctggagcaccatgg

SEQ ID NO: 540 - DNA, 22 bp, homo sapiens
agccctgcctggagcaccaggg

SEQ ID NO: 541 - DNA, 22 bp, homo sapiens
atcagttcctggagcacctggg

SEQ ID NO: 542 - DNA, 22 bp, homo sapiens
aaccatgcctggaacaccatgg

SEQ ID NO: 543 - DNA, 22 bp, homo sapiens
atcaaatcctggagccccaggg

SEQ ID NO: 544 - DNA, 22 bp, homo sapiens
ggcaatgcctggagcaacaaag

SEQ ID NO: 545 - DNA, 22 bp, homo sapiens
atgagtgcctgaagcaccaagg

SEQ ID NO: 546 - DNA, 22 bp, homo sapiens
agaagtggcagacttatcgagg

SEQ ID NO: 547 - DNA, 22 bp, homo sapiens
agaagcagcagacttatccagg

SEQ ID NO: 548 - DNA, 22 bp, homo sapiens
ggaagttgcaaacttatcgagg

SEQ ID NO: 549 - DNA, 22 bp, homo sapiens
ggatgtggcagacttatcttag

SEQ ID NO: 550 - DNA, 22 bp, homo sapiens
ctgagtggcaggcttatcgggg

SEQ ID NO: 551 - DNA, 22 bp, homo sapiens
agaacaggcagacttatcttag

SEQ ID NO: 552 - DNA, 22 bp, homo sapiens
agaagaggcatacttatctgag

SEQ ID NO: 553 - DNA, 22 bp, homo sapiens
gaaactggcagacttatctagg

SEQ ID NO: 554 - DNA, 22 bp, homo sapiens
agaagtggcagagttatcctgg

SEQ ID NO: 555 - DNA, 22 bp, homo sapiens
aggagtggctgacttatctaag

SEQ ID NO: 556 - DNA, 22 bp, homo sapiens
aaaaatggtagacttatcaaag

SEQ ID NO: 557 - DNA, 22 bp, homo sapiens
agaagtcgcaggcttatgggag

SEQ ID NO: 558 - DNA, 22 bp, homo sapiens
agaagaggaagacttatggaag

SEQ ID NO: 559 - DNA, 22 bp, homo sapiens
agtgctggcagacttattgcag

SEQ ID NO: 560 - DNA, 22 bp, homo sapiens
aggaggggcagatttatcgaag

SEQ ID NO: 561 - DNA, 22 bp, homo sapiens
agaagtagaaaacttatcatag

SEQ ID NO: 562 - DNA, 22 bp, homo sapiens
agcagtggcagacatattgaag

SEQ ID NO: 563 - DNA, 22 bp, homo sapiens
agaagtggatgacttattgcag

SEQ ID NO: 564 - DNA, 22 bp, homo sapiens
gcaagtggcaggcttatctggg

SEQ ID NO: 565 - DNA, 22 bp, homo sapiens
gcaagtggcagacttttccaag

SEQ ID NO: 566 - DNA, 22 bp, homo sapiens
aagagtggcagacttctcatgg

SEQ ID NO: 567 - DNA, 23 bp, canis familiaris
acgctgttggtgtatcctattgg

SEQ ID NO: 568 - DNA, 23 bp, canis familiaris
gacctgaattcaggcctcattgg

SEQ ID NO: 569 - DNA, 17 bp, homo sapiens
tggaactgtcatgggac

SEQ ID NO: 570 - DNA, 20 bp, homo sapiens
tccacaggcagctcaccgag

SEQ ID NO: 571 - DNA, 20 bp, homo sapiens
tctgtttgtagaattcacgg

SEQ ID NO: 572 - DNA, 20 bp, homo sapiens
tctgtttgtagaattcacgg

SEQ ID NO: 573 - DNA, 19 bp, homo sapiens
tacacttaagaacatggct

SEQ ID NO: 574 - DNA, 20 bp, homo sapiens
tacaccaacagcatgaagac

SEQ ID NO: 575 - DNA, 22 bp, homo sapiens
tgtgccttacctactcatatct

SEQ ID NO: 576 - DNA, 21 bp, homo sapiens
tgaattcaagtcttttaccag

SEQ ID NO: 577 - DNA, 24 bp, homo sapiens
tctggccaaggaaaagacacagac

SEQ ID NO: 578 - DNA, 24 bp, homo sapiens
ttcatcaaatacagcaaaaagtag

SEQ ID NO: 579 - DNA, 17 bp, homo sapiens
tgctgcatctgctcggg

SEQ ID NO: 580 - DNA, 22 bp, homo sapiens
tttacataaccattgactgtgt

SEQ ID NO: 581 - DNA, 18 bp, homo sapiens
tctcgccaataactttcc

SEQ ID NO: 582 - DNA, 20 bp, homo sapiens
tgtccaaggtccatcaagag

SEQ ID NO: 583 - DNA, 23 bp, homo sapiens
tcagttgccaagaagcatcctaa

SEQ ID NO: 584 - DNA, 22 bp, homo sapiens
tcctcctcttcagcagcaatgt

SEQ ID NO: 585 - DNA, 18 bp, homo sapiens
ttcagcatgaatcaggaa

SEQ ID NO: 586 - DNA, 18 bp, homo sapiens
tctccaacttccccataa

SEQ ID NO: 587 - DNA, 20 bp, homo sapiens
tataacatctaccctcacgg

SEQ ID NO: 588 - DNA, 20 bp, homo sapiens
tctccttgaatacaaaggac

SEQ ID NO: 589 - DNA, 18 bp, homo sapiens
tctagcttcaggactcat

SEQ ID NO: 590 - DNA, 21 bp, homo sapiens
tctacagattctttgtagcag

SEQ ID NO: 591 - DNA, 19 bp, homo sapiens
tcacagagaatatacaacg

SEQ ID NO: 592 - DNA, 21 bp, homo sapiens
tcctcaagctgcactccagct

SEQ ID NO: 593 - DNA, 17 bp, homo sapiens
tgtcttcttctctggat

SEQ ID NO: 594 - DNA, 21 bp, homo sapiens
tgtgtcttcatagaccatttt

SEQ ID NO: 595 - DNA, 24 bp, homo sapiens
tcaaaagaaaacacgacactattt

SEQ ID NO: 596 - DNA, 24 bp, homo sapiens
tcatcccataatcccagagcctct

SEQ ID NO: 597 - DNA, 20 bp, homo sapiens
tcagcccttataccgtggag

SEQ ID NO: 598 - DNA, 20 bp, homo sapiens
tatggccccaggagtcccaa

SEQ ID NO: 599 - DNA, 22 bp, homo sapiens
tatggcacccactaaagatgag

SEQ ID NO: 600 - DNA, 18 bp, homo sapiens
tcagagaaataagcccag

SEQ ID NO: 601 - DNA, 17 bp, homo sapiens
tctttgatgagaccaaa

SEQ ID NO: 602 - DNA, 17 bp, homo sapiens
tctttccatattttcag

SEQ ID NO: 603 - DNA, 19 bp, homo sapiens
tctattcatttcagtggac

SEQ ID NO: 604 - DNA, 19 bp, homo sapiens
tatactcctctttttttcg

SEQ ID NO: 605 - DNA, 17 bp, homo sapiens
tgttaccatccaaagct

SEQ ID NO: 606 - DNA, 21 bp, homo sapiens
tgctcgccaataaggcattcc

SEQ ID NO: 607 - DNA, 21 bp, homo sapiens
tcccctgggaatggcttctgg

SEQ ID NO: 608 - DNA, 22 bp, homo sapiens
tgtcctgaagctgtaatctgaa

SEQ ID NO: 609 - DNA, 20 bp, homo sapiens
tgggccccaaagctggccag

SEQ ID NO: 610 - DNA, 20 bp, homo sapiens
tgctccaggcattgattgat

SEQ ID NO: 611 - DNA, 19 bp, homo sapiens
tctacatctctcagtttat

SEQ ID NO: 612 - DNA, 22 bp, homo sapiens
tctgccacttcttcccatcaag

SEQ ID NO: 613 - DNA, 24 bp, canis familiaris
tctggccaaagaaaggacacagac

SEQ ID NO: 614 - DNA, 24 bp, canis familiaris
ttcatcaaatacagcaaaaagtag

SEQ ID NO: 615 - DNA, 23 bp, canis familiaris
tcagttgccaagaagcatcctaa

SEQ ID NO: 616 - DNA, 22 bp, canis familiaris
tcctcctcctcagcagcaatat

SEQ ID NO: 617 - DNA, 18 bp, canis familiaris
tctagcttcaggactcat

SEQ ID NO: 618 - DNA, 21 bp, canis familiaris
tctacagattctttgtagcag

SEQ ID NO: 619 - DNA, 17 bp, canis familiaris
tgtcttcttctctggat

SEQ ID NO: 620 - DNA, 21 bp, canis familiaris
tgtgtcttcatagaccatttt

SEQ ID NO: 621 - DNA, 24 bp, canis familiaris
tcaaaagaaaacacgacactattt

SEQ ID NO: 622 - DNA, 24 bp, canis familiaris
tcatcccataatcccagagacgct

SEQ ID NO: 623 - DNA, 20 bp, canis familiaris
tcagcccttataccgtggag

SEQ ID NO: 624 - DNA, 20 bp, canis familiaris
tatggccccaagagtcccaa

SEQ ID NO: 625 - DNA, 22 bp, canis familiaris
tatggcacccactaaagatgag

SEQ ID NO: 626 - DNA, 18 bp, canis familiaris
tcagaaaaataagcccag

SEQ ID NO: 627 - DNA, 20 bp, canis familiaris
tgggccccaaagctggccag

SEQ ID NO: 628 - DNA, 20 bp, canis familiaris
tgctccaggcattgattgat

SEQ ID NO: 629 - DNA, 20 bp, homo sapiens
tccacaggcagctcaccgag

SEQ ID NO: 630 - DNA, 17 bp, homo sapiens
tggaactctcatggaac

SEQ ID NO: 631 - DNA, 17 bp, homo sapiens
aggagctgtcagtcaac

SEQ ID NO: 632 - DNA, 17 bp, homo sapiens
tggaactgtcatggtgc

SEQ ID NO: 633 - DNA, 17 bp, homo sapiens
tgaaactgtcatatgac

SEQ ID NO: 634 - DNA, 17 bp, homo sapiens
tgaagctgtcatgaaac

SEQ ID NO: 635 - DNA, 17 bp, homo sapiens
taaaactataatggaag

SEQ ID NO: 636 - DNA, 17 bp, homo sapiens
tgaagctgtcatgaaac

SEQ ID NO: 637 - DNA, 17 bp, homo sapiens
tggacccagcatggggc

SEQ ID NO: 638 - DNA, 17 bp, homo sapiens
tggaactgtcatgtgag

SEQ ID NO: 639 - DNA, 17 bp, homo sapiens
taggactgtcctgagcc

SEQ ID NO: 640 - DNA, 17 bp, homo sapiens
tagaactatcatgggaa

SEQ ID NO: 641 - DNA, 17 bp, homo sapiens
tggcattgtcatggaac

SEQ ID NO: 642 - DNA, 17 bp, homo sapiens
tgaaaccctcatgagcc

SEQ ID NO: 643 - DNA, 17 bp, homo sapiens
tgtaaatgtcatggaac

SEQ ID NO: 644 - DNA, 17 bp, homo sapiens
tggaaatgtcatagaac

SEQ ID NO: 645 - DNA, 17 bp, homo sapiens
taggtctgtcttgggtc

SEQ ID NO: 646 - DNA, 17 bp, homo sapiens
tataactgtcaagagac

SEQ ID NO: 647 - DNA, 17 bp, homo sapiens
tggagctgaaaagcaac

SEQ ID NO: 648 - DNA, 17 bp, homo sapiens
tagaactaacataaaac

SEQ ID NO: 649 - DNA, 17 bp, homo sapiens
tagaaatatcctgggat

SEQ ID NO: 650 - DNA, 17 bp, homo sapiens
gtcccatgacagttcca

SEQ ID NO: 651 - DNA, 17 bp, homo sapiens
gagcaatgactgttcca

SEQ ID NO: 652 - DNA, 17 bp, homo sapiens
gtctcatgacagttaca

SEQ ID NO: 653 - DNA, 20 bp, homo sapiens
ctcagagagttgcctggtta

SEQ ID NO: 654 - DNA, 17 bp, homo sapiens
tgcccatgactcctcca

SEQ ID NO: 655 - DNA, 17 bp, homo sapiens
cttccattatagtttta

SEQ ID NO: 656 - DNA, 17 bp, homo sapiens
gtttcatgacagcttca

SEQ ID NO: 657 - DNA, 17 bp, homo sapiens
cttccattatagtttta

SEQ ID NO: 658 - DNA, 17 bp, homo sapiens
gttccttgacatttcca

SEQ ID NO: 659 - DNA, 17 bp, homo sapiens
gttccatggcagatact

SEQ ID NO: 660 - DNA, 17 bp, homo sapiens
ggctcaggacagtccca

SEQ ID NO: 661 - DNA, 17 bp, homo sapiens
ggcttctgagacttcca

SEQ ID NO: 662 - DNA, 17 bp, homo sapiens
gtcctaggtaggttcca

SEQ ID NO: 663 - DNA, 17 bp, homo sapiens
gtcctatgagatttcta

SEQ ID NO: 664 - DNA, 17 bp, homo sapiens
gtctcatttcagtgtaa

SEQ ID NO: 665 - DNA, 17 bp, homo sapiens
ctccattgacagatctt

SEQ ID NO: 666 - DNA, 17 bp, homo sapiens
gctccatgtcagtttca

SEQ ID NO: 667 - DNA, 17 bp, homo sapiens
gtccaatttcagtccaa

SEQ ID NO: 668 - DNA, 17 bp, homo sapiens
gtctcctgacagctcca

SEQ ID NO: 669 - DNA, 17 bp, homo sapiens
gtgtcattatagttgca

SEQ ID NO: 670 - DNA, 17 bp, homo sapiens
attgcatgatagttcca

SEQ ID NO: 671 - DNA, 18 bp, homo sapiens
tggccttggcttagcgat

SEQ ID NO: 672 - DNA, 18 bp, homo sapiens
tggacttggcttcgcgct

SEQ ID NO: 673 - DNA, 18 bp, homo sapiens
tagccttggcttagaaaa

SEQ ID NO: 674 - DNA, 18 bp, homo sapiens
ggaacttgacttagccct

SEQ ID NO: 675 - DNA, 18 bp, homo sapiens
tgccctggacttggaggt

SEQ ID NO: 676 - DNA, 18 bp, homo sapiens
tggccttggtttagaaaa

SEQ ID NO: 677 - DNA, 20 bp, homo sapiens
tctatttgtagaattactag

SEQ ID NO: 678 - DNA, 20 bp, homo sapiens
tctttttgtaaaaatgacga

SEQ ID NO: 679 - DNA, 18 bp, homo sapiens
tgtccatggcctgggggt

SEQ ID NO: 680 - DNA, 18 bp, homo sapiens
cagccttggcttgtggat

SEQ ID NO: 681 - DNA, 18 bp, homo sapiens
tgacctctccttaaccat

SEQ ID NO: 682 - DNA, 18 bp, homo sapiens
tggcaatgccttagaaat

SEQ ID NO: 683 - DNA, 18 bp, homo sapiens
tttccttggcttagtgat

SEQ ID NO: 684 - DNA, 18 bp, homo sapiens
tttccttggcttagtgat

SEQ ID NO: 685 - DNA, 18 bp, homo sapiens
tgtgattgagttagcaat

SEQ ID NO: 686 - DNA, 18 bp, homo sapiens
ctgccctggctgaaccat

SEQ ID NO: 687 - DNA, 18 bp, homo sapiens
ttgcatttactcagccat

SEQ ID NO: 688 - DNA, 18 bp, homo sapiens
tggcctgagctttggggt

SEQ ID NO: 689 - DNA, 20 bp, homo sapiens
tgtgcttataaaattcactg

SEQ ID NO: 690 - DNA, 18 bp, homo sapiens
tagggctggcttggcgag

SEQ ID NO: 691 - DNA, 18 bp, homo sapiens
tggcagtagctctgccat

SEQ ID NO: 692 - DNA, 20 bp, homo sapiens
ccgtgaattctacaaacaga

SEQ ID NO: 693 - DNA, 18 bp, homo sapiens
atggaaaagccaaggaga

SEQ ID NO: 694 - DNA, 18 bp, homo sapiens
ctggctaagcaaagataa

SEQ ID NO: 695 - DNA, 18 bp, homo sapiens
cctgctaagccaaggcta

SEQ ID NO: 696 - DNA, 18 bp, homo sapiens
agagataagccaaggtca

SEQ ID NO: 697 - DNA, 18 bp, homo sapiens
agcgataaggaaaggtta

SEQ ID NO: 698 - DNA, 18 bp, homo sapiens
ttggttaagccaattcca

SEQ ID NO: 699 - DNA, 18 bp, homo sapiens
attccgaagccaaggata

SEQ ID NO: 700 - DNA, 18 bp, homo sapiens
atcttgaagccaaggcta

SEQ ID NO: 701 - DNA, 18 bp, homo sapiens
tttactaagacaaggcca

SEQ ID NO: 702 - DNA, 18 bp, homo sapiens
atttctaaactaaggtca

SEQ ID NO: 703 - DNA, 18 bp, homo sapiens
attgctaagtcaaatcaa

SEQ ID NO: 704 - DNA, 18 bp, homo sapiens
attgctaactcaatcaca

SEQ ID NO: 705 - DNA, 18 bp, homo sapiens
attgctaactcaatcaca

SEQ ID NO: 706 - DNA, 18 bp, homo sapiens
atcactaagccaaggaaa

SEQ ID NO: 707 - DNA, 18 bp, homo sapiens
atcgctaagccagtgtta

SEQ ID NO: 708 - DNA, 18 bp, homo sapiens
atcttttagccaatgcca

SEQ ID NO: 709 - DNA, 18 bp, homo sapiens
actgctaagacaagccca

SEQ ID NO: 710 - DNA, 20 bp, homo sapiens
cagttatttctactaccaga

SEQ ID NO: 711 - DNA, 18 bp, homo sapiens
gtagctaagtcaaggcta

SEQ ID NO: 712 - DNA, 18 bp, homo sapiens
aattctaagctaaggcca

SEQ ID NO: 713 - DNA, 20 bp, homo sapiens
tacaccaacagcatgaagac

SEQ ID NO: 714 - DNA, 19 bp, homo sapiens
aacaatcaggctcatggca

SEQ ID NO: 715 - DNA, 19 bp, homo sapiens
tatacttaaaaacatagct

SEQ ID NO: 716 - DNA, 19 bp, homo sapiens
tacatttaaaaacatgtct

SEQ ID NO: 717 - DNA, 19 bp, homo sapiens
tacgctgcagagctgggca

SEQ ID NO: 718 - DNA, 19 bp, homo sapiens
tacacataacaacatggct

SEQ ID NO: 719 - DNA, 19 bp, homo sapiens
aatccttaagaacatgact

SEQ ID NO: 720 - DNA, 19 bp, homo sapiens
cacactgaagaccatggct

SEQ ID NO: 721 - DNA, 19 bp, homo sapiens
tagacttaatcatgtagct

SEQ ID NO: 722 - DNA, 19 bp, homo sapiens
tacacttgtgaagatggat

SEQ ID NO: 723 - DNA, 19 bp, homo sapiens
tacacttagaaaaaaagct

SEQ ID NO: 724 - DNA, 19 bp, homo sapiens
tacacttatgtgtttggct

SEQ ID NO: 725 - DNA, 19 bp, homo sapiens
aacacttaaaaacagggct

SEQ ID NO: 726 - DNA, 19 bp, homo sapiens
aacaaataatatcatcact

SEQ ID NO: 727 - DNA, 19 bp, homo sapiens
cgcacaaaaaaacatggat

SEQ ID NO: 728 - DNA, 19 bp, homo sapiens
cacatttgagaacatggct

SEQ ID NO: 729 - DNA, 19 bp, homo sapiens
tgcacttaaaaatatgaca

SEQ ID NO: 730 - DNA, 19 bp, homo sapiens
tacactgaagagaatggag

SEQ ID NO: 731 - DNA, 19 bp, homo sapiens
ttcattgaagaaaaaagct

SEQ ID NO: 732 - DNA, 19 bp, homo sapiens
tacatttaagcatatggct

SEQ ID NO: 733 - DNA, 19 bp, homo sapiens
tacccctgtgaacatggaa

SEQ ID NO: 734 - DNA, 19 bp, homo sapiens
agccatgttcttaagtgta

SEQ ID NO: 735 - DNA, 19 bp, homo sapiens
agccatgtttttaagagta

SEQ ID NO: 736 - DNA, 19 bp, homo sapiens
agtgatttttttcaatgaa

SEQ ID NO: 737 - DNA, 19 bp, homo sapiens
agctatcttattcatttta

SEQ ID NO: 738 - DNA, 19 bp, homo sapiens
agacatttttttaagtgta

SEQ ID NO: 739 - DNA, 19 bp, homo sapiens
agccagacactaaaatata

SEQ ID NO: 740 - DNA, 19 bp, homo sapiens
atccttgttcttatgttca

SEQ ID NO: 741 - DNA, 20 bp, homo sapiens
gtcttcatcatgttagtgtc

SEQ ID NO: 742 - DNA, 19 bp, homo sapiens
ggctatgttcttaagtgtc

SEQ ID NO: 743 - DNA, 19 bp, homo sapiens
aggcttgtacttaattgta

SEQ ID NO: 744 - DNA, 20 bp, homo sapiens
gtttgtttgctgttgttgtt

SEQ ID NO: 745 - DNA, 19 bp, homo sapiens
atccatgttgttgagtgta

SEQ ID NO: 746 - DNA, 19 bp, homo sapiens
atctatttactaaattgtt

SEQ ID NO: 747 - DNA, 19 bp, homo sapiens
atctatgttcttaagtcta

SEQ ID NO: 748 - DNA, 19 bp, homo sapiens
aggcatgtttttaagtggg

SEQ ID NO: 749 - DNA, 20 bp, homo sapiens
gctttcagtctggtggttta

SEQ ID NO: 750 - DNA, 19 bp, homo sapiens
agcacagtgcttaagtgca

SEQ ID NO: 751 - DNA, 19 bp, homo sapiens
agcaatgtttttaagtgat

SEQ ID NO: 752 - DNA, 20 bp, homo sapiens
atttttatgctgttggagta

SEQ ID NO: 753 - DNA, 19 bp, homo sapiens
tgctttagtcttaattgta

SEQ ID NO: 754 - DNA, 19 bp, homo sapiens
tgctttgttcttaaatgta

SEQ ID NO: 755 - DNA, 21 bp, homo sapiens
tgaattcaagtcttttaccag

SEQ ID NO: 756 - DNA, 21 bp, homo sapiens
tgaattcaaatctttttcctg

SEQ ID NO: 757 - DNA, 21 bp, homo sapiens
tgatttctagttttgtgccaa

SEQ ID NO: 758 - DNA, 21 bp, homo sapiens
tgacattaagacatttaacag

SEQ ID NO: 759 - DNA, 21 bp, homo sapiens
gaaaggcaagccatatactag

SEQ ID NO: 760 - DNA, 21 bp, homo sapiens
tgcatacaattcctttaccaa

SEQ ID NO: 761 - DNA, 21 bp, homo sapiens
taaagtcacttcctttacgac

SEQ ID NO: 762 - DNA, 21 bp, homo sapiens
tgaatccaaaacttttacctg

SEQ ID NO: 763 - DNA, 21 bp, homo sapiens
ggaattaaagtccttcacata

SEQ ID NO: 764 - DNA, 21 bp, homo sapiens
ggaattcaattcaataacaag

SEQ ID NO: 765 - DNA, 21 bp, homo sapiens
tgtattcaagtccttaaaaag

SEQ ID NO: 766 - DNA, 21 bp, homo sapiens
ataattctagtcttaggacag

SEQ ID NO: 767 - DNA, 21 bp, homo sapiens
tgaattccttccttgtaccat

SEQ ID NO: 768 - DNA, 21 bp, homo sapiens
tgaaagcaaatctttccccag

SEQ ID NO: 769 - DNA, 21 bp, homo sapiens
tgacttcaagtctttaaacaa

SEQ ID NO: 770 - DNA, 22 bp, homo sapiens
ggtcactaatctactcttatct

SEQ ID NO: 771 - DNA, 21 bp, homo sapiens
tgaattcaactctttagacag

SEQ ID NO: 772 - DNA, 21 bp, homo sapiens
tgaattcaaagctttccttac

SEQ ID NO: 773 - DNA, 21 bp, homo sapiens
tgaaattgaatctgattccag

SEQ ID NO: 774 - DNA, 21 bp, homo sapiens
tgaattcaattcttcagccag

SEQ ID NO: 775 - DNA, 21 bp, homo sapiens
tgaattcaattcttcagccag

SEQ ID NO: 776 - DNA, 22 bp, homo sapiens
agatatgagtaggtaaggcaca

SEQ ID NO: 777 - DNA, 21 bp, homo sapiens
ttggaaaaatcccttaataca

SEQ ID NO: 778 - DNA, 21 bp, homo sapiens
ttagtaaatgacctgaattca

SEQ ID NO: 779 - DNA, 21 bp, homo sapiens
ctgggaaaagaagtggattca

SEQ ID NO: 780 - DNA, 21 bp, homo sapiens
ctgataaatgacttgtattca

SEQ ID NO: 781 - DNA, 21 bp, homo sapiens
ctgataaacaattttaattta

SEQ ID NO: 782 - DNA, 21 bp, homo sapiens
atggttgatgacttgaattca

SEQ ID NO: 783 - DNA, 21 bp, homo sapiens
ctgggttaatacatttattta

SEQ ID NO: 784 - DNA, 21 bp, homo sapiens
ttggttacagacttgaagtca

SEQ ID NO: 785 - DNA, 21 bp, homo sapiens
tgggcaaaagacctgaattga

SEQ ID NO: 786 - DNA, 21 bp, homo sapiens
ttggttaaaattttgaactca

SEQ ID NO: 787 - DNA, 21 bp, homo sapiens
ctgggaaaagtttggaattta

SEQ ID NO: 788 - DNA, 21 bp, homo sapiens
tggattaaagacttgaatgca

SEQ ID NO: 789 - DNA, 21 bp, homo sapiens
ctggtcaatgccttgcttgca

SEQ ID NO: 790 - DNA, 21 bp, homo sapiens
atcagaaaatctttgaatcca

SEQ ID NO: 791 - DNA, 22 bp, homo sapiens
agatatgaacaggtaaggcact

SEQ ID NO: 792 - DNA, 21 bp, homo sapiens
gtaaggaaagctttgaattca

SEQ ID NO: 793 - DNA, 21 bp, homo sapiens
ctgtctaaagagttgaattca

SEQ ID NO: 794 - DNA, 21 bp, homo sapiens
tttgtcaaagacttgtattta

SEQ ID NO: 795 - DNA, 21 bp, homo sapiens
tcagaaaaatctttgaatcca

SEQ ID NO: 796 - DNA, 21 bp, homo sapiens
tcagaaaaatctttgaatcca

SEQ ID NO: 797 - DNA, 24 bp, homo sapiens
ttcatcaaatacagcaaaaagtag

SEQ ID NO: 798 - DNA, 24 bp, homo sapiens
tctagccaagccagaggcactgac

SEQ ID NO: 799 - DNA, 24 bp, homo sapiens
ttcaacaacaacaacaaaaaagca

SEQ ID NO: 800 - DNA, 24 bp, homo sapiens
ctcaccaagcattgcataaagctg

SEQ ID NO: 801 - DNA, 24 bp, homo sapiens
ttcatcaactccaggaaaaacaac

SEQ ID NO: 802 - DNA, 24 bp, homo sapiens
ttcataatatcaagtaatacgtga

SEQ ID NO: 803 - DNA, 24 bp, homo sapiens
tctggccatgacagataaacgctc

SEQ ID NO: 804 - DNA, 24 bp, homo sapiens
tcaaaccaagggaaagacagagaa

SEQ ID NO: 805 - DNA, 24 bp, homo sapiens
tcttgccacaaaaaatacacagaa

SEQ ID NO: 806 - DNA, 24 bp, homo sapiens
ttaataaagtgaaactaaaagtaa

SEQ ID NO: 807 - DNA, 24 bp, homo sapiens
tcagaccaagccagaggtgcacac

SEQ ID NO: 808 - DNA, 24 bp, homo sapiens
tctggccacaaaaactacacagaa

SEQ ID NO: 809 - DNA, 24 bp, homo sapiens
tccacccaaggaataggcagagag

SEQ ID NO: 810 - DNA, 24 bp, homo sapiens
gccaacagcaacagcaacaaaaag

SEQ ID NO: 811 - DNA, 24 bp, homo sapiens
tcatgaaaaataaaagaaacagta

SEQ ID NO: 812 - DNA, 24 bp, homo sapiens
tctgtccaaaaaaaaaaaaaaaaa

SEQ ID NO: 813 - DNA, 24 bp, homo sapiens
tcaggccaataaaaaacaacaaac

SEQ ID NO: 814 - DNA, 24 bp, homo sapiens
tctagcaaagaaaaataaacaaaa

SEQ ID NO: 815 - DNA, 24 bp, homo sapiens
tcaaacaaaaaaaaaaaaacaaac

SEQ ID NO: 816 - DNA, 24 bp, homo sapiens
tacataaaacacaacaagaaatag

SEQ ID NO: 817 - DNA, 24 bp, homo sapiens
tctggcaaaaggatagccacagat

SEQ ID NO: 818 - DNA, 24 bp, homo sapiens
gtctgtgtcttttccttggccaga

SEQ ID NO: 819 - DNA, 24 bp, homo sapiens
ggctctgtcttttcctctgccaca

SEQ ID NO: 820 - DNA, 24 bp, homo sapiens
ctctgtgccatgtacttggccaga

SEQ ID NO: 821 - DNA, 24 bp, homo sapiens
ctacttttaggtgtattttatgaa

SEQ ID NO: 822 - DNA, 24 bp, homo sapiens
gtatatgtgttttcactggacaga

SEQ ID NO: 823 - DNA, 24 bp, homo sapiens
gtttgggtttttttctttgacaga

SEQ ID NO: 824 - DNA, 24 bp, homo sapiens
atactttttgctgtgtttgattca

SEQ ID NO: 825 - DNA, 24 bp, homo sapiens
gtctgtgcctctgcatgggcgtgt

SEQ ID NO: 826 - DNA, 24 bp, homo sapiens
ctacgttgtgatgtgtttactcaa

SEQ ID NO: 827 - DNA, 24 bp, homo sapiens
gtctgtatgttttattttgctaga

SEQ ID NO: 828 - DNA, 24 bp, homo sapiens
ggctttgtcatttccttggcctgt

SEQ ID NO: 829 - DNA, 24 bp, homo sapiens
ctacgttgtgatgtgtttactcaa

SEQ ID NO: 830 - DNA, 24 bp, homo sapiens
ctaatctttgctgtattttattga

SEQ ID NO: 831 - DNA, 24 bp, homo sapiens
gtttttgtcttttttttagacaga

SEQ ID NO: 832 - DNA, 24 bp, homo sapiens
gttttttttttttcttgggacaga

SEQ ID NO: 833 - DNA, 24 bp, homo sapiens
gttttgtttcctgaatttgataaa

SEQ ID NO: 834 - DNA, 24 bp, homo sapiens
tgcctttttttttttttttttgaa

SEQ ID NO: 835 - DNA, 24 bp, homo sapiens
ttatttttcttttttttttccagc

SEQ ID NO: 836 - DNA, 24 bp, homo sapiens
atatatatatattccttggccgga

SEQ ID NO: 837 - DNA, 24 bp, homo sapiens
ttacttacatttgtatttgaagat

SEQ ID NO: 838 - DNA, 24 bp, homo sapiens
ttgtgtttctttttcctgggcatg

SEQ ID NO: 839 - DNA, 22 bp, homo sapiens
tttacataaccattgactgtgt

SEQ ID NO: 840 - DNA, 17 bp, homo sapiens
tgctgcattagctcaga

SEQ ID NO: 841 - DNA, 17 bp, homo sapiens
tactgcatcttctctgg

SEQ ID NO: 842 - DNA, 17 bp, homo sapiens
ggtggcatctgctcttg

SEQ ID NO: 843 - DNA, 17 bp, homo sapiens
ttctacttctgctttag

SEQ ID NO: 844 - DNA, 17 bp, homo sapiens
ttctacttctgctttag

SEQ ID NO: 845 - DNA, 17 bp, homo sapiens
tgcagcctctgctcaga

SEQ ID NO: 846 - DNA, 17 bp, homo sapiens
tgttacacctgctctgg

SEQ ID NO: 847 - DNA, 17 bp, homo sapiens
tgatgcatctgtttcga

SEQ ID NO: 848 - DNA, 22 bp, homo sapiens
tttacctaaccaatgaaagtgt

SEQ ID NO: 849 - DNA, 17 bp, homo sapiens
tgctgcagcaactcggg

SEQ ID NO: 850 - DNA, 17 bp, homo sapiens
ttttccatcagctcaga

SEQ ID NO: 851 - DNA, 17 bp, homo sapiens
tgccacatcagctctgg

SEQ ID NO: 852 - DNA, 17 bp, homo sapiens
tgctgcctctgccttca

SEQ ID NO: 853 - DNA, 17 bp, homo sapiens
ttctacatctgctcaga

SEQ ID NO: 854 - DNA, 17 bp, homo sapiens
tgctgtctctgctcagg

SEQ ID NO: 855 - DNA, 17 bp, homo sapiens
tgcagcctctgctccag

SEQ ID NO: 856 - DNA, 17 bp, homo sapiens
tgctgcatctactcctg

SEQ ID NO: 857 - DNA, 17 bp, homo sapiens
tgcagcctctgcccggg

SEQ ID NO: 858 - DNA, 17 bp, homo sapiens
tactgcatcttctcaga

SEQ ID NO: 859 - DNA, 17 bp, homo sapiens
tgttgcatcagctcggg

SEQ ID NO: 860 - DNA, 17 bp, homo sapiens
cccgagcagatgcagca

SEQ ID NO: 861 - DNA, 17 bp, homo sapiens
ccagagcagaggcagct

SEQ ID NO: 862 - DNA, 17 bp, homo sapiens
ctggagtaggcgctgca

SEQ ID NO: 863 - DNA, 17 bp, homo sapiens
ctggagtaggcgctgca

SEQ ID NO: 864 - DNA, 17 bp, homo sapiens
tctgtgtagatgcagca

SEQ ID NO: 865 - DNA, 17 bp, homo sapiens
ttttacaagatgcagca

SEQ ID NO: 866 - DNA, 17 bp, homo sapiens
cctgagctggggttgca

SEQ ID NO: 867 - DNA, 17 bp, homo sapiens
gctgagcatatgcagga

SEQ ID NO: 868 - DNA, 17 bp, homo sapiens
cctgagcaggtgcatca

SEQ ID NO: 869 - DNA, 17 bp, homo sapiens
cctttgtagatgcagaa

SEQ ID NO: 870 - DNA, 17 bp, homo sapiens
ctcgagcaggggccgcc

SEQ ID NO: 871 - DNA, 17 bp, homo sapiens
cctgagcagcttcagca

SEQ ID NO: 872 - DNA, 17 bp, homo sapiens
ccagagcagcaggagca

SEQ ID NO: 873 - DNA, 17 bp, homo sapiens
catgagcaggtgcagca

SEQ ID NO: 874 - DNA, 17 bp, homo sapiens
tcctgggaagtgcagca

SEQ ID NO: 875 - DNA, 17 bp, homo sapiens
cctgagcggaagcagag

SEQ ID NO: 876 - DNA, 17 bp, homo sapiens
tcccaggagatgtagaa

SEQ ID NO: 877 - DNA, 17 bp, homo sapiens
cccgggcagatctattg

SEQ ID NO: 878 - DNA, 17 bp, homo sapiens
tcccagcaggcggagca

SEQ ID NO: 879 - DNA, 17 bp, homo sapiens
caaaagcagtttcaaca

SEQ ID NO: 880 - DNA, 17 bp, homo sapiens
cctgatcagcgacagcc

SEQ ID NO: 881 - DNA, 20 bp, homo sapiens
tgtccaaggtccatcaagag

SEQ ID NO: 882 - DNA, 20 bp, homo sapiens
tgtcaaaaatcaatcaaaaa

SEQ ID NO: 883 - DNA, 20 bp, homo sapiens
tgtccaaagtccattttgag

SEQ ID NO: 884 - DNA, 20 bp, homo sapiens
tgtcacaggtccttaaagag

SEQ ID NO: 885 - DNA, 18 bp, homo sapiens
tcttaccaatcactttct

SEQ ID NO: 886 - DNA, 20 bp, homo sapiens
tgtccaaagtcacttgagag

SEQ ID NO: 887 - DNA, 20 bp, homo sapiens
tatccagactccatccacag

SEQ ID NO: 888 - DNA, 20 bp, homo sapiens
agtccaacatccagcaagaa

SEQ ID NO: 889 - DNA, 18 bp, homo sapiens
tcttttcaataactgtcc

SEQ ID NO: 890 - DNA, 18 bp, homo sapiens
tctggccaataaccgttc

SEQ ID NO: 891 - DNA, 20 bp, homo sapiens
tgaccctgatccatccagag

SEQ ID NO: 892 - DNA, 20 bp, homo sapiens
tgccaaagagccatcaagaa

SEQ ID NO: 893 - DNA, 20 bp, homo sapiens
tgtccaaggtcccacagttg

SEQ ID NO: 894 - DNA, 20 bp, homo sapiens
tcctcaagggcaatctagag

SEQ ID NO: 895 - DNA, 20 bp, homo sapiens
tgtccaaggcccctcagcag

SEQ ID NO: 896 - DNA, 18 bp, homo sapiens
tccagagactaactttgc

SEQ ID NO: 897 - DNA, 20 bp, homo sapiens
tgtccagagtccaagaaaaa

SEQ ID NO: 898 - DNA, 20 bp, homo sapiens
tatcaaaggtctctcaaaac

SEQ ID NO: 899 - DNA, 20 bp, homo sapiens
tgtccacattccctccagag

SEQ ID NO: 900 - DNA, 20 bp, homo sapiens
cttccaaggcccacagagag

SEQ ID NO: 901 - DNA, 18 bp, homo sapiens
tctctccaataactgtga

SEQ ID NO: 902 - DNA, 18 bp, homo sapiens
ggaaagttattggcgaga

SEQ ID NO: 903 - DNA, 20 bp, homo sapiens
ttattgattgatttttgaca

SEQ ID NO: 904 - DNA, 20 bp, homo sapiens
tttttcatggacactgggca

SEQ ID NO: 905 - DNA, 18 bp, homo sapiens
ataaagttattggggtga

SEQ ID NO: 906 - DNA, 18 bp, homo sapiens
ggaaaggcagtggtgaga

SEQ ID NO: 907 - DNA, 18 bp, homo sapiens
ggaaagttgtttgagagt

SEQ ID NO: 908 - DNA, 18 bp, homo sapiens
ggaaagttgtttgagagt

SEQ ID NO: 909 - DNA, 20 bp, homo sapiens
ctcttgattgagcttagaac

SEQ ID NO: 910 - DNA, 20 bp, homo sapiens
ctattgatggacattagact

SEQ ID NO: 911 - DNA, 20 bp, homo sapiens
ctcttaatgatctttggata

SEQ ID NO: 912 - DNA, 18 bp, homo sapiens
gacaagttagtggccaga

SEQ ID NO: 913 - DNA, 18 bp, homo sapiens
ttaaagttatgggaaaga

SEQ ID NO: 914 - DNA, 20 bp, homo sapiens
ctcttgatgcccattgtaga

SEQ ID NO: 915 - DNA, 20 bp, homo sapiens
ctcttgattgtcttgggtca

SEQ ID NO: 916 - DNA, 18 bp, homo sapiens
gggaagtaaaaggtgaga

SEQ ID NO: 917 - DNA, 20 bp, homo sapiens
cccttgattgacctaggaca

SEQ ID NO: 918 - DNA, 20 bp, homo sapiens
cacttgaaggatggtggaaa

SEQ ID NO: 919 - DNA, 20 bp, homo sapiens
ctcttgaattattttgggca

SEQ ID NO: 920 - DNA, 20 bp, homo sapiens
cccatgatggacccagccca

SEQ ID NO: 921 - DNA, 20 bp, homo sapiens
cccctgattgcctttggata

SEQ ID NO: 922 - DNA, 20 bp, homo sapiens
tgcttcatgtatcttggcca

SEQ ID NO: 923 - DNA, 22 bp, homo sapiens
tcctcctcttcagcagcaatgt

SEQ ID NO: 924 - DNA, 23 bp, homo sapiens
ccagattccaagagacatcataa

SEQ ID NO: 925 - DNA, 22 bp, homo sapiens
tcctcctcatcagtaataatgt

SEQ ID NO: 926 - DNA, 22 bp, homo sapiens
tcctcctcatcagtaataatgt

SEQ ID NO: 927 - DNA, 23 bp, homo sapiens
tccttttcctggaagctttctca

SEQ ID NO: 928 - DNA, 23 bp, homo sapiens
tcaaaagccaaaaaacaagcaaa

SEQ ID NO: 929 - DNA, 23 bp, homo sapiens
tgagataccattacacatcctag

SEQ ID NO: 930 - DNA, 22 bp, homo sapiens
tctgcctcctccccacccatat

SEQ ID NO: 931 - DNA, 23 bp, homo sapiens
ttaaaagccaggaagcatcctaa

SEQ ID NO: 932 - DNA, 23 bp, homo sapiens
acaattccacagaatcatccaaa

SEQ ID NO: 933 - DNA, 23 bp, homo sapiens
tcagctaccaagagaaattctaa

SEQ ID NO: 934 - DNA, 22 bp, homo sapiens
tctccctcatcagcagaaatga

SEQ ID NO: 935 - DNA, 22 bp, homo sapiens
ttttcatcttcatctgtgattt

SEQ ID NO: 936 - DNA, 22 bp, homo sapiens
tactcctctaaaaccacaatgg

SEQ ID NO: 937 - DNA, 23 bp, homo sapiens
tcatttaccaaacagaattataa

SEQ ID NO: 938 - DNA, 22 bp, homo sapiens
tcatcctcctcatcagtaataa

SEQ ID NO: 939 - DNA, 22 bp, homo sapiens
tccccatcattagcagcaatgc

SEQ ID NO: 940 - DNA, 22 bp, homo sapiens
acctccacttcagtaataatga

SEQ ID NO: 941 - DNA, 23 bp, homo sapiens
tcaaatgacataacacattctaa

SEQ ID NO: 942 - DNA, 23 bp, homo sapiens
ttaggatgcttcttggcaactga

SEQ ID NO: 943 - DNA, 22 bp, homo sapiens
acatggcagctgaagaggatgt

SEQ ID NO: 944 - DNA, 23 bp, homo sapiens
ttagaatgttcagttgcaattgt

SEQ ID NO: 945 - DNA, 22 bp, homo sapiens
acatggaggaggaggaggagga

SEQ ID NO: 946 - DNA, 23 bp, homo sapiens
tttggatgcttttgggaacctga

SEQ ID NO: 947 - DNA, 23 bp, homo sapiens
ttattatgctcatttgcaaatga

SEQ ID NO: 948 - DNA, 22 bp, homo sapiens
aaagtgctggtgaagatgtgga

SEQ ID NO: 949 - DNA, 23 bp, homo sapiens
ttaggctgcttcttggcaccttc

SEQ ID NO: 950 - DNA, 22 bp, homo sapiens
ttattgattatgaatgcgagga

SEQ ID NO: 951 - DNA, 22 bp, homo sapiens
ttattgattatgaatgcgagga

SEQ ID NO: 952 - DNA, 23 bp, homo sapiens
ttgggacatttatttgcacctgg

SEQ ID NO: 953 - DNA, 22 bp, homo sapiens
gcatgacagctgtagtggaggg

SEQ ID NO: 954 - DNA, 22 bp, homo sapiens
caattgctgctgaaggtgagga

SEQ ID NO: 955 - DNA, 23 bp, homo sapiens
acaggatggttctcagccactga

SEQ ID NO: 956 - DNA, 23 bp, homo sapiens
gtaagatgtttcctgatttctga

SEQ ID NO: 957 - DNA, 23 bp, homo sapiens
ttagaatgttcagttgcaattgt

SEQ ID NO: 958 - DNA, 23 bp, homo sapiens
ttagaatgttcagttgcaattgt

SEQ ID NO: 959 - DNA, 23 bp, homo sapiens
ttagaatgtgttatgtcatttga

SEQ ID NO: 960 - DNA, 22 bp, homo sapiens
tcattattactgaagtggaggt

SEQ ID NO: 961 - DNA, 23 bp, homo sapiens
tctgaggccaaaaagaaaaataa

SEQ ID NO: 962 - DNA, 23 bp, homo sapiens
acagttaccaaaaagcaaaataa

SEQ ID NO: 963 - DNA, 22 bp, homo sapiens
atgtgtctgttcaagaggagga

SEQ ID NO: 964 - DNA, 22 bp, homo sapiens
gcaagatggctgaataggaaga

SEQ ID NO: 965 - DNA, 18 bp, homo sapiens
tctccaacttccccataa

SEQ ID NO: 966 - DNA, 18 bp, homo sapiens
ttcagcttaacactggat

SEQ ID NO: 967 - DNA, 18 bp, homo sapiens
tgcagcatagatcaggga

SEQ ID NO: 968 - DNA, 18 bp, homo sapiens
ttcatcataaagctaaaa

SEQ ID NO: 969 - DNA, 18 bp, homo sapiens
ttcagaatgaaacaggaa

SEQ ID NO: 970 - DNA, 18 bp, homo sapiens
tttcctccttccccatac

SEQ ID NO: 971 - DNA, 18 bp, homo sapiens
tttcctccttccccatac

SEQ ID NO: 972 - DNA, 18 bp, homo sapiens
ttcagcttgaattaggaa

SEQ ID NO: 973 - DNA, 18 bp, homo sapiens
ttcagcataaataataaa

SEQ ID NO: 974 - DNA, 18 bp, homo sapiens
ttcaccatctatctgaaa

SEQ ID NO: 975 - DNA, 18 bp, homo sapiens
atcaacatgtaacagaaa

SEQ ID NO: 976 - DNA, 18 bp, homo sapiens
ttcaatatgattaagtat

SEQ ID NO: 977 - DNA, 18 bp, homo sapiens
tgcagcagtaaacatgaa

SEQ ID NO: 978 - DNA, 18 bp, homo sapiens
tgcagcataaattaagga

SEQ ID NO: 979 - DNA, 18 bp, homo sapiens
ttccacataaaaaaggac

SEQ ID NO: 980 - DNA, 18 bp, homo sapiens
ttaaaaatgaatcaaaac

SEQ ID NO: 981 - DNA, 18 bp, homo sapiens
tgcagcatgaattaggag

SEQ ID NO: 982 - DNA, 18 bp, homo sapiens
ttcaggagaaatcgagaa

SEQ ID NO: 983 - DNA, 18 bp, homo sapiens
tgcagcatagatcaggag

SEQ ID NO: 984 - DNA, 18 bp, homo sapiens
tgcagcatagatcaggag

SEQ ID NO: 985 - DNA, 18 bp, homo sapiens
atcagcaaaaaccaggga

SEQ ID NO: 986 - DNA, 18 bp, homo sapiens
ttcctgattcatgctgaa

SEQ ID NO: 987 - DNA, 18 bp, homo sapiens
ttcctgattcctgatgaa

SEQ ID NO: 988 - DNA, 18 bp, homo sapiens
tccctggtttctgctgaa

SEQ ID NO: 989 - DNA, 18 bp, homo sapiens
ttcttaattaatgctgaa

SEQ ID NO: 990 - DNA, 18 bp, homo sapiens
ttcctgagacaagatggg

SEQ ID NO: 991 - DNA, 18 bp, homo sapiens
gttctgattcatgatgaa

SEQ ID NO: 992 - DNA, 18 bp, homo sapiens
ttcctgaaacatcaacaa

SEQ ID NO: 993 - DNA, 18 bp, homo sapiens
cttgtgtttaatgatgaa

SEQ ID NO: 994 - DNA, 18 bp, homo sapiens
ttcttgtttaattctcaa

SEQ ID NO: 995 - DNA, 18 bp, homo sapiens
tttctgaggcatgttgaa

SEQ ID NO: 996 - DNA, 18 bp, homo sapiens
ttgatgatttatgctgaa

SEQ ID NO: 997 - DNA, 18 bp, homo sapiens
tttctaattcatgctaaa

SEQ ID NO: 998 - DNA, 18 bp, homo sapiens
atcctgggtcatgttgaa

SEQ ID NO: 999 - DNA, 18 bp, homo sapiens
ttcctgttgtaggctgaa

SEQ ID NO: 1000 - DNA, 18 bp, homo sapiens
tttcagatcaatgctgaa

SEQ ID NO: 1001 - DNA, 18 bp, homo sapiens
tccctggtttctgctgat

SEQ ID NO: 1002 - DNA, 18 bp, homo sapiens
ttcctgatatatgttgag

SEQ ID NO: 1003 - DNA, 18 bp, homo sapiens
tccctggtttttgctgat

SEQ ID NO: 1004 - DNA, 18 bp, homo sapiens
tccctggtttttgctgat

SEQ ID NO: 1005 - DNA, 18 bp, homo sapiens
ctcctgatctatgctgca

SEQ ID NO: 1006 - DNA, 20 bp, homo sapiens
tctccttgaatacaaaggac

SEQ ID NO: 1007 - DNA, 20 bp, homo sapiens
tgtccttaaaaacaaaggac

SEQ ID NO: 1008 - DNA, 20 bp, homo sapiens
tttccttggatacaaagaac

SEQ ID NO: 1009 - DNA, 20 bp, homo sapiens
tataagatataccctaatgg

SEQ ID NO: 1010 - DNA, 20 bp, homo sapiens
tctccctgcatacagagatc

SEQ ID NO: 1011 - DNA, 20 bp, homo sapiens
tctccaaaaataaaaaagaa

SEQ ID NO: 1012 - DNA, 20 bp, homo sapiens
tatcttcaaattcaaaggac

SEQ ID NO: 1013 - DNA, 20 bp, homo sapiens
tgtccttgcatgcaaaatac

SEQ ID NO: 1014 - DNA, 20 bp, homo sapiens
tcttattatttacaaagagc

SEQ ID NO: 1015 - DNA, 20 bp, homo sapiens
acatcttaaatacaaagaac

SEQ ID NO: 1016 - DNA, 20 bp, homo sapiens
tctccataaatacaaaggga

SEQ ID NO: 1017 - DNA, 20 bp, homo sapiens
gctctttgtgaacaaaggcc

SEQ ID NO: 1018 - DNA, 20 bp, homo sapiens
gctctttgtgaacaaaggcc

SEQ ID NO: 1019 - DNA, 20 bp, homo sapiens
gctctttgtgaacaaaggcc

SEQ ID NO: 1020 - DNA, 20 bp, homo sapiens
tgtaatattttcccccaagc

SEQ ID NO: 1021 - DNA, 20 bp, homo sapiens
tcaccatcagtgcaagagac

SEQ ID NO: 1022 - DNA, 20 bp, homo sapiens
tctcgtagaaagcaaagaaa

SEQ ID NO: 1023 - DNA, 20 bp, homo sapiens
tatcttatctcccctaatag

SEQ ID NO: 1024 - DNA, 20 bp, homo sapiens
tctcctggggaatgaaggtc

SEQ ID NO: 1025 - DNA, 21 bp, homo sapiens
tttcctagtatacaaaagatv

SEQ ID NO: 1026 - DNA, 20 bp, homo sapiens
catccttggatacagagggc

SEQ ID NO: 1027 - DNA, 20 bp, homo sapiens
ccgtgagggtagatgttata

SEQ ID NO: 1028 - DNA, 20 bp, homo sapiens
ctttgaggttacatgttaga

SEQ ID NO: 1029 - DNA, 20 bp, homo sapiens
aaaatttatatgcaaggagg

SEQ ID NO: 1030 - DNA, 20 bp, homo sapiens
ttcctgtgtcttcaaagaga

SEQ ID NO: 1031 - DNA, 20 bp, homo sapiens
gttctttgtataagaggagg

SEQ ID NO: 1032 - DNA, 20 bp, homo sapiens
gcctattgtattccaggaaa

SEQ ID NO: 1033 - DNA, 20 bp, homo sapiens
atcctttgtagtcaaggatg

SEQ ID NO: 1034 - DNA, 20 bp, homo sapiens
ctcttttgtttttttggaga

SEQ ID NO: 1035 - DNA, 20 bp, homo sapiens
gtctcttttattgaaggaga

SEQ ID NO: 1036 - DNA, 20 bp, homo sapiens
ggcatctgtaatcaagtgga

SEQ ID NO: 1037 - DNA, 20 bp, homo sapiens
cagagggggaaaattttaca

SEQ ID NO: 1038 - DNA, 20 bp, homo sapiens
ttcctttgtatttactgaga

SEQ ID NO: 1039 - DNA, 20 bp, homo sapiens
ttcctttgtatttactgaga

SEQ ID NO: 1040 - DNA, 20 bp, homo sapiens
ttcctttgtatttactgaga

SEQ ID NO: 1041 - DNA, 20 bp, homo sapiens
gtattttgtattcaatgtga

SEQ ID NO: 1042 - DNA, 20 bp, homo sapiens
ggctttggtattaaatgaga

SEQ ID NO: 1043 - DNA, 20 bp, homo sapiens
tttttctgtatttaaagaga

SEQ ID NO: 1044 - DNA, 20 bp, homo sapiens
gtcctttgtattcattgaaa

SEQ ID NO: 1045 - DNA, 20 bp, homo sapiens
gatacttgtattcaaggaga

SEQ ID NO: 1046 - DNA, 20 bp, homo sapiens
atcttttgtatactaggaaa

SEQ ID NO: 1047 - DNA, 20 bp, homo sapiens
gattttggtattcatggagt

SEQ ID NO: 1048 - DNA, 21 bp, homo sapiens
tctacagattctttgtagcag

SEQ ID NO: 1049 - DNA, 18 bp, homo sapiens
tctagtttcagcagtatt

SEQ ID NO: 1050 - DNA, 18 bp, homo sapiens
tccatcttcaggactcac

SEQ ID NO: 1051 - DNA, 21 bp, homo sapiens
tttacagatgctttatagcag

SEQ ID NO: 1052 - DNA, 21 bp, homo sapiens
cctacaaatcctttgtggcag

SEQ ID NO: 1053 - DNA, 18 bp, homo sapiens
ttcaccttcctgactcat

SEQ ID NO: 1054 - DNA, 18 bp, homo sapiens
tctcacttcaggacccag

SEQ ID NO: 1055 - DNA, 18 bp, homo sapiens
tccaacctcagaactcat

SEQ ID NO: 1056 - DNA, 18 bp, homo sapiens
tctaaaatcaggactcct

SEQ ID NO: 1057 - DNA, 18 bp, homo sapiens
tctcacttaaggacttac

SEQ ID NO: 1058 - DNA, 18 bp, homo sapiens
tctgtcttcagaagtcct

SEQ ID NO: 1059 - DNA, 18 bp, homo sapiens
tctaccttcagcactctg

SEQ ID NO: 1060 - DNA, 18 bp, homo sapiens
tctcgcatcaagacccat

SEQ ID NO: 1061 - DNA, 21 bp, homo sapiens
actacagctacttggaagcag

SEQ ID NO: 1062 - DNA, 18 bp, homo sapiens
tcaaactcctgacctcat

SEQ ID NO: 1063 - DNA, 18 bp, homo sapiens
tctctctccagtactcat

SEQ ID NO: 1064 - DNA, 18 bp, homo sapiens
tgtagcttctgtactact

SEQ ID NO: 1065 - DNA, 18 bp, homo sapiens
tcttgcctgaggactcat

SEQ ID NO: 1066 - DNA, 18 bp, homo sapiens
tcaagatccagaactcaa

SEQ ID NO: 1067 - DNA, 18 bp, homo sapiens
tatacatacagaacttat

SEQ ID NO: 1068 - DNA, 18 bp, homo sapiens
actagcttcattattcat

SEQ ID NO: 1069 - DNA, 18 bp, homo sapiens
atgagtcctgaagctaga

SEQ ID NO: 1070 - DNA, 18 bp, homo sapiens
atgagtcatgaagcttga

SEQ ID NO: 1071 - DNA, 18 bp, homo sapiens
aggagccctgaagtttgg

SEQ ID NO: 1072 - DNA, 21 bp, homo sapiens
ctggcaataaacatctgtaga

SEQ ID NO: 1073 - DNA, 18 bp, homo sapiens
atgggctctggagccaga

SEQ ID NO: 1074 - DNA, 18 bp, homo sapiens
atgagttctggggctaga

SEQ ID NO: 1075 - DNA, 18 bp, homo sapiens
ataagttttgaagcagga

SEQ ID NO: 1076 - DNA, 18 bp, homo sapiens
ctgagttctgaggttggg

SEQ ID NO: 1077 - DNA, 18 bp, homo sapiens
gtgattgttgaagccaga

SEQ ID NO: 1078 - DNA, 18 bp, homo sapiens
ttgagtccagaagtttga

SEQ ID NO: 1079 - DNA, 18 bp, homo sapiens
gtgagttctgaatctgga

SEQ ID NO: 1080 - DNA, 18 bp, homo sapiens
ttttgttctgaagccaga

SEQ ID NO: 1081 - DNA, 18 bp, homo sapiens
tggagttcagatgctaaa

SEQ ID NO: 1082 - DNA, 18 bp, homo sapiens
ttgagcccagaagtttga

SEQ ID NO: 1083 - DNA, 21 bp, homo sapiens
ttgtttcaaataatttgtata

SEQ ID NO: 1084 - DNA, 18 bp, homo sapiens
atgtgtgctgtgggtaga

SEQ ID NO: 1085 - DNA, 18 bp, homo sapiens
ataagtcttgaagtcaga

SEQ ID NO: 1086 - DNA, 18 bp, homo sapiens
tggggacttgaagttaga

SEQ ID NO: 1087 - DNA, 18 bp, homo sapiens
ataagtactgaagctatt

SEQ ID NO: 1088 - DNA, 18 bp, homo sapiens
atgagttctgaggttagg

SEQ ID NO: 1089 - DNA, 18 bp, homo sapiens
attagtcttgaagtatga

SEQ ID NO: 1090 - DNA, 21 bp, homo sapiens
tcctcaagctgcactccagct

SEQ ID NO: 1091 - DNA, 21 bp, homo sapiens
tccacaagctggactccaact

SEQ ID NO: 1092 - DNA, 19 bp, homo sapiens
tcacaaagaataaacaact

SEQ ID NO: 1093 - DNA, 19 bp, homo sapiens
tcccagtgaatataaaaat

SEQ ID NO: 1094 - DNA, 19 bp, homo sapiens
tcaaagtggaaatacaaca

SEQ ID NO: 1095 - DNA, 19 bp, homo sapiens
tcccagagaaaataccaag

SEQ ID NO: 1096 - DNA, 19 bp, homo sapiens
tcatagaaaataagaaact

SEQ ID NO: 1097 - DNA, 19 bp, homo sapiens
tcccagaggctataaacca

SEQ ID NO: 1098 - DNA, 21 bp, homo sapiens
tcctcaaactgctctccaaca

SEQ ID NO: 1099 - DNA, 19 bp, homo sapiens
ttacatagaatttacaata

SEQ ID NO: 1100 - DNA, 19 bp, homo sapiens
taatacagaatatacatag

SEQ ID NO: 1101 - DNA, 19 bp, homo sapiens
tcccaaagactgttctaag

SEQ ID NO: 1102 - DNA, 19 bp, homo sapiens
taaaagattatatacatag

SEQ ID NO: 1103 - DNA, 19 bp, homo sapiens
taaaagattatatacatag

SEQ ID NO: 1104 - DNA, 19 bp, homo sapiens
taaaagattatatacatag

SEQ ID NO: 1105 - DNA, 19 bp, homo sapiens
tcacaaagaataaacaaaa

SEQ ID NO: 1106 - DNA, 19 bp, homo sapiens
tcacaaagaataaacaaaa

SEQ ID NO: 1107 - DNA, 19 bp, homo sapiens
gcacagactataggcagcc

SEQ ID NO: 1108 - DNA, 21 bp, homo sapiens
tccccaacctttcctccttct

SEQ ID NO: 1109 - DNA, 19 bp, homo sapiens
tcaaatggagtaaacaact

SEQ ID NO: 1110 - DNA, 19 bp, homo sapiens
tcacagagcatatactcca

SEQ ID NO: 1111 - DNA, 19 bp, homo sapiens
cgttgtatattctctgtga

SEQ ID NO: 1112 - DNA, 19 bp, homo sapiens
atttgaacactttctgtga

SEQ ID NO: 1113 - DNA, 19 bp, homo sapiens
ctatgtatataatctttta

SEQ ID NO: 1114 - DNA, 19 bp, homo sapiens
tgttgtatatttaatgtga

SEQ ID NO: 1115 - DNA, 19 bp, homo sapiens
ctttgtatattttctttta

SEQ ID NO: 1116 - DNA, 19 bp, homo sapiens
cgttatctcttttttgtga

SEQ ID NO: 1117 - DNA, 19 bp, homo sapiens
tgttgtatattctgtgtca

SEQ ID NO: 1118 - DNA, 21 bp, homo sapiens
aactgttgtgaagcttgagga

SEQ ID NO: 1119 - DNA, 19 bp, homo sapiens
cttcctatttgttctttga

SEQ ID NO: 1120 - DNA, 19 bp, homo sapiens
cattgtaaattctatgtga

SEQ ID NO: 1121 - DNA, 19 bp, homo sapiens
tcttgtatatttcctgtga

SEQ ID NO: 1122 - DNA, 19 bp, homo sapiens
ggtgttatattctctgtga

SEQ ID NO: 1123 - DNA, 19 bp, homo sapiens
ttttgtttattctttgtga

SEQ ID NO: 1124 - DNA, 19 bp, homo sapiens
ttttgtttattctttgtga

SEQ ID NO: 1125 - DNA, 19 bp, homo sapiens
ttttgtttattctttgtga

SEQ ID NO: 1126 - DNA, 19 bp, homo sapiens
ctatgtatataatctttta

SEQ ID NO: 1127 - DNA, 19 bp, homo sapiens
ctatgtatataatctttta

SEQ ID NO: 1128 - DNA, 19 bp, homo sapiens
catggtatattctttgtga

SEQ ID NO: 1129 - DNA, 19 bp, homo sapiens
cgttgcttattctcagtga

SEQ ID NO: 1130 - DNA, 19 bp, homo sapiens
ctttgtacattttctgtgt

SEQ ID NO: 1131 - DNA, 19 bp, homo sapiens
tttagtatattcacagtca

SEQ ID NO: 1132 - DNA, 21 bp, homo sapiens
tgtgtcttcatagaccatttt

SEQ ID NO: 1133 - DNA, 17 bp, homo sapiens
tatcttcttctctggat

SEQ ID NO: 1134 - DNA, 21 bp, homo sapiens
tatctcttaatagcccatttt

SEQ ID NO: 1135 - DNA, 21 bp, homo sapiens
tgtgtctttatggaacaacta

SEQ ID NO: 1136 - DNA, 17 bp, homo sapiens
agttctcatccctgtat

SEQ ID NO: 1137 - DNA, 17 bp, homo sapiens
tttttacttctcagaat

SEQ ID NO: 1138 - DNA, 17 bp, homo sapiens
tgccttcttctctgaat

SEQ ID NO: 1139 - DNA, 21 bp, homo sapiens
tgagtcttcatagaacatttt

SEQ ID NO: 1140 - DNA, 17 bp, homo sapiens
tggcttctgctctgtgt

SEQ ID NO: 1141 - DNA, 17 bp, homo sapiens
actaaacttctctgaat

SEQ ID NO: 1142 - DNA, 17 bp, homo sapiens
tatattcttatatggag

SEQ ID NO: 1143 - DNA, 17 bp, homo sapiens
tgtcatcttctctgtat

SEQ ID NO: 1144 - DNA, 17 bp, homo sapiens
tgccttcttccctggat

SEQ ID NO: 1145 - DNA, 17 bp, homo sapiens
tgtcttcttctctgtct

SEQ ID NO: 1146 - DNA, 17 bp, homo sapiens
ggtcatcatctttggtt

SEQ ID NO: 1147 - DNA, 17 bp, homo sapiens
tgttttcttctctgcat

SEQ ID NO: 1148 - DNA, 17 bp, homo sapiens
tctcttcttctctgtat

SEQ ID NO: 1149 - DNA, 17 bp, homo sapiens
tctcttcttctctgtat

SEQ ID NO: 1150 - DNA, 17 bp, homo sapiens
tgccatcatctatgcct

SEQ ID NO: 1151 - DNA, 17 bp, homo sapiens
cttcttcttctctgaat

SEQ ID NO: 1152 - DNA, 17 bp, homo sapiens
tgtctcccactctgctg

SEQ ID NO: 1153 - DNA, 17 bp, homo sapiens
atccagagaagaagaca

SEQ ID NO: 1154 - DNA, 17 bp, homo sapiens
ctccatggaagaaaaaa

SEQ ID NO: 1155 - DNA, 17 bp, homo sapiens
atacagagaagaaaaca

SEQ ID NO: 1156 - DNA, 17 bp, homo sapiens
attcagagaataagaca

SEQ ID NO: 1157 - DNA, 17 bp, homo sapiens
ctccagagaagaagaca

SEQ ID NO: 1158 - DNA, 17 bp, homo sapiens
attcagagaagcagata

SEQ ID NO: 1159 - DNA, 17 bp, homo sapiens
ctctagaccaaaagtca

SEQ ID NO: 1160 - DNA, 17 bp, homo sapiens
agctggaagagaagacc

SEQ ID NO: 1161 - DNA, 17 bp, homo sapiens
agccagagatgaagtca

SEQ ID NO: 1162 - DNA, 17 bp, homo sapiens
agccagagatgaagaca

SEQ ID NO: 1163 - DNA, 21 bp, homo sapiens
aaggtggtttatgaagacaca

SEQ ID NO: 1164 - DNA, 17 bp, homo sapiens
tttaagagaaaaatact

SEQ ID NO: 1165 - DNA, 17 bp, homo sapiens
cgatagaggaggagaca

SEQ ID NO: 1166 - DNA, 17 bp, homo sapiens
atctagagaatgggagt

SEQ ID NO: 1167 - DNA, 17 bp, homo sapiens
agccagggaagaagaca

SEQ ID NO: 1168 - DNA, 21 bp, homo sapiens
taaataatctatgatgagata

SEQ ID NO: 1169 - DNA, 17 bp, homo sapiens
aggcatagatgatggca

SEQ ID NO: 1170 - DNA, 17 bp, homo sapiens
aggcatagatgatggca

SEQ ID NO: 1171 - DNA, 17 bp, homo sapiens
atacagagaagaagaga

SEQ ID NO: 1172 - DNA, 17 bp, homo sapiens
atatatagaagaatata

SEQ ID NO: 1173 - DNA, 17 bp, homo sapiens
atacagagaagaaggca

SEQ ID NO: 1174 - DNA, 24 bp, homo sapiens
tcatcccataatcccagagcctct

SEQ ID NO: 1175 - DNA, 24 bp, homo sapiens
tgaaaaaaaaaaaaaacactatta

SEQ ID NO: 1176 - DNA, 24 bp, homo sapiens
taaaaaaaaaaaaccagaataatt

SEQ ID NO: 1177 - DNA, 24 bp, homo sapiens
tcaaaaaaaaccaaaacacttatt

SEQ ID NO: 1178 - DNA, 24 bp, homo sapiens
tagaaaggagacaagggtctagtt

SEQ ID NO: 1179 - DNA, 24 bp, homo sapiens
tcaaaaaaaaaaaaaaaaaaaatt

SEQ ID NO: 1180 - DNA, 24 bp, homo sapiens
tcaaaaaaaaaaaaggcactatct

SEQ ID NO: 1181 - DNA, 24 bp, homo sapiens
tcagaaaataatatgacacttttg

SEQ ID NO: 1182 - DNA, 24 bp, homo sapiens
aaaaaaaaaaaaaaaagaatatat

SEQ ID NO: 1183 - DNA, 24 bp, homo sapiens
tcaaaaaaaaaaaagcccctgtcc

SEQ ID NO: 1184 - DNA, 24 bp, homo sapiens
aaaaaaaaaaaaaagaatatatat

SEQ ID NO: 1185 - DNA, 24 bp, homo sapiens
ttaaaagaaacagcgacactattt

SEQ ID NO: 1186 - DNA, 24 bp, homo sapiens
taacacagaatcacctcactatat

SEQ ID NO: 1187 - DNA, 24 bp, homo sapiens
ttaaaaaaaaaaaaaagcctattt

SEQ ID NO: 1188 - DNA, 24 bp, homo sapiens
tcaaaaaaaaaaaaaaaattattt

SEQ ID NO: 1189 - DNA, 24 bp, homo sapiens
aaaaaagaaaactggtgtcaattt

SEQ ID NO: 1190 - DNA, 24 bp, homo sapiens
taaaaaaaaaaaaattcactatgt

SEQ ID NO: 1191 - DNA, 24 bp, homo sapiens
tcaataaaaataaaaataaaattt

SEQ ID NO: 1192 - DNA, 24 bp, homo sapiens
taaaccaaaaactcaacaatcatt

SEQ ID NO: 1193 - DNA, 24 bp, homo sapiens
tcaaaaaaaaacaaaacaaagttt

SEQ ID NO: 1194 - DNA, 24 bp, homo sapiens
tcaaaagtaaaaagtaaactattt

SEQ ID NO: 1195 - DNA, 24 bp, homo sapiens
aaatagtgtcgtgttttcttttga

SEQ ID NO: 1196 - DNA, 24 bp, homo sapiens
aaatacctttttttttttttttga

SEQ ID NO: 1197 - DNA, 24 bp, homo sapiens
ttatagttttgtttctttttttga

SEQ ID NO: 1198 - DNA, 24 bp, homo sapiens
aattaattttatttatttttttga

SEQ ID NO: 1199 - DNA, 24 bp, homo sapiens
agaagctctgtgagtttgggatga

SEQ ID NO: 1200 - DNA, 24 bp, homo sapiens
acatattgtcttttttttttttaa

SEQ ID NO: 1201 - DNA, 24 bp, homo sapiens
agtaggttagggtttctgaaatga

SEQ ID NO: 1202 - DNA, 24 bp, homo sapiens
aaattttgtcatgtttgctttaga

SEQ ID NO: 1203 - DNA, 24 bp, homo sapiens
aattagtgttgtcttttcctgtga

SEQ ID NO: 1204 - DNA, 24 bp, homo sapiens
atatatttttttttttttttttga

SEQ ID NO: 1205 - DNA, 24 bp, homo sapiens
aattagtgttgtcttttcctgtga

SEQ ID NO: 1206 - DNA, 24 bp, homo sapiens
ataaaatagcattttctcttctga

SEQ ID NO: 1207 - DNA, 24 bp, homo sapiens
taatagttttttttttttttttga

SEQ ID NO: 1208 - DNA, 24 bp, homo sapiens
aaattgtgacatctttttttttaa

SEQ ID NO: 1209 - DNA, 24 bp, homo sapiens
tttttttgttttgttttgttttgt

SEQ ID NO: 1210 - DNA, 24 bp, homo sapiens
aaaaagtgtcgggttttttttttt

SEQ ID NO: 1211 - DNA, 24 bp, homo sapiens
aaatattgtggggtttttttgttg

SEQ ID NO: 1212 - DNA, 24 bp, homo sapiens
taatagtattttttttttttttga

SEQ ID NO: 1213 - DNA, 24 bp, homo sapiens
aaatatggttggtttgttttttga

SEQ ID NO: 1214 - DNA, 24 bp, homo sapiens
gaaaagtcttttcttttttattta

SEQ ID NO: 1215 - DNA, 24 bp, homo sapiens
taatagtgaggtaatttctttata

SEQ ID NO: 1216 - DNA, 20 bp, homo sapiens
tatggccccaggagtcccaa

SEQ ID NO: 1217 - DNA, 20 bp, homo sapiens
tatggccccagagatcccaa

SEQ ID NO: 1218 - DNA, 20 bp, homo sapiens
tatagcccccatggtcacca

SEQ ID NO: 1219 - DNA, 20 bp, homo sapiens
tcaatccttatgccaaggag

SEQ ID NO: 1220 - DNA, 20 bp, homo sapiens
tatgacccaaagaaaccaaa

SEQ ID NO: 1221 - DNA, 20 bp, homo sapiens
tgtgaccccaggagtcataa

SEQ ID NO: 1222 - DNA, 20 bp, homo sapiens
tcaaccctaattccttagag

SEQ ID NO: 1223 - DNA, 20 bp, homo sapiens
tctgaccctaagaatcacca

SEQ ID NO: 1224 - DNA, 20 bp, homo sapiens
tgtggccccaggggcccaac

SEQ ID NO: 1225 - DNA, 20 bp, homo sapiens
taagcccttataatgggtag

SEQ ID NO: 1226 - DNA, 20 bp, homo sapiens
tgggccccaaggagtcccac

SEQ ID NO: 1227 - DNA, 20 bp, homo sapiens
tgtgggcccaggagtcacga

SEQ ID NO: 1228 - DNA, 20 bp, homo sapiens
tgtggccccaggaagcccag

SEQ ID NO: 1229 - DNA, 20 bp, homo sapiens
taccaccccagcagtcacaa

SEQ ID NO: 1230 - DNA, 20 bp, homo sapiens
tctagctccataagtcccta

SEQ ID NO: 1231 - DNA, 20 bp, homo sapiens
tcagcacttatccagtggac

SEQ ID NO: 1232 - DNA, 20 bp, homo sapiens
catcacaccaggattcccaa

SEQ ID NO: 1233 - DNA, 20 bp, homo sapiens
ccaccccccctacaggggag

SEQ ID NO: 1234 - DNA, 20 bp, homo sapiens
tctggccccatggatcccaa

SEQ ID NO: 1235 - DNA, 20 bp, homo sapiens
tctgcccttttactgtggag

SEQ ID NO: 1236 - DNA, 20 bp, homo sapiens
tcagccattccaccgtggaa

SEQ ID NO: 1237 - DNA, 20 bp, homo sapiens
ctccacggtataagggctga

SEQ ID NO: 1238 - DNA, 20 bp, homo sapiens
tcccacggtcatacagctga

SEQ ID NO: 1239 - DNA, 20 bp, homo sapiens
cttcagggcataggggctga

SEQ ID NO: 1240 - DNA, 20 bp, homo sapiens
tctggtctcctgtggtcaca

SEQ ID NO: 1241 - DNA, 20 bp, homo sapiens
ttctatgttaaaagtgatga

SEQ ID NO: 1242 - DNA, 20 bp, homo sapiens
cttcaagggctatgggagga

SEQ ID NO: 1243 - DNA, 20 bp, homo sapiens
ctctaggggataaggcttca

SEQ ID NO: 1244 - DNA, 20 bp, homo sapiens
ttggggttcctggagtcatg

SEQ ID NO: 1245 - DNA, 20 bp, homo sapiens
aggggacttttgggggcgta

SEQ ID NO: 1246 - DNA, 20 bp, homo sapiens
ttccatggtatttggtatga

SEQ ID NO: 1247 - DNA, 20 bp, homo sapiens
ttgggaatcttggagcctaa

SEQ ID NO: 1248 - DNA, 20 bp, homo sapiens
ccccagggtatcagggtggc

SEQ ID NO: 1249 - DNA, 20 bp, homo sapiens
ttggggctctggccgacaga

SEQ ID NO: 1250 - DNA, 20 bp, homo sapiens
tggcagggaacaagggctga

SEQ ID NO: 1251 - DNA, 20 bp, homo sapiens
ttgggtctcttgggatctga

SEQ ID NO: 1252 - DNA, 20 bp, homo sapiens
ttgggacaccagagaacaca

SEQ ID NO: 1253 - DNA, 20 bp, homo sapiens
tggggtcccctggggtcagg

SEQ ID NO: 1254 - DNA, 20 bp, homo sapiens
tgggcactcctgggcccata

SEQ ID NO: 1255 - DNA, 20 bp, homo sapiens
cagagcctcctggggcacaa

SEQ ID NO: 1256 - DNA, 20 bp, homo sapiens
acgggacacctgatgtcaca

SEQ ID NO: 1257 - DNA, 20 bp, homo sapiens
acggctctccgggggccact

SEQ ID NO: 1258 - DNA, 18 bp, homo sapiens
tcagagaaataagcccag

SEQ ID NO: 1259 - DNA, 18 bp, homo sapiens
tctgtgcaataatctcag

SEQ ID NO: 1260 - DNA, 18 bp, homo sapiens
taaaaaaaaaaagcccag

SEQ ID NO: 1261 - DNA, 18 bp, homo sapiens
tccaagaaacaaacccaa

SEQ ID NO: 1262 - DNA, 22 bp, homo sapiens
tatgacatcaactaaagatgcg

SEQ ID NO: 1263 - DNA, 18 bp, homo sapiens
ccagacagagaaccccag

SEQ ID NO: 1264 - DNA, 18 bp, homo sapiens
tcaaaaaagaaagccaag

SEQ ID NO: 1265 - DNA, 18 bp, homo sapiens
tcagtgacataagcccag

SEQ ID NO: 1266 - DNA, 18 bp, homo sapiens
tcatagtaataaacagag

SEQ ID NO: 1267 - DNA, 18 bp, homo sapiens
gaaggggaataggcccaa

SEQ ID NO: 1268 - DNA, 18 bp, homo sapiens
tcatagaaatatgcccaa

SEQ ID NO: 1269 - DNA, 18 bp, homo sapiens
acatataaataagaacat

SEQ ID NO: 1270 - DNA, 18 bp, homo sapiens
tcagagaaatgagcccct

SEQ ID NO: 1271 - DNA, 18 bp, homo sapiens
ttggaaaaataatcccag

SEQ ID NO: 1272 - DNA, 18 bp, homo sapiens
tcagagacaatatcaaag

SEQ ID NO: 1273 - DNA, 18 bp, homo sapiens
tcagggcaatcagcccag

SEQ ID NO: 1274 - DNA, 18 bp, homo sapiens
tcaaatgaataagacaat

SEQ ID NO: 1275 - DNA, 18 bp, homo sapiens
tcaggaaaaaaatcccag

SEQ ID NO: 1276 - DNA, 18 bp, homo sapiens
tcatataaatgagctcat

SEQ ID NO: 1277 - DNA, 18 bp, homo sapiens
taaaattaatgagcccag

SEQ ID NO: 1278 - DNA, 18 bp, homo sapiens
agagagaaaaaggccgag

SEQ ID NO: 1279 - DNA, 22 bp, homo sapiens
ctcatctttagtgggtgccata

SEQ ID NO: 1280 - DNA, 18 bp, homo sapiens
ctgtgcttatttatttga

SEQ ID NO: 1281 - DNA, 18 bp, homo sapiens
ctgggctttcttctggga

SEQ ID NO: 1282 - DNA, 18 bp, homo sapiens
caggtgttacttctctga

SEQ ID NO: 1283 - DNA, 18 bp, homo sapiens
aggggcttaattcccaga

SEQ ID NO: 1284 - DNA, 18 bp, homo sapiens
ctgggtttattgcactga

SEQ ID NO: 1285 - DNA, 18 bp, homo sapiens
ctgtgcttatatctctca

SEQ ID NO: 1286 - DNA, 18 bp, homo sapiens
ctgtgcttgttaaaatga

SEQ ID NO: 1287 - DNA, 18 bp, homo sapiens
ttgtgtttatttctctaa

SEQ ID NO: 1288 - DNA, 18 bp, homo sapiens
ctggccttatttctctgt

SEQ ID NO: 1289 - DNA, 18 bp, homo sapiens
ctgagctcatgggtttga

SEQ ID NO: 1290 - DNA, 18 bp, homo sapiens
cttggcttattttactga

SEQ ID NO: 1291 - DNA, 18 bp, homo sapiens
tcggctttaatcctctga

SEQ ID NO: 1292 - DNA, 18 bp, homo sapiens
tagggcttatttgcttga

SEQ ID NO: 1293 - DNA, 18 bp, homo sapiens
gtgggtttatttgtctga

SEQ ID NO: 1294 - DNA, 18 bp, homo sapiens
ctggggtttcttgtctgg

SEQ ID NO: 1295 - DNA, 18 bp, homo sapiens
ttggttttgtttctctga

SEQ ID NO: 1296 - DNA, 18 bp, homo sapiens
cttgttttattctcctga

SEQ ID NO: 1297 - DNA, 18 bp, homo sapiens
cttggcttctttcactga

SEQ ID NO: 1298 - DNA, 18 bp, homo sapiens
tttggcttatttccatga

SEQ ID NO: 1299 - DNA, 18 bp, homo sapiens
ttgggtttatttttctaa

SEQ ID NO: 1300 - DNA, 17 bp, homo sapiens
tctttccatattttcag

SEQ ID NO: 1301 - DNA, 17 bp, homo sapiens
actttccatagtttcag

SEQ ID NO: 1302 - DNA, 17 bp, homo sapiens
tcttagaggacaccaaa

SEQ ID NO: 1303 - DNA, 17 bp, homo sapiens
tctatcctttttttcag

SEQ ID NO: 1304 - DNA, 17 bp, homo sapiens
tttttccatattttcag

SEQ ID NO: 1305 - DNA, 17 bp, homo sapiens
tctttccacattttcag

SEQ ID NO: 1306 - DNA, 17 bp, homo sapiens
tctttgaaaagaccaaa

SEQ ID NO: 1307 - DNA, 17 bp, homo sapiens
tcatttaatatttttgg

SEQ ID NO: 1308 - DNA, 17 bp, homo sapiens
tctttaatgataccaaa

SEQ ID NO: 1309 - DNA, 17 bp, homo sapiens
tcatttcatattttcag

SEQ ID NO: 1310 - DNA, 17 bp, homo sapiens
attttgataagatcaaa

SEQ ID NO: 1311 - DNA, 17 bp, homo sapiens
tatttgatttaatcaaa

SEQ ID NO: 1312 - DNA, 17 bp, homo sapiens
tctttccacattctctg

SEQ ID NO: 1313 - DNA, 17 bp, homo sapiens
ttttagaagaaaacaaa

SEQ ID NO: 1314 - DNA, 17 bp, homo sapiens
tctctccatattctcca

SEQ ID NO: 1315 - DNA, 17 bp, homo sapiens
tttttaataatcccaaa

SEQ ID NO: 1316 - DNA, 17 bp, homo sapiens
tcctgggtgagacccaa

SEQ ID NO: 1317 - DNA, 17 bp, homo sapiens
tatttgatgagaccaac

SEQ ID NO: 1318 - DNA, 17 bp, homo sapiens
tcatttaatattttcag

SEQ ID NO: 1319 - DNA, 17 bp, homo sapiens
tctttgatgaaaaccaa

SEQ ID NO: 1320 - DNA, 17 bp, homo sapiens
tctttccatattttaat

SEQ ID NO: 1321 - DNA, 17 bp, homo sapiens
tttggtctcatcaaaga

SEQ ID NO: 1322 - DNA, 17 bp, homo sapiens
ctgaaaatattgaatga

SEQ ID NO: 1323 - DNA, 17 bp, homo sapiens
tttggtgtcatctaaga

SEQ ID NO: 1324 - DNA, 17 bp, homo sapiens
ttgaaaatattgaaaga

SEQ ID NO: 1325 - DNA, 17 bp, homo sapiens
tcagttttcttcaaaga

SEQ ID NO: 1326 - DNA, 17 bp, homo sapiens
ctgaaaatattaaataa

SEQ ID NO: 1327 - DNA, 17 bp, homo sapiens
ctgacaacagggaaaaa

SEQ ID NO: 1328 - DNA, 17 bp, homo sapiens
ctgaaaatgtggaaaga

SEQ ID NO: 1329 - DNA, 17 bp, homo sapiens
ttaggtcttttcagaaa

SEQ ID NO: 1330 - DNA, 17 bp, homo sapiens
tttggtgtcatctaaga

SEQ ID NO: 1331 - DNA, 17 bp, homo sapiens
tttggtttcatgaaaga

SEQ ID NO: 1332 - DNA, 17 bp, homo sapiens
gttggtttcatccaaga

SEQ ID NO: 1333 - DNA, 17 bp, homo sapiens
tttggtgtcagcaaaga

SEQ ID NO: 1334 - DNA, 17 bp, homo sapiens
ctgaaaatacagaaaga

SEQ ID NO: 1335 - DNA, 17 bp, homo sapiens
ttggggctcattaaaga

SEQ ID NO: 1336 - DNA, 17 bp, homo sapiens
tctggtttcatcaagga

SEQ ID NO: 1337 - DNA, 17 bp, homo sapiens
atgagaatatagcaaga

SEQ ID NO: 1338 - DNA, 17 bp, homo sapiens
ctgaaattatggaaagc

SEQ ID NO: 1339 - DNA, 17 bp, homo sapiens
tttgttttcacaaatga

SEQ ID NO: 1340 - DNA, 17 bp, homo sapiens
tagaatatatgaataga

SEQ ID NO: 1341 - DNA, 19 bp, homo sapiens
tatactcctctttttttcg

SEQ ID NO: 1342 - DNA, 19 bp, homo sapiens
tctattcattactgtttac

SEQ ID NO: 1343 - DNA, 19 bp, homo sapiens
tttattcatttcattgaaa

SEQ ID NO: 1344 - DNA, 19 bp, homo sapiens
tctatacatttcagaacac

SEQ ID NO: 1345 - DNA, 19 bp, homo sapiens
aatactcctccttcttttt

SEQ ID NO: 1346 - DNA, 19 bp, homo sapiens
tataaacgtttatttttct

SEQ ID NO: 1347 - DNA, 19 bp, homo sapiens
tatacttctcttttgttca

SEQ ID NO: 1348 - DNA, 19 bp, homo sapiens
catactcctcttattttca

SEQ ID NO: 1349 - DNA, 19 bp, homo sapiens
tctatccatgtcaaagaac

SEQ ID NO: 1350 - DNA, 19 bp, homo sapiens
tctatccatttctgtgtac

SEQ ID NO: 1351 - DNA, 19 bp, homo sapiens
tatacttctatttttttat

SEQ ID NO: 1352 - DNA, 19 bp, homo sapiens
tctattactgacactgaac

SEQ ID NO: 1353 - DNA, 19 bp, homo sapiens
aatccccctcatttttctg

SEQ ID NO: 1354 - DNA, 19 bp, homo sapiens
taatcacctctttttctcc

SEQ ID NO: 1355 - DNA, 19 bp, homo sapiens
tctattaatatcagtaaac

SEQ ID NO: 1356 - DNA, 19 bp, homo sapiens
tcttcccatttctgtgaaa

SEQ ID NO: 1357 - DNA, 19 bp, homo sapiens
tctattaaaatcaatagac

SEQ ID NO: 1358 - DNA, 19 bp, homo sapiens
tctattaaaatcaatagac

SEQ ID NO: 1359 - DNA, 19 bp, homo sapiens
tctattaaaatcaatagac

SEQ ID NO: 1360 - DNA, 19 bp, homo sapiens
actatacatttcaatagtc

SEQ ID NO: 1361 - DNA, 19 bp, homo sapiens
ttaaattatttccgtggaa

SEQ ID NO: 1362 - DNA, 19 bp, homo sapiens
gtccactgaaatgaataga

SEQ ID NO: 1363 - DNA, 19 bp, homo sapiens
gtccattgaattgcataaa

SEQ ID NO: 1364 - DNA, 19 bp, homo sapiens
gtctatttaaataaagaga

SEQ ID NO: 1365 - DNA, 19 bp, homo sapiens
gcacactaaaaagaacaga

SEQ ID NO: 1366 - DNA, 19 bp, homo sapiens
atacactgaaatggataga

SEQ ID NO: 1367 - DNA, 19 bp, homo sapiens
gttaactgaaatgactaga

SEQ ID NO: 1368 - DNA, 19 bp, homo sapiens
tgaaaaaatgtgtactaga

SEQ ID NO: 1369 - DNA, 19 bp, homo sapiens
tgccactgaaatgactttt

SEQ ID NO: 1370 - DNA, 19 bp, homo sapiens
gtcttctcaaatgtacaga

SEQ ID NO: 1371 - DNA, 19 bp, homo sapiens
tgcaaataaaagtagtatt

SEQ ID NO: 1372 - DNA, 19 bp, homo sapiens
agaaaaagagagtagtaca

SEQ ID NO: 1373 - DNA, 19 bp, homo sapiens
gtctactgaagtgaactga

SEQ ID NO: 1374 - DNA, 19 bp, homo sapiens
ttccactgaaatgattata

SEQ ID NO: 1375 - DNA, 19 bp, homo sapiens
gtatattgaaatgtagaga

SEQ ID NO: 1376 - DNA, 19 bp, homo sapiens
ggccaatgaaacaaatggc

SEQ ID NO: 1377 - DNA, 19 bp, homo sapiens
gttaaatggaatgaataga

SEQ ID NO: 1378 - DNA, 19 bp, homo sapiens
gtttacttacattattaga

SEQ ID NO: 1379 - DNA, 19 bp, homo sapiens
gtttacttacattattaga

SEQ ID NO: 1380 - DNA, 19 bp, homo sapiens
gtttacttacattattaga

SEQ ID NO: 1381 - DNA, 19 bp, homo sapiens
ttccactgtaattagtata

SEQ ID NO: 1382 - DNA, 19 bp, homo sapiens
gtccacagatatgaatata

SEQ ID NO: 1383 - DNA, 21 bp, homo sapiens
tgctcgccaataaggcattcc

SEQ ID NO: 1384 - DNA, 17 bp, homo sapiens
tgtgaccatccaaggct

SEQ ID NO: 1385 - DNA, 17 bp, homo sapiens
tgttcccacccaaatct

SEQ ID NO: 1386 - DNA, 17 bp, homo sapiens
aattacaaacaaaagcc

SEQ ID NO: 1387 - DNA, 17 bp, homo sapiens
tgttactttgcaaatgc

SEQ ID NO: 1388 - DNA, 17 bp, homo sapiens
tgttaccagccaaacct

SEQ ID NO: 1389 - DNA, 17 bp, homo sapiens
tgtttcctttaaaatct

SEQ ID NO: 1390 - DNA, 17 bp, homo sapiens
tattgccttcatcagct

SEQ ID NO: 1391 - DNA, 17 bp, homo sapiens
tttcaccatccaaatct

SEQ ID NO: 1392 - DNA, 17 bp, homo sapiens
tactaccatacaaagct

SEQ ID NO: 1393 - DNA, 17 bp, homo sapiens
tattactgtctatatct

SEQ ID NO: 1394 - DNA, 17 bp, homo sapiens
tgtgaacatccaaggct

SEQ ID NO: 1395 - DNA, 17 bp, homo sapiens
tggtgcctaccacacct

SEQ ID NO: 1396 - DNA, 17 bp, homo sapiens
tgataacttccaagact

SEQ ID NO: 1397 - DNA, 17 bp, homo sapiens
tattaccatcaaaatca

SEQ ID NO: 1398 - DNA, 17 bp, homo sapiens
aattccaacccaaaggt

SEQ ID NO: 1399 - DNA, 17 bp, homo sapiens
ttttaccctccaaaccc

SEQ ID NO: 1400 - DNA, 17 bp, homo sapiens
tgttaaaatcctaatcc

SEQ ID NO: 1401 - DNA, 17 bp, homo sapiens
tgttcccacccaaatct

SEQ ID NO: 1402 - DNA, 17 bp, homo sapiens
ttttcccatttagatct

SEQ ID NO: 1403 - DNA, 17 bp, homo sapiens
tagaaccatccaaaact

SEQ ID NO: 1404 - DNA, 17 bp, homo sapiens
agctttggatggtaaca

SEQ ID NO: 1405 - DNA, 17 bp, homo sapiens
agcattggaggggaaca

SEQ ID NO: 1406 - DNA, 17 bp, homo sapiens
agatttgggtggggaca

SEQ ID NO: 1407 - DNA, 17 bp, homo sapiens
tgcttttgatggtaata

SEQ ID NO: 1408 - DNA, 17 bp, homo sapiens
aattttggatggtaaca

SEQ ID NO: 1409 - DNA, 17 bp, homo sapiens
agggatggagggttgca

SEQ ID NO: 1410 - DNA, 17 bp, homo sapiens
agctttgtctggtaaca

SEQ ID NO: 1411 - DNA, 17 bp, homo sapiens
agctttggaaggtatca

SEQ ID NO: 1412 - DNA, 17 bp, homo sapiens
attgttgggtggtaaga

SEQ ID NO: 1413 - DNA, 17 bp, homo sapiens
tggattggatgttcaca

SEQ ID NO: 1414 - DNA, 17 bp, homo sapiens
agctttgaatggtaaaa

SEQ ID NO: 1415 - DNA, 17 bp, homo sapiens
agctcttgatggtcact

SEQ ID NO: 1416 - DNA, 17 bp, homo sapiens
agctttggatggtcaga

SEQ ID NO: 1417 - DNA, 17 bp, homo sapiens
gtctttggaaggtaaca

SEQ ID NO: 1418 - DNA, 17 bp, homo sapiens
agcttttgtaggtaatg

SEQ ID NO: 1419 - DNA, 17 bp, homo sapiens
agccttggatggtaacc

SEQ ID NO: 1420 - DNA, 17 bp, homo sapiens
agctttggaaaataaca

SEQ ID NO: 1421 - DNA, 17 bp, homo sapiens
acctttggatggtaatt

SEQ ID NO: 1422 - DNA, 17 bp, homo sapiens
agagttggagggaagta

SEQ ID NO: 1423 - DNA, 17 bp, homo sapiens
atttttgtatggtaaca

SEQ ID NO: 1424 - DNA, 17 bp, homo sapiens
agatttgcatgtttaaa

SEQ ID NO: 1425 - DNA, 22 bp, homo sapiens
tgtcctgaagctgtaatctgaa

SEQ ID NO: 1426 - DNA, 21 bp, homo sapiens
tcccctaggactgacttcaga

SEQ ID NO: 1427 - DNA, 22 bp, homo sapiens
aatccagaagcagtaaccagta

SEQ ID NO: 1428 - DNA, 21 bp, homo sapiens
tccccagggaatgggatctgg

SEQ ID NO: 1429 - DNA, 21 bp, homo sapiens
tgccatgggatttgcttctgc

SEQ ID NO: 1430 - DNA, 21 bp, homo sapiens
tccactgaaatgaccttctgg

SEQ ID NO: 1431 - DNA, 21 bp, homo sapiens
tcccctgggacactcagcttt

SEQ ID NO: 1432 - DNA, 21 bp, homo sapiens
tcccttggcaattgcttctct

SEQ ID NO: 1433 - DNA, 21 bp, homo sapiens
ttccctgattatagctttctg

SEQ ID NO: 1434 - DNA, 21 bp, homo sapiens
tctccagagagaggcttctag

SEQ ID NO: 1435 - DNA, 21 bp, homo sapiens
tcctcagtcaatggcttctgg

SEQ ID NO: 1436 - DNA, 21 bp, homo sapiens
tgctcttgggatgtcttctgg

SEQ ID NO: 1437 - DNA, 21 bp, homo sapiens
taccctggccatggcctcagg

SEQ ID NO: 1438 - DNA, 21 bp, homo sapiens
tccattgaaaatagctcctga

SEQ ID NO: 1439 - DNA, 21 bp, homo sapiens
tcccttgtgaatggcttggtg

SEQ ID NO: 1440 - DNA, 22 bp, homo sapiens
tctcctgaagatgtaattagag

SEQ ID NO: 1441 - DNA, 21 bp, homo sapiens
tccacagagaatagtttatgc

SEQ ID NO: 1442 - DNA, 22 bp, homo sapiens
cgtccttacgctgtcatcagaa

SEQ ID NO: 1443 - DNA, 21 bp, homo sapiens
gcaccttgaaatgggcactgg

SEQ ID NO: 1444 - DNA, 21 bp, homo sapiens
tcccctgggaactgctgatgg

SEQ ID NO: 1445 - DNA, 21 bp, homo sapiens
tcacctgggaatagtatctag

SEQ ID NO: 1446 - DNA, 21 bp, homo sapiens
ccagaagccattcccagggga

SEQ ID NO: 1447 - DNA, 21 bp, homo sapiens
ccagactctattgccatgtgg

SEQ ID NO: 1448 - DNA, 21 bp, homo sapiens
cgtgaatcctttcccagggga

SEQ ID NO: 1449 - DNA, 21 bp, homo sapiens
ccagaagcagtcttcagggga

SEQ ID NO: 1450 - DNA, 21 bp, homo sapiens
cctgtagtcatgcccatggga

SEQ ID NO: 1451 - DNA, 21 bp, homo sapiens
ccagattccattccttgggga

SEQ ID NO: 1452 - DNA, 21 bp, homo sapiens
ccccattccattcacagggga

SEQ ID NO: 1453 - DNA, 21 bp, homo sapiens
ccagaagacatttcaaggaga

SEQ ID NO: 1454 - DNA, 21 bp, homo sapiens
cctgatgtccttcctttggga

SEQ ID NO: 1455 - DNA, 21 bp, homo sapiens
atggaaaccagtccaagggaa

SEQ ID NO: 1456 - DNA, 21 bp, homo sapiens
taagaatccattcctaggata

SEQ ID NO: 1457 - DNA, 21 bp, homo sapiens
agagaggccctcccctgggga

SEQ ID NO: 1458 - DNA, 21 bp, homo sapiens
gcaggagctattctcagacga

SEQ ID NO: 1459 - DNA, 21 bp, homo sapiens
accgtagtcattcccatgaga

SEQ ID NO: 1460 - DNA, 21 bp, homo sapiens
cctgaggtgatttctaggggg

SEQ ID NO: 1461 - DNA, 21 bp, homo sapiens
cttgtacccattccatgggga

SEQ ID NO: 1462 - DNA, 21 bp, homo sapiens
gcagaagctgtccattgggga

SEQ ID NO: 1463 - DNA, 21 bp, homo sapiens
ccggaagccattccaaatgga

SEQ ID NO: 1464 - DNA, 21 bp, homo sapiens
ccagaagggatggtaaaggga

SEQ ID NO: 1465 - DNA, 21 bp, homo sapiens
caagaagaaaacactagagga

SEQ ID NO: 1466 - DNA, 20 bp, homo sapiens
tgctccaggcattgattgat

SEQ ID NO: 1467 - DNA, 20 bp, homo sapiens
tggtcccacaggctggccag

SEQ ID NO: 1468 - DNA, 20 bp, homo sapiens
tactccaggcatagaaggag

SEQ ID NO: 1469 - DNA, 20 bp, homo sapiens
agagccccatatctcccaag

SEQ ID NO: 1470 - DNA, 20 bp, homo sapiens
tgcttcagacactgattgag

SEQ ID NO: 1471 - DNA, 20 bp, homo sapiens
tggtccccaaacctggccaa

SEQ ID NO: 1472 - DNA, 20 bp, homo sapiens
ggaggcccaaacgtggcctt

SEQ ID NO: 1473 - DNA, 20 bp, homo sapiens
tgctcattgcactggtggat

SEQ ID NO: 1474 - DNA, 20 bp, homo sapiens
tgtggcccatagctggccag

SEQ ID NO: 1475 - DNA, 20 bp, homo sapiens
tgcgccacaaagctggccac

SEQ ID NO: 1476 - DNA, 20 bp, homo sapiens
tgggccccaggcctgggcag

SEQ ID NO: 1477 - DNA, 20 bp, homo sapiens
taggcacctcagctggccaa

SEQ ID NO: 1478 - DNA, 20 bp, homo sapiens
tgagccactgagctggccag

SEQ ID NO: 1479 - DNA, 20 bp, homo sapiens
tggtctactgagctggcaag

SEQ ID NO: 1480 - DNA, 20 bp, homo sapiens
tggacccccaagatggccat

SEQ ID NO: 1481 - DNA, 20 bp, homo sapiens
tgctgcagagatttgtttat

SEQ ID NO: 1482 - DNA, 20 bp, homo sapiens
tggggccccagcctggccac

SEQ ID NO: 1483 - DNA, 20 bp, homo sapiens
tggtccccacagccagccag

SEQ ID NO: 1484 - DNA, 20 bp, homo sapiens
taagaaccaaagctaatcag

SEQ ID NO: 1485 - DNA, 20 bp, homo sapiens
tggggcccaaatgaagccag

SEQ ID NO: 1486 - DNA, 20 bp, homo sapiens
agcaccccaaacctggcctg

SEQ ID NO: 1487 - DNA, 20 bp, homo sapiens
ctggccagctttggggccca

SEQ ID NO: 1488 - DNA, 20 bp, homo sapiens
ctggacagctctgggcccca

SEQ ID NO: 1489 - DNA, 20 bp, homo sapiens
ttggaccactttggggccca

SEQ ID NO: 1490 - DNA, 20 bp, homo sapiens
atcagtcattgtctggagca

SEQ ID NO: 1491 - DNA, 20 bp, homo sapiens
attgccacatttggggccca

SEQ ID NO: 1492 - DNA, 20 bp, homo sapiens
ctggacagatgccagggcca

SEQ ID NO: 1493 - DNA, 20 bp, homo sapiens
ctagccagctctggggccca

SEQ ID NO: 1494 - DNA, 20 bp, homo sapiens
ctggcaagctttggggtctg

SEQ ID NO: 1495 - DNA, 20 bp, homo sapiens
ctggccagctatgggttttc

SEQ ID NO: 1496 - DNA, 20 bp, homo sapiens
agcaataaaaaccaggaaca

SEQ ID NO: 1497 - DNA, 20 bp, homo sapiens
ctgctcagctttctggctca

SEQ ID NO: 1498 - DNA, 20 bp, homo sapiens
ctgggcagcctgggagccct

SEQ ID NO: 1499 - DNA, 20 bp, homo sapiens
cttattccttgtctggagaa

SEQ ID NO: 1500 - DNA, 20 bp, homo sapiens
ttgtgcagctttggggcccg

SEQ ID NO: 1501 - DNA, 20 bp, homo sapiens
cagaacagctttggagctag

SEQ ID NO: 1502 - DNA, 20 bp, homo sapiens
ttggccagatttgggggcct

SEQ ID NO: 1503 - DNA, 20 bp, homo sapiens
ttgggtaccttgggggccca

SEQ ID NO: 1504 - DNA, 20 bp, homo sapiens
ctgcccaggctgggagtgca

SEQ ID NO: 1505 - DNA, 20 bp, homo sapiens
ttcttcagttttgtggccca

SEQ ID NO: 1506 - DNA, 20 bp, homo sapiens
cctgccagccttggggtcct

SEQ ID NO: 1507 - DNA, 20 bp, homo sapiens
ttgggcagcatttggcccca

SEQ ID NO: 1508 - DNA, 22 bp, homo sapiens
tctgccacttcttcccatcaag

SEQ ID NO: 1509 - DNA, 19 bp, homo sapiens
tcaacatctgtcagacgat

SEQ ID NO: 1510 - DNA, 19 bp, homo sapiens
tctacatctaacattttat

SEQ ID NO: 1511 - DNA, 19 bp, homo sapiens
tgcacatctctcactttaa

SEQ ID NO: 1512 - DNA, 19 bp, homo sapiens
tgtgcttatctaagtacat

SEQ ID NO: 1513 - DNA, 19 bp, homo sapiens
tttacatctatcagtttat

SEQ ID NO: 1514 - DNA, 19 bp, homo sapiens
tcttcgtctctcagcttat

SEQ ID NO: 1515 - DNA, 19 bp, homo sapiens
gctccatctcaaaaataat

SEQ ID NO: 1516 - DNA, 19 bp, homo sapiens
tatacatttctcattttat

SEQ ID NO: 1517 - DNA, 22 bp, homo sapiens
ttggccacttcttcccatcccg

SEQ ID NO: 1518 - DNA, 19 bp, homo sapiens
acgatatcactatgtttac

SEQ ID NO: 1519 - DNA, 19 bp, homo sapiens
tctagatctaactgacaat

SEQ ID NO: 1520 - DNA, 19 bp, homo sapiens
tctagatctctcaggttaa

SEQ ID NO: 1521 - DNA, 19 bp, homo sapiens
tctagttctctcagtttat

SEQ ID NO: 1522 - DNA, 19 bp, homo sapiens
agtacatcttttaatttat

SEQ ID NO: 1523 - DNA, 19 bp, homo sapiens
ccatcctcactaagtttaa

SEQ ID NO: 1524 - DNA, 19 bp, homo sapiens
tctccatctggcaattgag

SEQ ID NO: 1525 - DNA, 19 bp, homo sapiens
tatacatttctcagttgat

SEQ ID NO: 1526 - DNA, 19 bp, homo sapiens
catacatctctcactttat

SEQ ID NO: 1527 - DNA, 19 bp, homo sapiens
tctccatgtctcagtttgt

SEQ ID NO: 1528 - DNA, 19 bp, homo sapiens
tctacaaatgtaaaattct

SEQ ID NO: 1529 - DNA, 19 bp, homo sapiens
ataaactgagagatgtaga

SEQ ID NO: 1530 - DNA, 19 bp, homo sapiens
ataaaatgagagttgtagc

SEQ ID NO: 1531 - DNA, 19 bp, homo sapiens
ataaatggaaaactggaga

SEQ ID NO: 1532 - DNA, 19 bp, homo sapiens
aaaagctgagagaggttga

SEQ ID NO: 1533 - DNA, 19 bp, homo sapiens
gcaaattgagagatgtaga

SEQ ID NO: 1534 - DNA, 19 bp, homo sapiens
aaaaactgagctacagagg

SEQ ID NO: 1535 - DNA, 22 bp, homo sapiens
cttgggtggaggaagtggcttc

SEQ ID NO: 1536 - DNA, 19 bp, homo sapiens
ataaaatgatagatgcaga

SEQ ID NO: 1537 - DNA, 19 bp, homo sapiens
tttagtcgtgagatggaga

SEQ ID NO: 1538 - DNA, 19 bp, homo sapiens
ttaacctgaaacatggaga

SEQ ID NO: 1539 - DNA, 19 bp, homo sapiens
ataatctgagagttgtata

SEQ ID NO: 1540 - DNA, 19 bp, homo sapiens
ataaactgggaggcgtaga

SEQ ID NO: 1541 - DNA, 19 bp, homo sapiens
caactctgtgagatgaaga

SEQ ID NO: 1542 - DNA, 19 bp, homo sapiens
atagacttagtgctgatgt

SEQ ID NO: 1543 - DNA, 22 bp, homo sapiens
cctgatgggaagaattagaaga

SEQ ID NO: 1544 - DNA, 19 bp, homo sapiens
ttaaagtgagagatgtata

SEQ ID NO: 1545 - DNA, 19 bp, homo sapiens
ctaaactggaagatgtaga

SEQ ID NO: 1546 - DNA, 22 bp, homo sapiens
cttgttctgacgatgctgcaga

SEQ ID NO: 1547 - DNA, 19 bp, homo sapiens
ttaaacttagtgaggaagg

SEQ ID NO: 1548 - DNA, 19 bp, homo sapiens
atagactaagtgacttata

SEQ ID NO: 1549 - DNA, 19 bp, homo sapiens
aaaaactgagagattttga

SEQ ID NO: 1550 - DNA, 2477 bp, artificial sequence
  synthetic polynucleotide
ttaaggatctcagtctaataaggaaagcagaaaagcaaagcaaccttataatatggtgcaataatttgctataatgaagttatatacaaagtgaagtagaagcatagaagaagcagcactaaatttgtctgggtgagtcagagaaggctaaccaggaaaaatagtttctgaactaacacttgaaggaggtgtagcagttcatcactgacagtgatgttggggtgggtctggtttcaggagaggggaggaaattggctttggtctgaggctgaggtgtgggcaaagcattagcttatgtgggtccattagcttatgtgagtccacaaaaggtgtgtgtgtgtttgtgtgtatgtgtgtgtgtgtgtgtgtgtgtgtgtgtgtacgaaatgggggctcaatgatttggtagtggtttggtttgtcaagaagcaggctgggaactcaataagcatctttccattcatttctactgtgtatcccacagcttcacacacacatgcacatttcaacattggtgactgcttcacttgcacacctaaggtaatgatggacacacctgtagcaatgtagattcttcctaagctaataattagtttcaggaggtagcacatacatttaaaaataggttaaaataaagtgttattttaattggtaggtggatctgttggcaccaatgattattcacggcatcaagacccagggtgcccgtcagaagttctccagtctctatatctctcagtttatcatcatgtatagtctcgacggcaagaagtggcagacgtaccgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagtatgtaattagtcatttaaagggaatgcctgaatactttaaagaattttggcagatttcagatattggacaaacactcttagcttccacaaacttaattccaaaaaataatttttcacttatgagcaatagagttattacggacatatcagcaaaaatgtagtagtgtcaaggctcatagatgatagaaatgaagagatgctgtattgatagaaatatgtgattcaggactgtgtggattgatgattgtgagcttgcttatggatatcctaggtttgaggttatagtaggacaatcaggttgaaatgtccagcaggcagtaggtgaaagacaagtttagggggcaaaaccatggatggagatgaagattcatgacttccacataaaaggatgggtgaaactttgggaattgatgaattctctagaggtgagctcaagacccttaaaggcttaaaacctcagcgttattgtctactcttccctcatttttatgcccacaaatctggtcaatcctttatttgcaatgcctctcacatctctttcttctgtttccatttataccgctgttgccacagcccagggtcccatcacctcacacttgatctattgtattacattcctaactagtcttcccccgtttctaatctgttctccgataaaagctgcacatcattttcaggataatcatcagtcgcctgcctaaaacttttcaatgtcttcccattgtctttagaataaagttcaaagtcttcaaatgaccccaagcaagataacttttgtttgcccctttagatccatttt

SEQ ID NO: 1551 - DNA, 2677 bp, artificial sequence
  synthetic polynucleotide
gccctttacagaaaaagtttgccaacctatgttgttgtgaggtaaaaaaaaatcctcttgaaaaggaggcgtgagagttttacaccaaaatagtaacatttttcactaggtggaagggttacattttaaaatgtcttttatttgtatttttactaatttttacttttcattttctgatttttctacaatgaacatacattgcgtaataaataataggcggggcacgttggctcatgcctcccagcactttgcaaggctgaggcaagcagatcacctgaggtcaggagttcaagaccagcctggccaacatggtgaaactccgtctctactaaaaatacaaaaattagtcgggcatggtggtacgcgattgtagtcccagctacctaggagactgaggcaggagaattgcttgaactcaggaggtggaggttgcagtgagccaagatcatgccattgcactccagcctgggtgacaaagcaagactccatctcaaaaaaagaaagaaaagaagaaataatattattatttggtagtgttggtaacaaattgcagtatcagctagttagaggtgctaacaattaacaaaattataaattttagaaaataaaatggacaacaaggataagcaatatccttagatagtaattgatactggtatgccataaagcctttatgtttttctctattttcaccacagcttagattaacctttctcaagacaataattttattctcaagtgtctaggactaacccagctgaatttaatctctgtttctttacttgggcaaaggacagtgggccccaaagctggccagacttcactactctggatcaatcaatgcatggtctaccaaggagcccttttcttggatcaaggtgtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatagaaaatgtaatcaatgatgggaaatgtatcacattcaatcaattgcattacttattcctcttgcaagctcaaaggattctatgaatatgagaaaactaaagaacagaatgccttaatgatttgtacaaaagcagtcatgaacaaagagatatggggatagaattgagtatattgatatgtcctgtttctgtattttagtccttctactgggattagaacatctgaatattttctataatattgaactcgtcatctctcaagacagtatatgttattattagatgcttccaactgcccacgtgtccttaagtactccaatcccctttattttaacataaaacaaatggttcacaaatgcaaaccacatgtgtacttttacattttctgtagccacgttttcaaaaatgtgaaattcactttaataatacattttatttaactcaacatatctgaaaatactatcatttcaacatatgatcaatgaggccccttcaaagacagacagatggaaactcttgggtctcttccatgcctcacaaaagctgagggcagcttggaagtgcctgctcagcctctccacctaaacataaggctagatgccttctagaagcccaaacaggaaatggagaaaacattttggtttccatctttgcaaatagcatgtctattaatgccacagcattgttttgtagacactgccaattttgactcaatctgagctgctgttcactaatccctaagtattttttgttggtttgtgcttctgccaaacaa

SEQ ID NO: 1552 - DNA, 8751 bp, artificial sequence
  synthetic polynucleotide
ctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagtaaaggcatgtcctgtagggtctgatcggggccaggattgtggggatgtaagtctgcttggaggaaggtgcagacatcgggttaggatggttgtgatgctacctgggccccaaagaaacatttctgggtaaggtgtgcacacatctgtgttattagcagaaatgctaactgccaattcttttcataggtctgacctatttgttgatattttgttctgttttgtccattgcttctcttcgtcatatgctgctcctccagaatctagagactggagtagagggagggtgaagggacaaagacaaaacttccctctgcctgcccaagcttccatagagagaatcaaggcaatgaaatccaatcaatatcacacacaagtttcatgtctggttctcttgtgtgtacatgcaatgtgtgtttttataatatcttttcctactttgggtgtaaggataatatgagccttgagttcagaagcttttcgtgttttgggggttctggtgcatttaggcagagtattaaataactttatcaatattgtctatggtcatcagttgattcagatttttctacctcttcttcagtaaatattggtatattttggtctatactttcatagaaagcaatctactgtccctagatttgataatgtattggtatcaagttatgtaagagtctcctgtgattttgttaaactgttctgtgtctgtagttatattttctttttcattccttatgttgtatatgttctcttcctctcttttaaaaataatatttccaggagttttcttgattttattgg

SEQ ID NO: 1553 - DNA, 6090 bp, artificial sequence
  synthetic polynucleotide
ctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagtaaaggcatgtcctgtagggtctgatcggggccaggattgtggggatgtaagtctgcttggaggaaggtgcagacatcgggttaggatggttgtgatgctacctgggccccaaagaaacatttctgggtaaggtgtgcacacatctgtgttattagcagaaatgctaactgccaattcttttcataggtctgacctatttgttgatattttgttctgttttgtccattgcttctcttcgtcatatgctgctcctccagaatctagagactggagtagagggagggtgaagggacaaagacaaaacttccctctgcctgcccaagcttccatagagagaatcaaggcaatgaaatccaatcaatatcacacacaagtttcatgtctggttctcttgtgtgtacatgcaatgtgtgtttttataatatcttttcctactttgggtgtaaggataatatgagccttgagttcagaagcttttcgtgttttgggggttctggtgcatttaggcagagtattaaataactttatcaatattgtctatggtcatcagttgattcagatttttctacctcttcttcagtaaatattggtatattttggtctatactttcatagaaagcaatctactgtccctagatttgataatgtattggtatcaagttatgtaagagtctcctgtgattttgttaaactgttctgtgtctgtagttatattttctttttcattccttatgttgtatatgttctcttcctctcttttaaaaataatatttccaggagttttcttgattttattgg

SEQ ID NO: 1554 - DNA, 23 bp, homo sapiens
cactaaagcagaatcgcaaaagg

SEQ ID NO: 1555 - DNA, 23 bp, homo sapiens
aagatactacctgggtgcagtgg

SEQ ID NO: 1556 - DNA, 23 bp, homo sapiens
agtctttttgtacacgactgagg

SEQ ID NO: 1557 - DNA, 23 bp, homo sapiens
ttttcaacatcgctaagccaagg

SEQ ID NO: 1558 - DNA, 23 bp, homo sapiens
cagcatgaagactgacaggatgg

SEQ ID NO: 1559 - DNA, 23 bp, homo sapiens
atgctgttggtgtatcctactgg

SEQ ID NO: 1560 - DNA, 23 bp, homo sapiens
atgctgttggtgtatcctactgg

SEQ ID NO: 1561 - DNA, 23 bp, homo sapiens
tatgagtaggtaaggcacagtgg

SEQ ID NO: 1562 - DNA, 23 bp, homo sapiens
gacttgaattcaggcctcattgg

SEQ ID NO: 1563 - DNA, 23 bp, homo sapiens
aagtagtataaatttgtgcaagg

SEQ ID NO: 1564 - DNA, 23 bp, homo sapiens
ctttttgctgtatttgatgaagg

SEQ ID NO: 1565 - DNA, 23 bp, homo sapiens
gactgtgtgcattttaggccagg

SEQ ID NO: 1566 - DNA, 23 bp, homo sapiens
cagtcaatggttatgtaaacagg

SEQ ID NO: 1567 - DNA, 23 bp, homo sapiens
gcgagatttccaaggacgcctgg

SEQ ID NO: 1568 - DNA, 23 bp, homo sapiens
caaacactcttgatggaccttgg

SEQ ID NO: 1569 - DNA, 23 bp, homo sapiens
tcttggcaactgagcgaatttgg

SEQ ID NO: 1570 - DNA, 23 bp, homo sapiens
acattacattgctgctgaagagg

SEQ ID NO: 1571 - DNA, 23 bp, homo sapiens
aatagcttcacgagtcttaaagg

SEQ ID NO: 1572 - DNA, 23 bp, homo sapiens
gaagctattcagcatgaatcagg

SEQ ID NO: 1573 - DNA, 23 bp, homo sapiens
ggacatcagtgattccgtgaggg

SEQ ID NO: 1574 - DNA, 23 bp, homo sapiens
atgtccgtcctttgtattcaagg

SEQ ID NO: 1575 - DNA, 23 bp, homo sapiens
aacgaaactagagtaatagcggg

SEQ ID NO: 1576 - DNA, 23 bp, homo sapiens
gatctagcttcaggactcattgg

SEQ ID NO: 1577 - DNA, 23 bp, homo sapiens
agcgttgtatattctctgtgagg

SEQ ID NO: 1578 - DNA, 23 bp, homo sapiens
cgctttctccccaatccagctgg

SEQ ID NO: 1579 - DNA, 23 bp, homo sapiens
atagaccattttgtgtttgaagg

SEQ ID NO: 1580 - DNA, 23 bp, homo sapiens
agaaactgtcttcatgtcgatgg

SEQ ID NO: 1581 - DNA, 23 bp, homo sapiens
ttttcttttgaaagctgcggggg

SEQ ID NO: 1582 - DNA, 23 bp, homo sapiens
acactattttattgctgcagtgg

SEQ ID NO: 1583 - DNA, 23 bp, homo sapiens
acggtataagggctgagtaaagg

SEQ ID NO: 1584 - DNA, 23 bp, homo sapiens
aaatgaacatttgggactcctgg

SEQ ID NO: 1585 - DNA, 23 bp, homo sapiens
cagtcaaactcatctttagtggg

SEQ ID NO: 1586 - DNA, 23 bp, homo sapiens
atgagtttgactgcaaagcctgg

SEQ ID NO: 1587 - DNA, 23 bp, homo sapiens
ttcagtgaagtaccagcttttgg

SEQ ID NO: 1588 - DNA, 23 bp, homo sapiens
ggctccctgcaatatccagatgg

SEQ ID NO: 1589 - DNA, 23 bp, homo sapiens
gtccactgaaatgaatagaatgg

SEQ ID NO: 1590 - DNA, 23 bp, homo sapiens
gttcactgtacgaaaaaaagagg

SEQ ID NO: 1591 - DNA, 23 bp, homo sapiens
cgccaaattccagctttggatgg

SEQ ID NO: 1592 - DNA, 23 bp, homo sapiens
attggcgagcatctacatgctgg

SEQ ID NO: 1593 - DNA, 23 bp, homo sapiens
tgtccagaagccattcccagggg

SEQ ID NO: 1594 - DNA, 23 bp, homo sapiens
gattttcagattacagcttcagg

SEQ ID NO: 1595 - DNA, 23 bp, homo sapiens
tgatccggaataatgaagtctgg

SEQ ID NO: 1596 - DNA, 23 bp, homo sapiens
aatcaatgcctggagcaccaagg

SEQ ID NO: 1597 - DNA, 23 bp, homo sapiens
agataaactgagagatgtagagg

SEQ ID NO: 1598 - DNA, 23 bp, homo sapiens
aagaagtggcagacttatcgagg

SEQ ID NO: 1599 - DNA, 22 bp, homo sapiens
gcacccaggtagtatcttctgg

SEQ ID NO: 1600 - DNA, 22 bp, homo sapiens
actatatgcaaagtgatctcgg

SEQ ID NO: 1601 - DNA, 22 bp, homo sapiens
acatgagaaagatatgagtagg

SEQ ID NO: 1602 - DNA, 22 bp, homo sapiens
acttgaattcaggcctcattgg

SEQ ID NO: 1603 - DNA, 22 bp, homo sapiens
aaggtctgtgtcttttccttgg

SEQ ID NO: 1604 - DNA, 22 bp, homo sapiens
tttttgctgtatttgatgaagg

SEQ ID NO: 1605 - DNA, 22 bp, homo sapiens
ttttccctgatgagagagaagg

SEQ ID NO: 1606 - DNA, 22 bp, homo sapiens
acaaagaactccttgatgcagg

SEQ ID NO: 1607 - DNA, 22 bp, homo sapiens
gttattggcgagatttccaagg

SEQ ID NO: 1608 - DNA, 22 bp, homo sapiens
aaacactcttgatggaccttgg

SEQ ID NO: 1609 - DNA, 22 bp, homo sapiens
atagcttcacgagtcttaaagg

SEQ ID NO: 1610 - DNA, 22 bp, homo sapiens
tcttgggacctttactttatgg

SEQ ID NO: 1611 - DNA, 22 bp, homo sapiens
acgaaactagagtaatagcggg

SEQ ID NO: 1612 - DNA, 22 bp, homo sapiens
atctagcttcaggactcattgg

SEQ ID NO: 1613 - DNA, 22 bp, homo sapiens
tgttttcttttgaaagctgcgg

SEQ ID NO: 1614 - DNA, 22 bp, homo sapiens
gctgcagtggagaggctctggg

SEQ ID NO: 1615 - DNA, 22 bp, homo sapiens
agtcaaactcatctttagtggg

SEQ ID NO: 1616 - DNA, 22 bp, homo sapiens
tatttctctgatgttgacctgg

SEQ ID NO: 1617 - DNA, 22 bp, homo sapiens
cttttggtctcatcaaagatgg

SEQ ID NO: 1618 - DNA, 22 bp, homo sapiens
aatatggaaagaaactgcaggg

SEQ ID NO: 1619 - DNA, 22 bp, homo sapiens
gccaaattccagctttggatgg

SEQ ID NO: 1620 - DNA, 22 bp, homo sapiens
ttggcgagcatctacatgctgg

SEQ ID NO: 1621 - DNA, 22 bp, homo sapiens
tgtccagaagccattcccaggg

SEQ ID NO: 1622 - DNA, 22 bp, homo sapiens
ttacagcttcaggacaatatgg

SEQ ID NO: 1623 - DNA, 22 bp, homo sapiens
gatccggaataatgaagtctgg

SEQ ID NO: 1624 - DNA, 22 bp, homo sapiens
caccaaggagcccttttcttgg

SEQ ID NO: 1625 - DNA, 22 bp, homo sapiens
aggctggagaacttctgacggg

SEQ ID NO: 1626 - DNA, 22 bp, homo sapiens
tcatcatgtatagtcttgatgg

SEQ ID NO: 1627 - DNA, 24 bp, artificial sequence
  synthetic polynucleotide
ggtaatgatggacacacctgtagc

SEQ ID NO: 1628 - DNA, 22 bp, artificial sequence
  synthetic polynucleotide
ggttttgccccctaaacttgtc

SEQ ID NO: 1629 - DNA, 24 bp, artificial sequence
  synthetic polynucleotide
gttaggtgactcaaatgggttcac

SEQ ID NO: 1630 - DNA, 24 bp, artificial sequence
  synthetic polynucleotide
gaacaagaagcagggtagagaagc

SEQ ID NO: 1631 - DNA, 24 bp, artificial sequence
  synthetic polynucleotide
gaacaagaagcagggtagagaagc

SEQ ID NO: 1632 - DNA, 23 bp, artificial sequence
  synthetic polynucleotide
aagatggccatcagtggactctc

SEQ ID NO: 1633 - DNA, 64 bp, homo sapiens
atgacatgtgttagtattatagtttctcagattatcaccagtgatactatgggatgagttgcag

SEQ ID NO: 1634 - DNA, 64 bp, homo sapiens
tactgtacaccatcatattatcaaagagtctaatagtggtcactatgataccctactcaacgtc

SEQ ID NO: 1635 - DNA, 66 bp, canis familiaris
cagagataggtgacgaggactccatggatggatgacattcgtttgacgtgatggcagggcaatcaa

SEQ ID NO: 1636 - DNA, 66 bp, canis familiaris
gtctctatccactgctcctgaggtacctacctactgtaagcaaactgcactaccgtcccgttagtt

SEQ ID NO: 1637 - DNA, 23 bp, homo sapiens
caaattccactccaaccttaatc

SEQ ID NO: 1638 - DNA, 46 bp, homo sapiens
gtatgtaattactcatttaaacccaatccctcaatactttaaacaa

SEQ ID NO: 1639 - DNA, 69 bp, homo sapiens
caaattccactccaaccttaatcgtatgtaattactcatttaaacccaatccctcaatactttaaacaa

SEQ ID NO: 1640 - DNA, 23 bp, homo sapiens
ctttaaggtgaccttggaattac

SEQ ID NO: 1641 - DNA, 46 bp, homo sapiens
catacattaatcagtaaatttcccttacggacttatgaaatttctt

SEQ ID NO: 1642 - DNA, 69 bp, homo sapiens
ctttaaggtgaccttggaattaccatacattaatcagtaaatttcccttacggacttatgaaatttctt

SEQ ID NO: 1643 - DNA, 66 bp, homo sapiens
gaaattccactggaaccttaatggtatgtaattagtcatttaaagggaatgcctgaatactttaaa

SEQ ID NO: 1644 - DNA, 65 bp, homo sapiens
ctttaagtgaccttggaattaccatacattaatcagtaaatttcccttacggacttatgaaattt

SEQ ID NO: 1645 - DNA, 69 bp, homo sapiens
tcaccagtgatactatgggatgagttgcagatggcaagtaagacactggggagattaaatgacagtggc

SEQ ID NO: 1646 - DNA, 69 bp, homo sapiens
agtggtcactatgataccctactcaacgtctaccgttcattctgtgacccctctaatttactgtcaccg

SEQ ID NO: 1647 - DNA, 66 bp, homo sapiens
tcaccagtgatactatgggatgagttgcagatggcaagtaagacactggggagattaaatgacagt

SEQ ID NO: 1648 - DNA, 66 bp, homo sapiens
agtggtcactatgataccctactcaacgtctaccgttcattctgtgacccctctaatttactgtca

SEQ ID NO: 1649 - DNA, 20 bp, artificial sequence
  synthetic polynucleotide
catgtcctgtagggtctgat

SEQ ID NO: 1650 - DNA, 42 bp, homo sapiens
tccatttccgtacaggacatcccagactagccccggtcctaa

SEQ ID NO: 1651 - DNA, 42 bp, homo sapiens
aggtaaaggcatgtcctgtagggtctgatcggggccaggatt

SEQ ID NO: 1652 - DNA, 20 bp, artificial sequence
  synthetic polynucleotide
gatcaatgcctggagcacca

SEQ ID NO: 1653 - DNA, 73 bp, homo sapiens
accggtctgaagtaataaggcctagttagttacggacctcgtgggtcctcgggaaaagaacctagttccaatc

SEQ ID NO: 1654 - DNA, 73 bp, homo sapiens
tggccagacttcattattccggatcaatcaatgcctggagcaccaaggagcccttttcttggatcaaggttag

SEQ ID NO: 1655 - DNA, 20 bp, artificial sequence
  synthetic polynucleotide
ctgaagtaataaggcctagg

SEQ ID NO: 1656 - DNA, 18 bp, homo sapiens
tttttgattcatgtagga

SEQ ID NO: 1657 - DNA, 17 bp, homo sapiens
ctgaaattgtggaaagt

SEQ ID NO: 1658 - DNA, 21 bp, homo sapiens
acaggggtctctcccagtggt

SEQ ID NO: 1659 - DNA, 23 bp, homo sapiens
aaaatactacctcggtgcagtgg